(19) United States
(12) Patent Application Publication
(10) Pub. No.: US 2026/0279490 A1
(43) Pub. Date: Sep. 17, 2026

(54) METHODS OF IDENTIFYING GENETICALLY-ANCHORED MOLECULAR SIGNATURES FOR DISEASE AND TREATMENTS THEREOF

(71) Applicant: THE GENERAL HOSPITAL CORPORATION, Boston, MA (US)

(72) Inventors: Melina CLAUSSNITZER, Boston, MA (US); Miriam UDLER-AUBREY, Boston, MA (US)

(73) Assignee: THE GENERAL HOSPITAL CORPORATION, Boston, MA (US)

(21) Appl. No.: 18/869,123

(22) PCT Filed: May 26, 2023
(86) PCT No.: PCT/US2023/067527

§ 371 Date: Nov. 25, 2024

Related U.S. Application Data
(60) Provisional application No. 63/345,985, filed on May 26, 2022.

(51) Int. Cl. G16B 20/50; G16H 50/30

(52) U.S. Cl. CPC G16B 20/50; G16H 50/30

(57) ABSTRACT

The present invention provides a method for obtaining genetically-anchored molecular signatures that are molecular signatures linked to genetic risk variants and that define mechanistic disease pathways in order to facilitate improved disease sub-classification, develop of novel rational therapeutics, and implement targeted prevention practices. The signatures can be obtained using polygenic risk scores and gene expression data, such as partitioned polygenic risk scores. In certain embodiments, the method further comprises identifying one or more key regulatory features of the genetically-anchored molecular signature by matching the genetically-anchored molecular signature with one or more perturbation molecular signatures from a perturbation data set using similarity scoring

[symbol]

### CROSS-REFERENCE TO RELATED APPLICATIONS

This application claims the benefit of U.S. Provisional Application No. 63/345,985, filed May 26, 2022. The entire contents of the above-identified application are hereby fully incorporated herein by reference.

### REFERENCE TO AN ELECTRONIC SEQUENCE LISTING

The contents of the electronic sequence listing (“BROD-5505WP_ST26.xml”; Size is 17,964 bytes and it was created on May 24, 2023) is herein incorporated by reference in its entirety.

### TECHNICAL FIELD

The subject matter disclosed herein is generally directed to genetically-anchored molecular signatures that are molecular signatures linked to genetic risk variants.

### BACKGROUND

Many diseases, such as type 2 diabetes (T2D), are highly polygenic and influenced by multiple biological pathways. Current treatments, such as for type 2 diabetes (T2D), are rarely curative and could be revolutionized by targeting underlying causal mechanisms. For example, existing management for T2D focuses largely on addressing hyperglycemia, the final symptomatic manifestation of disease, rather than modifying its underlying pathogenesis. Additional strategies are needed to define mechanistic disease pathways in order to facilitate improved disease sub-classification, develop of novel rational therapeutics, and implement targeted prevention practices, such as, for the rapidly rising incidence of T2D worldwide (IDF Diabetes Atlas, 10th edn. Brussels, Belgium: International Diabetes Federation; 2021).

Citation or identification of any document in this application is not an admission that such a document is available as prior art to the present invention.

### SUMMARY

In one aspect, the present invention provides for a method for obtaining a genetically-anchored molecular signature for a disease comprising identifying, for a set of endotypes for the disease, a genetically anchored molecular signature correlated with each endotype by a regression/correlation analysis of a molecular data set, the molecular data set comprising at least endotype data type and one or more molecular profiles. In certain embodiments, the method further comprises identifying one or more key regulatory features of the genetically-anchored molecular signature by matching the genetically-anchored molecular signature with one or more perturbation molecular signatures from a perturbation data set using similarity scoring. In certain embodiments, the method further comprises conducting a therapeutic agent library screen to identify one or more therapeutic agents that reverse the genetically-anchored molecular signature.

In certain embodiments, the endotype is associated with a distinct mechanistic pathway of the disease. In certain embodiments, the endotype is defined by a polygenic risk score, thereby linking the polygenic risk score for that endotype to molecular data. In certain embodiments, the polygenic risk score is enriched for variants linked to DNA regulatory elements active in a tissue associated with the disease. In certain embodiments, the polygenic risk score is a partitioned polygenic risk score (pPS). In certain embodiments, the pPS risk score is obtained by clustering variants based on patterns of genome-wide associations across disease related traits for the disease. In certain embodiments, the clustering is a soft clustering analysis. In certain embodiments, the soft clustering analysis is a Bayesian non-negative factorization (bNMF) clustering analysis.

In certain embodiments, the one or more molecular profiles used to derive the genetically-anchored molecular signature comprise a transcriptomic profile, a proteomic profile, a metabolomic profile, a cell-imaging based profile, a spatial transcriptomic profile, a spatial proteomics profile, a spatial metabolomics profile, an epigenomic profile, a clinical imaging profile, or a combination thereof. In certain embodiments, the epigenomic profile comprises a chromatin accessibility profile, a chromatin modification profile, a chromatin binding profile, a DNA methylation profile, a DNase hypersensitivity profile, or a DNA-DNA contact profile.

In another aspect, the present invention provides for a method for identifying therapeutic agents for treating disease comprising: conducting a therapeutic agent library screen and generating a molecular signature for each therapeutic agent; selecting one or more therapeutic agents with a molecular signature that positively or negatively correlates with a genetically-anchored molecular signature for a disease. In certain embodiments, the selected one or more therapeutic agents have a molecular signature that negatively correlates with the genetically anchored molecular signature for the disease. In certain embodiments, the genetically-anchored molecular signature is derived from a regression/correlation analysis of a molecular data set, the molecular data set comprising at least endotype data and one or more molecular profiles.

In certain embodiments, the endotype data is associated with distinct mechanistic pathways of a disease. In certain embodiments, the endotype is defined by a polygenic risk score. In certain embodiments, the polygenic risk score is enriched for variants linked to DNA regulatory elements active in a tissue associated with the disease. In certain embodiments, the polygenic risk score is a pPS risk score. In certain embodiments, the pPS risk score is obtained by clustering variants based on patterns of genome-wide associations across disease related traits for the disease. In certain embodiments, the clustering is a soft clustering analysis. In certain embodiments, the soft clustering analysis is a Bayesian non-negative factorization (bNMF) clustering analysis.

In certain embodiments, the one or more molecular profiles comprise a transcriptomic profile, a proteomic profile, a metabolomic profile, a cell-imaging based profile, a spatial transcriptomic profile, a spatial proteomics profile, a spatial metabolomics profile, an epigenomic profile, a clinical imaging profile, or a combination thereof. In certain embodiments, the epigenomic profile comprises a chromatin accessibility profile, a chromatin modification profile, a chromatin binding profile, a DNA methylation profile, a DNase hypersensitivity profile, or a DNA-DNA contact profile.

In another aspect, the present invention provides for a method of treating a disease comprising: determining a genetically-anchored molecular signature for a subject in need of treatment; and administering one or more therapeutic agents capable of reversing the genetically-anchored molecular signature, wherein the one or more therapeutic agents are identified using the method of any embodiment herein. In certain embodiments, the disease is type 2 diabetes (T2D). In certain embodiments, the genetically-anchored molecular signature is correlated to a genetic risk variant cluster of T2D and one or more gene regulatory pathways associated with the genetic risk variant cluster. In certain embodiments, the genetic risk variant clusters comprise a Beta-cell 1, a Beta-cell 2, a proinsulin, a lipoprotein A, an obesity, a lipodystrophy, a liver/lipid, a hyper insulin secretion, an ALP negative, and a SHBG cluster.

In another aspect, the present invention provides for a method of monitoring and/or modifying a treatment comprising: (a) determining a genetically-anchored molecular signature for a subject in need of treatment wherein the genetically-anchored molecular signature is correlated to a disease type or disease sub-type; (b) administering an initial therapy capable of reversing the genetically-anchored molecular signature; (c) determining if there has been a change in the genetically-anchored molecular signature and continuing to administer the initial therapy if the genetically-anchored molecular signature has reversed. In certain embodiments, the disease is type 2 diabetes (T2D). In certain embodiments, the genetically-anchored molecular signature identifies a genetic risk variant cluster of T2D and one or more gene regulatory pathways associated with the genetic risk variant cluster. In certain embodiments, the genetic risk variant clusters comprise a Beta-cell 1, a Beta-cell 2, a proinsulin, a lipoprotein A, an obesity, a lipodystrophy, a liver/lipid, a hyper insulin secretion, an ALP negative, and a SHBG cluster.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Cluster associations with metabolic traits using GWAS. Standardized effect sizes with 95% confidence intervals of cluster pPS-trait associations derived from GWAS summary statistics shown in forest plot. Three metabolic traits (Fasting Insulin, Fasting proinsulin adjusted for fasting insulin, Disposition Index) that discriminate clusters are displayed. The numbers in the parenthesis next to cluster names indicate the number of variants included in the analysis in each cluster. “All SNPs” include all the variants that are top-weighted in at least one cluster. Filled points indicate P-values less than 0.05.

FIG. 2—Clusters of Type 2 Diabetes Loci. Top-weighted loci and traits in each of the ten clusters are represented in circular plots. Green bars represent top-weighted loci, red bars represent increased traits, and blue bars represent reduced traits in each cluster. A maximum of 35 elements (loci and traits) based on highest weights are displayed in each cluster. Blue outline indicates clusters associated with decreased fasting insulin levels, and red outline indicates clusters associated with increased fasting insulin levels.

FIG. 3A-3C—Enrichment for tissue-specific enhancers in T2D clusters. (FIG. 3A) Heatmap of tissue enhancer/promoter enrichment analysis result. (FIG. 3B) Heatmap of pancreatic islet cell enrichment analysis result. Significance was indicated as follows: *** FDR<0.001, ** FDR<0.01, * FDR<0.1, • P<0.05. (FIG. 3C) Comparison of Beta-cell 1 and Beta-cell 2 clusters in fgwas enrichment analysis in functional and stressed beta-cell states shown in a forest plot.

FIG. 4A-4B—Forest plot of cluster associations with outcomes using (A) GWAS and (B) individual-level data from MGB Biobank. (FIG. 4A) Standardized effect sizes with 95% confidence intervals of cluster pPS-outcome associations derived from GWAS summary statistics shown in forest plot. Three metabolic outcomes (T2D, CAD and CKD, all T2D unadjusted) are displayed. The numbers in the parenthesis next to cluster names indicate the number of variants included in the analysis in each cluster. Filled points indicate P-values less than 0.05. (FIG. 4B) Associations of pPSs in individuals in the MGB Biobank with clinical outcomes are shown in forest plot. Three outcomes including T2D are displayed.

FIG. 5A-5B—Overview of variant preprocessing pipeline. (FIG. 5A) Flowchart of the steps implemented in the variant preprocessing pipeline. Steps include: 1) extract variants from multiple GWAS datasets of an outcome of interest, 2) remove variants that do not pass a Bonferroni p-value from the largest GWAS dataset of the outcome, 3) find proxy variants for variants that are multi-allelic, ambiguous, or have low trait counts, 4) LD-prune to ensure independent signals and 5) align variants to risk increasing alleles of the outcome of interest. (FIG. 5B) Flowchart of trait preprocessing. Steps include: 1) filter trait GWAS by minimum sample size, 2) filter trait GWAS by a minimum Bonferroni p-value across the selected variants, 3) filter by correlation between traits and 4) generate a variant by trait association matrix.

FIG. 6—Method for determining cluster weight cutoff. The method of determining cluster weight cutoff shown in plot. First, the weights were aggregated from all the clusters and plotted in a descending order (x axis: scaled count, y axis: scaled cluster weight). Then a line (blue) was fitted to the top 1% of the weights and another line (red) to the bottom 80% of the weights. The point where the distance to the first line (blue) becomes shorter than the second line (red) is determined to be the cutoff for cluster weights.

FIG. 7—Cluster-defining characteristics. Standardized effect sizes of cluster pPS-trait association derived from GWAS summary statistics shown in bar plots. The black vertical line (y-axis) shows an estimate of 0, representing no association between the cluster pPS and trait. A subset of discriminatory traits are displayed. Bars pointing towards the right side represent positive cluster-trait associations, whereas those pointing towards the left side represent negative cluster-trait associations. Asterisks represent significant cluster-trait associations below a Bonferroni P-value.

FIG. 8A-8B—Forest plots of associations with clinical traits using a) GWAS and b) Individual-level data from MGB Biobank. (FIG. 8A) Standardized effect sizes with 95% confidence intervals of cluster pPS-trait associations derived from GWAS summary statistics shown in forest plot. Three glycemic traits (triglycerides, HbA1c, BMI) are displayed. “All SNPs” include all the variants that are top-weighted in at least one cluster. (FIG. 8B) Associations of pPSs in individuals adjusted for T2D status in the MGB Biobank with glycemic traits included in the clustering analysis are shown in forest plot. “GRS total” is the sum of pPS using betas from T2D GWAS (DIAMANTE) of all top-weighted variants in ten clusters. The numbers in the parenthesis next to cluster names indicate the number of variants included in the analysis in each cluster. Hollow points indicate the P-value is equal to or greater than 0.05, and otherwise filled.

FIG. 9A-9B—Clusters of a) CAD loci and b) CKD loci. (FIG. 9A) Top-weighted loci and traits in each of the five CAD clusters are represented in circular plots. Green bars represent top-weighted loci, red bars represent increased traits and blue bars represent reduced traits in each cluster. (FIG. 9B) Top-weighted loci and traits in each of the four CKD clusters are represented in circular plots.

FIG. 10—Clustering variant-trait associations using Bayesian non-negative matrix factorization (bNMF).

FIG. 11A-11C—Lipocyte cellular profiles visible using T2D/Lipodystrophy pPS. Comparison of lipocyte features in (FIG. 11A) extremes of T2D polygenic score and (FIG. 11B) with increased Lipodystrophy pPS. (FIG. 11C). View of lipocytes features in low and high Lipodystophy pPS. Blue=DNA, green=lipid, red=mitochondria, and yellow=arabinogalactan-proteins (AGP).

FIG. 12—Conceptual model for obtaining and using genetically-anchored molecular signatures.

FIG. 13—A combined rare and common variant score improved HbA1C-based T2D diagnosis compared to a common variant score. The odds ratio represents the accuracy of each risk score (see text). The size of the diamonds represents the number of reclassified T2D cases.

FIG. 14—Preliminary T2D rare variant polygenic score. True positive rate (% T2D patients in the UKB classified as positive by the score) is plotted vs positive predictive value (fraction of individuals classified as positive who have T2D).

FIG. 15—Enhanced Lipodystrophy pPS has larger associations with clinical phenotypes than original cluster in MGBB. PLOS refers to original Lipodystrophy pPS in Udler et al., PLoS Medicine, 2018.

FIG. 16—Gene set enrichment results for lipodystrophy pPS expression profile show enrichment for relevant processes.

FIG. 17—Top enriched gene sets (GO Biological Process 2021) for genes up- and down-regulated in relation to increased “Lipodystrophy” pPS in GTEx subcutaneous adipose tissue. Highly significant mitochondrial enrichment results are consistent with cellular image-based results for the “Lipodystrophy” pPS.

FIG. 18—The “Lipodystrophy” pPS down-regulated genes show much greater gene set enrichment than the full T2D global extended polygenic score (GO Biological Process 2021). Results are consistent with cellular image-based results.

FIG. 19—Gene set enrichment (GO Biological Process 2021) performed for Lipodystrophy pPS expression signatures generated using multiple GTEx tissues.

FIG. 20A-20G—Discovery of chemical modulators of lipodystrophy PRS signatures. (FIG. 20A) Shows that subjects having lipodystrophy have heterogenous polygenic risk scores (PRS). (FIG. 20B) Shows that cells can be treated with 300 compounds and cell imaging profiles for the treated cells can be obtained. (FIG. 20C) Shows a diagram for determining a connectivity score between the compounds and PRS signatures. (FIG. 20D) Shows lipodystrophy PRS signatures for genes and morphological signatures (features). (FIG. 20E) Shows a diagram of a cellular network of features for identifying key driver features and drugs that can cure symptoms caused by the driver. (FIG. 20F) Shows connectivity scores calculated between the top and bottom PRS scores and the compounds. (FIG. 20G) Shows a graph of a negatively connected drug, a null drug and a positively connected drug corresponding to FIG. 20F.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to Kim H, Westerman K E, Smith K, Chiou J, Cole J B, Majarian T, von Grotthuss M, Kwak S H, Kim J, Mercader J M, Florez J C, Gaulton K, Manning A K, Udler M S. High-throughput genetic clustering of type 2 diabetes loci reveals heterogeneous mechanistic pathways of metabolic disease. Diabetologia. 2023 March; 66(3):495-507.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide for genetically-anchored molecular signatures and uses thereof. Genetically-anchored molecular signatures are obtained by linking genetic subtypes of diseases to molecular signatures. The genetic subtypes of a disease can be defined by an endotype. As used herein the term “endotype” refers to a subtype of a health condition, which is defined by a distinct functional or pathobiological mechanism and is distinct from phenotype, which is any observable characteristic or trait of a disease without any implication of mechanism. For example, the resulting disease symptoms can be the same for a group of subjects having the disease, however, an endotype can indicate the underlying mechanism that led to the disease in different subjects. Thus, treatment for subjects having a different endotype can be tailored to the underlying mechanism and not the just the resulting symptoms of disease.

The genetic subtypes of disease (e.g., endotype) can be determined by genetic polymorphisms, such as monogenic variants or polygenic variants (e.g., a polygenic risk score (PS) or partitioned polygenic risk score (pPS), described further herein). In one aspect, the disease of the present invention is a highly polygenic disease that is influenced by multiple biological pathways, for example, Type 2 diabetes (T2D). As used herein, “polygenic disease” refers to a genetic disorder that is caused by the combined action of more than one gene. Most disease risk is polygenic in nature and the diseases applicable to the present invention include all diseases that are polygenic. Non-limiting examples of polygenic conditions include inflammatory bowel disease (IBD), autoimmune diseases, cancers, obesity, atherosclerosis, hypertension, coronary heart disease, and diabetes. Because such disorders depend on the simultaneous presence of several genes, they are not inherited as simply as are single-gene diseases. In example embodiments, the endotype is determined by a genotype and the presence of polygenic variants in the genotype.

In example embodiments, the endotype can be further separated (e.g., by a score) based on the strength or weakness of the endotype. For example, a subject can have a high polygenic risk score or a low polygenic risk score. As used herein, “polygenic risk score” refers to an assessment of the risk of a specific condition based on the collective influence of many genetic variants or a score based on the number of variants related to the disease a subject has. Variants can include variants associated with genes of known function and variants not known to be associated with genes relevant to the condition. In another example, the strength of the endotype depends on the presence or quantity of markers indicative of the distinct functional or pathobiological mechanism. The markers can be a metabolic panel, such as from a blood panel, cell morphological features, such as from cell painting, or a combination of markers, such as a combination including markers and genetic variants. In example embodiments, morphological and cellular features identify pathways and a score for different samples based on the combination of different features. A non-limiting example for type 2 diabetes is measuring corrected insulin response, which is an estimate of pancreatic beta cell function that is calculated using data from an oral glucose tolerance test (this endotype is captured by a “beta cell” pPS). Another non-limiting example for type 2 diabetes are measures of HDL cholesterol, triglycerides, and fasting insulin as well as imaging modalities capturing a greater proportion of visceral vs subcutaneous fat (this endotype is captured by the lipodystrophy pPS).

The molecular signatures may be obtained from any molecular profile that includes differentially observable molecular characteristics (e.g., differential gene expression, proteomic, metabolic, spatial, epigenetic, image-based profiling of morphology and cellular markers, or lipodomics signatures). In example embodiments, the endotype and molecular signature for individual subjects is present in a data set.

In example embodiments, the linking of endotypes to a molecular signature can be performed by a correlation analysis between endotypes and the molecular signatures (described further herein). As used herein “correlation” refers to observable molecular characteristics that increase or decrease with a specific condition (e.g., an endotype, such as a polygenic risk score). As used herein “correlation analysis” refers to a statistical method used to measure the strength of the linear relationship between two variables and compute their association. In other words, correlation analysis calculates the level of change in one variable due to the change in the other. Negative or inverse correlation describes when two variables tend to move in opposite size and direction from one another, such that when one increases the other variable decreases, and vice-versa. A positive correlation exists when one variable tends to decrease as the other variable decreases, or one variable tends to increase when the other increases. The molecular signatures can be positively or negatively correlated to the strength of an endotype (e.g., a high or low polygenic risk score). In example embodiments, the molecular signature increases with increasing endotype score.

Due to the rapid expansion in the number of disease loci the methods described herein can leverage the disease loci to identify pathways associated with disease subtypes, thus facilitating improved disease management. In example embodiments, the molecular signatures can be used to identify key regulatory genes and therapeutics. For example, key transcription factors can be identified that control genes in a genetically-anchored molecular signature or have been experimentally confirmed to control a genetically-anchored molecular signature (e.g., using perturbation assays targeting transcription factors). For example, therapeutic agents can be identified that alter a genetically-anchored molecular signature or have been experimentally confirmed to alter a genetically-anchored molecular signature (e.g., using drug screening). In example embodiments, genetically-anchored molecular signature is reversed. In example embodiments, reversing refers to becoming more similar to the low disease-risk signature (i.e., signature linked to a low disease risk score) or that the signature becomes more similar to the low-risk vs high-risk defined signature. In example embodiments, reversing refers to decreasing the high disease-risk signature (i.e., signature linked to a high disease risk score). In other example embodiments, the one or more therapeutic agents have a molecular signature that positively or negatively correlates with a genetically-anchored molecular signature. In other words, the one or more therapeutic agents have a molecular signature that maps to a genetically-anchored molecular signature (e.g., matches or inverse matches). The correlation or matching can be determined by similarity scoring. In example embodiments, the therapeutic agents can be used to treat subjects with a specific disease subtype (e.g., genotype, endotype). In example embodiments, a genetically-anchored molecular signature can used to monitor and/or modify a treatment (e.g., determining the efficacy of a therapeutic agent).

The present invention also provides for improved genetic subtyping of polygenic diseases by combining rare and common variation. Applicants developed a novel method to incorporate rare variants in T2D polygenic scores and assessed clinical translation of T2D subtyping by studying clinical phenotypes of people in subtypes.

The present invention also provides for improved genetic subtyping of polygenic diseases by providing a pipeline for generating polygenic scores that include greater numbers of variants. Applicants developed a high-throughput pipeline to enable clustering of T2D loci based on variant-trait associations. The pipeline extracted summary statistics from genome-wide association studies (GWAS) for T2D and related traits to generate a matrix of 336 variant x 64 trait associations and applied Bayesian Non-negative Factorization (bNMF) to identify genetic components of T2D. Applicants generated cluster-specific polygenic scores and performed regression analysis in an independent cohort (N=25,419) to assess for clinical relevance. Applicants identified ten clusters, replicating the five from prior analysis as well as novel clusters related to beta-cell dysfunction, pronounced insulin secretion, and levels of alkaline phosphatase, lipoprotein-A, and sex hormone-binding globulin. Four clusters related to mechanisms of insulin deficiency, five to insulin resistance, and one had an unclear mechanism. The clusters displayed tissue-specific epigenomic enrichment, notably with the two beta-cell clusters differentially enriched in functional and stressed pancreatic beta-cell states. Additionally, cluster-specific polygenic scores were differentially associated with patient clinical characteristics and outcomes. The pipeline was applied to coronary artery disease and chronic kidney disease, identifying multiple shared genetic pathways with T2D. The approach stratifies T2D loci into physiologically meaningful genetic clusters associated with distinct tissues and clinical outcomes (i.e., partitioned polygenic risk scores (pPS)). The pipeline allows for efficient updating as additional GWAS become available and can be readily applied to other conditions, facilitating clinical translation of GWAS findings.

The present invention also provides for the concept of a pPS expression signature, which has the potential elucidate genetic architecture and disease pathogenesis as well as aid drug development. The present invention also provides a method for high-throughput identification of T2D drugs. Currently the cost and time required to develop novel drugs creates a major bottleneck (Wouters O J, McKee M, Luyten J. Estimated Research and Development Investment Needed to Bring a New Medicine to Market, 2009-2018. JAMA 2020; 323(9): 844-53). Human genetic data has been credited with improving this bottleneck (Ringel M S, Scannell J W, Baedeker M, Schulze U. Breaking Eroom's Law. Nat Rev Drug Discov 2020; 19(12): 833-4), and Applicants offer a new approach with the potential for disease-modifying therapies and pathway-tailored, targeted treatment strategies.

### Genetically Anchored Signatures

### Datasets

In example embodiments, a genetically anchored signature is a signature linked to an endotype (e.g., a low risk endotype or high risk endotype) by performing a correlation analysis between an endotype and a molecular profile. In preferred embodiments, the endotype can be characterized by different degrees of the endotype (e.g., a polygenic risk score). In order to link a signature to an endotype a dataset that comprises both endotype and molecular profile data for individual samples is required. The dataset can be an existing dataset or can be generated de novo. In example embodiments, the dataset includes data from bulk tissue samples. The tissue samples are preferably derived from tissues associated with the disease of interest. In example embodiments, the dataset comprises endotype data and single cell data. The single cell data is preferably from single cells associated with the disease of interest.

In example embodiments, the dataset comprises genotype data and includes genetic variants. The genetic variants can be in the nuclear genome. The genetic variants may also be present in the mitochondrial genome. In an example embodiment, a polygenic risk score is determined for a population of subjects having a disease (e.g., using a database described herein: UK Biobank, MGB Biobank, TOPMed, and All of Us). The specific variants that make up the polygenic risk score can then be evaluated in a dataset comprising genotype data and molecular profiles (e.g., Genotype-Tissue Expression (GTEx) project). The specific variants that make up the polygenic risk score can then be evaluated in samples without sequencing the whole genome of each sample. The samples can then be evaluated for a molecular profile either simultaneously or after determining polygenic risk scores. The samples can be tissue samples obtained from a plurality of subjects. The samples can be cells that have the polygenic score and are modified to have different polygenic risk score values. The cells having different polygenic risk scores can then be evaluated for a molecular profile.

In example embodiments, the dataset can be a cell atlas or single cell atlas. As used herein “atlas” refers to a collection of data from any tissue sample of interest having a phenotype of interest (see, e.g., Rozenblatt-Rosen O, Stubbington M J T, Regev A, Teichmann S A., The Human Cell Atlas: from vision to reality., Nature. 2017 Oct. 18; 550(7677):451-453; and Regev, A. et al. The Human Cell Atlas Preprint available at bioRxiv at dx.doi.org/10.1101/121202 (2017)). The atlas can include biological information, including medical records, histology, single cell profiles, and genetic information.

### Endotype Data

In example embodiments, endotype data includes any data that defines a distinct functional or pathobiological mechanism, such as markers that contribute to a disease. In example embodiments, samples having different levels for the endotype can be distributed into categorical variables (e.g., samples having different numbers of markers).

### Polygenic Risk Scores

In example embodiments, endotype data is preferably genetic (i.e., genotype data). The endotype data can include genome variants that are associated with the distinct functional or pathobiological mechanism. In preferred embodiments, the genome variants can be used to generate a polygenic risk score. In example embodiments, the polygenic risk score is a partitioned polygenic risk score (pPS) and is enriched for variants that share a similar pattern of genome-wide associations across disease related traits for the disease (see, Udler M S, Kim J, von Grotthuss M, et al. Type 2 diabetes genetic loci informed by multi-trait associations point to disease mechanisms and subtypes: A soft clustering analysis. PLoS medicine 2018; 15(9): e1002654; and expanded pPS's described in Examples 1-3).

In example embodiments, the pPS is enriched for variants linked to DNA regulatory elements active (e.g., enhancers) in the tissue associated with the disease. Any methods of linking enhancers to genes expressed in tissues can be used. In example embodiments, an Activity-by-Contact (ABC) model is used to link variants to genes. This model is based on the simple biochemical notion that an element's quantitative effect on a gene should depend on its strength as an enhancer (“Activity”) weighted by how often it comes into 3D contact with the promoter of the gene (“Contact”), and that the relative contribution of an element on a gene's expression should depend on the element's effect divided by the total effect of all elements (see, e.g., Fulco, et al. Activity-by-contact model of enhancer-promoter regulation from thousands of CRISPR perturbations. Nat Genet. 2019; 51(12):1664-1669. doi:10.1038/s41588-019-0538-0; and Moonen, et al., 2020, KLF4 Recruits SWI/SNF to Increase Chromatin Accessibility and Reprogram the Endothelial Enhancer Landscape under Laminar Shear Stress. bioRxiv 2020.07.10.195768, doi.org/10.1101/2020.07.10.195768). In example embodiments, an epigenome model, such as Roadmap, is used to link variants to gene modules (see, e.g., Ernst, J., Kheradpour, P., Mikkelsen, T. et al. Mapping and analysis of chromatin state dynamics in nine human cell types. Nature 473, 43-49 (2011); Kundaje, A., Meuleman, W., Ernst, J. et al. Integrative analysis of 111 reference human epigenomes. Nature 518, 317-330 (2015); and egg2.wustl.edu/roadmap/web_portal/index.html). In example embodiments, an Enhancer-to-gene (E2G) strategy is a combined union of Activity-By-Contact and Roadmap Enhancer-to-gene (E2G) strategy (Roadmap-U-ABC E2G strategy) (see, e.g., US patent application publication US20210071255A1).

In example embodiments, the polygenic risk score comprises the most common variants associated with the disease related traits, optionally, including additional variants that are progressively less common for the disease. In example embodiments, the polygenic risk score comprises less than 100 variants. In example embodiments, the polygenic risk score comprises 100 or more variants. In example embodiments, the polygenic risk score comprises between 100 to 400 variants. In example embodiments, the polygenic risk score comprises 1000 or more variants. In example embodiments, the polygenic risk score is obtained by a pipeline applying Bayesian Non-negative Factorization (bNMF).

Identifying the presence of a risk loci can be done by any DNA detection method known in the art, including sequencing at least part of a genome of one or more cells from the subject. In certain example embodiments, detection of variants can be done by sequencing. Sequencing can be, for example, whole genome sequencing. In one example embodiment, the invention involves high-throughput and/or targeted nucleic acid profiling (for example, sequencing, quantitative reverse transcription polymerase chain reaction, and the like).

In example embodiments, sequencing comprises high-throughput (formerly “next-generation”) technologies to generate sequencing reads. In DNA sequencing, a read is an inferred sequence of base pairs (or base pair probabilities) corresponding to all or part of a single DNA fragment. A typical sequencing experiment involves fragmentation of the genome into millions of molecules or generating complementary DNA (cDNA) fragments, which are size-selected and ligated to adapters. The set of fragments is referred to as a sequencing library, which is sequenced to produce a set of reads. Methods for constructing sequencing libraries are known in the art (see, e.g., Head et al., Library construction for next-generation sequencing: Overviews and challenges. Biotechniques. 2014; 56(2): 61-77; Trombetta, J. J., Gennert, D., Lu, D., Satija, R., Shalek, A. K. & Regev, A. Preparation of Single-Cell RNA-Seq Libraries for Next Generation Sequencing. Curr Protoc Mol Biol. 107, 4 22 21-24 22 17, doi:10.1002/0471142727.mb0422s107 (2014). PMCID:4338574). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., genomic DNA, cDNA) may include sequencing adaptors that are compatible with use in, e.g., Illumina's reversible terminator method, long read nanopore sequencing, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Schneider and Dekker (Nat Biotechnol. 2012 Apr. 10; 30(4):326-8); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol. Biol. 2009; 553:79-108); Appleby et al (Methods Mol. Biol. 2009, 513:19-39); and Morozova et al (Genomics. 2008 92:255-64), which are incorporated by reference for the general descriptions of the methods and the particular steps of the methods, including all starting products, reagents, and final products for each of the steps.

In certain embodiments, the present invention includes whole genome sequencing. Whole genome sequencing (also known as WGS, full genome sequencing, complete genome sequencing, or entire genome sequencing) is the process of determining the complete DNA sequence of an organism's genome at a single time. This entails sequencing all of an organism's chromosomal DNA as well as DNA contained in the mitochondria and, for plants, in the chloroplast. “Whole genome amplification” (“WGA”) refers to any amplification method that aims to produce an amplification product that is representative of the genome from which it was amplified. Non-limiting WGA methods include Primer extension PCR (PEP) and improved PEP (I-PEP), Degenerated oligonucleotide primed PCR (DOP-PCR), Ligation-mediated PCR (LMP), T7-based linear amplification of DNA (TLAD), and Multiple displacement amplification (MDA).

In certain embodiments, targeted sequencing is used in the present invention (see, e.g., Mantere et al., PLoS Genet 12 e1005816 2016; and Carneiro et al. BMC Genomics, 2012 13:375). Targeted gene sequencing panels are useful tools for analyzing specific mutations in a given sample. Focused panels contain a select set of genes or gene regions that have known or suspected associations with the disease or phenotype under study. In certain embodiments, targeted sequencing is used to detect mutations associated with a disease in a subject in need thereof. Targeted sequencing can increase the cost-effectiveness of variant discovery and detection.

Variants may also be detected through hybridization-based methods, including dynamic allele-specific hybridization (DASH), molecular beacons, and SNP microarrays, enzyme-based methods including RFLP, PCR-based, e.g., allelic-specific polymerase chain reaction (AS-PCR), polymerase chain reaction—restriction fragment length polymorphism (PCR-RFLP), multiplex PCR real-time invader assay (mPCR-RETINA), (amplification refractory mutation system (ARMS), Flap endonuclease, primer extension, 5′ nuclease, e.g., Taqman or 5′nuclease allelic discrimination assay, and oligonucleotide ligation assay, and methods such as single strand conformation polymorphism, temperature gradient gel electrophoresis, denaturing high performance liquid chromatography, high-resolution melting of the entire amplicon, use of DNA mismatch-binding proteins, SNPlex, and Surveyor nuclease assay.

### Molecular Profile Data

In example embodiments, the molecular profiles in the data set comprise a transcriptomic profile, a proteomic profile, a metabolomic profile, a cell-imaging based profile, a spatial transcriptomic profile, a spatial proteomics profile, a spatial metabolomics profile, an epigenomic profile, a clinical imaging profile, a lipodomic profile, or a combination thereof.

In example embodiments, the molecular profiles are obtained from single cell data. The single cell data is preferably from single cells associated with the disease of interest (e.g., originating from a tissue associated with the disease or specific cell types). In example embodiments, an endotype is linked to a molecular profile in single cell types associated with the disease. In example embodiments, the molecular profile that is linked to an endotype is a molecular profile from a single cell type that has the highest correlation with the endotype. For example, a molecular profile from a plurality of single cells are compared to an endotype score and a molecular profile in a single cell type that most closely correlates with the endotype score is selected.

### Transcriptomic Profile

In example embodiments, the molecular profile comprises transcriptome data (e.g., gene expression). As used herein the term “transcriptome” refers to the set of transcript molecules. In some embodiments, transcript refers to RNA molecules, e.g., messenger RNA (mRNA) molecules, small interfering RNA (siRNA) molecules, transfer RNA (tRNA) molecules, ribosomal RNA (rRNA) molecules, and complimentary sequences, e.g., cDNA molecules. In some embodiments, a transcriptome refers to a set of mRNA molecules. In some embodiments, a transcriptome refers to a set of cDNA molecules. In some embodiments, a transcriptome refers to one or more of mRNA molecules, siRNA molecules, tRNA molecules, rRNA molecules, in a sample, for example, a single cell or a population of cells. In some embodiments, a transcriptome refers to cDNA generated from one or more of mRNA molecules, siRNA molecules, tRNA molecules, rRNA molecules, in a sample, for example, a single cell or a population of cells. In some embodiments, a transcriptome refers to 50%, 55, 60, 65, 70, 75, 80, 85, 90, 91, 92, 93, 94, 95, 96, 97, 98, 99, 99.9, or 100% of transcripts from a single cell or a population of cells. In some embodiments, transcriptome not only refers to the species of transcripts, such as mRNA species, but also the amount of each species in the sample. In some embodiments, a transcriptome includes each mRNA molecule in the sample, such as all the mRNA molecules in a single cell.

In example embodiments, transcriptome data comprises bulk RNA sequencing (e.g., RNA-seq). In example embodiments, transcriptome data comprises single cell RNA sequencing (e.g., scRNA-seq). In example embodiments, an endotype is linked to a signature in single cell types associated with the disease. In example embodiments, the signature that is linked to an endotype is a signature from a single cell type that has the highest correlation with the endotype. For example, a transcriptome from a plurality of single cells are compared to an endotype score and a gene signature in a single cell type that most closely correlates with the endotype score is selected.

In certain embodiments, the invention involves single cell RNA sequencing (see, e.g., Qi Z, Barrett T, Parikh A S, Tirosh I, Puram S V. Single-cell sequencing and its applications in head and neck cancer. Oral Oncol. 2019; 99:104441; Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. Jan; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International Patent Application No. PCT/US2016/059239, published as WO2017164936 on Sep. 28, 2017; International Patent Application No. PCT/US2018/060860, published as WO/2019/094984 on May 16, 2019; International Patent Application No. PCT/US2019/055894, published as WO/2020/077236 on Apr. 16, 2020; Drokhlyansky, et al., “The enteric nervous system of the human and mouse colon at a single-cell resolution,” bioRxiv 746743; doi: doi.org/10.1101/746743; and Drokhlyansky E, Smillie C S, Van Wittenberghe N, et al. The Human and Mouse Enteric Nervous System at Single-Cell Resolution. Cell. 2020; 182(6):1606-1622.e23, which are herein incorporated by reference in their entirety.

### Proteomic Profile

In example embodiments, the molecular profile comprises proteome data. Proteome data may include mass spectrometry. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc.) imprinted polymers, avimers, peptidomimetics, peptoids, peptide nucleic acids, threose nucleic acid, a hormone receptor, a cytokine receptor, and synthetic receptors, and modifications and fragments of these.

Single cells can be analyzed by mass cytometry (CyTOF) and tissue samples can be analyzed by Multiplexed Ion Beam Imaging (MIBI) (see, e.g., Hartmann F J, Bendall S C. Immune monitoring using mass cytometry and related high-dimensional imaging approaches. Nat Rev Rheumatol. 2020; 16(2):87-99). Non-limiting examples include multiplex analysis of single cell constituents (US20180340939A), single-cell proteomic assay using aptamers (US20180320224A1), and methods of identifying multiple epitopes in cells (US20170321251A1). In example embodiments, CITE-seq (cellular proteins) is used to generate single cell RNA-seq and proteomics data (see, e.g., Stoeckius, M. et al. Simultaneous epitope and transcriptome measurement in single cells. Nat. Methods 14, 865-868 (2017)).

### Epigenomic Profiles

In example embodiments, the molecular profile comprises epigenomic profiles. Epigenomic profiles have been described and are obtainable in databases (see, e.g., NIH Roadmap Epigenomics Mapping Consortium, ENCODE, Cistrome, and ChTP Atlas; ENCODE Project Consortium, Moore J E, Purcaro M J, et al. Expanded encyclopaedias of DNA elements in the human and mouse genomes. Nature. 2020; 583(7818):699-710; Li S, Wan C, Zheng R, et al. Cistrome-GO: a web server for functional enrichment analysis of transcription factor ChIP-seq peaks. Nucleic Acids Res. 2019; 47(W1):W206-W211; and Shinya Oki, Tazro Ohta, et al. ChIP-Atlas: a data-mining suite powered by full integration of public ChIP-seq data. EMBO Rep. (2018) e46255). The epigenomic profile can be a chromatin accessibility profile (e.g., ATAC-seq), a chromatin modification profile (e.g., ChIP-seq), a chromatin binding profile (e.g., ChIP-seq), a DNA methylation profile (e.g., Bisulfite-Seq), a DNase hypersensitivity profile (e.g., DNase-seq), or a DNA-DNA contact profile (e.g., Hi-C).

In example embodiments, epigenomic profiles are single cell profiles. In example embodiments, the invention involves the Assay for Transposase Accessible Chromatin using sequencing (ATAC-seq) (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aabl601. Epub 2015 May 7; US20160208323A1; US20160060691A1; and WO2017156336A1). In example embodiments, genome wide chromatin immunoprecipitation is used (ChIP) (see, e.g., Rotem, et al., Single-cell ChIP-seq reveals cell subpopulations defined by chromatin state, Nat Biotechnol 33, 1165-1172 (2015)). In example embodiments, epigenetic features can be chromatin contact domains, chromatin loops, superloops, or chromatin architecture data, such as obtained by single cell Hi-C(see, e.g., Rao et al., Cell. 2014 Dec. 18; 159(7):1665-80; and Ramani, et al., Sci-Hi-C: A single-cell Hi-C method for mapping 3D genome organization in large number of single cells Methods. 2020 Jan. 1; 170: 61-68). In example embodiments, SHARE-Seq is used to generate single cell RNA-seq and chromatin accessibility data (see, e.g., Ma, S. et al. Chromatin potential identified by shared single cell profiling of RNA and chromatin. bioRxiv 2020.06.17.156943 (2020) doi:10.1101/2020.06.17.156943).

### Spatial Detection Profiles

In example embodiments, the molecular profile comprises spatial detection data. In example embodiments, spatially resolved molecular profiles are anchored to an endotype. For example, a pPS can be linked to gene or protein expression in specific cells located at the sites of disease or the location where the disease manifests. An example spatial detection platform includes the digital spatial profiler (DSP), GeoMx DSP, which is built on Nanostring's digital molecular barcoding core technology and is further extended by linking the target complementary sequence probe to a unique DSP barcode through a UV cleavable linker (see, e.g., Li X, Wang CY. From bulk, single-cell to spatial RNA sequencing. Int J Oral Sci. 2021; 13(1):36). A pool of such barcode-labeled probes is hybridized to mRNA targets that are released from fresh or FFPE tissue sections mounted on a glass slide. The slide is also stained using fluorescent markers (i.e., fluorescently conjugated antibodies) and imaged to establish tissue “geography” using the GeoMx DSP instrument. After the regions-of-interest (ROIs) are selected, the DSP barcodes are released via UV exposure and collected from the ROIs on the tissue. These barcodes are sequenced through standard NGS procedures. The identity and number of sequenced barcodes can be translated into specific mRNA molecules and their abundance, respectively, and then mapped to the tissue section based on their geographic location. The DSP barcode can also be linked to antibodies to detect proteins. An example spatial detection platform includes the CosMx Spatial Molecular Imager (Nanostring) platform, which enables high-plex (~1,000 genes) spatial transcriptomics and proteomics at single cell and subcellular resolution (see, e.g., He, et al., High-plex Multiomic Analysis in FFPE at Subcellular Level by Spatial Molecular Imaging, bioRxiv 2021.11.03.467020). Other spatial detection methods or platform applicable to the present invention have been described (see, e.g., Li X, Wang CY. From bulk, single-cell to spatial RNA sequencing. Int J Oral Sci. 2021; 13(1):36. Published 2021 Nov. 15. doi:10.1038/s41368-021-00146-0). Additional non-limiting methods of generating spatial data of varying resolution are known in the art, for example, multiplexed ion beam imaging (MIBI) (see, e.g., Angelo et al., Nat Med. 2014 April; 20(4): 436-442), NanoString (DSP, digital spatial profiling) (see e.g., Li X, Wang C Y. From bulk, single-cell to spatial RNA sequencing. Int J Oral Sci. 2021; 13(1):36; and Geiss G K, et al., Direct multiplexed measurement of gene expression with color-coded probe pairs. Nat Biotechnol. 2008 March; 26(3):317-25), ISS (Ke, R. et al. In situ sequencing for RNA analysis in preserved tissue and cells. Nat. Methods 10, 857-860 (2013)), MERFISH (Chen, K. H., Boettiger, A. N., Moffitt, J. R., Wang, S. & Zhuang, X. Spatially resolved, highly multiplexed RNA profiling in single cells. Science 348, (2015)), smFISH (Codeluppi, S. et al. Spatial organization of the somatosensory cortex revealed by cyclic smFISH. biorxiv.org/lookup/doi/10.1101/276097 (2018) doi:10.1101/276097), osmFISH (Codeluppi, S. et al. Spatial organization of the somatosensory cortex revealed by osmFISH. Nat. Methods 15, 932-935 (2018)), STARMap (Wang, X. et al. Three-dimensional intact-tissue sequencing of single-cell transcriptional states. Science 361, eaat5691 (2018)), Targeted ExSeq (Alon, S. et al. Expansion Sequencing: Spatially Precise In Situ Transcriptomics in Intact Biological Systems. biorxiv.org/lookup/doi/10.1101/2020.05.13.094268 (2020) doi:10.1101/2020.05.13.094268), seqFISH+ (Eng, C.-H. L. et al. Transcriptome-scale super-resolved imaging in tissues by RNA seqFISH+. Nature (2019) doi:10.1038/s41586-019-1049-y.), Spatial Transcriptomics methods (e.g., Spatial Transcriptomics (ST))(see, e.g., Stihl, P. L. et al. Visualization and analysis of gene expression in tissue sections by spatial transcriptomics. Science 353, 78-82 (2016)) (commercially as Visium); Visium Spatial Capture Technology, 10× Genomics, Pleasanton, CA; WO2020047007A2; WO2020123317A2; WO2020047005A1; WO2020176788A1; and WO2020190509A9), Slide-seq (Rodriques, S. G. et al. Slide-seq: A scalable technology for measuring genome-wide expression at high spatial resolution. Science 363, 1463-1467 (2019)), or High Definition Spatial Transcriptomics (Vickovic, S. et al. High-definition spatial transcriptomics for in situ tissue profiling. Nat. Methods 16, 987-990 (2019)). In certain embodiments, proteomics and spatial patterning using antenna networks is used to spatially map a tissue specimen and this data can be further used to align single cell data to a larger tissue specimen (see, e.g., US20190285644A1). In certain embodiments, the spatial data can be immunohistochemistry data or immunofluorescence data.

### Metabolic Profiles

In example embodiments, the dataset includes cellular metabolic states obtained from analyzing tissue samples or single cells. In example embodiments, metabolites are detected (see, e.g., Rappez L, Stadler M, Triana S, et al. SpaceM reveals metabolic states of single cells. Nat Methods. 2021; 18(7):799-805. doi:10.1038/s41592-021-01198-0). In example embodiments, the dataset includes cellular metabolic states based on RNA-seq or single-cell RNA sequencing (see, e.g., Wagner A, Wang C, Fessler J, et al. Metabolic modeling of single Th17 cells reveals regulators of autoimmunity. Cell. 2021; 184(16):4168-4185.e21).

### Cell-Imaging Based Profiles

In example embodiments, the data set comprises morphological data obtained from differentiating stem cells for a plurality of subjects. The morphological data can be used to generate an endotype score for the subjects (e.g., by quantitating the number and intensity of features) or can be the molecular profile for the subjects. Morphological features can be identified by cell painting (see, e.g., Bray MA, Singh S, Han H, et al. Cell Painting, a high-content image-based assay for morphological profiling using multiplexed fluorescent dyes. Nat Protoc. 2016; 11(9):1757-1774); and Laber, et al., Discovering cellular programs of intrinsic and extrinsic drivers of metabolic traits using LipocyteProfiler, bioRxiv 2021.07.17.452050).

In example embodiments, the molecular profile comprises histology data. Histology, also known as microscopic anatomy or microanatomy, is the branch of biology which studies the microscopic anatomy of biological tissues. Histology is the microscopic counterpart to gross anatomy, which looks at larger structures visible without a microscope. Although one may divide microscopic anatomy into organology, the study of organs, histology, the study of tissues, and cytology, the study of cells, modem usage places these topics under the field of histology. In medicine, histopathology is the branch of histology that includes the microscopic identification and study of diseased tissue. Biological tissue has little inherent contrast in either the light or electron microscope. Staining is employed to give both contrast to the tissue as well as highlighting particular features of interest. When the stain is used to target a specific chemical component of the tissue (and not the general structure), the term histochemistry is used. Antibodies can be used to specifically visualize proteins, carbohydrates, and lipids. This process is called immunohistochemistry, or when the stain is a fluorescent molecule, immunofluorescence. This technique has greatly increased the ability to identify categories of cells under a microscope. Other advanced techniques, such as nonradioactive in situ hybridization (ISH), can be combined with immunochemistry to identify specific DNA or RNA molecules with fluorescent probes or tags that can be used for immunofluorescence and enzyme-linked fluorescence amplification.

### Linking Endotypes to Molecular Signatures

In example embodiments, genetically-anchored molecular signatures are obtained by linking genetic subtypes (i.e., endotypes) of diseases to molecular profiles by correlation analysis. In example embodiments, for each dataset linear regression models are run with each endotype value and every molecular profile variable as an outcome and producing an effect estimate (beta), Pvalue, and Qvalue. The regression beta represents the change in molecular profile variable level per change in the endotype value. In example embodiments, the analysis produces a vector of molecular profile variable changes per standard deviation change in the endotype value. This vector represents a genetically-anchored molecular signature for each sample in the dataset. The genetically-anchored molecular signatures can then be meta-analyzed in other datasets of tissues (e.g. SC adipose in MOBB and GTEx), shared cell types (e.g., single cell data sets), or shared spatial location of disease (e.g., spatial data sets). In example embodiments, the signatures with the largest magnitude of regression beta values, indicating largest mean expression changes are selected. In example embodiments, more than one signature is generated. In example embodiments, the top 1, 2, 3, 4, 5, 6, 7, 8, 9, or 10 signatures with the largest magnitude of regression beta values are used for identifying regulators, or therapeutic targets or agents. In example embodiments, the signatures with the lowest p values and/or q values are selected. In example embodiments, a multi-gene expression signature is selected rather than a single gene effect.

In example embodiments, a genetically-anchored molecular signature is further used to identify functional programs related to each disease endotype. In example embodiments, in silico functional characterization of the genetically-anchored molecular signatures can be performed. For example, enrichment of established gene set libraries including Gene Ontologies (GO), Reactome, BioPlanet, KEGG can be tested using established gene-set enrichment tools including GSEA, the Ingenuity Pathway Analysis Tool, and Gene-List Network Enrichment Analysis (GeLiNEA). In example embodiments, the genetically-anchored molecular signatures are analyzed for enrichment of transcriptional regulators using an analysis such as, the “epigenetic Landscape In Silico deletion Analysis” (Lisa), which incorporates chromatin profile data and transcription factor/chromatin regulator ChIP-seq datasets from human and mouse studies to assess for enrichment of transcription factor and chromatin regulator binding sites across the top genes in an expression signature or the Ensembl Variant Effect Predictor (VEP) to assess for pathway interactions between genes. In example embodiments, enrichment analyses can be focused on results reaching significance after accounting for multiple testing using a Qvalue <0.001, 0.01, 0.1, or 0.5 threshold.

The genetically-anchored molecular signatures may encompass any gene or genes, protein or proteins, epigenetic element(s), clinical features, or morphological features whose expression profile or whose occurrence is correlated with a specific endotype (e.g., a high or low disease risk polygenic score). For example, a specific endotype may be correlated with genes, proteins, epigenetic element(s), clinical features or morphological features. Further, therapeutic agents can have similar signatures of genes, proteins, epigenetic element(s), clinical features, or morphological features and can be identified (e.g., using perturbation studies). The signatures of the present invention may be microenvironment specific, such as their expression in a particular spatio-temporal context. The signature according to certain embodiments of the present invention may comprise or consist of one or more genes, proteins, epigenetic elements, and/or features, such as for instance 1, 2, 3, 4, 5, 6, 7, 8, 9, 10, 25, 50, 100, 500, 1000 or more. In certain embodiments, the signature may comprise or consist of two or more genes, proteins and/or epigenetic elements, such as for instance 2, 3, 4, 5, 6, 7, 8, 9, 10, 25, 50, 100, 500, 1000 or more. It is to be understood that a signature according to the invention may for instance also include genes or proteins as well as epigenetic elements combined. In this context, a signature consists of one or more differentially expressed genes/proteins or differential epigenetic elements or features when comparing different cells or cell (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art. As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells).

Signatures may be functionally validated as being uniquely associated with a particular phenotype. Induction or suppression of a particular signature may consequentially be associated with or causally drive a particular phenotype. Various aspects and embodiments of the invention may involve analyzing gene signatures, protein signature, and/or other genetic or epigenetic signature based on single cell analyses (e.g., single cell RNA sequencing) or alternatively based on cell population analyses, as is defined herein elsewhere. Particular advantageous uses include methods for identifying agents capable of inducing or suppressing particular pathways based on the gene signatures, protein signature, and/or other genetic or epigenetic signature as defined herein.

### Identifying Therapeutic Agents

The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature really was the proxy for a phenotype of interest, it could be used to find small molecules that effect that phenotype without knowledge of a validated drug target. The genetically-anchored molecular signatures of the present may be used to screen for drugs that reduce the signature in cells having a specific endotype as described herein. In example embodiments, the invention comprises identifying one or more key regulatory features of the genetically-anchored molecular signature by matching the genetically-anchored molecular signature with one or more perturbation molecular signatures from a perturbation dataset using similarity scoring. In example embodiments, the signatures with the highest similarity are selected. In example embodiments, the signatures that match have a similarity or connectivity score greater than 0.3, 0.4, 0.5, 0.6, 0.7, 0.8, 0.9, or 0.95. In an example embodiment, the similarity or connectivity score is greater than 0.9 or less than −0.9, or greater than 0.95 or less than −0.95. In an example embodiment, the similarity or connectivity score has a false discovery rate (FDR) of less than 0.5, 0.1, or 0.01.

### Perturbation Datasets

In example embodiments, the perturbation datasets can be generated by perturbation of cells (e.g., cell lines, or primary cells) or complex cell populations (e.g., multicellular systems, such as, organoid, tissue explant, or organ on a chip). The perturbation datasets can include signatures for therapeutic agents, such as drugs, small molecules, or antibodies. More generally, any compound screen with a molecular read-out as described herein (e.g., a read-out, such as differential gene expression, proteomic, metabolic, spatial, epigenetic, image-based profiling of morphology and cellular markers, or lipodomics, used to construct the genetically-anchored molecular signatures) can be used to nominate compounds by similarity or connectivity with the genetically-anchored molecular signatures. The perturbation datasets can include signatures for gene knockdown, gene knockout, gene overexpression, gene repression or gene activation. In example embodiments, regulatory proteins, such as transcription factors are perturbed (e.g., by overexpression or knockdown). In one embodiment, perturbation is by deletion of regulatory elements.

In example embodiments, the perturbation datasets include pooled perturbation assays. Methods and tools for genome-scale screening of perturbations in single cells using CRISPR-Cas9 have been described, herein referred to as perturb-seq (see e.g., Dixit et al., “Perturb-Seq: Dissecting Molecular Circuits with Scalable Single-Cell RNA Profiling of Pooled Genetic Screens” 2016, Cell 167, 1853-1866; Adamson et al., “A Multiplexed Single-Cell CRISPR Screening Platform Enables Systematic Dissection of the Unfolded Protein Response” 2016, Cell 167, 1867-1882; Feldman et al., Lentiviral co-packaging mitigates the effects of intermolecular recombination and multiple integrations in pooled genetic screens, bioRxiv 262121, doi: doi.org/10.1101/262121; Datlinger, et al., 2017, Pooled CRISPR screening with single-cell transcriptome readout. Nature Methods. Vol. 14 No. 3 DOI: 10.1038/nmeth.4177; Hill et al., On the design of CRISPR-based single cell molecular screens, Nat Methods. 2018 April; 15(4): 271-274; Replogle, et al., “Combinatorial single-cell CRISPR screens by direct guide RNA capture and targeted sequencing” Nat Biotechnol (2020). doi.org/10.1038/s41587-020-0470-y; Schraivogel D, Gschwind A R, Milbank J H, et al. “Targeted Perturb-seq enables genome-scale genetic screens in single cells”. Nat Methods. 2020; 17(6):629-635; Frangieh C J, Melms J C, Thakore P I, et al. Multimodal pooled Perturb-CITE-seq screens in patient models define mechanisms of cancer immune evasion. Nat Genet. 2021; 53(3):332-341; US patent application publication number US20200283843A1; and US Patent number U.S. Ser. No. 11/214,797B2).

In example embodiments, the genetically-anchored molecular signatures are compared to signatures obtained in prior perturbation assays. For example, the Connectivity Map (cmap) is a comprehensive catalog of cellular signatures representing systematic perturbation with genetic (thus reflecting protein function) and pharmacologic (thus reflecting small-molecule function) perturbagens. Simple pattern-matching algorithms allow the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). As of 2022, CMap has generated a library containing over 1.5M gene expression profiles from ~5,000 small-molecule compounds, and ~3,000 genetic reagents, tested in multiple cell types. Cmap can be used to screen for a matching signature in silico. In another example, the JUMP-Cell Painting Consortium is a data-driven approach to drug discovery based on cellular imaging, image analysis, and high dimensional data analytics (see, e.g., jump-cellpainting.broadinstitute.org). The consortium will create a massive cell-imaging dataset, displaying more than 1 billion cells responding to over 140,000 small molecules and genetic perturbations. JUMP-Target provides lists and 384-well plate maps of 306 compounds and corresponding genetic perturbations, designed to assess connectivity in profiling assays. JUMP-MOA provides a list and a 384-well plate map of 90 compounds in quadruplicate (corresponding to 47 mechanism-of-action classes), designed to assess connectivity in profiling assays.

In one embodiment, CRISPR systems may be used to perturb protein-coding genes or non-protein-coding DNA. CRISPR systems may be used to knockout protein-coding genes by frameshifts, point mutations, inserts, or deletions. In example embodiments, a CRISPR system is used to create an INDEL. CRISPRa/i/x technology may be used in perturbation assays (see, e.g., Konermann et al. “Genome-scale transcriptional activation by an engineered CRISPR-Cas9 complex” Nature. 2014 Dec. 10. doi: 10.1038/nature14136; Qi, L. S., et al. (2013). “Repurposing CRISPR as an RNA-guided platform for sequence-specific control of gene expression”. Cell. 152 (5): 1173-83; Gilbert, L. A., et al., (2013). “CRISPR-mediated modular RNA-guided regulation of transcription in eukaryotes”. Cell. 154 (2): 442-51; Komor et al., 2016, Programmable editing of a target base in genomic DNA without double-stranded DNA cleavage, Nature 533, 420-424; Nishida et al., 2016, Targeted nucleotide editing using hybrid prokaryotic and vertebrate adaptive immune systems, Science 353(6305); Yang et al., 2016, Engineering and optimising deaminase fusions for genome editing, Nat Commun. 7:13330; Hess et al., 2016, Directed evolution using dCas9-targeted somatic hypermutation in mammalian cells, Nature Methods 13, 1036-1042; and Ma et al., 2016, Targeted AID-mediated mutagenesis (TAM) enables efficient genomic diversification in mammalian cells, Nature Methods 13, 1029-1035).

In one embodiment, perturbation of genes is by RNAi. The RNAi may be shRNA's targeting genes. The shRNA's may be delivered by any methods known in the art. In one embodiment, the shRNA's may be delivered by a viral vector. The viral vector may be a lentivirus, adenovirus, or adeno associated virus (AAV).

In one embodiment, perturbation is performed using small molecules. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks. Numerous libraries are commercially available or can be readily produced; means for random and directed synthesis of a wide variety of organic compounds and biomolecules, including expression of randomized oligonucleotides, such as antisense oligonucleotides and oligopeptides, also are known. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Such libraries are useful for the screening of a large number of different compounds.

Epigenetic proteins can regulate many cellular pathways. In example embodiments, a perturbation signature identified using epigenetic protein targeting drugs are matched to a genetically-anchored molecular signature. Small molecules targeting epigenetic proteins are currently being developed and/or used in the clinic to treat disease (see, e.g., Qi et al., HEDD: the human epigenetic drug database. Database, 2016, 1-10; and Ackloo et al., Chemical probes targeting epigenetic proteins: Applications beyond oncology. Epigenetics 2017, VOL. 12, NO. 5, 378-400). In certain embodiments, the one or more agents comprise a histone acetylation inhibitor, histone deacetylase (HDAC) inhibitor, histone lysine methylation inhibitor, histone lysine demethylation inhibitor, DNA methyltransferase (DNMT) inhibitor, inhibitor of acetylated histone binding proteins, inhibitor of methylated histone binding proteins, sirtuin inhibitor, protein arginine methyltransferase inhibitor or kinase inhibitor. In certain embodiments, any small molecule exhibiting the functional activity described above may be used in the present invention. In certain embodiments, the DNA methyltransferase (DNMT) inhibitor is selected from the group consisting of azacitidine (5-azacytidine), decitabine (5-aza-2′-deoxycytidine), EGCG (epigallocatechin-3-gallate), zebularine, hydralazine, and procainamide. In certain embodiments, the histone acetylation inhibitor is C646. In certain embodiments, the histone deacetylase (HDAC) inhibitor is selected from the group consisting of vorinostat, givinostat, panobinostat, belinostat, entinostat, CG-1521, romidepsin, ITF-A, ITF-B, valproic acid, OSU-HDAC-44, HC-toxin, magnesium valproate, plitidepsin, tasquinimod, sodium butyrate, mocetinostat, carbamazepine, SB939, CHR-2845, CHR-3996, JNJ-26481585, sodium phenylbutyrate, pivanex, abexinostat, resminostat, dacinostat, droxinostat, and trichostatin A (TSA). In certain embodiments, the histone lysine demethylation inhibitor is selected from the group consisting of pargyline, clorgyline, bizine, GSK2879552, GSK-J4, KDM5-C70, JIB-04, and tranylcypromine. In certain embodiments, the histone lysine methylation inhibitor is selected from the group consisting of EPZ-6438, GSK126, CPI-360, CPI-1205, CPI-0209, DZNep, GSK343, Ell, BIX-01294, UNC0638, EPZ004777, GSK343, UNC1999 and UNC0224. In certain embodiments, the inhibitor of acetylated histone binding proteins is selected from the group consisting of AZD5153 (see e.g., Rhyasen et al., AZD5153: A Novel Bivalent BET Bromodomain Inhibitor Highly Active against Hematologic Malignancies, Mol Cancer Ther. 2016 November; 15(11):2563-2574. Epub 2016 Aug. 29), PFI-1, CPI-203, CPI-0610, RVX-208, OTX015, I-BET 151, I-BET762, I-BET-726, dBET1, ARV-771, ARV-825, BETd-260/ZBC260 and MZ1. In certain embodiments, the inhibitor of methylated histone binding proteins is selected from the group consisting of UNC669 and UNC1215. In certain embodiments, the sirtuin inhibitor comprises nicotinamide.

### Selecting Therapeutic Agents

In example embodiments, a therapeutic agent is selected if the therapeutic agent has a signature in a perturbation screen that matches a genetically-anchored molecular signature (e.g., positively or negatively correlates with a genetically-anchored molecular signature for a high or low risk endotype, such as a pPS). In example embodiments, the therapeutic agent generates a signature in the correct direction for treating a subject having the disease and the endotype. For example, reducing a high risk endotype signature or increasing a low risk endotype signature. In example embodiments, a therapeutic agent is selected if the therapeutic agent targets a gene or pathway identified in a perturbation screen. For example, if perturbation of a gene or pathway provides a signature that correlates (negatively or positively) with a genetically-anchored molecular signature any therapeutic agent targeting the gene or pathway could be used as a therapeutic agent. In example embodiments, the one or more agents comprises a small molecule inhibitor, small molecule degrader (e.g., ATTEC, AUTAC, LYTAC, or PROTAC), genetic modifying agent, antisense oligonucleotides (ASO), antibody, antibody fragment, antibody-like protein scaffold, aptamer, protein, or any combination thereof.

### Small Molecules

One type of small molecule applicable to the present invention is a degrader molecule (see, e.g., Ding, et al., Emerging New Concepts of Degrader Technologies, Trends Pharmacol Sci. 2020 July; 41(7):464-474). The terms “degrader” and “degrader molecule” refer to all compounds capable of specifically targeting a protein for degradation (e.g., ATTEC, AUTAC, LYTAC, or PROTAC, reviewed in Ding, et al. 2020). Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810). In certain embodiments, LYTACs are particularly advantageous for cell surface proteins.

### Genetic Modifying Agents

In example embodiments, a genetic modifying agent, such as a programmable nuclease, may be used to alter expression of a target gene, such as a regulator protein for a genetically-anchored molecular signature. Gene editing using programmable nucleases may utilize two different cell repair pathways, non-homologous end joining (NHEJ) and homology directed repair. Example programmable nucleases for use in this manner include zinc finger nucleases (ZFN), TALE nucleases (TALENS), meganucleases, and CRISPR-Cas systems.

### CRISPR-Cas

In one example embodiment, the gene editing system is a CRISPR-Cas system. The CRISPR-Cas system comprises a Cas polypeptide and a guide sequence, wherein the guide sequence is capable of forming a CRISPR-Cas complex with the Cas polypeptide and directing site-specific binding of the CRISPR-Cas sequence to a target sequence. The Cas polypeptide may induce a double- or single-stranded break at a designated site in the target sequence. The site of CRISPR-Cas cleavage, for most CRISPR-Cas systems, is dictated by distance from a protospacer-adjacent motif (PAM), discussed in further detail below. Accordingly, a guide sequence may be selected to direct the CRISPR-Cas system to induce cleavage at a desired target site at or near the one or more variants.

### NHEJ-Based Editing

In one example embodiment, the CRISPR-Cas system is used to introduce one or more insertions or deletions in a target gene. More than one guide sequence may be selected to insert multiple insertion, deletions, or combination thereof. Likewise, more than one Cas protein type may be used, for example, to maximize targets sites adjacent to different PAMs. In one example embodiment, a guide sequence is selected that directs the CRISPR-Cas system to make one or more insertions or deletions within an enhancer region in a target gene.

### HDR Template Based Editing

In one example embodiment, a donor template is provided to replace a genomic sequence in a target gene. A donor template may comprise an insertion sequence flanked by two homology regions. The insertion sequence comprises an edited sequence to be inserted in place of the target sequence (e.g., a portion of genomic DNA comprising the one or more variants). The homology regions comprise sequences that are homologous to the genomic DNA strands at the site of the CRISPR-Cas induced double-strand break. Cellular HDR mechanisms then facilitate insertion of the insertion sequence at the site of the DSB. The donor template may include a sequence which results in a change in sequence of 1, 2, 3, 4, 5, 6, 7, 8, 9, 10, 1 1, 12 or more nucleotides of the target sequence.

A donor template may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 1 10+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

The homology regions of the donor template may be complementary to a portion of a polynucleotide comprising the target sequence. When optimally aligned, a donor template might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The donor template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

Homology arms of the donor template may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

In one example embodiment, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

The donor template may further comprise a marker. Such a marker may make it easy to screen for targeted integrations. Examples of suitable markers include restriction sites, fluorescent proteins, or selectable markers. The donor template of the disclosure can be constructed using recombinant techniques (see, for example, Sambrook et al., 2001 and Ausubel et al., 1996).

In one example embodiment, a donor template is a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### Class 1 Systems

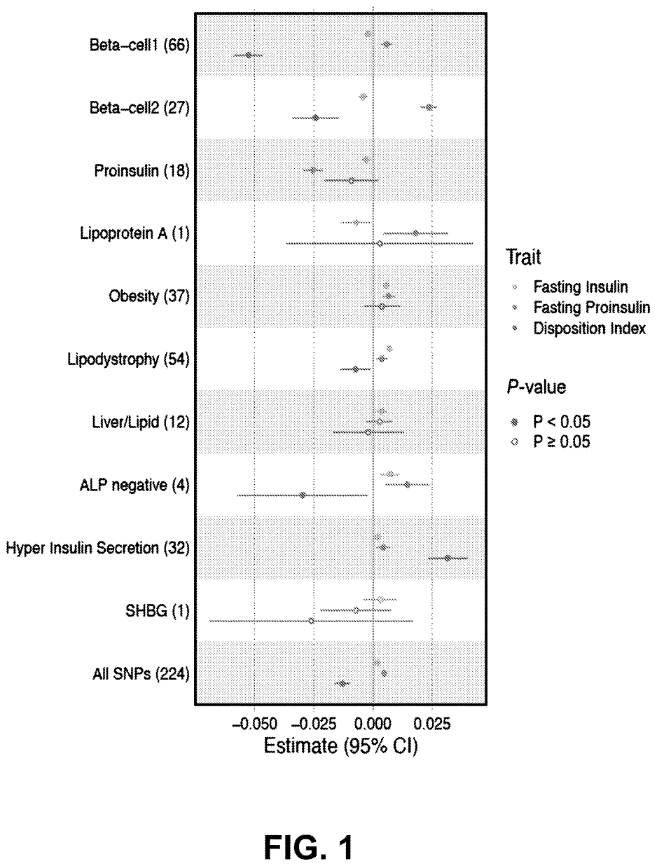

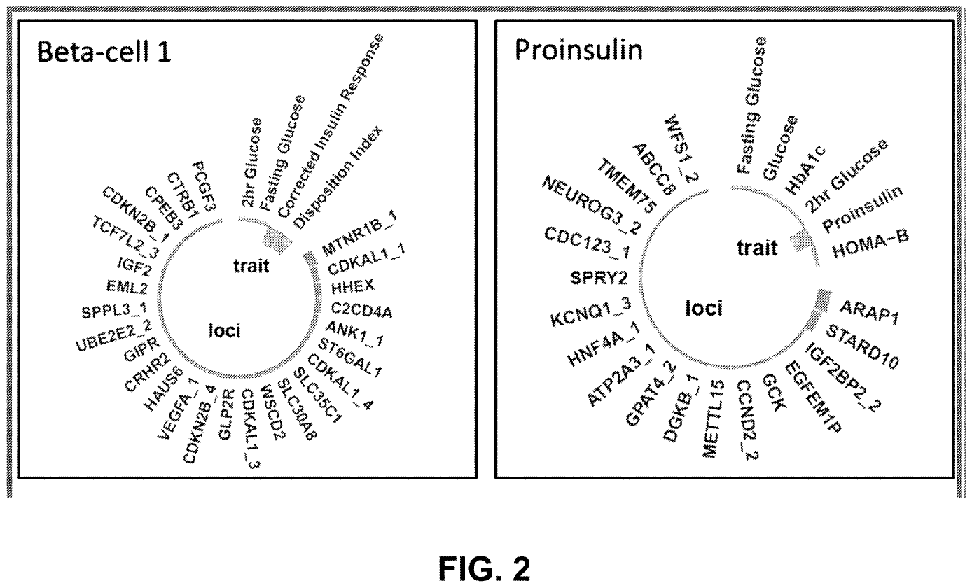

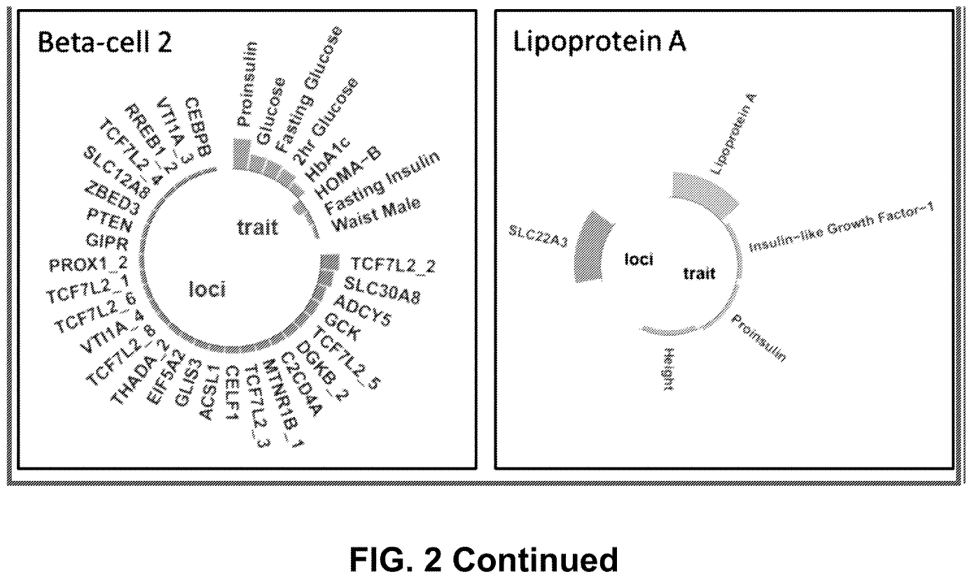

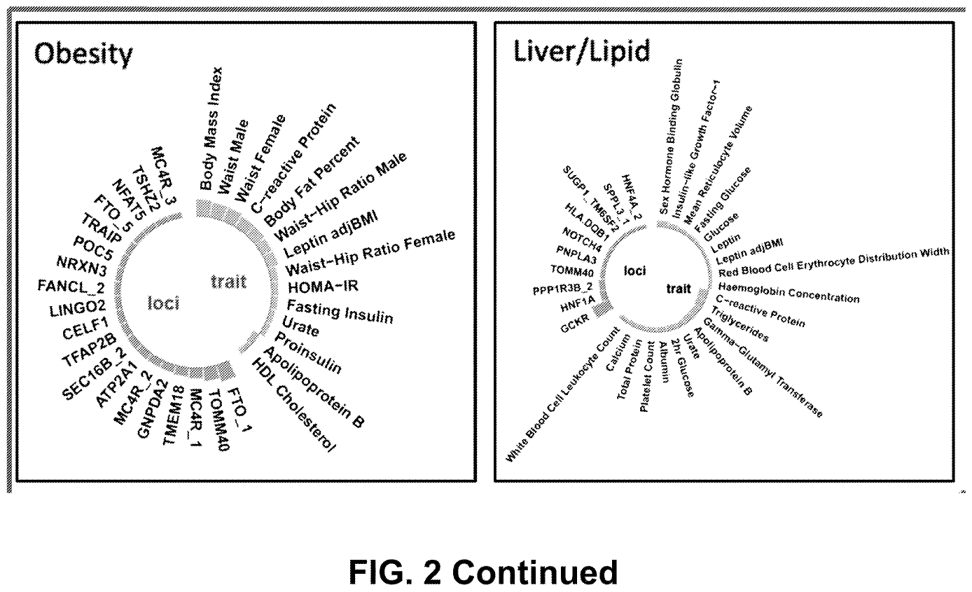

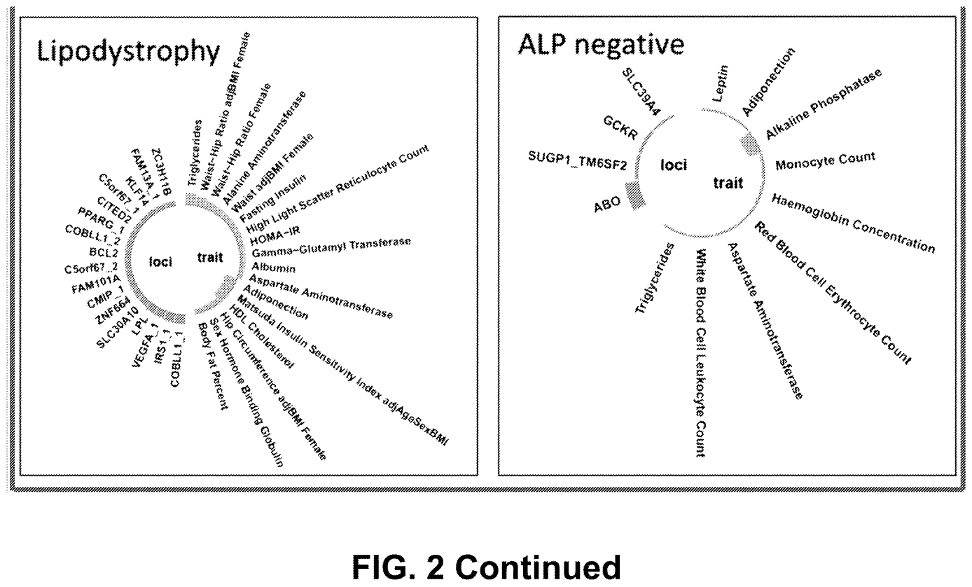

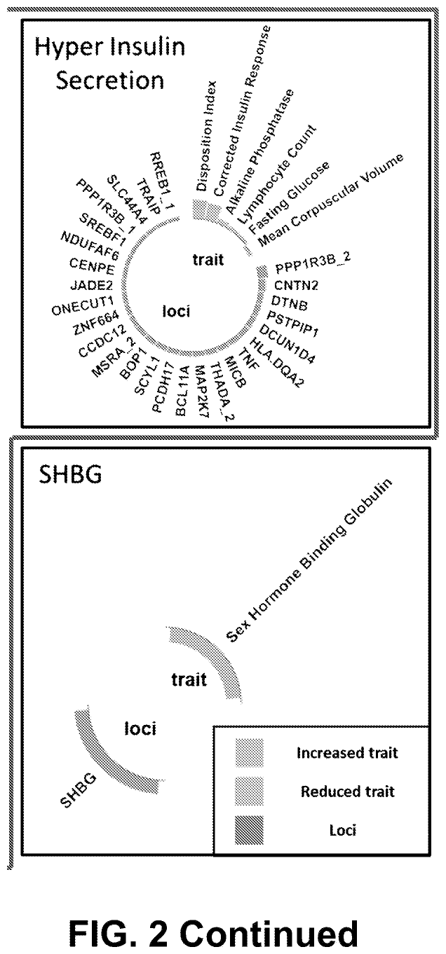

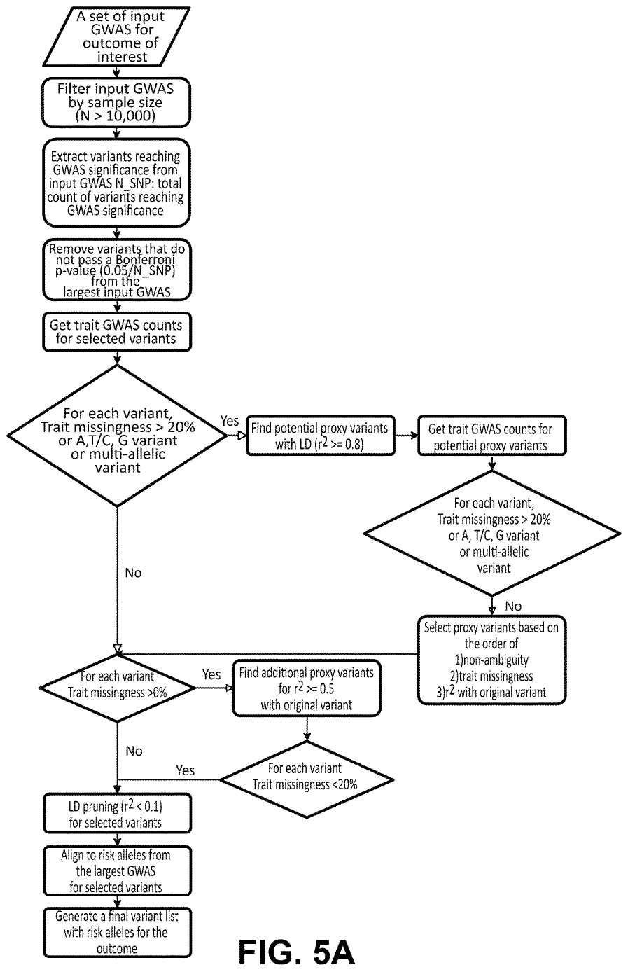

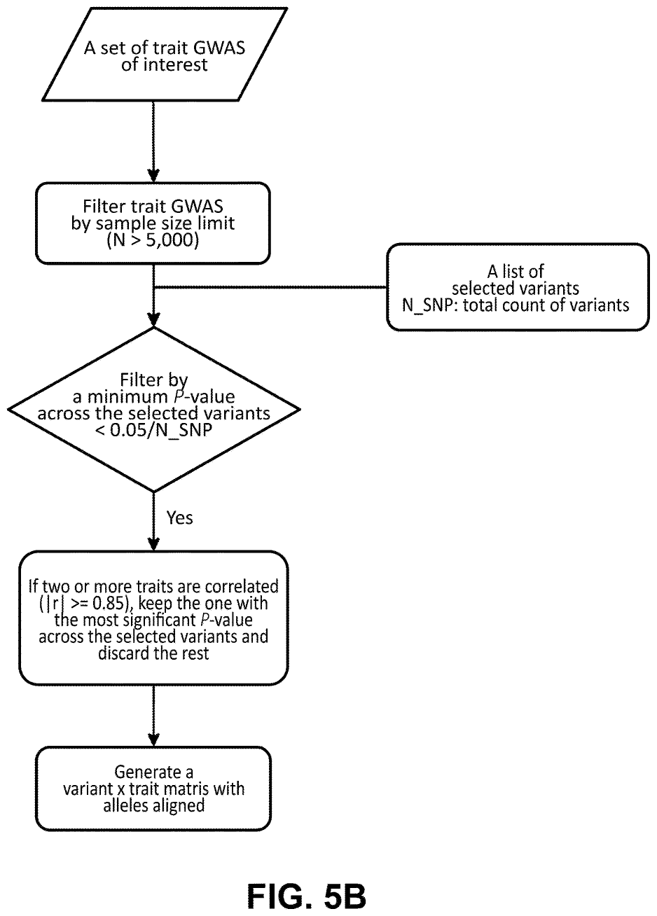

The CRISPR-Cas therapeutic methods disclosed herein may be designed for use with Class 1 CRISPR-Cas systems. In certain example embodiments, the Class I system may be Type I, Type III or Type IV CRISPR-Cas as described in Makarova et al. “Evolutionary classification of CRISPR-Cas systems: a burst of class 2 and derived variants” Nature Reviews Microbiology, 18:67-81 (February 2020)., incorporated in its entirety herein by reference and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g. Cas1, Cas2, RNA nuclease), and/or one or more accessory proteins (e.g. Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g., Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374: 20180087, DOI: 10.1098/rstb.2018.0087. In one aspect, Class 1 systems are characterized by the signature protein Cas3. The Cascade in particular Class1 proteins can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprises one or more Cas5 subunits and two or more Cas7 subunits. Class 1 subtypes include Type I-A, I-B, I-C, I-U, I-D, I-E, and I-F, Type IV-A and IV-B, and Type III-A, III-D, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1, n5, FIG. 5.

### Class 2 Systems

The CRISPR-Cas therapeutic methods disclosed herein may be designed for use with. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-Cl, and II-C2. Class 2, Type V systems can be divided into 17 subtypes: V-A, V-B1, V-B2, V-C, V-D, V-E, V-F 1, V-F 1(V-U3), V-F2, V-F3, V-G, V-H, V-I, V-K (V-US), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside a split Ruv-C like nuclease domain sequence. The Type V systems (e.g., Cas12) only contain a RuvC-like nuclease domain that cleaves both strands. Some Type V systems have also been found to possess this collateral activity with two single-stranded DNA in in vitro contexts.

In one example embodiment, the Class 2 system is a Type II system. In one example embodiment, the Type II CRISPR-Cas system is a II-A CRISPR-Cas system. In one example embodiment, the Type II CRISPR-Cas system is a II-B CRISPR-Cas system. In one example embodiment, the Type 11 CRISPR-Cas system is a II-C1 CRISPR-Cas system. In one example embodiment, the Type II CRISPR-Cas system is a II-C2 CRISPR-Cas system. In sone example embodiments, the Type II system is a Cas9 system. In some embodiments, the Type II system includes a Cas9.

In one example embodiment, the Class 2 system is a Type V system. In one example embodiment, the Type V CRISPR-Cas system is a V-A CRISPR-Cas system. In one example embodiment, the Type V CRISPR-Cas system is a V-B1 CRISPR-Cas system. In one example embodiment, the Type V CRISPR-Cas system is a V-B2 CRISPR-Cas system. In one example embodiment, the Type V CRISPR-Cas system is a V-C CRISPR-Cas system. In one example embodiment, the Type V CRISPR-Cas system is a V-D CRISPR-Cas system. In one example embodiment, the Type V CRISPR-Cas system is a V-E CRISPR-Cas system. In one example embodiment, the Type V CRISPR-Cas system is a V-F1 CRISPR-Cas system. In one example embodiment, the Type V CRISPR-Cas system is a V-F1 (V-U3) CRISPR-Cas system. In one example embodiment, the Type V CRISPR-Cas system is a V-F2 CRISPR-Cas system. In one example embodiment, the Type V CRISPR-Cas system is a V-F3 CRISPR-Cas system. In one example embodiment, the Type V CRISPR-Cas system is a V-G CRISPR-Cas system. In one example embodiment, the Type V CRISPR-Cas system is a V-H CRISPR-Cas system. In one example embodiment, the Type V CRISPR-Cas system is a V-I CRISPR-Cas system. In one example embodiment, the Type V CRISPR-Cas system is a V-K (V-U5) CRISPR-Cas system. In one example embodiment, the Type V CRISPR-Cas system is a V-U1 CRISPR-Cas system. In one example embodiment, the Type V CRISPR-Cas system is a V-U2 CRISPR-Cas system. In one example embodiment, the Type V CRISPR-Cas system is a V-U4 CRISPR-Cas system. In one example embodiment, the Type V CRISPR-Cas is a Cas12a (Cpf1), Cas12b (C2cl), Cas12c (C2c3), Cas12d (CasY), Cas12e (CasX), Cas14, and/or Cas(D.

### Guide Molecules

The following include general design principles that may be applied to the guide molecule. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In one example embodiment, a guide RNA or crRNA may comprise, consist essentially of, or consist of a direct repeat (DR) sequence and a guide sequence or spacer sequence. In another example embodiment, the guide RNA or crRNA may comprise, consist essentially of, or consist of a direct repeat sequence fused or linked to a guide sequence or spacer sequence. In another example embodiment, the direct repeat sequence may be located upstream (i.e., 5′) from the guide sequence or spacer sequence. In other embodiments, the direct repeat sequence may be located downstream (i.e., 3′) from the guide sequence or spacer sequence.

In one example embodiment, the crRNA comprises a stem loop, preferably a single stem loop. In one example embodiment, the direct repeat sequence forms a stem loop, preferably a single stem loop.

In one example embodiment, the spacer length of the guide RNA is from 15 to 35 nt. In another example embodiment, the spacer length of the guide RNA is at least 15 nucleotides. In another example embodiment, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All of (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

In the context of formation of a CRISPR complex, “target sequence” refers to a sequence to which a guide sequence is designed to have complementarity, where hybridization between a target sequence and a guide sequence promotes the formation of a CRISPR complex. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

PAM elements are sequences that can be recognized and bound by Cas proteins. Cas proteins/effector complexes can then unwind the dsDNA at a position adjacent to the PAM element. It will be appreciated that Cas proteins and systems target RNA do not require PAM sequences (Marraffini et al. 2010. Nature. 463:568-571). Instead, many rely on PFSs, which are discussed elsewhere herein. In one example embodiment, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table A (from Gleditzsch et al. 2019) below shows several Cas polypeptides and the PAM sequence they recognize.

TABLE A
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including LbCpf1 | TTTV |
| and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |
| Cas1 | 5′-CTT-3′ |
| Cas8e | 5′-ATG-3′ |
| Type I-A | 5′-CCN-3′ |
| Type I-B | TTC, ACT, TAA, TAT, TAG, and |
| | CAC |
| Type I-C | NTTC |
| Type I-E | 5′-AAG-3′ |
| TypeI-F | GG |

In a preferred embodiment, the CRISPR effector protein may recognize a 3′ PAM. In one example embodiment, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016.Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAIMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some embodiments, one or more components (e.g., the Cas protein) in the composition for engineering cells may comprise one or more sequences related to nucleus targeting and transportation. Such sequences may facilitate the one or more components in the composition for targeting a sequence within a cell. In order to improve targeting of the CRISPR-Cas protein used in the methods of the present disclosure to the nucleus, it may be advantageous to provide one or both of these components with one or more nuclear localization sequences (NLSs).

In one example embodiment, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO:1) or PKKKRKVEAS (SEQ ID NO:2); the NLS from nucleoplasmin (e.g., the nucleoplasmin bipartite NLS with the sequence KRPAATKKAGQAKKKK (SEQ ID NO:3)); the c-myc NLS having the amino acid sequence PAAKRVKLD (SEQ ID NO:4) or RQRRNELKRSP (SEQ ID NO:5); the hRNPA1 M9 NLS having the sequence NQSSNFGPMKGGNFGGRSSGPYGGGGQYFAKPRNQGGY (SEQ ID NO:6); the sequence RMRIZFKNKGKDTAELRRRRVEVSVELRKAKKDEQILKRRNV (SEQ ID NO:7) of the IBB domain from importin-alpha; the sequences VSRKRPRP (SEQ ID NO:8) and PPKKARED (SEQ ID NO:9) of the myoma T protein; the sequence PQPKKKPL (SEQ ID NO:10) of human p53; the sequence SALIKKKKKMAP (SEQ ID NO: 11) of mouse c-abl IV; the sequences DRLRR (SEQ ID NO:12) and PKQKKRK (SEQ ID NO:13) of the influenza virus NS1; the sequence RKLKKKIKKL (SEQ ID NO:14) of the Hepatitis virus delta antigen; the sequence REKKKFLKRR (SEQ ID NO:15) of the mouse Mxl protein; the sequence KRKGDEVDGVDEVAKKKSKK (SEQ ID NO:16) of the human poly(ADP-ribose) polymerase; and the sequence RKCLQAGMNLEARKTKK (SEQ ID NO:17) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the Cas protein, or exposed to a Cas protein lacking the one or more NLSs.

The Cas proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the Cas proteins, an NLS attached to the C-terminal of the protein.

### Zinc Finger Nucleases

Other preferred tools for genome editing for use in the context of this invention include zinc finger systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

Zinc Finger proteins can comprise a functional domain (e.g., activator domain). The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Talens

As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle EL. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church GM. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or ₃₄ or ₃₅, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or ₃₄ or ₃₅)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011). each of which is incorporated herein by reference in its entirety.

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in one example embodiment, the TALE polypeptides described herein further comprise an N-terminal capping region and/or a C-terminal capping region.

An exemplary amino acid sequence of a N-terminal
capping region is:
(SEQ ID NO: 18)
MDPIRSRTPSPARELLSGPQPDGVQPTADRGVSPPAGGP
LDGLPARRTMSRTRLPSPPAPSPAFSADSFSDLLRQFDPSLENTS
LFDSLPPFGAHHTEAATGEWDEVQSGLRAADAPPPTMRVAVTA
ARPPRAKPAPRRRAAQPSDASPAAQVDLRTLGYSQQQQEKIKP
KVRSTVAQHHEALVGHGFTHAHIVALSQHPAALGTVAVKYQD
MIAALPEATHEAIVGVGKQWSGARALEALLTVAGELRGPPLQL
DTGQLLKIAKRGGVTAVEAVHAWRNALTGAPLN
An exemplary amino acid sequence of a C-terminal
capping region is:
(SEQ ID NO: 19)
RPALESIVAQLSRPDPALAALTNDHLVALACLGGRPAL
DAVKKGLPHAPALIKRTNRRIPERTSHRVADHAQVVRVLGFFQ
CHSHPAQAFDDAMTQFGMSRHGLLQLFRRVGVTELEARSGTLP
PASQRWDRILQASGMKRAKPSPTSTQTPDQASLHAFADSLERD
LDAPSPMHEGDQTRAS

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in one example embodiment, fragments of the N-terminal and/or C-terminal capping regions are included in the TALE polypeptides described herein.

In one example embodiment, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190,200,210,220,230,240,250, 260 or 270 amino acids of an N-terminal capping region. In another example embodiment, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In one example embodiment, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In one example embodiment, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85% 90%, 91%, 92%, 93% 94%, 95% 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### Engineered Transcriptional Activators (CRISPRa)

In one example embodiment, a programmable nuclease system is used to recruit an activator protein to a target gene in order to enhance expression. In one example embodiment, the activator protein is recruited to the enhancer region of the target gene. For example, a catalytically inactive Cas protein (“dCas”) fused to an activator can be used to recruit that activator protein to the target sequence. Accordingly, a guide sequence is designed to direct binding of the dCas-activator fusion such that the activator can interact with the target genomic region and induce target gene expression. The Cas protein used may be any of the Cas proteins disclosed above. In one example protein, the Cas protein is a dCas9.

In one embodiment, the programmable nuclease system is a CRISPRa system (see, e.g., US20180057810A1; and Konermann et al. “Genome-scale transcriptional activation by an engineered CRISPR-Cas9 complex” Nature. 2014 Dec. 10. doi: 10.1038/nature14136). Numerous genetic variants associated with disease phenotypes are found to be in non-coding region of the genome, and frequently coincide with transcription factor (TF) binding sites and non-coding RNA genes. In one embodiment, a CRISPR system may be used to activate gene transcription. A nuclease-dead RNA-guided DNA binding domain, dCas9, tethered to transcriptional activator domains that promote gene activation (e.g., p65) may be used for “CRISPRa” that activates transcription. In one example embodiment, for use of dCas9 as an activator (CRISPRa), a guide RNA is engineered to carry RNA binding motifs (e.g., MS2) that recruit effector domains fused to RNA-motif binding proteins, increasing transcription. A key dendritic cell molecule, p65, may be used as a signal amplifier, but is not required.

In certain embodiments, one or more activator domains are recruited. In one example embodiment, the activation domain is linked to the CRISPR enzyme. In another example embodiment, the guide sequence includes aptamer sequences that bind to adaptor proteins fused to an activation domain. In general, the positioning of the one or more activator domains on the inactivated CRISPR enzyme or CRISPR complex is one which allows for correct spatial orientation for the activator domain to affect the target with the attributed functional effect. For example, the transcription activator is placed in a spatial orientation which allows it to affect the transcription of the target. This may include positions other than the N-/C-terminus of the CRISPR enzyme.

In another example embodiment, a zinc finger system is used to recruit an activation domain to the target gene. In one example embodiment, the activation domain is linked to the zinc finger system. In general, the positioning of the one or more activator domains on the zinc finger system is one which allows for correct spatial orientation for the activator domain to affect the target with the attributed functional effect.

In another example embodiment, a TALE system is used to recruit an activation domain to the target gene. In one example embodiment, the activation domain is linked to the TALE system. In general, the positioning of the one or more activator domains on the TALE system is one which allows for correct spatial orientation for the activator domain to affect the target with the attributed functional effect. For example, the transcription activator is placed in a spatial orientation which allows it to affect the transcription of the target.

In another example embodiment, a meganuclease system is used to recruit an activation domain to the target gene. In one example embodiment, the activation domain is linked to the meganuclease system. In general, the positioning of the one or more activator domains on the inactivated meganuclease system is one which allows for correct spatial orientation for the activator domain to affect the target with the attributed functional effect. For example, the transcription activator is placed in a spatial orientation which allows it to affect the transcription of the target.

### Base Editing

In one example embodiment, a method of treating subjects comprises administering a base editing system that is directed to a target gene (e.g., a regulator). A base-editing system may comprise a Cas polypeptide linked to a nucleobase deaminase (“base editing system”) and a guide molecule capable of forming a complex with the Cas polypeptide and directing sequence-specific binding of the base editing system at a target sequence. In one example embodiment, the Cas polypeptide is catalytically inactive. In another example embodiment, the Cas polypeptide is a nickase. The Cas polypeptide may be any of the Cas polypeptides disclosed above. In one example embodiment, the Cas polypeptide is a Type II Cas polypeptide. In one example embodiment, the Cas polypeptide is a Cas9 polypeptide. In another example embodiment, the Cas polypeptide is a Type V Cas polypeptide. In one example embodiment, the Cas polypeptide is a Cas12a or Cas12b polypeptide. The nucleobase deaminase may be cytosine base editor (CBE) or adenosine base editors (ABEs). CBEs convert C·G base pairs into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Example base editing systems are disclosed in Rees and Liu. 2018. Nat. Rev. Genet. 19(12): 770-788, particularly at FIGS. 1b, 2a-2c, 3a-3f, and Table 1, which is specifically incorporated herein by reference. In certain example embodiments, the base editing system may further comprise a DNA glycosylase inhibitor.

The editing window of a base editing system may range over a 5-8 nucleotide window, depending on the base editing system used. Id. Accordingly, given the base editing system used, a guide sequence may be selected to direct the base editing system to convert a base or base pair of one or more target genes.

### ARCUS Based Editing

In one example embodiment, a method of treating subjects comprises administering an ARCUS base editing system. Exemplary methods for using ARCUS can be found in U.S. Pat. No. 10,851,358, US Publication No. 2020-0239544, and WIPO Publication No. 2020/206231 which are incorporated herein by reference.

### Prime Editing

In one example embodiment, a method of treating subjects comprises administering a prime editing system directed to a target gene. In one example embodiment, a prime editing system comprises a Cas polypeptide having nickase activity, a reverse transcriptase, and a prime editing guide RNA (pegRNA). Cas polypeptide, and/or reverse transcriptase can be coupled together or otherwise associate with each other to form a prime editing complex and edit a target sequence. The Cas polypeptide may be any of the Cas polypeptides disclosed above. In one example embodiment, the Cas polypeptide is a Type II Cas polypeptide. In another example embodiment, the Cas polypeptide is a Cas9 nickase. In one example embodiment, the Cas polypeptide is a Type V Cas polypeptide. In another example embodiment, the Cas polypeptide is a Cas12a or Cas12b.

The prime editing guide molecule (pegRNA) comprises a primer binding site (PBS) configured to hybridize with a portion of a nicked strand on a target polynucleotide (e.g., genomic DNA) a reverse transcriptase (RT) template comprising the edit to be inserted in the genomic DNA and a spacer sequence designed to hybridize to a target sequence at the site of the desired edit. The nicking site is dependent on the Cas polypeptide used and standard cutting preference for that Cas polypeptide relative to the PAM. Thus, based on the Cas polypeptide used, a pegRNA can be designed to direct the prime editing system to introduce a nick where the desired edit should take place.

The pegRNA can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53,54,55, 56,57, 58,59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposases (CAST)

In one example embodiment, a method of treating a subject comprises administering a CAST system that replaces a genomic region in a target gene. In one example embodiment, a CAST system is used to replace all or a portion of an enhancer controlling target gene expression.

CAST systems comprise a Cas polypeptide, a guide sequence, a transposase, and a donor construct. The transposase is linked to or otherwise capable of forming a complex with the Cas polypeptide. The donor construct comprises a donor sequence to be inserted into a target polynucleotide and one or more transposase recognition elements. The transposase is capable of binding the donor construct and excising the donor template and directing insertion of the donor template into a target site on a target polynucleotide (e.g., genomic DNA). The guide molecule is capable of forming a CRISPR-Cas complex with the Cas polypeptide, and can be programmed to direct the entire CAST complex such that the transposase is positioned to insert the donor sequence at the target site on the target polynucleotide. For multimeric transposase, only those transposases needed for recognition of the donor construct and transposition of the donor sequence into the target polypeptide may be required. The Cas may be naturally catalytically inactive or engineered to be catalytically inactive.

In one example embodiment, the CAST system is a Tn7-like CAST system, wherein the transposase comprises one or more polypeptides from a Tn7 or Tn7-like transposase. The Cas polypeptide of the Tn7-like transposase may be a Class 1 (multimeric effector complex) or Class 2 (single protein effector) Cas polypeptide.

In one example embodiments, the Cas polypeptide is a Class 1 Type-if Cas polypeptide. In one example embodiment, the Cas polypeptide may comprise a cas6, a cas7, and a cas8-cas5 fusion. In one example embodiments, the Tn7 transposase may comprise TnsB, TnsC, and TniQ. In another example embodiment, the Tn7 transposase may comprise TnsB, TnsC, and TnsD. In certain example embodiments, the Tn7 transposase may comprise TnsD, TnsE, or both. As used herein, the terms “TnsAB”, “TnsAC”, “TnsBC”, or “TnsABC” refer to a transponson complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein. An example Type 1f-Tn7 CAST system is described in Klompe et al. Nature, 2019, 571:219-224 and Vo et al. bioRxiv, 2021, doi.org/10.1101/2021.02.11.430876, which are incorporated herein by reference.

In one example embodiment, the Cas polypeptide is a Class 1 Type-lb Cas polypeptide. In one example embodiment, the Cas polypeptide may comprise a cas6, a cas7, and a cas8b (e.g., a ca8b3). In one example embodiments, the Tn7 transposase may comprise TnsB, TnsC, and TniQ. In another example embodiment, the Tn7 transposase may comprise TnsB, TnsC, and TnsD. In certain example embodiments, the Tn7 transposase may comprise TnsD, TnsE, or both. As used herein, the terms “TnsAB”, “TnsAC”, “TnsBC”, or “TnsABC” refer to a transponson complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein.

In one example embodiment, the Cas polypeptide is Class 2, Type V Cas polypeptide. In one example embodiment, the Type V Cas polypeptide is a Cas12k. In one example embodiments, the Tn7 transposase may comprise TnsB, TnsC, and TniQ. In another example embodiment, the Tn7 transposase may comprise TnsB, TnsC, and TnsD. In certain example embodiments, the Tn7 transposase may comprise TnsD, TnsE, or both. As used herein, the terms “TnsAB”, “TnsAC”, “TnsBC”, or “TnsABC” refer to a transponson complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein. An example Cas12k-Tn7 CAST system is described in Strecker et al. Science, 2019 365:48-53, which is incorporated herein by reference.

In one example embodiment, the CAST system is a Mu CAST system, wherein the transposase comprises one or more polypeptides of a Mu transposase. An example Mu CAST system is disclosed in WO/2021/041922 which is incorporated herein by reference.

In one example embodiment, the CAST comprise a catalytically inactive Type II Cas polypeptide (e.g., dCas9) fused to one or more polypeptides of a Tn5 transposase. In another example embodiment, the CAST system comprises a catalytically inactive Type II Cas polypeptide (e.g. dCas9) fused to a piggyback transposase.

### Epigenetic Editing

In example embodiments, the one or more agents is an epigenetic modification polypeptide comprising a DNA binding domain linked to or otherwise capable of associating with an epigenetic modification domain such that binding of the DNA binding domain at target sequence on genomic DNA (e.g., chromatin) results in one or more epigenetic modifications by the epigenetic modification domain that increases or decreases expression of the one or more polypeptides. As used herein, “linked to or otherwise capable of associating with” refers to a fusion protein or a recruitment domain or an adaptor protein, such as an aptamer (e.g., MS2) or an epitope tag. The recruitment domain or the adaptor protein can be linked to an epigenetic modification domain or the DNA binding domain (e.g., an adaptor for an aptamer). The epigenetic modification domain can be linked to an antibody specific for an epitope tag fused to the DNA binding domain. An aptamer can be linked to a guide sequence.

In example embodiments, the DNA binding domain is a programmable DNA binding protein linked to or otherwise capable of associating with an epigenetic modification domain. Programmable DNA binding proteins for modifying the epigenome include, but are not limited to CRISPR systems, transcription activator-like effectors (TALEs), Zn finger proteins and meganucleases (see, e.g., Thakore P I, Black J B, Hilton I B, Gersbach C A. Editing the epigenome: technologies for programmable transcription and epigenetic modulation. Nat Methods. 2016; 13(2):127-137; and described further herein). In example embodiments, the DNA binding domain is a nuclease-deficient RNA-guided DNA endonuclease enzyme or a nuclease-deficient endonuclease enzyme. In example embodiments, a CRISPR system having an inactivated nuclease activity (e.g., dCas) is used as the DNA binding domain.

In example embodiments, the epigenetic modification domain is a functional domain and includes, but is not limited to a histone methyltransferase (HMT) domain, histone demethylase domain, histone acetyltransferase (HAT) domain, histone deacetylation (HDAC) domain, DNA methyltransferase domain, DNA demethylation domain, histone phosphorylation domain (e.g., serine and threonine, or tyrosine), histone ubiquitylation domain, histone sumoylation domain, histone ADP ribosylation domain, histone proline isomerization domain, histone biotinylation domain, histone citrullination domain (see, e.g., Epigenetics, Second Edition, 2015, Edited by C. David Allis; Marie-Laure Caparros; Thomas Jenuwein; Danny Reinberg; Associate Editor Monika Lachlan; Dawson M A, Kouzarides T. Cancer epigenetics: from mechanism to therapy. Cell. 2012; 150(1):12-27; Syding L A, Nickl P, Kasparek P, Sedlacek R. CRISPR/Cas9 Epigenome Editing Potential for Rare Imprinting Diseases: A Review. Cells. 2020; 9(4):993; and Zhang Y. Transcriptional regulation by histone ubiquitination and deubiquitination. Genes Dev. 2003; 17(22):2733-2740). Example epigenetic modification domains can be obtained from, but are not limited to chromatin modifying enzymes, such as, DNA methyltransferases (e.g., DNMT1, DNMT3a and DNMT3b), TETI, TET2, thymine-DNA glycosylase (TDG), GCN5-related N-acetyltransferases family (GNAT), MYST family proteins (e.g., MOZ and MORF), and CBP/p300 family proteins (e.g., CBP, p300), Class I HDACs (e.g., HDAC 1-3 and HDAC8), Class II HDACs (e.g., HDAC 4-7 and HDAC 9-10), Class III HDACs (e.g., sirtuins), HDAC11, SET domain containing methyltransferases (e.g., SET7/9 (KMT7, NCBI Entrez Gene: 80854), KMT5A (SET8), MMSET, EZH2, and MLL family members), DOT1L, LSD1, Jumonji demethylases (e.g., KDM5A (JARID1A), KDM5C (JARIDIC), and KDM6A (UTX)), kinases (e.g., Haspin, VRK1, PKCα, PKCβ, PIM1, IKKα, Rsk2, PKB/Akt, Aurora B, MSK1/2, JNK1, MLTKα, PRK1, Chk1, Dlk/ZIP, PKCδ, MST1, AMPK, JAK2, Ab1, BMK1, CaMK, S6K1, SIK1), Ubp8, ubiquitin C-terminal hydrolases (UCH), the ubiquitin-specific processing proteases (UBP), and poly(ADP-ribose) polymerase 1 (PARP-1). See, also, US Patent U.S. Ser. No. 11/001,829B2 for additional domains.

In example embodiments, histone acetylation is targeted to a target sequence using a CRISPR system (see, e.g., Hilton I B, et al. Epigenome editing by a CRISPR-Cas9-based acetyltransferase activates genes from promoters and enhancers. Nat Biotechnol. 2015). In example embodiments, histone deacetylation is targeted to a target sequence (see, e.g., Cong et al., 2012; and Konermann S, et al. Optical control of mammalian endogenous transcription and epigenetic states. Nature. 2013; 500:472-476). In example embodiments, histone methylation is targeted to a target sequence (see, e.g., Snowden A W, Gregory P D, Case C C, Pabo C O. Gene-specific targeting of H3K9 methylation is sufficient for initiating repression in vivo. Curr Biol. 2002; 12:2159-2166; and Cano-Rodriguez D, Gjaltema R A, Jilderda L J, et al. Writing of H3K4Me3 overcomes epigenetic silencing in a sustained but context-dependent manner. Nat Commun. 2016; 7:12284). In example embodiments, histone demethylation is targeted to a target sequence (see, e.g., Kearns N A, Pham H, TabakB, et al. Functional annotation of native enhancers with a Cas9-histone demethylase fusion. Nat Methods. 2015; 12(5):401-403). In example embodiments, histone phosphorylation is targeted to a target sequence (see, e.g., Li J, Mahata B, Escobar M, et al. Programmable human histone phosphorylation and gene activation using a CRISPR/Cas9-based chromatin kinase. Nat Commun. 2021; 12(1):896). In example embodiments, DNA methylation is targeted to a target sequence (see, e.g., Rivenbark A G, et al. Epigenetic reprogramming of cancer cells via targeted DNA methylation. Epigenetics. 2012; 7:350-360; Siddique A N, et al. Targeted methylation and gene silencing of VEGF-A in human cells by using a designed Dnmt3a-Dnmt3L single-chain fusion protein with increased DNA methylation activity. J Mol Biol. 2013; 425:479-491; Bernstein D L, Le Lay J E, Ruano E G, Kaestner K H. TALE-mediated epigenetic suppression of CDKN2A increases replication in human fibroblasts. J Clin Invest. 2015; 125:1998-2006; Liu X S, Wu H, Ji X, et al. Editing DNA Methylation in the Mammalian Genome. Cell. 2016; 167(1):233-247.e17; Stepper P, Kungulovski G, Jurkowska R Z, et al. Efficient targeted DNA methylation with chimeric dCas9-Dnmt3a-Dnmt3L methyltransferase. Nucleic Acids Res. 2017; 45(4):1703-1713; and Pflueger C., Tan D., Swain T., Nguyen T., Pflueger J., Nefzger C., Polo J. M., Ford E., Lister R. A modular dCas9-SunTag DNMT3A epigenome editing system overcomes pervasive off-target activity of direct fusion dCas9-DNMT3A constructs. Genome Res. 2018; 28:1193-1206). In example embodiments, DNA demethylation is targeted to a target sequence using a CRISPR system (see, e.g., TET1, see Xu et al, Cell Discov. 2016 May 3; 2: 16009; Choudhury et al, Oncotarget. 2016 Jul. 19; 7(29):46545-46556; and Kang J G, Park J S, Ko J H, Kim Y S. Regulation of gene expression by altered promoter methylation using a CRISPR/Cas9-mediated epigenetic editing system. Sci Rep. 2019; 9(1):11960). In example embodiments, DNA demethylation is targeted to a target sequence (see, e.g., TDG, see, Gregory D J, Zhang Y, Kobzik L, Fedulov A V. Specific transcriptional enhancement of inducible nitric oxide synthase by targeted promoter demethylation. Epigenetics. 2013; 8:1205-1212).

Example epigenetic modification domains can be obtained from, but are not limited to transcription activators, such as, VP64 (see, e.g., Ji Q, et al. Engineered zinc-finger transcription factors activate OCT4 (POU5F1), SOX2, KLF4, c-MYC (MYC) and miR302/367. Nucleic Acids Res. 2014; 42:6158-6167; Perez-Pinera P, et al. Synergistic and tunable human gene activation by combinations of synthetic transcription factors. Nat Methods. 2013; 10:239-242; Farzadfard F, Perli S D, Lu T K. Tunable and multifunctional eukaryotic transcription factors based on CRISPR/Cas. ACS Synth Biol. 2013; 2:604-613; Black J B, Adler A F, Wang H G, et al. Targeted Epigenetic Remodeling of Endogenous Loci by CRISPR/Cas9-Based Transcriptional Activators Directly Converts Fibroblasts to Neuronal Cells. Cell Stem Cell. 2016; 19(3):406-414; and Maeder M L, Linder S J, Cascio V M, Fu Y, Ho Q H, Joung J K. CRISPR RNA-guided activation of endogenous human genes. Nat Methods. 2013; 10(10):977-979), p65 (see, e.g., Liu P Q, et al. Regulation of an endogenous locus using a panel of designed zinc finger proteins targeted to accessible chromatin regions. Activation of vascular endothelial growth factor A. J Biol Chem. 2001; 276:11323-11334; and Konermann S, et al. Genome-scale transcriptional activation by an engineered CRISPR-Cas9 complex. Nature. 2015; 517:583-588), HSF1, and RTA (see, e.g., Chavez A, et al. Highly efficient Cas9-mediated transcriptional programming. Nat Methods. 2015; 12:326-328). Example epigenetic modification domains can be obtained from, but are not limited to transcription repressors, such as, KRAB (see, e.g., Beerli R R, Segal D J, Dreier B, Barbas C F., 3rd Toward controlling gene expression at will: specific regulation of the erbB-2/HER-2 promoter by using polydactyl zinc finger proteins constructed from modular building blocks. Proc Natl Acad Sci USA. 1998; 95:14628-14633; Cong L, Zhou R, Kuo Y C, Cunniff M, Zhang F. Comprehensive interrogation of natural TALE DNA-binding modules and transcriptional repressor domains. Nat Commun. 2012; 3:968; Gilbert L A, et al. CRISPR-mediated modular RNA-guided regulation of transcription in eukaryotes. Cell. 2013; 154:442-451; and Yeo N C, Chavez A, Lance-Byrne A, et al. An enhanced CRISPR repressor for targeted mammalian gene regulation. Nat Methods. 2018; 15(8):611-616).

In example embodiments, the epigenetic modification domain linked to a DNA binding domain recruits an epigenetic modification protein to a target sequence. In example embodiments, a transcriptional activator recruits an epigenetic modification protein to a target sequence. For example, VP64 can recruit DNA demethylation, increased H3K27ac and H3K4me. In example embodiments, a transcriptional repressor protein recruits an epigenetic modification protein to a target sequence. For example, KRAB can recruit increased H3K9me3 (see, e.g., Thakore P I, D'Ippolito A M, Song L, et al. Highly specific epigenome editing by CRISPR-Cas9 repressors for silencing of distal regulatory elements. Nat Methods. 2015; 12(12):1143-1149). In an example embodiment, methyl-binding proteins linked to a DNA binding domain, such as MBD1, M1BD2, MBD3, and MeCP2 recruits an epigenetic modification protein to a target sequence. In an example embodiment, Mi2/NuRD, Sin3A, or Co-REST recruit HDACs to a target sequence.

In example embodiments, the epigenetic modification domain can be a eukaryotic or prokaryotic (e.g., bacteria or Archaea) protein. In example embodiments, the eukaryotic protein can be a mammalian, insect, plant, or yeast protein and is not limited to human proteins (e.g., a yeast, insect, plant chromatin modifying protein, such as yeast HATs, HDACs, methyltransferases, etc.

In one aspect of the invention, is provided a fusion protein (epigenetic modification polypeptide) comprising from N-terminus to C-terminus, an epigenetic modification domain, an XTEN linker, and a nuclease-deficient RNA-guided DNA endonuclease enzyme or a nuclease-deficient endonuclease enzyme.

In aspects, the epigenetic modification polypeptide further comprises a transcriptional activator. In aspects, the transcriptional activator is VP64, p65, RTA, or a combination of two or more thereof. In another aspect, the epigenetic modification polypeptide further comprises one or more nuclear localization sequences. In embodiments, the epigenetic modification polypeptide comprises the nuclease-deficient RNA-guided DNA endonuclease enzyme. In embodiments, the fusion protein comprises the nuclease-deficient DNA endonuclease enzyme.

In some embodiments, the functional domains associated with the adaptor protein or the CRISPR enzyme is a transcriptional activation domain comprising VP64, p65, MyoD1, HSF1, RTA or SET7/9. Other references herein to activation (or activator) domains in respect of those associated with the adaptor protein(s) include any known transcriptional activation domain and specifically VP64, p65, MyoD1, HSF1, RTA or SET7/9 (see, e.g., US patent, U.S. Ser. No. 11/001,829B2).

In certain embodiments, the present invention provides a fusion protein comprising from N-terminus to C-terminus, an RNA-binding sequence, an XTEN linker, and a transcriptional activator. In aspects, the transcriptional activator is VP64, p65, RTA, or a combination of two or more thereof. In aspects, the fusion protein further comprises a demethylation domain, a nuclease-deficient RNA-guided DNA endonuclease enzyme or a nuclease-deficient endonuclease enzyme, a nuclear localization sequence, or a combination of two or more thereof. In embodiments, the fusion protein comprises the nuclease-deficient RNA-guided DNA endonuclease enzyme. In embodiments, the fusion protein comprises the nuclease-deficient DNA endonuclease enzyme.

In certain embodiments, the present invention provides a method of activating a target nucleic acid sequence in a cell, the method comprising: (i) delivering a first polynucleotide encoding a epigenetic modification polypeptide described herein including embodiments thereof to a cell containing the silenced target nucleic acid; and (ii) delivering to the cell a second polynucleotide comprising: (a) a sgRNA or (b) a cr:tracrRNA; thereby reactivating the silenced target nucleic acid sequence in the cell. In aspects, the sgRNA comprises at least one MS2 stem loop. In aspects, the second polynucleotide comprises a transcriptional activator. In aspects, the second polynucleotide comprises two or more sgRNA.

### Donor Polynucleotides

The system may further comprise one or more donor polynucleotides (e.g., for insertion into the target polynucleotide). A donor polynucleotide may be an equivalent of a transposable element that can be inserted or integrated to a target site. The donor polynucleotide may be or comprise one or more components of a transposon. A donor polynucleotide may be any type of polynucleotides, including, but not limited to, a gene, a gene fragment, a non-coding polynucleotide, a regulatory polynucleotide, a synthetic polynucleotide, etc. The donor polynucleotide may include a transposon left end (LE) and transposon right end (RE). The LE and RE sequences may be endogenous sequences for the CAST used or may be heterologous sequences recognizable by the CAST used, or the LE or RE may be synthetic sequences that comprise a sequence or structure feature recognized by the CAST and sufficient to allow insertion of the donor polynucleotide into the target polynucleotides. In certain example embodiments, the LE and RE sequences are truncated. In certain example embodiments may be between 100-200 bps, between 100-190 base pairs, 100-180 base pairs, 100-170 base pairs, 100-160 base pairs, 100-150 base pairs, 100-140 base pairs, 100-130 base pairs, 100-120 base pairs, 100-110 base pairs, 20-100 base pairs, 20-90 base pairs, 20-80 base pairs, 20-70 base pairs, 20-60 base pairs, 20-50 base pairs, 20-40 base pairs, 20-30 base pairs, 50 to 100 base pairs, 60-100 base pairs, 70-100 base pairs, 80-100 base pairs, or 90-100 base pairs in length.

The donor polynucleotide may be inserted at a position upstream or downstream of a PAM on a target polynucleotide. In some embodiments, a donor polynucleotide comprises a PAM sequence. Examples of PAM sequences include TTTN, ATTN, NGTN, RGTR, VGTD, or VGTR.

The donor polynucleotide may be inserted at a position between 10 bases and 200 bases, e.g., between 20 bases and 150 bases, between 30 bases and 100 bases, between 45 bases and 70 bases, between 45 bases and 60 bases, between 55 bases and 70 bases, between 49 bases and 56 bases or between 60 bases and 66 bases, from a PAM sequence on the target polynucleotide. In some cases, the insertion is at a position upstream of the PAM sequence. In some cases, the insertion is at a position downstream of the PAM sequence. In some cases, the insertion is at a position from 49 to 56 bases or base pairs downstream from a PAM sequence. In some cases, the insertion is at a position from 60 to 66 bases or base pairs downstream from a PAM sequence.

The donor polynucleotide may be used for editing the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the donor polynucleotide alters a stop codon in the target polynucleotide. For example, the donor polynucleotide may correct a premature stop codon. The correction may be achieved by deleting the stop codon or introduces one or more mutations to the stop codon. In other example embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g., sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of a corresponding wild-type gene. In certain example embodiments, these defective genes may be associated with one or more disease phenotypes. In certain example embodiments, the defective gene or gene fragment is not replaced but the systems described herein are used to insert donor polynucleotides that encode gene or gene fragments that compensate for or override defective gene expression such that cell phenotypes associated with defective gene expression are eliminated or changed to a different or desired cellular phenotype.

In certain embodiments of the invention, the donor may include, but not be limited to, genes or gene fragments, encoding proteins or RNA transcripts to be expressed, regulatory elements, repair templates, and the like. According to the invention, the donor polynucleotides may comprise left end and right end sequence elements that function with transposition components that mediate insertion.

In certain cases, the donor polynucleotide manipulates a splicing site on the target polynucleotide. In some examples, the donor polynucleotide disrupts a splicing site. The disruption may be achieved by inserting the polynucleotide to a splicing site and/or introducing one or more mutations to the splicing site. In certain examples, the donor polynucleotide may restore a splicing site. For example, the polynucleotide may comprise a splicing site sequence.

The donor polynucleotide to be inserted may have a size from 10 bases to 50 kb in length, e.g., from 50 to 40 kb, from 100 to 30 kb, from 100 bases to 300 bases, from 200 bases to 400 bases, from 300 bases to 500 bases, from 400 bases to 600 bases, from 500 bases to 700 bases, from 600 bases to 800 bases, from 700 bases to 900 bases, from 800 bases to 1000 bases, from 900 bases to from 1100 bases, from 1000 bases to 1200 bases, from 1100 bases to 1300 bases, from 1200 bases to 1400 bases, from 1300 bases to 1500 bases, from 1400 bases to 1600 bases, from 1500 bases to 1700 bases, from 600 bases to 1800 bases, from 1700 bases to 1900 bases, from 1800 bases to 2000 bases, from 1900 bases to 2100 bases, from 2000 bases to 2200 bases, from 2100 bases to 2300 bases, from 2200 bases to 2400 bases, from 2300 bases to 2500 bases, from 2400 bases to 2600 bases, from 2500 bases to 2700 bases, from 2600 bases to 2800 bases, from 2700 bases to 2900 bases, or from 2800 bases to 3000 bases in length.

The components in the systems herein may comprise one or more mutations that alter their (e.g., the transposase(s)) binding affinity to the donor polynucleotide. In some examples, the mutations increase the binding affinity between the transposase(s) and the donor polynucleotide. In certain examples, the mutations decrease the binding affinity between the transposase(s) and the donor polynucleotide. The mutations may alter the activity of the Cas and/or transposase(s).

In certain embodiments, the systems disclosed herein are capable of unidirectional insertion, that is the system inserts the donor polynucleotide in only one orientation.

Delivery mechanisms for CAST systems includes those discussed above for CRISPR-Cas systems.

### Treating Disease Subtypes

The invention further relates to administering agents capable of inducing or suppressing particular pathways underlying a disease based on the genetically anchored signature (e.g., gene signatures, morphological signature, protein signature, and/or other genetic or epigenetic signature as defined herein), as well as their use for modulating, such as inducing or repressing, a particular genetically anchored signature. In example embodiments, the treatment is monitored by detecting expression of the genetically anchored signature during treatment.

In example embodiments, therapeutic agents that decrease a signature associated with a high risk score is administered, such as to reverse the signature (e.g., the signatures negatively correlate). In example embodiments, therapeutic agents that increase a signature associated with a low risk score is administered, such as to increase the signature (e.g., the signatures positively correlate). In example embodiments, subjects having a high risk endotype are treated with a therapeutic agent targeting a genetically-anchored molecular signature associated with the endotype. Thus, a treatment can be tailored to subjects having a specific endotype. In example embodiments, the methods comprise treating a subject having a high polygenic risk score for a disease with one or more agents capable of modifying the genetically anchored signature.

In example embodiments, the disease treated is type 2 diabetes (T2D). In example embodiments, the gene signature is linked to a beta-cell polygenic risk score and the one or more agents comprise ranolazine. In example embodiments, the gene signature is linked to a beta-cell polygenic risk score comprising variants active in adipocytes and associated with beta-cell traits; and wherein the one or more agents comprise a sulfonylurea (SU), such as glipizide. In example embodiments, the gene signature is linked to a lipodystrophy polygenic risk score comprising variants active in adipocytes and associated with lipodystrophy traits; and wherein the one or more agents comprise a thiazolidinedione, such as ciglitazone.

Further embodiments are illustrated in the following Examples which are given for illustrative purposes only and are not intended to limit the scope of the invention.

### EXAMPLES

### Example 1—High-Throughput Genetic Clustering of Type 2 Diabetes Loci Reveals Heterogeneous Mechanistic Pathways of Metabolic Disease

Diabetes is one of the most common complex diseases, afflicting approximately 460 million adults worldwide, with prevalence estimated to more than double by 2045. Type 2 diabetes (T2D) accounts for more than 90% of diabetes [1], with variable contributions of insulin resistance and beta cell dysfunction, and influenced by multiple risk factors, including genetics [2]. Hence, untangling the heterogeneity of T2D may be fundamental to improving patient management and facilitating precision medicine.

Hundreds of loci associated with T2D have been identified in large-scale genetic studies, including genome-wide association studies (GWAS), accounting for approximately 20% of the variance in individual predisposition to T2D [3,4,5]; however, the translation of these established T2D genetic loci into improved understanding of disease pathophysiology and clinical relevance has been challenging, due in large part to the non-coding nature and the small effect sizes of the genetic variants. Recent studies have leveraged a growing number of available GWAS datasets to connect genetic loci to mechanistic pathways by clustering loci based on shared patterns of associations across multiple traits. In previous work [6], clustering was performed on 94 T2D variants identified by manual curation of published T2D GWAS manuscripts. Soft clustering analysis with Bayesian Non-negative Matrix Factorization (bNMF) of the associations of these 94 T2D variants with 47 diabetes-related traits identified five distinct clusters, recognizable as biological pathways of T2D. A similar set of five clusters of T2D loci were independently identified by Mahajan et al., along with a sixth cluster of “undetermined” physiological impact [3,7]. Of these five shared clusters, two related to beta-cell dysfunction, and the other three clusters represented different mechanisms of insulin resistance: obesity-mediated, abnormal lipodystrophy-like fat distribution, and altered hepatic lipid metabolism [3].

With new T2D loci continuously being discovered and additional GWAS trait summary statistics becoming publicly available, Applicants sought to expand prior work which involved manual curation of a smaller set of T2D loci. Applicants developed a high-throughput pipeline to enable extraction of hundreds of genetic variants and traits from multiple GWAS to be used for cluster analysis in order to identify new genetic pathways of disease.

### Results

### Ten T2D Genetic Clusters Identified by High-Throughput Approach

Applicants employed a high-throughput pipeline to enable extraction of loci from GWAS summary statistics files and generate a variant-trait association input matrix for clustering analysis (FIG. 5). The pipeline started with summary statistics from 13 T2D GWAS studies available in the AMP-CMDKP [9], from which Applicants extracted 21,666 variants associated with T2D reaching genome-wide significance. After performing stringent LD-pruning and optimizing variants for inclusion in the variant-trait association matrix (see Materials and methods), 336 variants remained, representing independent T2D risk loci. For these 336 variants, Applicants extracted summary statistics for traits linked to T2D from 75 GWAS studies of measures of glycemic, anthropometric, and laboratory traits as well as vital signs, all housed in either the AMP-CMDKP [9] or publicly available analysis of the UK Biobank [17]. Applicants let the T2D genetics guide selection of 64 relevant traits to include in subsequent cluster analysis, such that each trait was significantly associated with at least one T2D variant. Soft clustering of the resulting 336 by 64 variant-trait association matrix was performed using bNMF.

The plurality of bNMF iteration results converged on ten clusters (34.3%), which captured the five clusters identified in the previous work [6]as well as five novel clusters (Table 5, 6). The remaining bNMF iterations converged on nested clusters, with 6 clusters in 0.4%, 7 clusters in 2.2%, 8 clusters in 12%, 9 clusters in 29.7%, 11 clusters in 18.6%, 12 clusters in 2.6% and 13 clusters in 0.2%. The same six clusters (Beta-cell 1, Beta-cell 2, Proinsulin, Obesity, Lipodystrophy, Liver/Lipid, as described below) were identified in 100% of iterations, based on inspection of constituent variants and traits. This high-throughput approach therefore robustly replicated clusters previously identified via a manual approach [6].

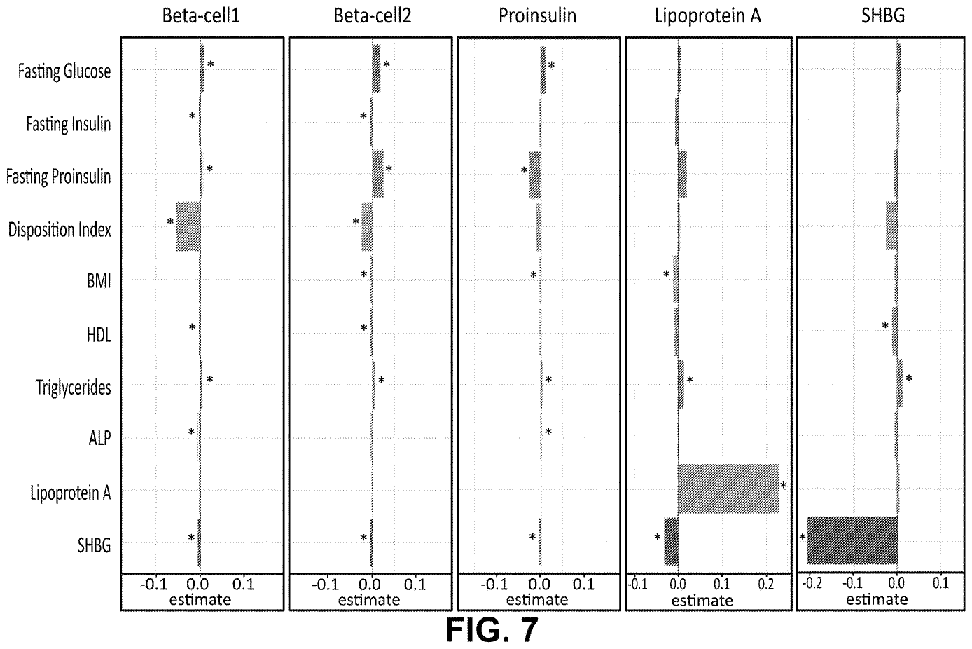

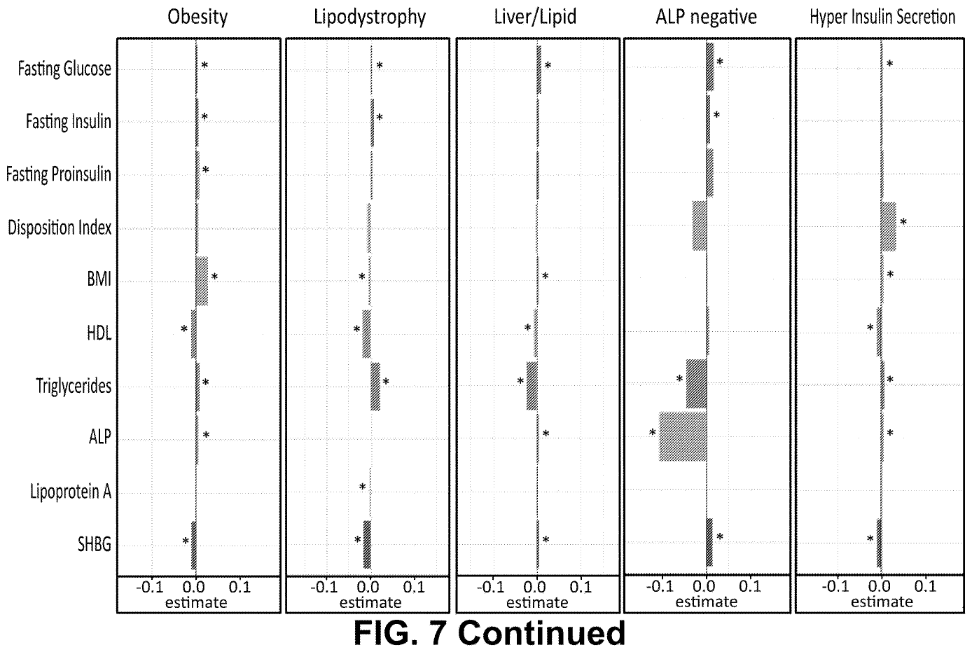

The clusters were named after their most defining traits. To further interpret these clusters, Applicants examined their most highly weighted loci and traits, as well as the aggregate associations of cluster loci with the traits via GWAS pPS (Table 16, Table 7, FIG. 7). In four of the clusters (Beta-cell 1, Beta-cell 2, Proinsulin, and Lipoprotein A), the T2D risk-increasing alleles were associated with reduced fasting insulin and reduced homeostatic assessment of beta cell function (HOMA-B; GWAS pPS P-values<0.05), consistent with pathways related to insulin deficiency (FIG. 1). Another five clusters (Obesity, Lipodystrophy, Liver/Lipid, ALP negative, Hyper Insulin Secretion) reflected mechanisms of impaired insulin action, with the T2D risk alleles in these clusters associated with increased fasting insulin and homeostatic assessment of insulin resistance (HOMA-IR; GWAS pPS P-values<0.05). The remaining cluster (SHBG) was driven by one T2D allele which was not significantly associated with fasting insulin, but trended toward increased levels (Table 16, FIG. 1, FIG. 7, Table 7).

Of the four clusters related to insulin deficiency (Beta-cell 1, Beta-cell 2, Proinsulin, Lipoprotein A), Beta-cell 1 and Beta-cell 2 appeared to be a combination of the single Beta-cell cluster in the previous work [6], both including several well-known loci and traits related to pancreatic beta cell function, indicating mechanisms of beta cell dysfunction leading to T2D. In Beta-cell 1, the top-weighted traits were decreased corrected insulin response (CIR) and decreased disposition index (DI) (Table 4); the most strongly weighted loci included known beta cell loci MTNR1B, CDKAL1, HHEX, C2CD4A, ANK1, ST6GAL1, SLC35C1, and SLC30A8 [34](Table 16, Table 5, Table 6). In Beta-cell 2 cluster, the top-weighted traits included increased fasting proinsulin adjusted for fasting insulin, and reduced HOMA-B and fasting insulin; TCF7L2, SLC30A8, ADCY5, GCK, DGKB, C2CD4A and MTNR1B were among the top-weighted loci (Table 4, 5).

The Beta-cell 1 and Beta-cell 2 clusters differed from each other with regard to the magnitude of glycemic trait effects, with Beta-cell 1 (N loci=66) having more marked association with reduced DI (beta=−0.05, P=7.36×10⁻⁶³ vs beta=−0.02, P=8.98×10⁻⁷), whereas Beta-cell 2 (N loci=27) had a more marked association with increased fasting proinsulin adjusted for fasting insulin (beta=0.02, P=3.42×10⁻⁴² vs beta=0.006, P=1.97×10⁻⁷) and fasting glucose (beta=0.02, P=6.67×10⁻⁸⁸ vs beta=0.008, P=4.64×10⁻⁴¹) (FIG. 1, Table 7). Proinsulin is a prohormone precursor to insulin, and elevated fasting proinsulin levels relative to fasting insulin levels indicates defective proinsulin processing, particularly related to beta cell stress [36,37]. The stronger association with increased proinsulin levels for Beta-cell 2 vs Beta-cell 1 could therefore indicate that Beta-cell 2 relates more specifically to beta cell stress.

The Proinsulin cluster, also identified in the previous work, had top-weighted traits of decreased fasting proinsulin adjusted for fasting insulin and reduced HOMA-B (Table 4, 5). The top-weighted loci included distinct signals in the ARAP1/STARD10 locus; beta cell-selective deletion of StarD10 in mice has previously been shown to cause impaired insulin secretion [38]. In contrast to the other insulin deficiency clusters, the Proinsulin cluster (N loci=18) was significantly associated with decreased fasting proinsulin adjusted for fasting insulin (GWAS pPS P=3.51×10⁻³⁶) (FIG. 1, Table 7), indicating a mechanism of lack of proinsulin substrate for insulin synthesis.

The Lipoprotein A cluster was novel to the present analysis and had a single highly weighted trait, increased serum lipoprotein A (Lp(a)), and a single highly weighted locus, SLC22A3 LPA (Table 4, 5). SLC22A3 LPA has been previously associated with serum Lp(a)levels [39] and contains the gene LPA encoding Lp(a). The T2D-risk-increasing allele of Lipoprotein A cluster variant (rs487152) was strongly associated with increased Lp(a) levels in the UK Biobank (GWAS pPS P=4.06×10⁻¹⁵⁸⁶) (Table 7), but the underlying mechanism relating to insulin deficiency is unknown.

Of the five clusters related to mechanisms of insulin response (Obesity, Lipodystrophy, Liver/Lipid, Hyper Insulin Secretion, ALP negative), three (Obesity, Lipodystrophy, and Liver/Lipid) were also identified in the previous work, but gained additional loci (and traits) in this expanded analysis.

The Obesity cluster had most-strongly weighted traits of increased body mass index (BMI), waist circumference, percent body fat, and C-reactive protein (CRP), and key genetic signals included the well-known obesity loci FTO and MC4R [40](Table 4, 5). GWAS pPS for the Obesity cluster (N loci=37) identified significant associations with increased fasting insulin (P=5.12×10⁻²¹), HOMA-IR (P=1.11×10⁻¹⁷), BMI (P=5.41×10⁻¹⁴⁶³), percent body fat (P=4.36×10⁻⁸⁶) and CRP (P=5.63×10⁻²⁵⁷), supporting a mechanism of obesity-mediated insulin resistance.

The Lipodystrophy cluster had top-weighted traits and loci suggestive of “lipodystrophy-like” or fat distribution-mediated insulin resistance as in the prior work [6]; these included decreased adiponectin, HDL cholesterol, and modified Stumvoll insulin sensitivity index (adjusted for age, sex and BMI), and increased triglycerides and waist-hip ratio, as well as top-weighted loci IRS1, KLF14, and PPARG [41,42](Table 4, 5). The Lipodystrophy cluster (N loci=54) was associated with increased fasting insulin (P=8.85×10⁻⁴³), HOMA-IR (P=9.08×10⁻²⁷) and triglycerides (P=5.78×10⁻⁶¹⁸), decreased insulin sensitivity index (P=5.26×10⁻³⁸) and HDL (P=2.91×10⁻⁵¹³)

The Liver/Lipid cluster, also identified in the previous work [6], was defined by decreased triglycerides and gamma-glutamyl transferase levels, and multiple loci previously connected to hepatic lipid or glycogen metabolism, including GCKR, HNF1A, PPP1R3B, TOMM40/APOE, and PNPLA3 (Table 4, 5) [43,44,45,46,47,48]. GWAS pPS for this cluster (N loci=12) were associated with reduced triglycerides (P=2.72×10⁻¹⁷⁰) and interestingly also reduced CRP (P=1.34×10⁻⁹⁹) and white blood cell count (P=1.25×10⁻⁸⁹).

The two remaining insulin response clusters were novel (labeled ALP negative and Hyper Insulin Secretion). The ALP negative cluster had decreased alkaline phosphatase (ALP) level as its top-weighed trait, and the ABO locus as the top-weighted locus (Table 4, 5). GWAS pPS in this cluster (N loci=4) was associated with decreased serum ALP (P=1.97×10⁻¹⁴³¹) and triglycerides (P=4.49×10⁻²⁴⁷). The ABO locus has previously been connected to T2D risk, but the mechanism is unknown: ABO gene knock-out in a murine pancreatic beta-cell line has been shown to alter insulin secretion [49], and there may be differential risk of T2D by ABO blood group [50,51]. The Hyper Insulin Secretion cluster included top-weighted traits of increased DI and CIR, and loci PPP1R3B, CNTN2, DTNB, SREBFJ, and TNF (Table 4, 5). The Hyper Insulin Secretion GWAS pPS (N loci=32) was associated with increased CIR (P=9.49×10⁻¹⁵), DI (P=2.02×10⁻¹⁴), BMI (P=2.58×10⁻²⁹), and reduced HDL (P=2.92×10⁻⁹⁸) and SHBG (P=5.14×10⁻⁹⁵)

The final cluster, labeled SHBG, was novel to the current work and not significantly associated with fasting insulin (GWAS pPS P=0.36). The cluster was driven by a single trait and locus: decreased SHBG levels and the SHBG locus (Table 4, 5). GWAS pPS in the SHBG cluster (N loci=1) was significantly associated with reduced SHBG (P=1.2×10⁻¹⁷⁸⁴) and reduced IGF-1 (P=4.12×10⁻¹³)

### T2D Clusters Differ in Tissue Enrichment Including Single Cell Islets

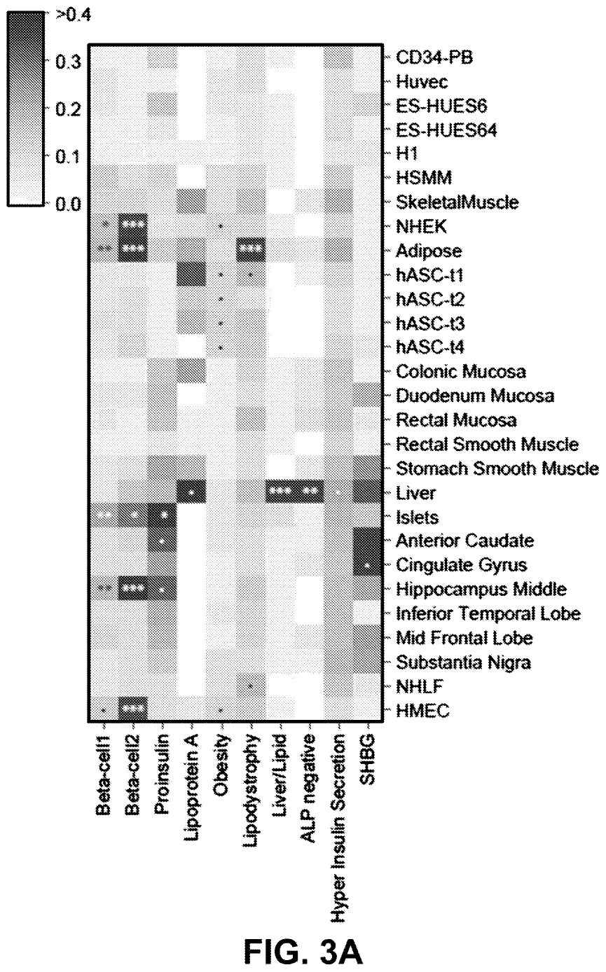

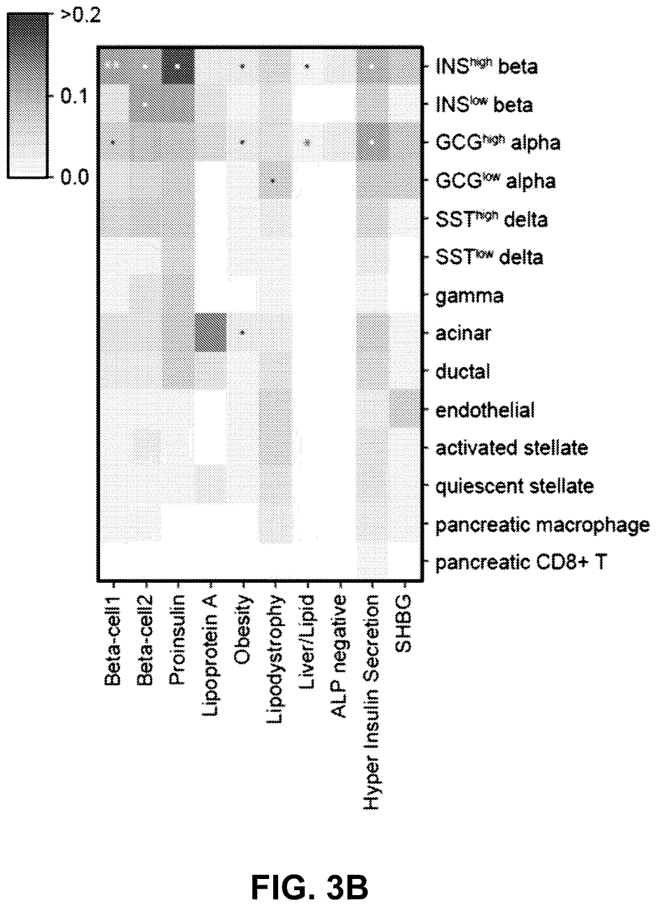

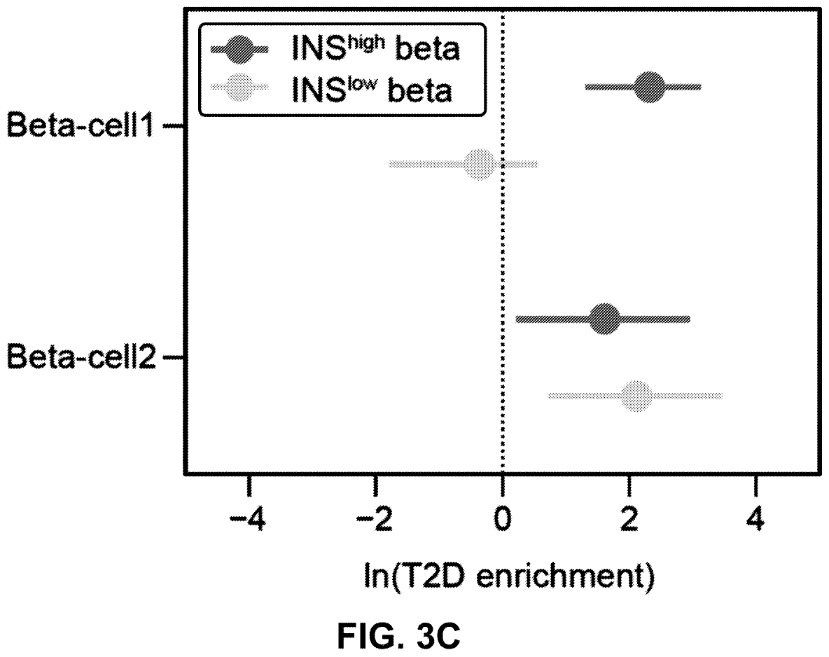

To acquire further evidence for the suspected mechanistic pathways represented by clusters and assess the biological difference between the clusters, Applicants analyzed the top weighted loci in each cluster for enrichment of epigenomic annotations across 28 tissues. The T2D clusters displayed clearly different patterns of tissue enhancer/promoter enrichment (FIG. 3A, Table 8a). In line with expected mechanisms, the Beta-cell 1, Beta-cell 2, and Proinsulin clusters were significantly enriched for pancreatic islet tissue (FDR<0.05). The Liver/Lipid and ALP negative clusters were significantly enriched in liver tissue (FDR<0.01). The Lipodystrophy cluster was strongly enriched for adipose tissue (FDR<0.01). Additionally, both Beta-cell 1 and 2 clusters had enrichment in adipose and the brain hippocampus (FDR<0.01). The Obesity cluster was most transcriptionally enriched in human epidermal keratinocytes (NHEK) and hASC-t3 pre-adipose cells, both at nominal significance (P<0.05, FDR=0.11); of note, a stigmata of insulin resistance commonly seen in obese patients with T2D is acanthosis nigricans, which is hyperpigmentation of skin driven by proliferation of epidermal keratinocytes [52].

Applicants also interrogated newly available chromatin profiles from 14.3 k pancreatic islet cells, which Chiou et al. subsetted based on their chromatin profiles [8]. There were two epigenomic subsets of islet beta cells, with the insulin gene INS among the genes with the most variable promoter accessibility, such that subsets were labeled Beta INS^{high} and Beta INS^{low}. The Beta INS^{high} islet cells were noted to be enriched for promoter accessibility for genes involved in insulin secretion, whereas the Beta INS^{low} was enriched for genes involved in stress-induced signaling response [8]. When assessing enrichment of the genetic clusters, Applicants found that the Beta-cell 1 genetic cluster was enriched only in Beta INS^{high} cells (P=0.0001, FDR=0.0014), whereas the Beta-cell 2 genetic cluster was nominally enriched in both Beta INS^{high} and Beta INS^{low} cells (P=0.025, P=0.013, respectively, FDR=0.18 for both), (FIG. 3B, Table 8b). Further supporting the delineation of the Beta cell loci into two separate sub-pathways, the same trend was observed in the fgwas enrichment analysis: Beta-cell I was significantly enriched only in INS^{high} (ln(enrichment) (95% CI) INS^{high} 2.32 (1.31-3.12); INS^{low} −0.36 (−1.79-0.55)) whereas Beta-cell 2 was significantly enriched in both single cell subsets (ln(enrichment) (95% CI), INS^{high} 1.61 (0.22-2.96); INS^{low} 2.11 (0.73-3.46)) (FIG. 3C). Together these results supported that Beta-cell 1 and Beta-cell 2 clusters relate to distinct physiological mechanisms, with Beta-cell 2 again connected to a stress-induced pancreatic state.

Also of interest, within the pancreas single cell data, the Liver/Lipid cluster was most enriched for alpha cells, (P=0.007, FDR=0.099); alpha cells secrete glucagon, which acts to release glucose from the glycogen stores in the liver, providing further connection between these T2D genetic loci with liver function.

T2D Clusters are Differentially Associated with Clinical Traits and Outcomes

Applicants next tested whether the pPS derived from the T2D genetic clusters were associated with clinical traits and outcomes in independent datasets. To do this, Applicants generated cluster pPSs in the hospital based MGB Biobank (N=25,419), on samples restricted to individuals from European ancestry based on self-reported ancestry and genetic PCs.

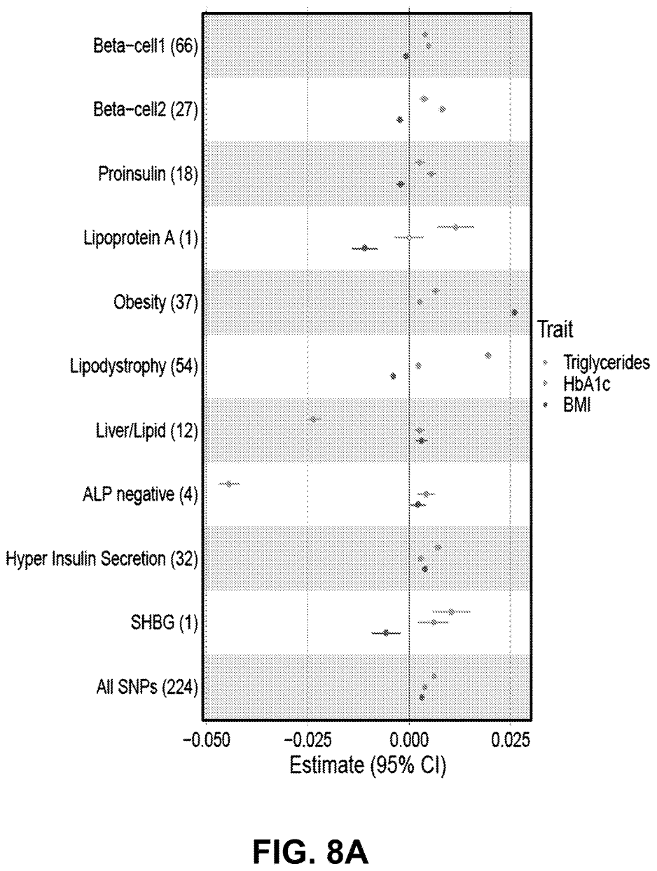

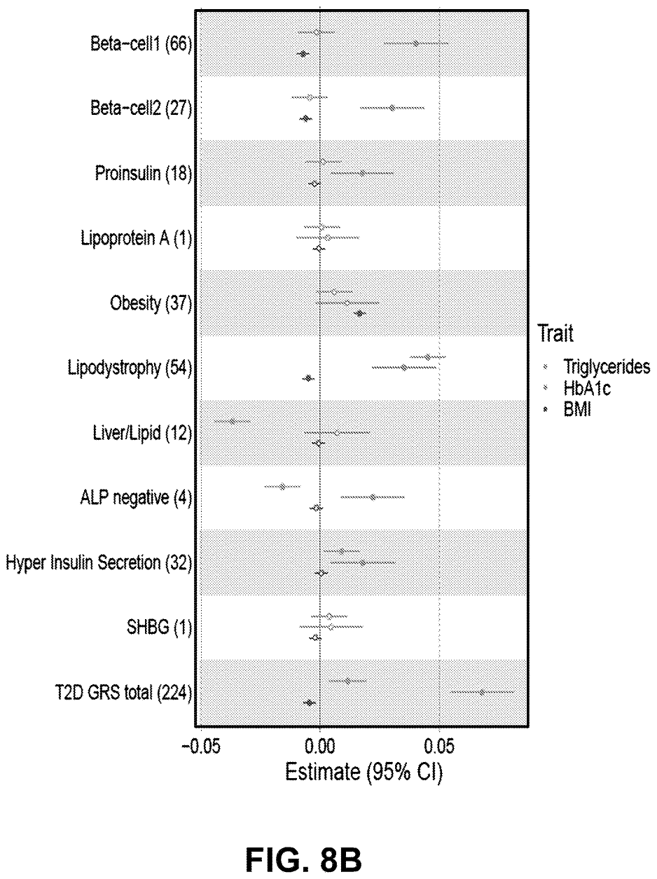

Applicants first confirmed that cluster pPSs were associated with expected traits, as available, in this study population (FIG. 8b). For example, increased Obesity pPS was associated with increased BMI (beta ln(BMI) per SD pPS=0.017, P=5.36×10⁻³⁸) and the Lipoprotein A cluster was associated with increased Lipoprotein A levels (beta per SD pPS=18.76, P=5.95×10⁻⁷) (Table 9).

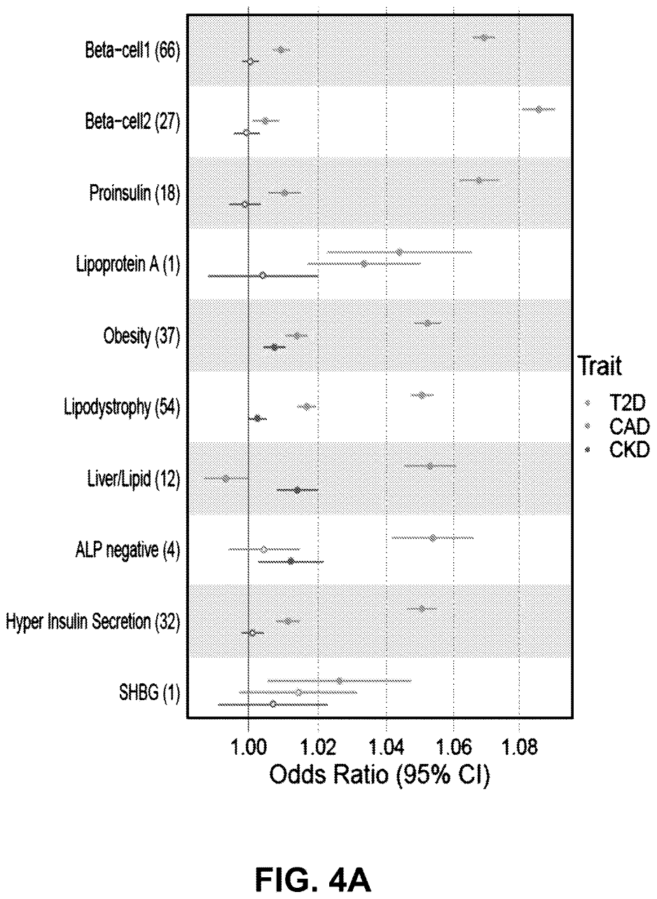

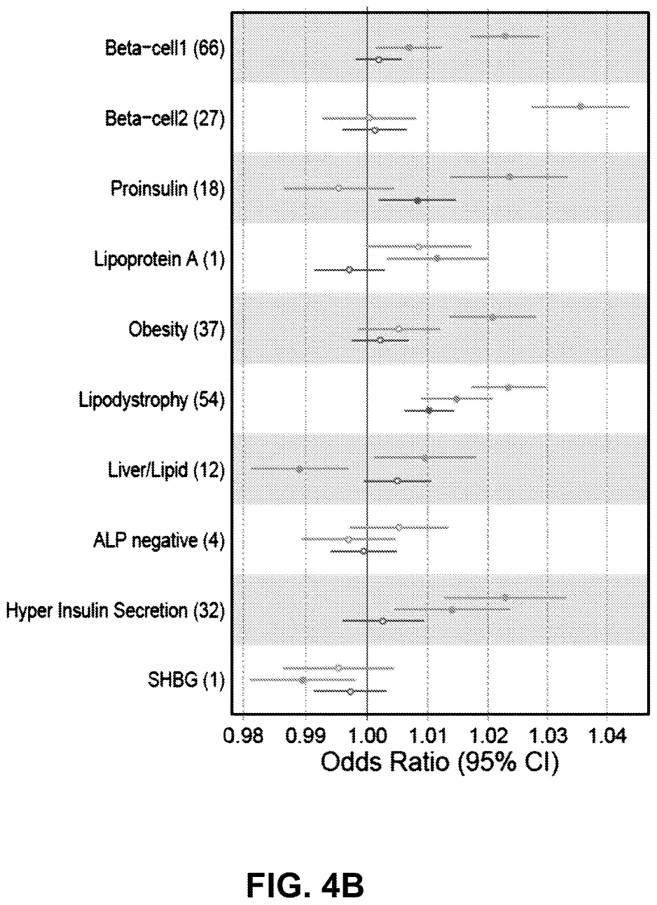

Next Applicants assessed whether the cluster pPS were associated cardiometabolic clinical outcomes related to T2D: CAD, CKD, eGFR, hypertension, ischemic stroke, diabetic retinopathy, and diabetic neuropathy (Table 4, Table 10, FIG. 4a, FIG. 8a). Starting with GWAS (which were not included in the clustering), Applicants identified GWAS pPS associations with all outcomes except diabetic retinopathy risk. Increased GWAS pPSs from the Beta-cell1, Proinsulin, Obesity, Lipodystrophy, and Lipoprotein A clusters were most significantly associated with increased risk of CAD (P<5×10⁻⁵). Increased GWAS pPSs in the Obesity and Liver/Lipid clusters were associated with increased risk of CKD (P<5×10⁻⁶), and GWAS pPSs in the Liver/Lipid, ALP negative, and SHBG clusters were associated with reduced eGFR (P<10⁻⁴). Increased GWAS pPSs of the Beta-cell1, Beta-cell2, Proinsulin, Obesity, Lipodystrophy clusters were significantly associated with increased risk of ischemic stroke (P<5×10⁻⁴). Increased Beta-cell1 and Lipodystrophy cluster GWAS pPS were associated with increased risk of neuropathy in T2D (P<5×10⁻⁴).

Several of these cardiometabolic outcomes were available to study in MGB Biobank: eGFR, CKD, CAD and hypertension. Applicants were able to replicate several of the GWAS pPS outcome associations, notably all the most significant associations (GWAS pPS P<10⁻⁵). Individuals with increased Obesity cluster pPS had significantly increased risk of hypertension (P=3.11×10-), those with increased Lipodystrophy cluster pPS had significantly increased risk of CAD (P=6.24×10⁻⁷), CKD (P=7.08×10⁻⁷), and hypertension (P=6.46×10−7), and those with increased Liver/Lipid cluster pPS had significantly reduced eGFR (P=6.18×10⁻⁴) (FIG. 4b, Table 11). Of note, in both analyses, the Liver/Lipid pPS was nominally associated with reduced risk of CAD (FIG. 4, Table 9, Table 11). The data thus supported that genetic pathways leading to T2D have distinct effects on other cardiometabolic conditions.

Loci from CAD and CKD Share Mechanistic Pathways with T2D

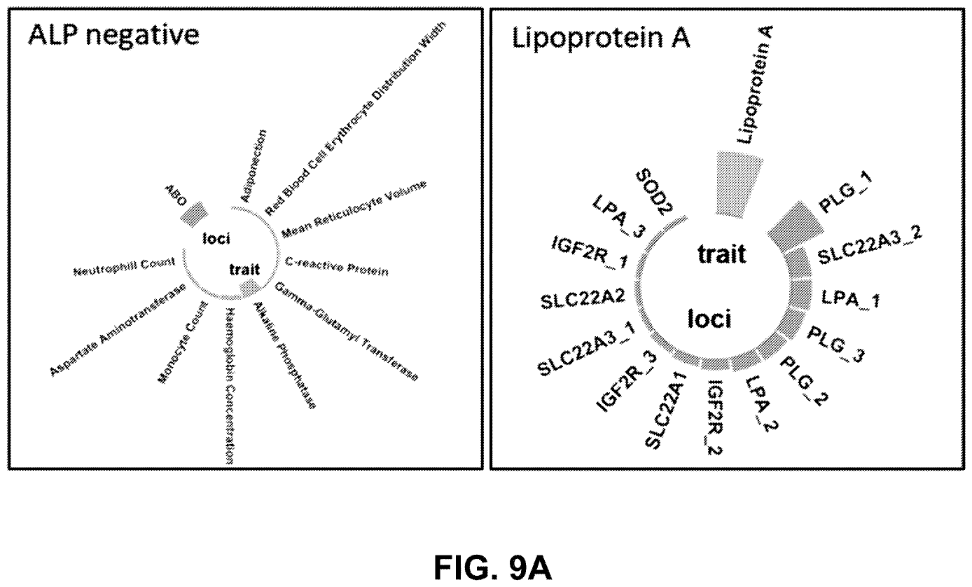

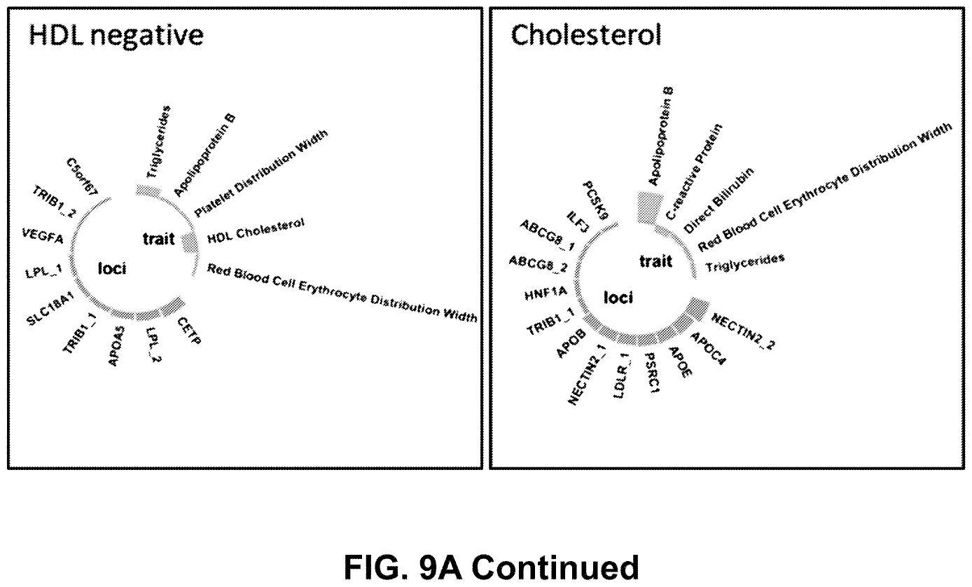

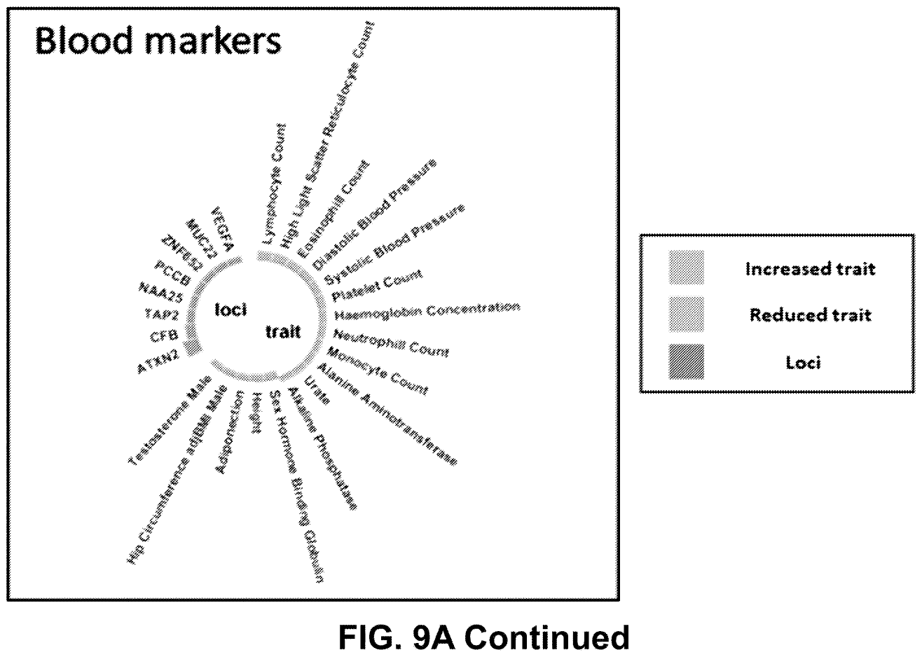

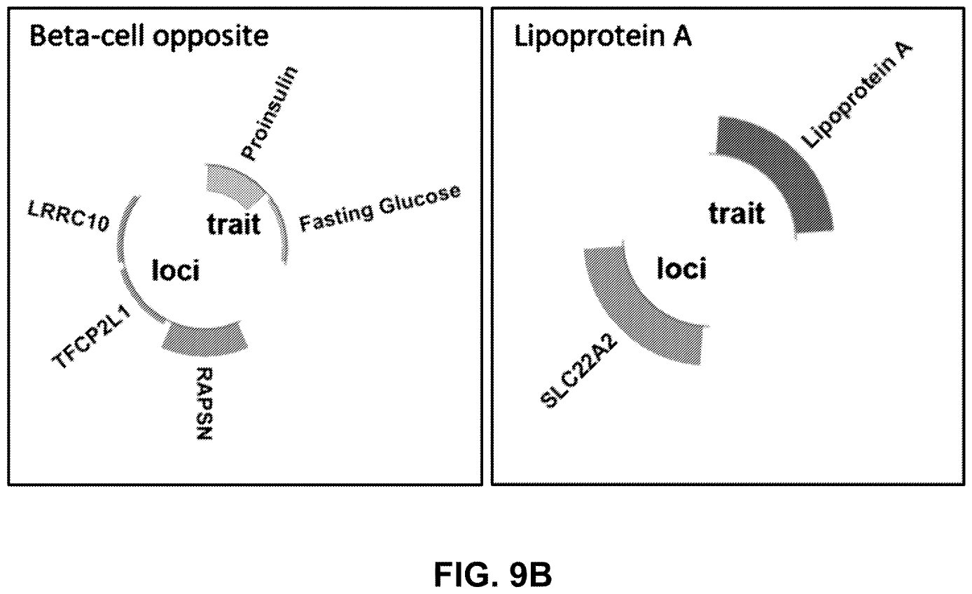

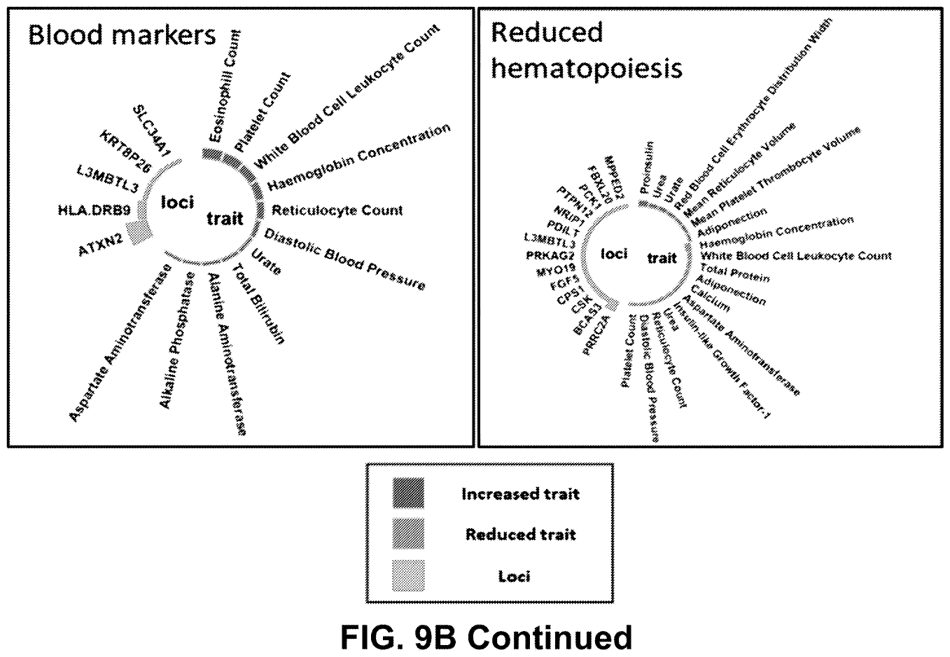

To identify shared pathways among cardiometabolic outcomes and demonstrate portability of the high-throughput pipeline, Applicants applied the same clustering approach to 222 CAD loci and 71 CKD loci. Five clusters were identified in clustering analysis of 222 CAD loci (ALP negative, Lipoprotein A, HDL negative, Cholesterol and Blood markers) and four clusters were identified for the 71 CKD loci (Beta-cell opposite, Lipoprotein A, Blood markers, and Reduced hematopoiesis) (Tables 12-15, FIG. 9). Based on inspection of constituent variants and traits in the clusters of T2D, CAD, and CKD, one cluster, Lipoprotein A, was shared by all three diseases. Additionally, the ALP negative cluster was shared between T2D and CAD, and the Blood markers cluster between CAD and CKD.

### Discussion

Novel approaches are needed to connect the currently identified hundreds of T2D genetic loci to disease pathways and also accommodate the rapid pace of new loci discovery. Here, Applicants describe expanded clustering of T2D variants, using a high-throughput pipeline for extracting and preprocessing variants from multiple GWAS datasets and generating a variant-trait association matrix. The resulting matrix consisted of 336 T2D genetic variants and 64 diabetes-related metabolic traits from publicly available GWAS datasets. By applying bNMF soft clustering to this matrix, Applicants identified ten robust clusters of T2D variants, each representing biologically meaningful mechanistic pathways.

Among the ten clusters, Applicants replicated the five identified in the previous work of 94 T2D variants (Beta-cell, Proinsulin, Obesity, Lipodystrophy, Liver/Lipid) [6], with the Beta-cell cluster now subdivided into two distinct clusters, and also identified four additional novel clusters related to pronounced insulin secretion, levels of alkaline phosphatase, lipoprotein-A, and sex hormone-binding globulin. In contrast to the prior work, which involved manual curation of loci published in GWAS manuscripts to generate the input list of variants, the current approach allowed for use of uncurated GWAS summary statistics files and included additional newly available datasets, more than tripling the number of input genetic loci. Additionally, the current work incorporated 17 more relevant GWAS traits (included only if associated with at least one T2D variant). Thus, replication of the previously identified five clusters provides strong validation of this high-throughput approach, with the newly identified clusters driven by traits or loci not available in the prior analysis. Applicants provide relevant code for use by the broader complex genetic disease community.

Three of the ten T2D clusters identified in this work (Beta-cell 1, Beta-cell 2, and Proinsulin) clearly related to pancreatic beta-cell function, with the two Beta-cell clusters differing from the Proinsulin cluster with regard to the direction of association with fasting proinsulin adjusted for fasting insulin. The association of the Proinsulin cluster with lower fasting proinsulin levels indicated a proximal defect in the insulin synthesis pathway. All three clusters were enriched in pancreatic islet tissue enhancers and promoters in the epigenomics analysis. Additionally, loci in the Beta-cell 1 cluster were significantly enriched for a subset of single beta cells predicted independent of the work based on RNA transcript levels to represent a normal state of pancreatic beta cell function, whereas loci in Beta-cell 2 cluster had a unique signal of enrichment for single beta cells predicted to be in a stressed state [8]; these functional distinctions between Beta-cell 1 and 2 supported the independent approach of phenotypically informed clustering T2D loci.

Three other T2D genetic clusters (Obesity, Lipodystrophy, Liver/Lipid) replicated findings from the prior work related to pathways of insulin resistance, gaining additional loci and traits compared to the prior analysis. Loci in these three clusters were most enriched for enhancers in tissues for the suspected mechanisms: pre-adipocytes, adipocytes, and liver tissue, respectively. The distinction between fat accumulation in the Obesity cluster and abnormal fat compartmentalization in the Lipodystrophy cluster may be supported by the differential enhancer enrichment shown for different developmental stages of the same adipocyte lineage.

In addition to a second Beta-cell cluster, there were four newly identified T2D genetic clusters from this work: ALP negative (containing the ABO locus), Lipoprotein A, SHBG, and Hyper Insulin Secretion.

The ALP negative cluster was driven by reduced serum alkaline phosphatase levels and four genetic loci, with the ABO locus most strongly weighted. Alkaline phosphatase is a circulating enzyme with isoforms that have been shown to vary in level by blood group [53]. This same cluster was also identified in the CAD clustering, with the sentinel SNP rs649129 in weak LD with the T2D SNP rs545971 (r²=0.29 in 1000 Genomes European populations). The ABO gene has previously been connected to T2D, CAD, and thromboembolic risk [49,50,51,54], although the underlying mechanisms are not fully understood. In Trégouët et al. [55], rs657152 in ABO locus, which is in LD with the ABO variant (rs545971, r²=0.98 in CEU) was associated with increased risk of venous thromboembolism at genome-wide significance (P=2.22×10⁻¹³). While Applicants found that the ALP negative cluster was significantly associated with increased fasting insulin, suggesting a mechanistic pathway of insulin resistance, and enriched for transcriptional activity in liver tissue, further research will be needed to better understand how this cluster relates to T2D risk.

The Lipoprotein A cluster included a single locus (SLC22A3/LPA) and biomarker Lp(a), pointing to a genetic pathway whereby the genetic alteration at this locus is associated with increased Lp(a) levels and also increased risk of T2D. The relationship between Lp(a) and cardiometabolic disease is complex, and genetic interrogation of LPA has been complicated by the fact that plasma concentration of Lp(a) is influenced by kringle IV type 2 (KIV-2) repeats in addition to other genetic variation. While rs487152, marking the SLC22A3/LPA locus in the analysis was associated with both increased Lp(a) levels and T2D risk, several epidemiological studies have shown an inverse association between Lp(a) concentrations and risk of T2D [56,57]. In fact, it has been shown that almost half of the physiologic variation in Lp(a) concentrations can be explained by the number of kringle IV type 2 (KIV-2) repeats on LPA gene, with the number of KIV-2 repeats inversely associated with Lp(a) levels. Rare loss of function genetic variants associated with increased number of KIV-2 repeats (and thus lower levels of Lp(a)) have been shown by Mendelian Randomization to be causally associated with increased risk of T2D [58]. Another Mendelian Randomization study that failed to show an association with T2D, included a variant in very weak LD with the variant used in the analysis, rs487152 (rs10455872, r²=0.091) [59]. Thus, the differential relationship between Lp(a) and T2D observed for the variant in the work (rs487152) vs seen in prior epidemiological and genetic studies (which included variants not in LD with the variant) highlights that there are likely multiple genetic pathways impacting Lp(a) level that may have differential effects on T2D risk.

The SHBG cluster was also driven by a single locus and biomarker. The results point to a genetic pathway whereby alteration of the SHBG locus leads to reduced SHBG levels and increased T2D risk. Consistent with the findings, previous studies have suggested that low circulating levels of SHBG are causally related to increased risk of T2D in women and men [60,61].

Applicants generated pPSs of top-weighted cluster loci and identified significant associations in individuals with expected traits (Table 7, 9) as well as with clinical outcomes for several of the clusters (Beta-cell1, Beta-cell2, Proinsulin, Obesity, Lipodystrophy, Liver/Lipid) (Table 10, 11). Such findings point to marked heterogeneity in T2D loci associations that could be missed without delineation of loci into clusters and suggest important mechanistic differences between clusters. At the same time, the effect sizes of the pPSs on clinical outcomes were likely too small to be of clinical relevance. Nevertheless, as Applicants have seen with traditional polygenic scores, expansion in the number of loci with time, can lead to improved disease risk prediction [3]; thus, perhaps with further development of the pPS in the future, there will be clearer clinical utility.

The strengths of this study include the high-throughput approach for preprocessing variants and traits from multiple GWAS datasets in an automated way. The high-throughput pipeline allows for efficient updating as additional GWAS results become available. This method can also be readily applied to other diseases beyond T2D to identify key pathways and code has been made freely available. Applicants included here application of the pipeline to CAD and CKD, demonstrating transferability of the approach and shared pathways among the three cardiometabolic outcomes.

Limitations of this study include clustering of only available phenotypes from GWAS. It is possible that additional pathways exist that are not captured using the set of traits included in the analysis. Additionally, due to methodological challenges and data availability Applicants have focused on GWAS datasets from populations of European ancestry. The clustering approach currently requires relative homogeneity across GWAS studies with regard to ancestral population; inclusion of GWAS from more than one ancestral population may lead to either genetic variants not being present due allele frequency differences across populations (introducing missing data, which the clustering method cannot tolerate) or lead to results from clustering method being artifactually driven by patterns reflective of ancestry rather than disease biology. Applicants are actively pursuing application of this method in non-European populations through additional efforts. It is worth noting that bNMF generates weights for all included elements in the matrix, and it is not known how best determine a cut-off threshold for cluster membership; Applicants have applied a reasonable strategy to maximize signal to noise. Finally, whether the associations of specific genetically derived clusters with metabolic traits remain constant throughout the disease course has not been examined in this cross-sectional analysis.

In summary, Applicants have identified ten robust genetic clusters pointing to mechanistic pathways of T2D using a high-throughput clustering pipeline of GWAS summary statistics. These clusters were readily interpretable, even if not yet fully understood, and displayed tissue-specific enrichment patterns even within single cell pancreatic tissue, supporting a biological basis of the genetic cluster assignment. Applicants further utilized cluster pPSs to stratify patients genetically that resulted in associations with distinct clinical features and cardiometabolic outcomes. Applicants demonstrate that the approach can be readily applied to other complex diseases, with identification of shared genetic pathways between T2D, CAD, and CKD. Thus, Applicants contribute to further delineation of cardiometabolic disease genetic pathways using a data-driven approach informed by physiology.

### Material and Methods

### Pipeline for Input Variant-Trait Association Matrix for Clustering.

An overview of preprocessing steps for variants and traits used for generating the input matrix for variant-trait association clustering analysis is illustrated in FIG. 5.

### Variant Selection.

To obtain a comprehensive set of independent genetic variants associated with T2D, Applicants gathered T2D GWAS, exome-chip, and whole-genome sequencing summary statistic datasets deposited in the AMP-Common Metabolic Disease Knowledge Portal (CMDKP) [9]. Applicants required the GWAS to have sample sizes larger than 10,000 to reduce false positive associations and focused on studies of predominantly European ancestry to minimize heterogeneity across studies and reduce artifactual clustering results. Thirteen GWAS datasets were included as input for identifying T2D genetic loci for clustering (Table 1).

From the selected thirteen T2D GWAS datasets, Applicants first extracted 21,666 variants reaching genome-wide significance (P<5×10⁻⁸) and then ensured variant signals replicated in the largest of these studies at a Bonferroni significance level (P-value<0.05/21,666 tests). Next Applicants identified the set of T2D variants (N=16,074) that were multi-allelic, ambiguous (A/T or C/G) or represented in less than 80% of trait GWAS datasets, and found proxy variants in linkage disequilibrium (LD) using HaploReg v4.1 [10,11]. One proxy variant was selected to represent each locus based on prioritization of 1) non-ambiguity, 2) trait GWAS representation and 3) strong LD (r²≥0.8) with the initial variant. Ambiguous variants were avoided to reduce the chance of using the incorrect allele when extracting data from multiple summary statistic files. For one variant (rs3918298), an additional proxy variant (rs11827026) was used to fill in missing data for one trait association in the matrix (the modified Stumvoll insulin sensitivity index adjusted for age, sex and BMI) [12,13]; for this single proxy, Applicants selected the variant in strongest LD with the initial variant among non-ambiguous and not multi-allelic variants.

This expanded set of T2D-associated variants was then LD-pruned based on LD data of European ancestry (CEU) using the LDlinkR package in R by LD Link [11,14,15,16]. Applicants performed stringent LD-pruning of variants using r²<0.1; when performing LD-pruning, variants with lower P-values for T2D were preferentially retained. Following LD-pruning, 340 variants remained. Four of these 340 variants did not have LD data available within LDlinkR, meaning that they could not be pruned. For each of these four variants, Applicants identified the closest neighbor by chromosomal location from the set of pruned variants and assessed their pairwise correlation across GWAS trait association Z-scores using Pearson's test of correlation; all four variants had Pearson r²>0.1 with the other variant and were removed. The final LD-pruned list of T2D variants consisted of 336 variants (Table 2). For each variant, Applicants identified the T2D risk allele with odds ratio (OR)>1 using the largest available T2D GWAS and used this allele in assessing associations with traits.

### Trait Selection.

For trait selection, Applicants utilized summary statistics available for GWAS of glycemic traits, anthropometric traits, vital signs, and additional laboratory measures in the AMP-CMDKP. Applicants restricted the analysis to GWAS of continuous traits with sample sizes larger than 5,000 to reduce false positive associations and selected studies from predominantly European populations to minimize heterogeneity, as noted above. Additionally, to incorporate additional potentially relevant biomarkers, Applicants included GWAS summary statistics of serum biomarkers available from the UK Biobank [17]. The goal was to let the genetics guide which traits were included in the clustering analysis, and thus traits were used only if the minimum P-value across the final set of variants was lower than a Bonferroni P-value cutoff of 0.05/N_final_variants (N=336); therefore, all the traits included in the clustering analysis had robust associations with at least one T2D variant. If two or more traits were highly correlated (|r|≥0.85), Applicants kept the one with the most significant P-value across the selected variants and discarded the rest. Among an initial set of 75 trait GWAS datasets, two traits were dropped during the minimum P-value filtering process and nine were dropped due to high correlation with other traits for the selected T2D variant set, leaving 64 traits (Table 3).

For the selected lists of variants and traits, Applicants utilized the GWAS summary statistics to generate a matrix of standardized Z-scores, choosing the T2D risk-increasing allele for each variant and dividing the estimated regression coefficient beta by the standard error. To account for the differences in sample size across trait GWAS studies, Applicants scaled the standardized Z-scores in a two-step process: each value was divided by the square root of the sample size for each variant in each trait GWAS, then all elements were multiplied by the mean of square root of median sample size across all SNPs in each GWAS.

bNMF Clustering.

The variant-trait association matrix Z (m by n, m: # of variants, n: # of traits) was constructed as above. Applicants then generated a non-negative input matrix X (2m by n) by concatenating two separate modifications of the original Z matrix: one containing all positive standardized Z-scores (zero otherwise) and the other all negative standardized Z-scores multiplied by −1.

The bNMF procedure factorizes X into two matrices, W (2m by K) and H^{T} (n by K), as X~WH with an optimal rank K, corresponding to the association matrix of variants and traits to the number of clusters, respectively. While conventional nonnegative matrix factorization (NMF) requires the desired model order K as an input, bNMF determines an optimal K which best balances between an error measure ∥X−WH∥² and a penalty for model complexity derived from a nonnegative half-normal prior for W and H [18,19,20]. Furthermore, bNMF iteratively regresses out irrelevant components in representing X with an automatic relevance determination technique, which enables an optimal inference for the number of clusters K. The key features for each cluster are determined by the most strongly associated traits, a natural output of the bNMF approach. bNMF algorithm was performed in R Studio for 1,000 iterations with maximum number of cluster K set to 20, and the maximum posterior solution at the most probable K was selected for downstream analyses. The output of this clustering consists of matrices of cluster-specific weights for each variant (W) and trait (H) [6].

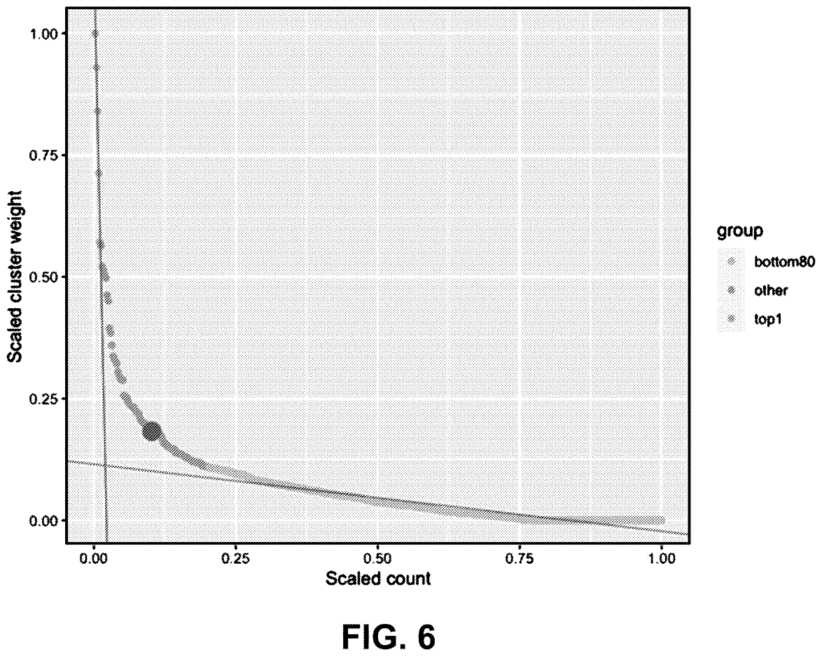

To define a set of strongest-weighted variants in each cluster and maximize the signal to noise ratio of weights, Applicants developed a method to determine a cluster weight cutoff for the clusters (FIG. 6). This involved aggregating the weights from all the clusters and plotting them in descending order. Applicants fitted a line to the top 1% of the weights and another line to the bottom 80% of the weights. Applicants identified the point where the distance to the first line became shorter than to the second line and made this the cutoff for cluster weights. For T2D, cluster weight cutoff was thus set at 0.833.

Cluster Associations with Relevant Phenotypes Using GWAS Summary Statistics.

To better characterize each cluster, particularly with regard to the associations of the loci with glycemic traits included in the clustering process, Applicants generated GWAS-partitioned polygenic scores (GWAS pPS) for each cluster, utilizing inverse-variance weighted fixed effects meta-analysis of GWAS summary statistics. For these analyses, the set of strongest-weighted variants above the weight cutoff for each cluster (above the weight cutoff, described above) were included in the model. Applicants performed these meta-analyses with the dmetar package in R [21] using GWAS summary statistics.

Additionally, Applicants applied this same approach to test cluster associations with relevant cardiometabolic outcomes studied in GWAS. As opposed to above, these outcomes were independent of the traits included in the bNMF clustering analysis. Applicants tested associations between each cluster and seven relevant cardiometabolic disease outcomes; coronary artery disease (CAD), chronic kidney disease (CKD), estimated glomerular filtration rate (eGFR), hypertension, ischemic stroke, diabetic retinopathy, and diabetic neuropathy (Table 4). The significance threshold was set to 0.05/(7×K), representing a Bonferroni correction for 7 outcomes and K clusters.

### Functional Annotation and Enrichment Analysis.

At each locus, Applicants calculated approximate Bayes Factors (aBF) for all variants 500 kb upstream and downstream with r²≥0.1, with the index variant (100% credible set) from effect size estimates and standard errors, using the approach of Wakefield [22]. Applicants then calculated a posterior probability for each variant by dividing the aBF by the sum of all aBF in the credible set.

Applicants obtained previously published 13-state ChromHMM [23]chromatin state calls for 28 cell types, excluding cancer cell lines [24]. For each cell type, Applicants extracted chromatin state annotations for enhancer (Active Enhancer 1, Active Enhancer 2, Weak Enhancer, Genic Enhancer) and promoter (Active Promoter) elements. Applicants also compiled candidate cis-regulatory elements (cCREs) for 14 cell types and subtypes from published single cell chromatin accessibility datasets [25,26].

Applicants assessed enrichment of annotations within clusters by overlapping 100% credible set variants for signals in each cluster with cell type epigenomic annotations (chromatin states and cCREs). Applicants calculated cell type probabilities for each cluster by summing the posterior probabilities of variants in cell type enhancers or promoters, divided by the number of signals in the cluster. Applicants derived significance for cell type probabilities for each cluster using a permutation-based test. Applicants permuted signals and cell type labels within each cluster and then recalculated cell type probabilities, as above. Applicants then used cell type probabilities derived from 10,000 permutations as a background distribution and performed a one-tailed test to ascertain significance for each cell type.

Applicants also assessed epigenomic enrichment in single cell pancreatic tissue using a second method. As previously described [8], Applicants subset loci from the Beta-cell 1 and 2 clusters, annotated variants using cCREs from INS^{high} and INS^{low} beta cells, and applied fgwas [27] in the fine mapping mode. Applicants considered annotations significantly enriched if the lower bound of the 95% confidence interval of the natural log enrichment was greater than 0.

Partitioned Polygenic Score (pPS) analysis in the Mass General Brigham Biobank.

The Mass General Brigham (MGB) Biobank (formerly Partners Biobank) provides banked samples (plasma, serum, DNA and genomics data) collected from more than 120,000 consented patients seen at hospitals and clinics across the MGB system, including Brigham and Women's Hospital, Massachusetts General Hospital, Massachusetts Eye and Ear Infirmary, Faulkner Hospital, Newton-Wellesley Hospital, McLean Hospital, North Shore Medical Center and Spaulding Rehabilitation Hospital, all in the Boston area of Massachusetts [28,29]. Patients are recruited at clinical care appointments at more than 40 sites and clinics, and also electronically through the patient portal at MGB. Biobank subjects provide consent for the use of their samples and data in clinical research. Written consent was provided by all study participants. Approval for analysis of Biobank data was obtained by the MGB IRB, study 2016P001018.

T2D status was defined based on algorithmically defined phenotypes developed by the Biobank Portal team using both structured and unstructured electronic medical record data and clinical, computational, and statistical methods [30]. Cases were selected by this curated phenotype to have T2D with PPV of 99% and required to be of at least age 35 to further minimize misclassification of T2D diagnosis. Additional phenotypic data (laboratory measures, vital signs, and anthropometric measures) were extracted, from which Applicants generated median values over the most recent 5 years available within the years of 2015-2020.

Up to 36,000 samples were genotyped using three versions of the Biobank SNP array offered by Illumina that is designed to capture the diversity of genetic backgrounds across the globe. The first batch of data was generated on the Multi-Ethnic Genotyping Array (MEGA) array, and the second, third, and fourth batches were generated on the Expanded Multi-Ethnic Genotyping Array (MEGA Ex) array. All remaining data were generated on the Multi-Ethnic Global (MEG) BeadChip. The genotyping data were harmonized, and quality controlled with a three-step protocol, including two stages of SNP removal and an intermediate stage of sample exclusion. The exclusion criteria for genetic variants were 1) missing call rate≥0.05, 2) significant deviation from Hardy-Weinberg equilibrium (P≤10⁻⁶ for controls and P≤10⁻²⁰ for the entire cohort), 3) significant differences in the proportion of missingness between cases and controls P≤10⁻⁶, and 4) minor allele frequency (MAF)<0.001. The exclusion criteria for samples were 1) gender discordance between the reported and genetically predicted sex, 2) subject relatedness (pairs with ≥0.125, from which Applicants removed the individuals with the highest proportion of missingness), 3) missing call rates per sample ≥0.02, and 4) population structure showing more than four standard deviations within the distribution of the study population, according to the first four principal components (PCs). Phasing was performed with SHAPEIT [31] and then imputed with the Haplotype Consortium Reference Panel [32] using the Michigan Imputation Server [33].

Applicants performed individual-level analyses on samples restricted to individuals from European ancestry based on self-reported ancestry and genetic PC's, totaling 25,419 individuals. SNPs were included in genetic risk scores as allele dosages. All SNPs were genotyped or imputed with high quality (r² values >0.95). T2D partitioned polygenic scores (pPSs) for each cluster were generated by multiplying a variant's genotype dosage by its cluster weight. For each cluster pPS, only the top-weighted variants were included, as defined above. Logistic and linear regression were performed in R v3.6.2, adjusting for age, sex, and PCs.

### Example 1 References

1. Saeedi P, Petersohn I, Salpea P, et al (2019) Global and regional diabetes prevalence estimates for 2019 and projections for 2030 and 2045: Results from the International Diabetes Federation Diabetes Atlas, 9 edition. Diabetes Res Clin Pract 157:107843
2. Redondo M J, Hagopian W A, Oram R, et al (2020) The clinical consequences of heterogeneity within and between different diabetes types. Diabetologia 63(10):2040-2048
3. Udler M S, McCarthy M I, Florez J C, Mahajan A (2019) Genetic Risk Scores for Diabetes Diagnosis and Precision Medicine. Endocr Rev 40(6):1500-1520
4. Mahajan A, Taliun D, Thurner M, et al (2018) Fine-mapping type 2 diabetes loci to single-variant resolution using high-density imputation and islet-specific epigenome maps. Nat Genet 50(11):1505-1513
5. Vujkovic M, Keaton J M, Lynch J A, et al (2020) Discovery of 318 new risk loci for type 2 diabetes and related vascular outcomes among 1.4 million participants in a multi-ancestry meta-analysis. Nat Genet 52(7):680-691
6. Udler M S, Kim J, von Grotthuss M, et al (2018) Type 2 diabetes genetic loci informed by multi-trait associations point to disease mechanisms and subtypes: A soft clustering analysis. PLoS Med 15(9):e1002654
7. Mahajan A, Wessel J, Willems S M, et al (2018) Refining the accuracy of validated target identification through coding variant fine-mapping in type 2 diabetes. Nat Genet 50(4):559-571
8. Chiou J, Zeng C, Cheng Z, et al (2021) Single-cell chromatin accessibility identifies pancreatic islet cell type- and state-specific regulatory programs of diabetes risk. Nat Genet 53(4):455-466
9. Human Genetics Knowledge Portal—Home. hugeamp.org/. Accessed 19 Mar. 2021
10. HaploReg v4.1. pubs.broadinstitute.org/mammals/haploreg/haploreg.php. Accessed 19 Mar. 2021
11. 1000 Genomes. www.internationalgenome.org/home. Accessed 19 Mar. 2021
12. Walford G A, Gustafsson S, Rybin D, et al (2016) Genome-Wide Association Study of the Modified Stumvoll Insulin Sensitivity Index Identifies BCL2 and FAM19A2 as Novel Insulin Sensitivity Loci. Diabetes 65(10):3200-3211
13. Magic Investigators—Home Page. magicinvestigators.org/. Accessed 24 Mar. 2021
14. Home—SNP—NCBI. www.ncbi.nlm.nih.gov/snp/. Accessed 19 Mar. 2021
15. Myers T A, Chanock S J, Machiela M J (2020): An R Package for Rapidly Calculating Linkage Disequilibrium Statistics in Diverse Populations. Front Genet 11:157
16. NCI, CBIIT, DCEG, Machiela LDlink. ldlink.nci.nih.gov/?tab=home. Accessed 18 Mar. 2021
17. UK Biobank—Neale lab. www.nealelab.is/uk-biobank. Accessed 18 Mar. 2021
18. Tan VYF, Févotte C (2013) Automatic relevance determination in nonnegative matrix factorization with the β-divergence. IEEE Trans Pattern Anal Mach Intell 35(7):1592-1605
19. Kim J, Mouw K W, Polak P, et al (2016) Somatic ERCC2 mutations are associated with a distinct genomic signature in urothelial tumors. Nat Genet 48(6):600-606
20. Kasar S, Kim J, Improgo R, et al (2015) Whole-genome sequencing reveals activation-induced cytidine deaminase signatures during indolent chronic lymphocytic leukaemia evolution. Nat Commun 6(1):1-12
21. Harrer, M., Cuijpers, P., Furukawa, T. & Ebert, D. D. (2019). dmetar: Companion R Package For The Guide “Doing Meta-Analysis in R”. R package version 0.0.9000. URL dmetar.protectlab.org/. Accessed 29 Apr. 2021
22. Wakefield J (2007) A Bayesian measure of the probability of false discovery in genetic epidemiology studies. Am J Hum Genet 81(2):208-227
23. Ernst J, Kellis M (2012) ChromHMM: automating chromatin-state discovery and characterization. Nat Methods 9(3):215-216
24. Varshney A, Scott L J, Welch R P, et al (2017) Genetic regulatory signatures underlying islet gene expression and type 2 diabetes. Proc Natl Acad Sci USA 114(9):2301-2306
25. Zhang K, Hocker J D, Miller M, et al (2021) A cell atlas of chromatin accessibility across 25 adult human tissues. Cold Spring Harbor Laboratory 2021.02.17.431699
26. Chiou J, Geusz R J, Okino M-L, et al (2021) Interpreting type 1 diabetes risk with genetics and single-cell epigenomics. Nature 1-5
27. Pickrell J K (2014) Joint Analysis of Functional Genomic Data and Genome-wide Association Studies of 18 Human Traits. Am J Hum Genet 94(4):559
28. Smoller J W, Karlson E W, Green R C, et al (2016) An eMERGE Clinical Center at Partners Personalized Medicine. Journal of Personalized Medicine 6(1):5
29. Karlson E W, Boutin N T, Hoffnagle A G, Allen N L (2016) Building the Partners HealthCare Biobank at Partners Personalized Medicine: Informed Consent, Return of Research Results, Recruitment Lessons and Operational Considerations. J Pers Med 6(1). doi.org/10.3390/jpm6010002
30. Yu S, Liao K P, Shaw S Y, et al (2015) Toward high-throughput phenotyping: unbiased automated feature extraction and selection from knowledge sources. J Am Med Inform Assoc 22(5):993-1000
31. Delaneau O, Zagury J-F, Marchini J (2013) Improved whole-chromosome phasing for disease and population genetic studies. Nat Methods 10(1):5-6
32. McCarthy S, Das S, Kretzschmar W, et al (2016) A reference panel of 64,976 haplotypes for genotype imputation. Nat Genet 48(10):1279-1283
33. Das S, Forer L, Sch6nherr S, et al (2016) Next-generation genotype imputation service and methods. Nat Genet 48(10):1284-1287
34. Rosengren A H, Braun M, Mahdi T, et al (2012) Reduced Insulin Exocytosis in Human Pancreatic β-Cells With Gene Variants Linked to Type 2 Diabetes. Diabetes 61(7):1726-1733
35. Zhou Y, Park S-Y, Su J, et al (2014) TCF7L2 is a master regulator of insulin production and processing. Hum Mol Genet 23(24):6419-6431
36. Mezza T, Ferraro P M, Sun V A, et al (2018) Increased β-Cell Workload Modulates Proinsulin-to-Insulin Ratio in Humans. Diabetes 67(11):2389-2396
37. Roder M E, Porte D Jr, Schwartz R S, Kahn S E (1998) Disproportionately elevated proinsulin levels reflect the degree of impaired B cell secretory capacity in patients with noninsulin-dependent diabetes mellitus. J Clin Endocrinol Metab 83(2):604-608
38. Carrat G R, Hu M, Nguyen-Tu M-S, et al (2017) Decreased STARD1O Expression Is Associated with Defective Insulin Secretion in Humans and Mice. Am J Hum Genet 100(2):238-256
39. Qi Q, Workalemahu T, Zhang C, Hu F B, Qi L (2011) Genetic variants, plasma lipoprotein(a) levels, and risk of cardiovascular morbidity and mortality among two prospective cohorts of type 2 diabetes. Eur Heart J 33(3):325-334
40. Choquet H, Meyre D (2011) Genetics of Obesity: What have Applicants Learned? Curr Genomics 12(3):169-179
41. Yaghootkar H, Scott R A, White C C, et al (2014) Genetic Evidence for a Normal-Weight “Metabolically Obese” Phenotype Linking Insulin Resistance, Hypertension, Coronary Artery Disease, and Type 2 Diabetes. Diabetes 63(12):4369-4377
42. Yaghootkar H, Lotta L A, Tyrrell J, et al (2016) Genetic Evidence for a Link Between Favorable Adiposity and Lower Risk of Type 2 Diabetes, Hypertension, and Heart Disease. Diabetes 65(8):2448-2460
43. Speliotes E K, Yerges-Armstrong L M, Wu J, et al (2011) Genome-Wide Association Analysis Identifies Variants Associated with Nonalcoholic Fatty Liver Disease That Have Distinct Effects on Metabolic Traits. PLoS Genet 7(3):e1001324
44. Mahdessian H, Taxiarchis A, Popov S, et al (2014) TM6SF2 is a regulator of liver fat metabolism influencing triglyceride secretion and hepatic lipid droplet content. Proc Natl Acad Sci USA 111(24):8913-8918
45. Kozlitina J, Smagris E, Stender S, et al (2014) Exome-wide association study identifies a TM6SF2 variant that confers susceptibility to nonalcoholic fatty liver disease. Nat Genet 46(4):352-356
46. Smagris E, Gilyard S, BasuRay S, Cohen J C, Hobbs H H (2016) Inactivation of Tm6sf2, a Gene Defective in Fatty Liver Disease, Impairs Lipidation but Not Secretion of Very Low Density Lipoproteins. J Biol Chem 291(20):10659-10676
47. Raimondo A, Rees M G, Gloyn A L (2015) Glucokinase regulatory protein: complexity at the crossroads of triglyceride and glucose metabolism. Curr Opin Lipidol 26(2):88-95
48. Smagris E, BasuRay S, Li J, et al (2015) Pnpla3I148M knockin mice accumulate PNPLA3 on lipid droplets and develop hepatic steatosis. Hepatology 61(1):108-118
49. Li-Gao R, Carlotti F, de Mutsert R, et al (2019) Genome-Wide Association Study on the Early-Phase Insulin Response to a Liquid Mixed Meal: Results From the NEO Study. Diabetes 68(12):2327-2336
50. Fagherazzi G, Gusto G, Clavel-Chapelon F, Balkau B, Bonnet F (2014) ABO and Rhesus blood groups and risk of type 2 diabetes: evidence from the large E3N cohort study. Diabetologia 58(3):519-522
51. Qi L, Cornelis M C, Kraft P, et al (2010) Genetic variants in ABO blood group region, plasma soluble E-selectin levels and risk of type 2 diabetes. Hum Mol Genet 19(9):1856-1862
52. Higgins S P, Freemark M, Prose N S (2008) Acanthosis nigricans: a practical approach to evaluation and management. Dermatol Online J 14(9):2
53. Domar U, Hirano K, Stigbrand T (1991) Serum levels of human alkaline phosphatase isozymes in relation to blood groups. Clin Chim Acta 203(2-3):305-313
54. Groot H E, Villegas Sierra L E, Abdullah Said M, Lipsic E, Karper J C, van der Harst P (2020) Genetically Determined ABO Blood Group and its Associations With Health and Disease. Arteriosclerosis, Thrombosis, and Vascular Biology 40:830-838
55. Tregouet D-A, Heath S, Saut N, et al (2009) Common susceptibility alleles are unlikely to contribute as strongly as the F V and ABO loci to VTE risk: results from a GWAS approach. Blood 113(21):5298-5303
56. Mora S, Kamstrup P R, Rifai N, Nordestgaard B G, Buring J E, Ridker P M (2010) Lipoprotein(a) and Risk of Type 2 Diabetes. Clin Chem 56(8):1252
57. Habib S S, Aslam M, Shah S F A, Naveed A K (2009) Lipoprotein (a) is associated with basal insulin levels in patients with type 2 Diabetes Mellitus. Arq Bras Cardiol 93(1):28-33
58. Tolbus A, Mortensen M B, Nielsen S F, Kamstrup P R, Bojesen S, Nordestgaard B G (2017) Kringle I V Type 2, Not LowLipoprotein(a), as a Cause of Diabetes: A Novel Genetic Approach Using SNPs Associated Selectively with Lipoprotein(a) Concentrations or with Kringle IV Type 2 Repeats. Clin Chem 63(12):1866-1876
59. Ye Z, Haycock D P C, Gurdasan D, et al (2014) The Association Between Circulating Lipoprotein(a) and Type 2 Diabetes: Is It Causal? Diabetes 63(1):332-342
60. Ding E L, Song Y, Manson 4E, et al (2009) Sex hormone-binding globulin and risk of type 2 diabetes in women and men. N Engl J Med 361(12):1152-1163
61. Perry J R B, Weedon M N, Langenberg C, et al (2010) Genetic evidence that raised sex hormone binding globulin (SHBG) levels reduce the risk of etype 2 diabetes. Hum Mol Genet 19(3):535-544

### Tables

TABLE 1
T2D GWAS datasets used for variant extraction

| Study | Datatype | Phenotype | Population | Sex | N | Reference |
|---|---|---|---|---|---|---|
| DIAMANTE | GWAS | T2DadjBMI | European | both | 898,130 | Mahajan A, et al. Nat Genet |
| | | | | | | 2018b November; 50(11): 1505-1513 |
| DIAMANTE | GWAS | T2D | European | both | 898,130 | Mahajan A, et al. Nat Genet |
| | | | | | | 2018b November; 50(11): 1505-1513 |
| DIAMANTE | GWAS | T2D | European | both | 442,817 | Mahajan A, et al. Nat Genet |
| | | | | | | 2018b November; 50(11): 1505-1513 |
| ExTexT2D | ExChip | T2D | Mixed | both | 452,244 | Mahajan A, et al. Nat Genet 2018a April; 50(4): 559-571 |
| ExTexT2D | ExChip | T2D | European | both | 228,653 | Mahajan A, et al. Nat Genet 2018a April; 50(4): 559-571 |
| ExTexT2D | ExChip | T2DadjBMI | European | both | 228,653 | Mahajan A, et al. Nat Genet 2018a April; 50(4): 559-571 |
| DIAGRAM | GWAS | T2D | European | both | 159,208 | Scott R A, et al. Diabetes. 2017 November; 66(11): 2888-2902 |
| FinnGen | GWAS | T2D | European | both | 96,499 | The FinnGen research project collects and analyzes |
| | | | | | | health registry and genetic data from a nationwide |
| | | | | | | network of Finnish biobanks. |
| 70kForT2D | GWAS | T2D | Mixed | both | 70,127 | Bonàs-Guarch S, et al. Nature Communications 9, |
| | | | | | | Article number: 321 (2018) |
| GoT2D | WGS | T2D | European | both | 44,414 | Flannick J, Fuchsberger C, Mahajan A, et al. Sci Data. |
| | | | | | | 2017 Dec. 19; 4: 170179 |
| GERA | GWAS | T2D | European | both | 56,637 | Guindo-Martínez M, et al. Submitted for publication, |
| | | | | | | 2020. BioRxiv |
| GERAX | GWAS | T2D | European | female | 33,921 | Guindo-Martínez M, et al. Submitted for publication, |
| | | | | | | 2020. BioRxiv |
| GERAX | GWAS | T2D | European | male | 22,716 | Guindo-Martínez M, et al. Submitted for publication, |
| | | | | | | 2020. BioRxiv |

TABLE 2
List of T2D-associated SNPs identified in the analysis
a) SNPs included in analyses

| | | Locus | Variant ID |
|---|---|---|---|
| Count | rsID | name | (hg19) |
| 1 | rs10801922 | PTGFRN | 1_117529458_G_A |
| 2 | rs10923931 | NOTCII2 | 1_120517959_G_T |
| 3 | rs1359939 | SEC16B_1 | 1_177820861_G_A |
| 4 | rs506589 | SEC16B_2 | 1_177894287_T_C |
| 5 | rs1572993 | CNTN2 | 1_205045087_G_A |
| 6 | rs2297539 | SRGAP2 | 1_206603535_G_A |
| 7 | rs13376660 | PROX1_1 | 1_214154985_G_A |
| 8 | rs340874 | PROX1_2 | 1_214159256_T_C |
| 9 | rs2494192 | ZC3H11B | 1_219749261_G_A |
| 10 | rs4846567 | SLC30A10 | 1_219750717_G_T |
| 11 | rs499689 | ACTA1 | 1_229565732_A_G |
| 12 | rs291370 | B3GALNT2 | 1_235693041_A_G |
| 13 | rs4660293 | PABPC4 | 1_40028180_A_G |
| 14 | rs12076849 | FAF1 | 1_51362441_C_T |
| 15 | rs11677557 | FLJ14816 | 2_121317747_A_G |
| 16 | rs2084674 | ACVR2A | 2_147874140_T_C |
| 17 | rs6714881 | CYTIP | 2_158321735_C_T |
| 18 | rs7583157 | ITGB6 | 2_161114552_A_G |
| 19 | rs13389219 | COBLL1_1 | 2_165528876_C_T |
| 20 | rs13410987 | COBLL1_2 | 2_165580775_C_T |
| 21 | rs7607124 | NYAP2 | 2_226840888_C_A |
| 22 | rs2972144 | IRS1_1 | 2_227101411_A_G |
| 23 | rs1515103 | IRS1_2 | 2_227144141_C_T |
| 24 | rs6739187 | DNMT3A | 2_25513652_G_A |
| 25 | rs17802463 | DTNB | 2_25643221_G_T |
| 26 | rs780093 | GCKR | 2_27742603_T_C |
| 27 | rs4952672 | THADA_1 | 2_43422557_G_A |
| 28 | rs7607777 | THADA_2 | 2_43629931_G_T |
| 29 | rs7596101 | FANCL_1 | 2_58978436_A_G |
| 30 | rs7575523 | FANCL_2 | 2_59335104_T_G |
| 31 | rs243019 | BCL11A | 2_60585806_T_C |
| 32 | rs12463617 | TMEM18 | 2_629244_A_C |
| 33 | rs2249105 | CEP68 | 2_65287896_A_G |
| 34 | rs7569113 | SPRED2 | 2_65657041_C_T |
| 35 | rs598747 | SYN2 | 3_12112010_G_A |
| 36 | rs2877716 | ADCY5 | 3_123094451_T_C |
| 37 | rs13083375 | PPARG_1 | 3_12365308_G_T |
| 38 | rs4135275 | PPARG_2 | 3_12443844_A_G |
| 39 | rs4679370 | SLC12A8 | 3_124919777_T_C |
| 40 | rs9847824 | PLXND1 | 3_129317585_C_T |
| 41 | rs2347252 | MRAS | 3_138095525_T_C |
| 42 | rs2868425 | TSC22D2 | 3_150083011_C_T |
| 43 | rs13094115 | MBNL1 | 3_152103745_A_G |
| 44 | rs6765166 | P2RY1 | 3_152497886_G_A |
| 45 | rs1449348 | EGFEM1P | 3_168225055_C_T |
| 46 | rs6792607 | EIF5A2 | 3_170610316_A_C |
| 47 | rs4148563 | ABCC5 | 3_183725999_T_C |
| 48 | rs2290066 | IGF2BP2_1 | 3_185445842_T_C |
| 49 | rs4481184 | IGF2BP2_2 | 3_185505787_C_T |
| 50 | rs3887925 | ST6GAL1 | 3_186665645_C_T |
| 51 | rs6767001 | LPP | 3_187751389_G_A |
| 52 | rs11710420 | UBE2E2_1 | 3_23221931_G_A |
| 53 | rs1496653 | UBE2E2_2 | 3_23454790_A_G |
| 54 | rs17013433 | UBE2E2_3 | 3_23578594_G_A |
| 55 | rs10461018 | CCDC12 | 3_46995242_C_T |
| 56 | rs6446298 | TRAIP | 3_49860854_T_C |
| 57 | rs11242 | RFT1 | 3_53125922_T_C |
| 58 | rs1317838 | CACNA2D3 | 3_54823598_A_G |
| 59 | rs13434089 | ATXN7 | 3_63948566_T_C |
| 60 | rs6772129 | ADAMTS9 | 3_64700425_A_G |
| 61 | rs2272163 | ROBO2 | 3_77671721_C_A |
| 62 | rs6854709 | CENPE | 4_104129786_T_C |
| 63 | rs1296328 | PCDH18 | 4_137083193_A_C |
| 64 | rs10011838 | FBXW7 | 4_153520279_G_A |
| 65 | rs12641088 | PDGFC | 4_157680199_C_T |
| 66 | rs7667864 | DCAF16 | 4_17811914_A_C |
| 67 | rs735949 | ACSL1 | 4_185716232_T_C |
| 68 | rs13130484 | GNPDA2 | 4_45175691_C_T |
| 69 | rs6829655 | DCUN1D4 | 4_52810307_T_C |
| 70 | rs4361456 | WFS1_1 | 4_6256629_G_A |
| 71 | rs6446480 | WFS1_2 | 4_6295565_C_T |
| 72 | rs1531583 | PCGF3 | 4_744972_G_T |
| 73 | rs993380 | SCD5 | 4_83584496_A_G |
| 74 | rs13133548 | FAM13A_1 | 4_89740128_G_A |
| 75 | rs7660000 | FAM13A_2 | 4_89751858_C_T |
| 76 | rs7678054 | SMARCAD1 | 4_95093855_G_A |
| 77 | rs370176 | SLC04C1 | 5_101595558_C_T |
| 78 | rs7729395 | PAM_1 | 5_102100576_C_T |
| 79 | rs17154889 | PAM_2 | 5_102305065_C_A |
| 80 | rs329124 | JADE2 | 5_133865452_A_G |
| 81 | rs2270561 | ANKII | 5_14847628_G_A |
| 82 | rs2963449 | EBF1 | 5_157934530_A_G |
| 83 | rs12187196 | MRPS30 | 5_44683819_A_C |
| 84 | rs17211038 | ITGA1 | 5_52118488_C_T |
| 85 | rs6876198 | ARL15_1 | 5_53303595_T_C |
| 86 | rs151804 | ARL15_2 | 5_53452060_A_G |
| 87 | rs459193 | C5orf67_1 | 5_55806751_A_G |
| 88 | rs9686661 | C5orf67_2 | 5_55861786_C_T |
| 89 | rs1469398 | PIK3R1 | 5_67726055_G_A |
| 90 | rs2307111 | POC5 | 5_75003678_T_C |
| 91 | rs4457053 | ZBED3 | 5_76424949_G_A |
| 92 | rs955897 | BHMT | 5_78431486_A_C |
| 93 | rs1665901 | BEND3 | 6_107433400_A_T |
| 94 | rs4273712 | CENPW | 6_126964510_A_G |
| 95 | rs719727 | RSPO3 | 6_127414838_A_G |
| 96 | rs9494624 | NHEG1 | 6_137300960_A_G |
| 97 | rs632057 | CITED2 | 6_139834012_T_G |
| 98 | rs614730 | HIVEP2 | 6_143066053_T_A |
| 99 | rs487152 | SLC22A3 | 6_160774486_C_A |
| 100 | rs17630640 | QKI | 6_164107529_A_G |
| 101 | rs2328524 | E2F3 | 6_20488234_G_A |
| 102 | rs9368222 | CDKAL1_1 | 6_20686996_C_A |
| 103 | rs6928012 | CDKAL1_2 | 6_20728513_C_T |
| 104 | rs4493738 | CDKAL1_3 | 6_20775660_A_G |
| 105 | rs12209877 | CDKAL1_4 | 6_21027761_G_T |
| 106 | rs1265089 | PSORS1C2 | 6_31108607_G_A |
| 107 | rs2855804 | MICB | 6_31467365_C_T |
| 108 | rs1799964 | TNF | 6_31542308_T_C |
| 109 | rs9267658 | SLC44A4 | 6_31845985_T_C |
| 110 | rs715299 | NOTCH4 | 6_32189841_T_G |
| 111 | rs3806155 | BTNL2 | 6_32373378_A_T |
| 112 | rs9273363 | HLA-DQB1 | 6_32626272_C_A |
| 113 | rs7764856 | HLA-DQA2 | 6_32680640_T_A |
| 114 | rs998584 | VEGFA_1 | 6_43757896_C_A |
| 115 | rs10456526 | VEGFA_2 | 6_43814625_G_A |
| 116 | rs987237 | TFAP2B | 6_50803050_A_G |
| 117 | rs10458103 | RREB1_1 | 6_7037738_G_A |
| 118 | rs9379084 | RREB1_2 | 6_7231843_G_A |
| 119 | rs9505086 | RREB1_3 | 6_7232186_T_C |
| 120 | rs11496066 | FBXL13 | 7_102486254_T_C |
| 121 | rs39328 | RELN | 7_103444978_C_T |
| 122 | rs6976111 | CTTNBP2 | 7_117495667_C_A |
| 123 | rs287621 | KLF14 | 7_130435181_T_C |
| 124 | rs10276674 | DGKB_1 | 7_14922007_T_C |
| 125 | rs6977081 | AOC1 | 7_150542515_G_T |
| 126 | rs10244051 | DGKB_2 | 7_15063833_T_G |
| 127 | rs2301916 | UBE3C | 7_156974127_G_T |
| 128 | rs4279506 | IGF2BP3 | 7_23512896_G_C |
| 129 | rs6948511 | JAZF1_1 | 7_27978715_T_C |
| 130 | rs849133 | JAZF1_2 | 7_28192280_C_T |
| 131 | rs2267716 | CRHR2 | 7_30716643_T_C |
| 132 | rs2971669 | GCK | 7_44231778_C_T |
| 133 | rs11765592 | C1GALT1 | 7_7250753_T_C |
| 134 | rs17151859 | MSRA_2 | 8_10237980_A_C |
| 135 | rs10903337 | XKR6 | 8_10905594_G_C |
| 136 | rs4129682 | TRHR | 8_110122139_C_T |
| 137 | rs3802177 | SLC30A8 | 8_118185025_G_A |
| 138 | rs1561927 | TMEM75 | 8_129568078_C_T |
| 139 | rs7828303 | BOP1 | 8_145509128_T_C |
| 140 | rs2272662 | SLC39A4 | 8_145639726_T_C |
| 141 | rs997258 | ARHGAP39 | 8_145903077_C_A |
| 142 | rs10096633 | LPL | 8_19830921_C_T |
| 143 | rs4733067 | PURG | 8_30856943_A_G |
| 144 | rs2354575 | GPAT4_1 | 8_41462898_A_G |
| 145 | rs11995712 | GPAT1_2 | 8_41472375_G_A |
| 146 | rs12544241 | ANK1_1 | 8_41514888_G_A |
| 147 | rs16890723 | ANK1_2 | 8_41517064_T_C |
| 148 | rs3102078 | PPP1R3B_1 | 8_9050725_C_A |
| 149 | rs9987289 | PPP1R3B_2 | 8_9183358_A_G |
| 150 | rs17662402 | TNKS | 8_9265105_T_C |
| 151 | rs9297945 | DPY19L4 | 8_95749023_A_G |
| 152 | rs13255935 | NDUFAF6 | 8_95965685_C_A |
| 153 | rs17689007 | MSRA_1 | 8_9974824_G_A |
| 154 | rs545971 | ABO | 9_136143372_C_T |
| 155 | rs3829109 | DNLZ | 9_139256766_G_A |
| 156 | rs11145846 | SNAPC4 | 9_139285655_A_G |
| 157 | rs7467618 | HAUS6 | 9_19065295_T_C |
| 158 | rs7867635 | MLLT3 | 9_20241069_T_C |
| 159 | rs523096 | CDKN2B_1 | 9_22019129_A_G |
| 160 | rs17694572 | CDKN2B_2 | 9_22054356_G_A |
| 161 | rs10811658 | CDKN2B_3 | 9_22128600_G_A |
| 162 | rs10811661 | CDKN2B_4 | 9_22134094_T_C |
| 163 | rs1412234 | LINGO2 | 9_28410683_T_C |
| 164 | rs10758593 | GLIS3 | 9_4292083_G_A |
| 165 | rs11137820 | PSAT1 | 9_81359113_C_G |
| 166 | rs17791513 | TLE4 | 9_81905590_A_G |
| 167 | rs2796441 | TLE1 | 9_84308948_G_A |
| 168 | rs7872931 | ZNF169 | 9_96970197_G_A |
| 169 | rs17746088 | VTI1A_1 | 10_114598202_T_C |
| 170 | rs11815670 | VTI1A_2 | 10_114613439_G_A |
| 171 | rs1927157 | VTI1A_3 | 10_114635381_T_C |
| 172 | rs1885283 | VTI1A_4 | 10_114648499_C_A |
| 173 | rs7079711 | TCF7L2_1 | 10_114745788_G_A |
| 174 | rs17747324 | TCF7L2_2 | 10_114752503_T_C |
| 175 | rs11196187 | TCF7L2_3 | 10_114759445_G_A |
| 176 | rs12354626 | TCF7L2_4 | 10_114772429_G_A |
| 177 | rs7087006 | TCF7L2_5 | 10_114823426_A_G |
| 178 | rs10885410 | TCF7L2_6 | 10_114824473_G_A |
| 179 | rs6585206 | TCF7L2_7 | 10_114859251_A_G |
| 180 | rs10885414 | TCF7L2_8 | 10_114861304_A_G |
| 181 | rs4918796 | TCF7L2_9 | 10_114880342_T_C |
| 182 | rs290483 | TCF7L2_10 | 10_114915214_G_T |
| 183 | rs7895525 | CDC123_1 | 10_12250620_T_C |
| 184 | rs10906115 | CDC123_2 | 10_12314997_A_G |
| 185 | rs11200594 | PLEKHA1 | 10_124139393_C_T |
| 186 | rs2616055 | NEUROG3_1 | 10_71317835_G_A |
| 187 | rs177045 | NEUROG3_2 | 10_71321279_A_G |
| 188 | rs2812533 | FAM241B_1 | 10_71452285_T_C |
| 189 | rs2642588 | FAM241B_2 | 10_71466578_T_G |
| 190 | rs697239 | ZMIZ1_1 | 10_80947438_T_C |
| 191 | rs10509406 | ZMIZ1_2 | 10_80959517_T_C |
| 192 | rs11202627 | PTEN | 10_89769340_C_T |
| 193 | rs7094274 | CPEB3 | 10_93945476_C_T |
| 194 | rs2209972 | MARK2P9 | 10_94179028_C_T |
| 195 | rs5015480 | HHEX | 10_94465559_C_T |
| 196 | rs10893829 | ETS1_1 | 11_128042575_T_C |
| 197 | rs10750397 | ETS1_2 | 11_128234144_A_G |
| 198 | rs11819995 | ETS1_3 | 11_128389391_C_T |
| 199 | rs757110 | ABCC8 | 11_17418477_C_A |
| 200 | rs11600952 | IGF2 | 11_2101283_G_A |
| 201 | rs7111341 | TH | 11_2213166_C_T |
| 202 | rs231349 | KCNQ1_1 | 11_2672821_C_T |
| 203 | rs231362 | KCNQ1_2 | 11_2691471_A_G |
| 204 | rs2283222 | KCNQ1_3 | 11_2755601_T_C |
| 205 | rs2074197 | KCNQ1_4 | 11_2825279_C_T |
| 206 | rs163177 | KCNQ1_5 | 11_2838413_T_C |
| 207 | rs4923543 | METTL15 | 11_28534898_G_A |
| 208 | rs504034 | PDHX | 11_34997562_T_C |
| 209 | rs1061810 | HSD17B12 | 11_43877934_C_A |
| 210 | rs12805422 | SLC35C1 | 11_45839889_G_A |
| 211 | rs7124681 | CELF1 | 11_47529947_C_A |
| 212 | rs694243 | FRMD8 | 11_65172197_C_T |
| 213 | rs947791 | SCYL1 | 11_65302893_G_A |
| 214 | rs3814707 | OVOL1 | 11_65560785_G_A |
| 215 | rs3918298 | CCND1 | 11_69463273_G_A |
| 216 | rs11603334 | ARAP1 | 11_72432985_G_A |
| 217 | rs663015 | STARD10 | 11_72499035_T_C |
| 218 | rs1387153 | MTNR1B_1 | 11_92673828_C_T |
| 219 | rs9971402 | MTNR1B_2 | 11_92756551_C_A |
| 220 | rs1900385 | SLC36A4 | 11_93014343_T_G |
| 221 | rs3764002 | WSCD2 | 12_108618630_C_T |
| 222 | rs2241737 | RFC5 | 12_118454833_C_G |
| 223 | rs550349 | SPPL3_1 | 12_121236160_A_C |
| 224 | rs11065299 | SPPL3_2 | 12_121297815_G_A |
| 225 | rs1169302 | HNF1A | 12_121432302_T_G |
| 226 | rs7298521 | P2RX7_1 | 12_121541831_C_T |
| 227 | rs208302 | P2RX7_2 | 12_121602760_G_A |
| 228 | rs11057276 | SBNO1 | 12_123820397_T_C |
| 229 | rs11837287 | ZNF664 | 12_124470059_C_T |
| 230 | rs863750 | FAM101A | 12_124505444_C_T |
| 231 | rs1882297 | FBRSL1 | 12_133069134_G_A |
| 232 | rs2343872 | SSPN_1 | 12_26398707_C_T |
| 233 | rs1872992 | SSPN_2 | 12_26457190_A_G |
| 234 | rs10492373 | KLHL42 | 12_27959998_G_A |
| 235 | rs7963651 | CCND2_1 | 12_4297831_C_A |
| 236 | rs11063069 | CCND2_2 | 12_4374373_A_G |
| 237 | rs3217795 | CCND2_3 | 12_4386064_A_G |
| 238 | rs3217862 | CCND2_4 | 12_4399087_T_G |
| 239 | rs12299509 | CCND2_5 | 12_4406281_A_G |
| 240 | rs10849090 | DYRK4 | 12_4669954_A_G |
| 241 | rs17101712 | RPSAP52_1 | 12_66166746_T_A |
| 242 | rs2612075 | RPSAP52_2 | 12_66209646_C_T |
| 243 | rs1042725 | HMGA2 | 12_66358347_C_T |
| 244 | rs1051334 | TSPAN8 | 12_71523134_A_C |
| 245 | rs2197973 | USP44 | 12_95928560_C_T |
| 246 | rs1592519 | RMST | 12_97847018_C_T |
| 247 | rs9555581 | MYO16 | 13_109944192_C_T |
| 218 | rs10507349 | RNF6 | 13_26781528_G_A |
| 249 | rs1045411 | IIMGB1 | 13_31033232_C_T |
| 250 | rs576674 | KL | 13_33554302_G_A |
| 251 | rs9316500 | DLEU1 | 13_51094114_T_G |
| 252 | rs7988007 | PCDH17 | 13_58988271_A_C |
| 253 | rs1359790 | SPRY2 | 13_80717156_G_A |
| 254 | rs1028613 | MARK3 | 14_103888324_G_A |
| 255 | rs7145828 | AKAP6 | 14_33303298_A_G |
| 256 | rs8017808 | CLEC14A | 14_38848419_G_T |
| 257 | rs10150332 | NRXN3 | 14_79936964_T_C |
| 258 | rs1742546 | CCDC88C | 14_91883499_G_A |
| 259 | rs8032939 | RASGRP1 | 15_38834033_T_C |
| 260 | rs11637681 | BMF | 15_40387971_A_G |
| 261 | rs2289739 | LTK | 15_41801512_G_T |
| 262 | rs2456530 | ONECUT1 | 15_53091553_C_T |
| 263 | rs12440878 | TCF12 | 15_57535215_G_A |
| 264 | rs7163757 | C2CD4A | 15_62391608_C_T |
| 265 | rs7178762 | USP3 | 15_63871292_C_T |
| 266 | rs4776970 | MAP2K5 | 15_68080886_A_T |
| 267 | rs7166281 | IMP3 | 15_75928192_T_G |
| 268 | rs4886508 | PSTPIP1 | 15_77307910_C_T |
| 269 | rs1005752 | HMG20A | 15_77818128_C_A |
| 270 | rs17242083 | ARPIN | 15_90449476_C_T |
| 271 | rs2290203 | PRC1 | 15_91512067_G_A |
| 272 | rs7189927 | ATP2A1 | 16_28913787_T_C |
| 273 | rs11248914 | FAM234A | 16_293562_T_C |
| 274 | rs12325539 | DOC2A | 16_30033633_T_C |
| 275 | rs12445430 | NLRC3 | 16_3613126_C_T |
| 276 | rs3095571 | AKTIP | 16_53544993_G_A |
| 277 | rs1421085 | FTO_1 | 16_53800954_T_C |
| 278 | rs16952522 | FTO_2 | 16_53807498_C_G |
| 279 | rs6499646 | FTO_3 | 16_53843533_T_C |
| 280 | rs17218700 | FTO_4 | 16_53844579_G_A |
| 281 | rs6499653 | FTO_5 | 16_53877592_T_C |
| 282 | rs889398 | NFAT5 | 16_69556715_C_T |
| 283 | rs7202844 | CTRB1 | 16_75247391_A_G |
| 284 | rs2925979 | CMIP_1 | 16_81534790_T_C |
| 285 | rs8063007 | CMIP_2 | 16_81541587_C_A |
| 286 | rs4925109 | RAI1 | 17_17661802_A_G |
| 287 | rs1108511 | SREBF1 | 17_17726469_T_C |
| 288 | rs10962 | HNF1B_1 | 17_36046451_G_C |
| 289 | rs3110641 | HNF1B_2 | 17_36047417_A_G |
| 290 | rs2189301 | HNF1B_3 | 17_36063685_G_A |
| 291 | rs4239217 | HNF1B_4 | 17_36098987_G_A |
| 292 | rs12453022 | ATP2A3_1 | 17_3840055_C_T |
| 293 | rs3826482 | ATP2A3_2 | 17_3860356_A_T |
| 294 | rs4790581 | CYB5D2 | 17_4066657_T_C |
| 295 | rs646123 | MLX | 17_40720632_G_A |
| 296 | rs9906944 | IGF2BP1 | 17_47091420_C_T |
| 297 | rs9899520 | BPTF | 17_65957568_A_G |
| 298 | rs727428 | SHBG | 17_7537792_T_C |
| 299 | rs7219033 | GLP2R | 17_9787958_G_A |
| 300 | rs7505951 | CELF4 | 18_36313311_G_A |
| 301 | rs17594301 | TCF4 | 18_53048885_T_C |
| 302 | rs17684074 | WDR7 | 18_54675384_G_C |
| 303 | rs9957145 | GRP | 18_56876228_G_A |
| 304 | rs2168711 | MC4R_1 | 18_57848531_T_C |
| 305 | rs9675376 | MC4R_2 | 18_57969244_G_A |
| 306 | rs9947450 | MC4R_3 | 18_58052665_C_T |
| 307 | rs12454712 | BCL2 | 18_60845884_T_C |
| 308 | rs7240767 | LAMA1 | 18_7070642_T_C |
| 309 | rs1049481 | CALR | 19_13054781_G_T |
| 310 | rs10401969 | SUGP1_TM6SF2 | 19_19407718_T_C |
| 311 | rs11667352 | PEPD_1 | 19_33918423_A_G |
| 312 | rs7258937 | PEPD_2 | 19_33938800_C_T |
| 313 | rs2075650 | TOMM40 | 19_45395619_A_G |
| 314 | rs10408179 | EML2 | 19_46157004_T_C |
| 315 | rs11671664 | GIPR | 19_46172278_G_A |
| 316 | rs2075142 | SNRPD2 | 19_46191021_A_G |
| 317 | rs3810291 | ZC3H4 | 19_47569003_G_A |
| 318 | rs17175860 | INSR | 19_7235146_A_G |
| 319 | rs4804833 | MAP2K7 | 19_7970635_A_G |
| 320 | rs813393 | NKX2-2 | 20_21451139_G_A |
| 321 | rs2065703 | CDK5RAP1 | 20_31966698_C_T |
| 322 | rs736953 | CHMP4B | 20_32415089_G_T |
| 323 | rs6120487 | RALY | 20_32622608_A_G |
| 324 | rs2144908 | HNF4A_1 | 20_42985717_G_A |
| 325 | rs1800961 | HNF4A_2 | 20_43042364_C_T |
| 326 | rs1412956 | EYA2_1 | 20_45557017_A_G |
| 327 | rs6063048 | EYA2_2 | 20_45598564_G_A |
| 328 | rs6012876 | CEBPB | 20_48830265_T_C |
| 329 | rs17733217 | TSHZ2 | 20_51222704_A_G |
| 330 | rs6064709 | GNAS | 20_57379850_G_A |
| 331 | rs2023681 | LIF | 22_30599562_A_G |
| 332 | rs2006867 | YWHAH | 22_32384307_C_T |
| 333 | rs5758223 | EP300 | 22_41489920_G_A |
| 334 | rs738408 | PNPLA3 | 22_44324730_C_T |
| 335 | rs713669 | IL17REL | 22_50451771_C_T |
| 336 | rs12184907 | MOV10L1 | 22_50588313_G_A |

b) SNPs excluded from analyses

| | | Locus | Variant ID | | | Closest |
|---|---|---|---|---|---|---|
| Count | rsID | name | (hg19) | Chromosome | Position | variant |
| 1 | rs25946 | ANKH_1 | 5_14787064_A_C | 5 | 14787064 | rs2270561 |
| 2 | rs7454108 | HLA-DQA2_2 | 6_32681483_T_C | 6 | 32681483 | rs7764856 |
| 3 | rs2466298 | SLC30A8_1 | 8_118183658_C_A | 8 | 1.18E+08 | rs3802177 |
| 4 | rs7227255 | MC4R_4 | 18_58055731_G_A | 18 | 58055731 | rs9947450 |

| | Closest | | Top | | Closest | |
|---|---|---|---|---|---|---|
| | variant | Distance | weighted | | variant | Correlation |
| Count | locus | (bp) | cluster | Weight | weight | (r) |
| 1 | ANKH_2 | 60564 | NA | NA | NA | 0.5 |
| 2 | HLA-DQA2_1 | 843 | blood_markers | 1.07207 | 2.161281 | 0.75 |
| 3 | SLC30A8_2 | 1367 | beta-cell2 | 1.735474 | 2.612552 | 0.91 |
| 4 | MC4R_3 | 3066 | obesity | 1.341851 | 1.334814 | 0.89 |

TABLE 3
GWAS datasets for traits used in T2D loci clustering

| | | | | Kept after | Reason for | |
|---|---|---|---|---|---|---|
| Study | Trait Name | Population | N | filtering | filtering | Correlated trait |
| MAGIC | HOMA-B | European | 46,186 | yes | | |
| MAGIC | HOMA-IR | European | 46,186 | yes | | |
| MAGIC | Fasting proinsulin adjusted for | European | 10,701 | yes | | |
| | fasting insulin | | | | | |
| MAGIC | 2-hour glucose | European | 15,252 | yes | | |
| MAGIC | Fasting glucose | European | 46,186 | yes | | |
| MAGIC | Fasting insulin | European | 58,074 | yes | | |
| MAGIC | Modified Stumvoll insulin | European | 16,753 | yes | | |
| | sensitivity index adjAgeSexBMI | | | | | |
| MAGIC | Disposition index | European | 5,318 | yes | | |
| MAGIC | Corrected insulin response | European | 5,318 | yes | | |
| | (CIR) to glucose at 30 | | | | | |
| MAGIC | HbA1c | European | 123,665 | yes | | |
| ADIPOGen | Adiponectin | Mixed | 45,891 | yes | | |
| BFpercent | Body fat percent | Mixed | 100,716 | yes | | |
| GiantUKBB | Body mass index | European | 795,640 | yes | | |
| GiantUKBB | Height | European | 795,640 | yes | | |
| GIANT | Hip circumference Female | European | 379,501 | no | correlation | Body mass index |
| GIANT | Hip circumference Male | European | 315,284 | no | correlation | Waist circumference |
| | | | | | | Male |
| GIANT | Hip circumference adjBMI Female | European | 379,501 | yes | | |
| GIANT | Hip circumference adjBMI Male | European | 315,284 | yes | | |
| GIANT | Waist circumference Female | European | 379,501 | yes | | |
| GIANT | Waist circumference Male | European | 315,284 | yes | | |
| GIANT | Waist circumference adjBMI Female | European | 379,501 | yes | | |
| GIANT | Waist circumference adjBMI Male | European | 315,284 | yes | | |
| GIANT | Waist-to-hip ratio Female | European | 379,501 | yes | | |
| GIANT | Waist-to-hip ratio Male | European | 315,284 | yes | | |
| GIANT | Waist-to-hip ratio adjBMI Female | European | 379,501 | yes | | |
| GIANT | Waist-to-hip ratio adjBMI Male | European | 315,284 | yes | | |
| HRgene | Heart rate | Mixed | 181,171 | no | p-value | |
| Leptin | Leptin | European | 32,161 | yes | | |
| Leptin | Leptin adjBMI | European | 32,161 | yes | | |
| UKBB | White blood cell count | European | 350,470 | yes | | |
| UKBB | Red blood cell count | European | 350,475 | yes | | |
| UKBB | Hemoglobin concentration | European | 350,474 | yes | | |
| UKBB | Mean corpuscular volume | European | 350,473 | yes | | |
| UKBB | Red blood cell distribution width | European | 350,473 | yes | | |
| UKBB | Platelet count | European | 350,474 | yes | | |
| UKBB | Mean platelet volume | European | 350,470 | yes | | |
| UKBB | Platelet distribution width | European | 350,470 | yes | | |
| UKBB | Lymphocyte count | European | 349,856 | yes | | |
| UKBB | Monocyte count | European | 349,856 | yes | | |
| UKBB | Neutrophill count | European | 349,856 | no | correlation | White blood cell |
| | | | | | | count |
| UKBB | Eosinophill count | European | 349,856 | yes | | |
| UKBB | Basophill count | European | 349,856 | yes | | |
| UKBB | Nucleated red blood cell count | European | 349,848 | no | p-value | |
| UKBB | Reticulocyte count | European | 344,729 | no | correlation | High light scatter |
| | | | | | | reticulocyte count |
| UKBB | Mean reticulocyte volume | European | 344,728 | yes | | |
| UKBB | Mean sphered cell volume | European | 344,729 | no | correlation | Mean reticulocyte |
| | | | | | | volume |
| UKBB | High light scatter | European | 344,729 | yes | | |
| | reticulocyte count | | | | | |
| UKBB | Albumin | European | 315,268 | yes | | |
| UKBB | Alkaline phosphotase | European | 344,292 | yes | | |
| UKBB | Alanine aminotransferase | European | 344,136 | yes | | |
| UKBB | Apolipoprotein A | European | 313,387 | no | correlation | HDL cholesterol |
| UKBB | Apolipoprotein B | European | 342,590 | yes | | |
| UKBB | Aspartate aminotransferase | European | 342,990 | yes | | |
| UKBB | Direct bilirubin | European | 292,933 | yes | | |
| UKBB | Urea | European | 344,052 | yes | | |
| UKBB | Calcium | European | 315,153 | yes | | |
| UKBB | Cholesterol | European | 344,278 | no | correlation | Apolipoprotein B |
| UKBB | C-reactive protein | European | 343,524 | yes | | |
| UKBB | Gamma glutamyltransferase | European | 344,104 | yes | | |
| UKBB | Random glucose | European | 314,916 | yes | | |
| UKBB | IGF-1 | European | 342,439 | yes | | |
| UKBB | Lipoprotein A | European | 273,896 | yes | | |
| UKBB | Phosphate | European | 314,658 | yes | | |
| UKBB | Sex hormone-binding globulin | European | 312,215 | yes | | |
| UKBB | Total bilirubin | European | 342,829 | yes | | |
| UKBB | Testosterone Female | European | 154,364 | yes | | |
| UKBB | Testosterone Male | European | 157,738 | no | correlation | Sex hormone- |
| | | | | | | binding globulin |
| UKBB | Total protein | European | 314,921 | yes | | |
| UKBB | Triglycerides | European | 343,992 | yes | | |
| UKBB | Urate | European | 343,836 | yes | | |
| UKBB | Vitamin D | European | 329,247 | yes | | |
| UKBB | Diastolic blood pressure | European | 340,162 | yes | | |
| UKBB | Systolic blood pressure | European | 340,159 | yes | | |
| UKBB | LDL cholesterol | European | 343,621 | no | correlation | Apolipoprotein B |
| UKBB | HDL cholesterol | European | 315,133 | yes | | |

* p-value: no T2D variant had association with the trait (p < 0.05/336)
* correlation: correlated with another trait (|r| >= 0.85)

TABLE 4
Outcome GWAS datasets tested for T2D cluster associations

| Study | Phenotype | Population | N | Reference |
|---|---|---|---|---|
| UKBB_CARDIo | CAD | European | 336,924 | Identification of 64 Novel Genetic Loci Provides an Expanded View on the |
| GRAM | | | | Genetic Architecture of Coronary Artery Disease. van der Harst P, Verweij |
| | | | | N. Circ Res. 2018 Feb. 2; 122(3): 433-443. |
| CKDGen | CKD | Mixed | 621,324 | A catalog of genetic loci associated with kidney function from analyses of |
| | | | | a million individuals. Wuttke M, et al. Nat Genet. 2019 June; 51(6): 957-972. |
| CKDGen | eGFR | Mixed | 765,348 | A catalog of genetic loci associated with kidney function from analyses of |
| | | | | a million individuals. Wuttke M, et al. Nat Genet. 2019 June; 51(6): 957-972. |
| MEGASTROKE | Ischemic Stroke | Mixed | 511,623 | Multiancestry genome-wide association study of 520,000 subjects |
| | | | | identifies 32 loci associated with stroke and stroke subtypes. Malik R, et |
| | | | | al. Nat Genet. 2018 April; 50(4): 524-537. |
| Diabetic | Diabetic | European | 3,246 | Multiethnic Genome-Wide Association Study of Diabetic Retinopathy |
| Retinopathy | Retinopathy | | | Using Liability Threshold Modeling of Duration of Diabetes and Glycemic |
| GWAS | | | | Control. Pollack S, et al. Diabetes. 2019 February; 68(2): 441-456. |
| FinnGen | Neuropathy in | European | 96,499 | The FinnGen research project collects and analyzes health registry and |
| | T2D | | | genetic data from a nationwide network of Finnish biobanks. |
| FinnGen | Hypertension | European | 96,499 | The FinnGen research project collects and analyzes health registry and |
| | | | | genetic data from a nationwide network of Finnish biobanks. |

TABLE 5
Cluster weights for T2D Loci

| | | | | | | Lipo- | | | | ALP | Hyper | |
|---|---|---|---|---|---|---|---|---|---|---|---|---|
| | | | Beta- | Beta- | Pro- | protein | | Lipo- | Liver/ | neg- | Insulin | |
| rsID | locus | VAR_ID_hg19 | cell1 | cell2 | insulin | A | Obesity | dystrophy | Lipid | ative | Secretion | SHBG |
| rs10801922 | PTGFRN | 1_117529458_G_A | 0.915 | 0.451 | 0.099 | 0.006 | 0.000 | 0.206 | 0.029 | 0.000 | 0.059 | 0.000 |
| rs10923931 | NOTCH2 | 1_120517959_G_T | 0.777 | 0.738 | 0.000 | 0.023 | 0.283 | 0.104 | 0.081 | 0.097 | 0.053 | 0.131 |
| rs1359939 | SEC16B_1 | 1_177820861_G_A | 0.000 | 0.000 | 0.000 | 0.051 | 0.577 | 0.080 | 0.000 | 0.000 | 0.564 | 0.000 |
| rs506589 | SEC16B_2 | 1_177894287_T_C | 0.000 | 0.000 | 0.034 | 0.000 | 1.848 | 0.000 | 0.000 | 0.000 | 0.709 | 0.000 |
| rs1572993 | CNTN2 | 1_205045087_G_A | 0.000 | 0.492 | 0.000 | 0.000 | 0.455 | 0.864 | 0.000 | 0.000 | 1.900 | 0.077 |
| rs2297539 | SRGAP2 | 1_206603535_G_A | 0.000 | 0.000 | 0.686 | 0.055 | 0.033 | 0.153 | 0.140 | 0.171 | 0.423 | 0.150 |
| rs13376660 | PROX1_1 | 1_214154985_G_A | 0.866 | 0.096 | 0.157 | 0.148 | 0.151 | 0.265 | 0.147 | 0.126 | 0.152 | 0.026 |
| rs340874 | PROX1_2 | 1_214159256_T_C | 0.606 | 0.968 | 0.692 | 0.000 | 0.017 | 0.098 | 0.017 | 0.170 | 0.189 | 0.569 |
| rs2494192 | ZC3H11B | 1_219749261_G_A | 0.327 | 0.213 | 0.000 | 0.021 | 0.203 | 1.564 | 0.000 | 0.000 | 0.000 | 0.059 |
| rs4846567 | SLC30A10 | 1_219750717_G_T | 0.000 | 0.106 | 0.000 | 0.252 | 0.000 | 2.377 | 0.000 | 0.138 | 0.281 | 0.000 |
| rs499689 | ACTA1 | 1_229565732_A_G | 0.739 | 0.141 | 0.268 | 0.000 | 0.117 | 0.243 | 0.009 | 0.070 | 0.008 | 0.000 |
| rs291370 | B3GALNT2 | 1_235693041_A_G | 0.154 | 0.165 | 0.000 | 0.028 | 0.576 | 0.104 | 0.393 | 0.000 | 0.000 | 0.000 |
| rs4660293 | PABPC4 | 1_40028180_A_G | 0.000 | 0.401 | 0.000 | 0.000 | 0.982 | 0.655 | 0.250 | 0.051 | 0.884 | 0.649 |
| rs12076849 | FAF1 | 1_51362441_C_T | 0.679 | 0.157 | 0.149 | 0.291 | 0.074 | 0.782 | 0.009 | 0.139 | 0.205 | 0.296 |
| rs11677557 | FLJ14816 | 2_121317747_A_G | 1.148 | 0.270 | 0.000 | 0.000 | 0.194 | 0.859 | 0.000 | 0.235 | 0.000 | 0.182 |
| rs2084674 | ACVR2A | 2_147874140_T_C | 0.000 | 0.537 | 0.000 | 0.029 | 0.581 | 0.325 | 0.000 | 0.000 | 0.412 | 0.051 |
| rs6714881 | CYTIP | 2_158321735_C_T | 0.846 | 0.000 | 0.304 | 0.066 | 0.173 | 0.274 | 0.038 | 0.154 | 0.000 | 0.000 |
| rs7583157 | ITGB6 | 2_161114552_A_G | 0.099 | 0.104 | 0.000 | 0.230 | 0.918 | 0.544 | 0.000 | 0.000 | 0.295 | 0.041 |
| rs13389219 | COBLL1_1 | 2_165528876_C_T | 0.125 | 0.493 | 0.000 | 0.000 | 0.000 | 3.308 | 0.000 | 0.191 | 0.000 | 0.295 |
| rs13410987 | COBLL1_2 | 2_165580775_C_T | 0.000 | 0.097 | 0.005 | 0.000 | 0.000 | 1.993 | 0.002 | 0.189 | 0.217 | 0.177 |
| rs7607124 | NYAP2 | 2_226840888_C_A | 0.000 | 0.149 | 0.144 | 0.000 | 0.241 | 0.876 | 0.000 | 0.000 | 0.535 | 0.123 |
| rs2972144 | IRS1_1 | 2_227101411_A_G | 0.000 | 0.461 | 0.000 | 0.000 | 0.000 | 3.182 | 0.000 | 0.032 | 0.105 | 0.337 |
| rs1515103 | IRS1_2 | 2_227144141_C_T | 0.503 | 0.000 | 0.199 | 0.000 | 0.000 | 0.743 | 0.000 | 0.040 | 0.000 | 0.185 |
| rs6739187 | DNMT3A | 2_25513652_G_A | 0.367 | 0.168 | 0.000 | 0.000 | 0.348 | 1.072 | 0.226 | 0.129 | 0.000 | 0.000 |
| rs17802463 | DTNB | 2_25643221_G_T | 0.000 | 0.078 | 0.000 | 0.000 | 0.251 | 0.252 | 0.000 | 0.041 | 1.558 | 0.072 |
| rs780093 | GCKR | 2_27742603_T_C | 0.263 | 0.752 | 0.000 | 0.150 | 0.346 | 0.000 | 6.885 | 1.532 | 0.000 | 0.000 |
| rs4952672 | THADA_1 | 2_43422557_G_A | 0.560 | 0.421 | 0.712 | 0.000 | 0.000 | 0.376 | 0.028 | 0.359 | 0.000 | 0.032 |
| rs7607777 | THADA_2 | 2_43629931_G_T | 0.036 | 1.265 | 0.269 | 0.098 | 0.000 | 0.251 | 0.000 | 0.222 | 1.453 | 0.044 |
| rs7596101 | FANCL_1 | 2_58978436_A_G | 0.356 | 0.000 | 0.057 | 0.000 | 1.006 | 0.236 | 0.000 | 0.000 | 0.168 | 0.162 |
| rs7575523 | FANCL_2 | 2_59335104_T_G | 0.800 | 0.076 | 0.000 | 0.052 | 1.481 | 0.376 | 0.000 | 0.000 | 0.000 | 0.000 |
| rs243019 | BCL11A | 2_60585806_T_C | 0.000 | 0.830 | 0.000 | 0.130 | 0.123 | 0.000 | 0.148 | 0.000 | 1.414 | 0.000 |
| rs12463617 | TMEM18 | 2_629244_A_C | 0.093 | 0.129 | 0.000 | 0.034 | 2.305 | 0.000 | 0.000 | 0.000 | 0.096 | 0.058 |
| rs2249105 | CEP68 | 2_65287896_A_G | 0.134 | 0.719 | 0.000 | 0.000 | 0.480 | 1.357 | 0.000 | 0.116 | 0.251 | 0.060 |
| rs7569113 | SPRED2 | 2_65657041_C_T | 0.216 | 0.386 | 0.050 | 0.018 | 0.040 | 0.650 | 0.201 | 0.185 | 0.360 | 0.387 |
| rs598747 | SYN2 | 3_12112010_G_A | 0.000 | 0.000 | 0.070 | 0.000 | 0.347 | 1.306 | 0.005 | 0.033 | 0.411 | 0.119 |
| rs2877716 | ADCY5 | 3_123094451_T_C | 0.764 | 2.258 | 0.157 | 0.000 | 0.000 | 0.426 | 0.249 | 0.392 | 0.000 | 0.306 |
| rs13083375 | PPARG_1 | 3_12365308_G_T | 0.321 | 0.682 | 0.072 | 0.000 | 0.000 | 1.918 | 0.152 | 0.046 | 0.000 | 0.552 |
| rs4135275 | PPARG_2 | 3_12443844_A_G | 0.154 | 0.094 | 0.000 | 0.059 | 0.241 | 1.267 | 0.069 | 0.092 | 0.015 | 0.457 |
| rs4679370 | SLC12A8 | 3_124919777_T_C | 0.735 | 0.883 | 0.182 | 0.000 | 0.152 | 0.006 | 0.341 | 0.000 | 0.027 | 0.018 |
| rs9847824 | PLXND1 | 3_129317585_C_T | 0.390 | 0.050 | 0.000 | 0.082 | 0.253 | 0.828 | 0.109 | 0.007 | 0.019 | 0.000 |
| rs2347252 | MRAS | 3_138095525_T_C | 0.056 | 0.253 | 0.037 | 0.077 | 0.087 | 1.028 | 0.000 | 0.072 | 0.025 | 0.067 |
| rs2868425 | TSC22D2 | 3_150083011_C_T | 0.000 | 0.000 | 0.499 | 0.000 | 0.137 | 0.593 | 0.062 | 0.030 | 0.540 | 0.006 |
| rs13094115 | MBNL1 | 3_152103745_A_G | 0.000 | 0.049 | 0.495 | 0.044 | 0.234 | 0.181 | 0.290 | 0.000 | 0.563 | 0.077 |
| rs6765166 | P2RY1 | 3_152497886_G_A | 0.668 | 0.599 | 0.037 | 0.011 | 0.117 | 0.223 | 0.184 | 0.202 | 0.000 | 0.028 |
| rs1449348 | EGFEM1P | 3_168225055_C_T | 0.320 | 0.000 | 1.169 | 0.000 | 0.284 | 0.000 | 0.000 | 0.000 | 0.000 | 0.000 |
| rs6792607 | EIF5A2 | 3_170610316_A_C | 0.000 | 1.293 | 0.207 | 0.000 | 0.000 | 0.000 | 0.601 | 0.037 | 0.583 | 0.067 |
| rs4148563 | ABCC5 | 3_183725999_T_C | 0.000 | 0.544 | 0.000 | 0.000 | 0.574 | 0.343 | 0.186 | 0.077 | 0.540 | 0.000 |
| rs2290066 | IGF2BP2_1 | 3_185445842_T_C | 0.393 | 0.024 | 0.486 | 0.010 | 0.162 | 0.432 | 0.000 | 0.000 | 0.036 | 0.183 |
| rs4481184 | IGF2BP2_2 | 3_185505787_C_T | 0.452 | 0.457 | 1.192 | 0.000 | 0.000 | 0.612 | 0.093 | 0.000 | 0.085 | 0.201 |
| rs3887925 | ST6GAL1 | 3_186665645_C_T | 2.103 | 0.000 | 0.028 | 0.000 | 0.000 | 0.194 | 0.147 | 0.007 | 0.000 | 0.000 |
| rs6767001 | LPP | 3_187751389_G_A | 0.215 | 0.386 | 0.381 | 0.162 | 0.292 | 0.121 | 0.101 | 0.027 | 0.277 | 0.000 |
| rs11710420 | UBE2E2_1 | 3_23221931_G_A | 0.120 | 0.006 | 0.037 | 0.107 | 0.105 | 0.324 | 0.155 | 0.090 | 0.345 | 0.000 |
| rs1496653 | UBE2E2_2 | 3_23454790_A_G | 1.560 | 0.052 | 0.168 | 0.166 | 0.000 | 0.168 | 0.052 | 0.010 | 0.095 | 0.153 |
| rs17013433 | UBE2E2_3 | 3_23578594_G_A | 0.038 | 0.038 | 0.511 | 0.007 | 0.145 | 0.000 | 0.272 | 0.061 | 0.281 | 0.013 |
| rs10461018 | CCDC12 | 3_46995242_C_T | 0.000 | 0.000 | 0.015 | 0.077 | 0.377 | 0.964 | 0.082 | 0.025 | 1.256 | 0.323 |
| rs6446298 | TRAIP | 3_49860854_T_C | 0.000 | 0.253 | 0.000 | 0.064 | 1.361 | 0.166 | 0.163 | 0.051 | 0.960 | 0.375 |
| rs11242 | RFT1 | 3_53125922_T_C | 0.525 | 0.066 | 0.059 | 0.285 | 0.479 | 0.841 | 0.000 | 0.144 | 0.194 | 0.275 |
| rs1317838 | CACNA2D3 | 3_54823598_A_G | 0.334 | 0.284 | 0.000 | 0.025 | 0.064 | 0.201 | 0.120 | 0.075 | 0.070 | 0.050 |
| rs13434089 | ATXN7 | 3_63948566_T_C | 0.534 | 0.171 | 0.175 | 0.000 | 0.208 | 0.179 | 0.216 | 0.054 | 0.087 | 0.106 |
| rs6772129 | ADAMTS9 | 3_64700425_A_G | 0.320 | 0.263 | 0.000 | 0.083 | 0.072 | 1.258 | 0.000 | 0.000 | 0.337 | 0.000 |
| rs2272163 | ROBO2 | 3_77671721_C_A | 0.000 | 0.174 | 0.000 | 0.000 | 0.690 | 0.209 | 0.000 | 0.006 | 0.744 | 0.000 |
| rs6854709 | CENPE | 4_104129786_T_C | 0.000 | 0.085 | 0.028 | 0.089 | 0.318 | 0.293 | 0.000 | 0.000 | 1.092 | 0.203 |
| rs1296328 | PCDH18 | 4_137083193_A_C | 0.607 | 0.140 | 0.000 | 0.000 | 0.737 | 0.281 | 0.000 | 0.074 | 0.000 | 0.053 |
| rs10011838 | FBXW7 | 4_153520279_G_A | 0.073 | 0.096 | 0.124 | 0.151 | 0.022 | 0.222 | 0.214 | 0.298 | 0.363 | 0.000 |
| rs12641088 | PDGFC | 4_157680199_C_T | 0.004 | 0.178 | 0.000 | 0.068 | 0.322 | 1.462 | 0.000 | 0.091 | 0.539 | 0.158 |
| rs7667864 | DCAF16 | 4_17811914_A_C | 0.417 | 0.301 | 0.061 | 0.366 | 0.606 | 0.935 | 0.024 | 0.145 | 0.245 | 0.185 |
| rs735949 | ACSL1 | 4_185716232_T_C | 0.762 | 1.454 | 0.000 | 0.118 | 0.027 | 0.013 | 0.138 | 0.063 | 0.012 | 0.063 |
| rs13130484 | GNPDA2 | 4_45175691_C_T | 0.077 | 0.000 | 0.000 | 0.158 | 1.986 | 0.170 | 0.000 | 0.000 | 0.035 | 0.000 |
| rs6829655 | DCUN1D4 | 4_52810307_T_C | 0.008 | 0.111 | 0.223 | 0.066 | 0.451 | 0.050 | 0.000 | 0.000 | 1.530 | 0.167 |
| rs4361456 | WFS1_1 | 4_6256629_G_A | 0.491 | 0.001 | 0.751 | 0.000 | 0.221 | 0.053 | 0.168 | 0.012 | 0.000 | 0.151 |
| rs6446480 | WFS1_2 | 4_6295565_C_T | 0.134 | 0.304 | 0.860 | 0.000 | 0.293 | 0.079 | 0.079 | 0.000 | 0.497 | 0.141 |
| rs1531583 | PCGF3 | 4_744972_G_T | 1.433 | 0.000 | 0.013 | 0.064 | 0.000 | 0.013 | 0.356 | 0.386 | 0.000 | 0.000 |
| rs993380 | SCD5 | 4_83584496_A_G | 0.107 | 0.670 | 0.028 | 0.000 | 0.082 | 0.519 | 0.200 | 0.237 | 0.000 | 0.064 |
| rs13133548 | FAM13A_1 | 4_89740128_G_A | 0.000 | 0.144 | 0.000 | 0.000 | 0.000 | 1.678 | 0.027 | 0.007 | 0.000 | 0.000 |
| rs7660000 | FAM13A_2 | 4_89751858_C_T | 0.612 | 0.000 | 0.028 | 0.137 | 0.045 | 1.093 | 0.065 | 0.000 | 0.000 | 0.000 |
| rs7678054 | SMARCAD1 | 4_95093855_G_A | 0.044 | 0.000 | 0.233 | 0.032 | 0.568 | 0.170 | 0.060 | 0.055 | 0.033 | 0.061 |
| rs370176 | SLCO4C1 | 5_101595558_C_T | 0.864 | 0.000 | 0.619 | 0.140 | 0.178 | 0.272 | 0.020 | 0.023 | 0.000 | 0.000 |
| rs7729395 | PAM_1 | 5_102100576_C_T | 1.190 | 0.000 | 0.767 | 0.040 | 0.281 | 0.185 | 0.031 | 0.000 | 0.000 | 0.336 |
| rs17154889 | PAM_2 | 5_102305065_C_A | 0.550 | 0.000 | 0.491 | 0.148 | 0.535 | 0.240 | 0.235 | 0.017 | 0.000 | 0.105 |
| rs329124 | JADE2 | 5_133865452_A_G | 0.000 | 0.015 | 0.397 | 0.177 | 0.000 | 0.268 | 0.313 | 0.213 | 1.182 | 0.469 |
| rs2270561 | ANKH | 5_14847628_G_A | 0.201 | 0.284 | 0.449 | 0.050 | 0.100 | 0.175 | 0.032 | 0.000 | 0.063 | 0.175 |
| rs2963449 | EBF1 | 5_157934530_A_G | 1.090 | 0.000 | 0.139 | 0.034 | 0.022 | 0.825 | 0.127 | 0.000 | 0.000 | 0.232 |
| rs12187196 | MRPS30 | 5_44683819_A_C | 0.663 | 0.221 | 0.054 | 0.054 | 0.000 | 0.217 | 0.192 | 0.040 | 0.000 | 0.000 |
| rs17211038 | ITGA1 | 5_52118488_C_T | 0.000 | 0.175 | 0.082 | 0.000 | 0.258 | 0.085 | 0.442 | 0.138 | 0.242 | 0.000 |
| rs6876198 | ARL15_1 | 5_53303595_T_C | 0.069 | 0.028 | 0.057 | 0.040 | 0.077 | 1.468 | 0.162 | 0.069 | 0.060 | 0.337 |
| rs151804 | ARL15_2 | 5_53452060_A_G | 0.000 | 0.000 | 0.730 | 0.052 | 0.098 | 0.739 | 0.193 | 0.000 | 0.238 | 0.179 |
| rs459193 | C5orf67_1 | 5_55806751_A_G | 0.000 | 0.432 | 0.118 | 0.000 | 0.000 | 1.711 | 0.265 | 0.021 | 0.622 | 0.666 |
| rs9686661 | C5orf67_2 | 5_55861786_C_T | 0.416 | 0.299 | 0.000 | 0.000 | 0.110 | 2.043 | 0.001 | 0.462 | 0.000 | 0.321 |
| rs1469398 | PIK3R1 | 5_67726055_G_A | 0.000 | 0.000 | 0.372 | 0.153 | 0.000 | 0.889 | 0.000 | 0.027 | 0.728 | 0.000 |
| rs2307111 | POC5 | 5_75003678_T_C | 0.006 | 0.000 | 0.054 | 0.000 | 1.433 | 0.369 | 0.287 | 0.051 | 0.375 | 0.210 |
| rs4457053 | ZBED3 | 5_76424949_G_A | 0.411 | 0.902 | 0.223 | 0.000 | 0.000 | 0.600 | 0.000 | 0.083 | 0.234 | 0.000 |
| rs955897 | BHMT | 5_78431486_A_C | 0.094 | 0.000 | 0.539 | 0.001 | 0.859 | 0.275 | 0.419 | 0.000 | 0.245 | 0.231 |
| rs1665901 | BEND3 | 6_107433400_A_T | 0.065 | 0.400 | 0.025 | 0.000 | 0.226 | 0.543 | 0.124 | 0.204 | 0.139 | 0.222 |
| rs4273712 | CENPW | 6_126964510_A_G | 0.767 | 0.425 | 0.035 | 0.159 | 0.000 | 0.394 | 0.618 | 0.308 | 0.000 | 0.469 |
| rs719727 | RSPO3 | 6_127414838_A_G | 0.750 | 0.278 | 0.155 | 0.105 | 0.000 | 0.000 | 0.603 | 0.397 | 0.012 | 0.124 |
| rs9494624 | NHEG1 | 6_137300960_A_G | 0.040 | 0.026 | 0.376 | 0.114 | 0.000 | 0.250 | 0.000 | 0.000 | 0.378 | 0.189 |
| rs632057 | CITED2 | 6_139834012_T_G | 0.000 | 0.000 | 0.257 | 0.000 | 0.181 | 1.802 | 0.800 | 0.293 | 0.388 | 0.000 |
| rs614730 | HIVEP2 | 6_143066053_T_A | 0.419 | 0.000 | 0.448 | 0.119 | 0.287 | 0.279 | 0.000 | 0.021 | 0.041 | 0.000 |
| rs487152 | SLC22A3 | 6_160774486_C_A | 0.000 | 0.385 | 0.000 | 7.628 | 0.000 | 0.367 | 0.000 | 0.000 | 0.000 | 0.552 |
| rs17630640 | QKI | 6_164107529_A_G | 0.302 | 0.173 | 0.021 | 0.130 | 0.036 | 1.514 | 0.000 | 0.009 | 0.000 | 0.100 |
| rs2328524 | E2F3 | 6_20488234_G_A | 0.324 | 0.055 | 0.015 | 0.024 | 0.006 | 0.127 | 0.390 | 0.000 | 0.282 | 0.000 |
| rs9368222 | CDKAL1_1 | 6_20686996_C_A | 3.186 | 0.817 | 0.027 | 0.043 | 0.000 | 0.215 | 0.000 | 0.000 | 0.003 | 0.218 |
| rs6928012 | CDKAL1_2 | 6_20728513_C_T | 0.020 | 0.792 | 0.162 | 0.054 | 0.000 | 0.224 | 0.212 | 0.000 | 0.147 | 0.100 |
| rs4493738 | CDKAL1_3 | 6_20775660_A_G | 1.879 | 0.226 | 0.051 | 0.108 | 0.000 | 0.192 | 0.000 | 0.001 | 0.024 | 0.000 |
| rs12209877 | CDKAL1_4 | 6_21027761_G_T | 2.049 | 0.017 | 0.000 | 0.000 | 0.432 | 0.000 | 0.000 | 0.093 | 0.000 | 0.012 |
| rs1265089 | PSORS1C2 | 6_31108607_G_A | 0.095 | 0.000 | 0.654 | 0.238 | 0.057 | 0.566 | 0.094 | 0.089 | 0.530 | 0.000 |
| rs2855804 | MICB | 6_31467365_C_T | 0.000 | 0.374 | 0.051 | 0.000 | 0.000 | 0.184 | 0.845 | 0.716 | 1.458 | 0.033 |
| rs1799964 | TNF | 6_31542308_T_C | 0.007 | 0.000 | 0.084 | 0.342 | 0.383 | 0.661 | 0.000 | 0.031 | 1.470 | 0.100 |
| rs9267658 | SLC44A4 | 6_31845985_T_C | 0.012 | 0.168 | 0.101 | 0.494 | 0.360 | 0.816 | 0.012 | 0.294 | 0.966 | 0.611 |
| rs715299 | NOTCH4 | 6_32189841_T_G | 0.000 | 0.130 | 0.134 | 0.000 | 0.039 | 0.000 | 1.347 | 0.755 | 0.917 | 0.401 |
| rs3806155 | BTNL2 | 6_32373378_A_T | 0.000 | 0.152 | 0.147 | 0.042 | 0.138 | 0.601 | 0.196 | 0.000 | 0.525 | 0.000 |
| rs9273363 | HLA-DQB1 | 6_32626272_C_A | 0.021 | 0.408 | 0.227 | 0.033 | 0.000 | 0.000 | 1.257 | 0.585 | 0.260 | 0.136 |
| rs7764856 | HLA-DQA2 | 6_32680640_T_A | 0.000 | 0.041 | 0.000 | 0.222 | 0.534 | 1.202 | 0.000 | 0.000 | 1.512 | 0.000 |
| rs998584 | VEGFA_1 | 6_43757896_C_A | 1.768 | 0.000 | 0.000 | 0.000 | 0.000 | 2.844 | 0.000 | 0.000 | 0.000 | 0.000 |
| rs10456526 | VEGFA_2 | 6_43814625_G_A | 0.874 | 0.394 | 0.441 | 0.000 | 0.000 | 0.000 | 0.661 | 0.473 | 0.052 | 0.031 |
| rs987237 | TFAP2B | 6_50803050_A_G | 0.000 | 0.000 | 0.511 | 0.000 | 1.685 | 0.000 | 0.000 | 0.000 | 0.468 | 0.189 |
| rs10458103 | RREB1_1 | 6_7037738_G_A | 0.000 | 0.179 | 0.109 | 0.059 | 0.000 | 0.412 | 0.153 | 0.195 | 0.940 | 0.171 |
| rs9379084 | RREB1_2 | 6_7231843_G_A | 0.863 | 0.864 | 0.000 | 0.000 | 0.001 | 0.049 | 0.806 | 0.169 | 0.000 | 0.000 |
| rs9505086 | RREB1_3 | 6_7232186_T_C | 0.683 | 0.654 | 0.129 | 0.003 | 0.000 | 0.050 | 0.492 | 0.277 | 0.072 | 0.000 |
| rs11496066 | FBXL13 | 7_102486254_T_C | 0.912 | 0.000 | 0.772 | 0.047 | 0.442 | 0.000 | 0.416 | 0.122 | 0.076 | 0.150 |
| rs39328 | RELN | 7_103444978_C_T | 0.000 | 0.000 | 0.250 | 0.000 | 0.553 | 0.000 | 0.115 | 0.159 | 0.670 | 0.000 |
| rs6976111 | CTTNBP2 | 7_117495667_C_A | 0.067 | 0.000 | 0.029 | 0.000 | 0.544 | 0.195 | 0.028 | 0.000 | 0.375 | 0.000 |
| rs287621 | KLF14 | 7_130435181_T_C | 0.000 | 0.430 | 0.000 | 0.000 | 0.000 | 1.685 | 0.000 | 0.145 | 0.000 | 0.214 |
| rs10276674 | DGKB_1 | 7_14922007_T_C | 0.471 | 0.718 | 1.064 | 0.000 | 0.028 | 0.000 | 0.225 | 0.000 | 0.229 | 0.148 |
| rs6977081 | AOC1 | 7_150542515_G_T | 0.000 | 0.046 | 0.018 | 0.000 | 0.076 | 0.856 | 0.197 | 0.000 | 0.730 | 0.078 |
| rs10244051 | DGKB_2 | 7_15063833_T_G | 0.494 | 1.777 | 0.333 | 0.000 | 0.000 | 0.000 | 0.142 | 0.071 | 0.198 | 0.000 |
| rs2301916 | UBE3C | 7_156974127_G_T | 0.982 | 0.325 | 0.001 | 0.059 | 0.460 | 0.059 | 0.000 | 0.010 | 0.000 | 0.000 |
| rs4279506 | IGF2BP3 | 7_23512896_G_C | 0.048 | 0.057 | 0.757 | 0.254 | 0.263 | 0.230 | 0.000 | 0.000 | 0.627 | 0.036 |
| rs6948511 | JAZF1_1 | 7_27978715_T_C | 0.343 | 0.701 | 0.000 | 0.050 | 0.143 | 0.261 | 0.000 | 0.034 | 0.000 | 0.090 |
| rs849133 | JAZF1_2 | 7_28192280_C_T | 0.486 | 0.451 | 0.033 | 0.000 | 0.238 | 0.710 | 0.206 | 0.291 | 0.000 | 0.494 |
| rs2267716 | CRHR2 | 7_30716643_T_C | 1.571 | 0.000 | 0.424 | 0.000 | 0.284 | 0.185 | 0.000 | 0.035 | 0.001 | 0.130 |
| rs2971669 | GCK | 7_44231778_C_T | 1.186 | 2.101 | 1.117 | 0.000 | 0.034 | 0.284 | 0.136 | 0.000 | 0.209 | 0.301 |
| rs11765592 | C1GALT1 | 7_7250753_T_C | 0.128 | 0.282 | 0.026 | 0.000 | 0.300 | 0.345 | 0.078 | 0.058 | 0.216 | 0.096 |
| rs17151859 | MSRA_2 | 8_10237980_A_C | 0.015 | 0.054 | 0.045 | 0.093 | 0.508 | 0.020 | 0.197 | 0.103 | 1.281 | 0.000 |
| rs10903337 | XKR6 | 8_10905594_G_C | 0.038 | 0.151 | 0.168 | 0.191 | 0.378 | 0.152 | 0.247 | 0.061 | 0.722 | 0.053 |
| rs4129682 | TRHR | 8_110122139_C_T | 0.000 | 0.626 | 0.000 | 0.053 | 0.395 | 0.057 | 0.000 | 0.000 | 0.781 | 0.000 |
| rs3802177 | SLC30A8 | 8_118185025_G_A | 1.987 | 3.125 | 0.000 | 0.021 | 0.000 | 0.024 | 0.000 | 0.000 | 0.000 | 0.256 |
| rs1561927 | TMEM75 | 8_129568078_C_T | 0.369 | 0.136 | 0.871 | 0.000 | 0.150 | 0.000 | 0.486 | 0.256 | 0.000 | 0.008 |
| rs7828303 | BOP1 | 8_145509128_T_C | 0.000 | 0.568 | 0.139 | 0.010 | 0.000 | 0.256 | 0.047 | 0.299 | 1.337 | 0.198 |
| rs2272662 | SLC39A4 | 8_145639726_T_C | 0.000 | 0.163 | 0.103 | 0.031 | 0.000 | 0.322 | 0.000 | 0.990 | 0.000 | 0.321 |
| rs997258 | ARHGAP39 | 8_145903077_C_A | 0.000 | 0.024 | 0.381 | 0.000 | 0.191 | 0.303 | 0.144 | 0.367 | 0.584 | 0.351 |
| rs10096633 | LPL | 8_19830921_C_T | 0.000 | 0.022 | 0.000 | 0.000 | 0.202 | 2.692 | 0.019 | 0.000 | 0.000 | 0.000 |
| rs4733067 | PURG | 8_30856943_A_G | 0.000 | 0.000 | 0.169 | 0.035 | 0.987 | 0.313 | 0.007 | 0.000 | 0.438 | 0.046 |
| rs2354575 | GPAT4_1 | 8_41462898_A_G | 0.815 | 0.000 | 0.065 | 0.000 | 0.131 | 0.058 | 0.266 | 0.086 | 0.000 | 0.000 |
| rs11995712 | GPAT4_2 | 8_41472375_G_A | 0.216 | 0.138 | 1.029 | 0.000 | 0.047 | 0.038 | 0.194 | 0.000 | 0.029 | 0.015 |
| rs12544241 | ANK1_1 | 8_41514888_G_A | 2.353 | 0.072 | 0.522 | 0.000 | 0.000 | 0.000 | 0.361 | 0.081 | 0.000 | 0.000 |
| rs16890723 | ANK1_2 | 8_41517064_T_C | 1.189 | 0.000 | 0.782 | 0.035 | 0.010 | 0.000 | 0.184 | 0.047 | 0.000 | 0.000 |
| rs3102078 | PPP1R3B_1 | 8_9050725_C_A | 0.000 | 0.299 | 0.000 | 0.040 | 0.637 | 0.007 | 0.246 | 0.063 | 0.997 | 0.035 |
| rs9987289 | PPP1R3B_2 | 8_9183358_A_G | 0.000 | 0.336 | 0.000 | 0.000 | 0.613 | 0.630 | 1.994 | 0.000 | 2.844 | 0.506 |
| rs17662402 | TNKS | 8_9265105_T_C | 0.000 | 0.442 | 0.000 | 0.033 | 0.187 | 0.329 | 0.385 | 0.014 | 0.605 | 0.018 |
| rs9297945 | DPY19L4 | 8_95749023_A_G | 0.085 | 0.208 | 0.646 | 0.000 | 0.000 | 0.264 | 0.277 | 0.095 | 0.075 | 0.000 |
| rs13255935 | NDUFAF6 | 8_95965685_C_A | 0.000 | 0.341 | 0.310 | 0.000 | 0.265 | 0.197 | 0.462 | 0.000 | 1.079 | 0.000 |
| rs17689007 | MSRA_1 | 8_9974824_G_A | 0.161 | 0.229 | 0.040 | 0.104 | 0.948 | 0.100 | 0.396 | 0.148 | 0.667 | 0.004 |
| rs545971 | ABO | 9_136143372_C_T | 0.916 | 0.426 | 0.000 | 0.002 | 0.146 | 0.000 | 0.000 | 8.290 | 0.000 | 0.264 |
| rs3829109 | DNLZ | 9_139256766_G_A | 1.127 | 0.821 | 0.091 | 0.157 | 0.000 | 0.196 | 0.112 | 0.670 | 0.000 | 0.072 |
| rs11145846 | SNAPC4 | 9_139285655_A_G | 0.922 | 0.107 | 0.480 | 0.104 | 0.145 | 0.000 | 0.123 | 0.134 | 0.000 | 0.002 |
| rs7467618 | HAUS6 | 9_19065295_T_C | 1.739 | 0.448 | 0.060 | 0.000 | 0.028 | 0.373 | 0.197 | 0.151 | 0.000 | 0.000 |
| rs7867635 | MLLT3 | 9_20241069_T_C | 0.249 | 0.414 | 0.000 | 0.146 | 0.566 | 0.298 | 0.000 | 0.009 | 0.000 | 0.000 |
| rs523096 | CDKN2B_1 | 9_22019129_A_G | 1.464 | 0.163 | 0.068 | 0.000 | 0.144 | 0.103 | 0.397 | 0.000 | 0.000 | 0.000 |
| rs17694572 | CDKN2B_2 | 9_22054356_G_A | 0.263 | 0.581 | 0.000 | 0.015 | 0.168 | 0.281 | 0.384 | 0.066 | 0.079 | 0.039 |
| rs10811658 | CDKN2B_3 | 9_22128600_G_A | 0.034 | 0.370 | 0.200 | 0.000 | 0.037 | 0.090 | 0.202 | 0.078 | 0.125 | 0.223 |
| rs10811661 | CDKN2B_4 | 9_22134094_T_C | 1.803 | 0.509 | 0.244 | 0.000 | 0.000 | 0.414 | 0.004 | 0.000 | 0.000 | 0.273 |
| rs1412234 | LINGO2 | 9_28410683_T_C | 0.034 | 0.214 | 0.000 | 0.000 | 1.501 | 0.411 | 0.095 | 0.000 | 0.000 | 0.000 |
| rs10758593 | GLIS3 | 9_4292083_G_A | 0.645 | 1.410 | 0.231 | 0.000 | 0.000 | 0.034 | 0.505 | 0.080 | 0.000 | 0.385 |
| rs11137820 | PSAT1 | 9_81359113_C_G | 0.325 | 0.000 | 0.261 | 0.000 | 0.475 | 0.210 | 0.136 | 0.040 | 0.060 | 0.075 |
| rs17791513 | TLE4 | 9_81905590_A_G | 0.721 | 0.342 | 0.000 | 0.042 | 0.176 | 0.000 | 0.120 | 0.026 | 0.030 | 0.081 |
| rs2796441 | TLE1 | 9_84308948_G_A | 0.000 | 0.647 | 0.000 | 0.000 | 0.068 | 0.170 | 0.129 | 0.000 | 0.730 | 0.000 |
| rs7872931 | ZNF169 | 9_96970197_G_A | 0.620 | 0.058 | 0.244 | 0.000 | 0.652 | 0.169 | 0.146 | 0.030 | 0.062 | 0.189 |
| rs17746088 | VTI1A_1 | 10_114598202_T_C | 0.531 | 0.131 | 0.211 | 0.151 | 0.000 | 0.277 | 0.252 | 0.186 | 0.001 | 0.000 |
| rs11815670 | VTI1A_2 | 10_114613439_G_A | 0.025 | 0.737 | 0.000 | 0.000 | 0.276 | 0.000 | 0.118 | 0.023 | 0.390 | 0.000 |
| rs1927157 | VTI1A_3 | 10_114635381_T_C | 0.026 | 0.864 | 0.000 | 0.149 | 0.191 | 0.057 | 0.015 | 0.099 | 0.580 | 0.000 |
| rs1885283 | VTI1A_4 | 10_114648499_C_A | 0.000 | 1.131 | 0.000 | 0.168 | 0.118 | 0.181 | 0.022 | 0.000 | 0.763 | 0.000 |
| rs7079711 | TCF7L2_1 | 10_114745788_G_A | 1.062 | 1.042 | 0.054 | 0.121 | 0.000 | 0.378 | 0.000 | 0.025 | 0.000 | 0.047 |
| rs17747324 | TCF7L2_2 | 10_114752503_T_C | 0.932 | 4.070 | 0.000 | 0.241 | 0.011 | 0.106 | 0.000 | 0.051 | 0.000 | 0.298 |
| rs11196187 | TCF7L2_3 | 10_114759445_G_A | 1.480 | 1.533 | 0.008 | 0.106 | 0.000 | 0.157 | 0.000 | 0.137 | 0.000 | 0.000 |
| rs12354626 | TCF7L2_4 | 10_114772429_G_A | 0.833 | 0.866 | 0.000 | 0.002 | 0.209 | 0.000 | 0.085 | 0.000 | 0.000 | 0.000 |
| rs7087006 | TCF7L2_5 | 10_114823426_A_G | 0.281 | 1.868 | 0.000 | 0.148 | 0.048 | 0.048 | 0.160 | 0.094 | 0.000 | 0.000 |
| rs10885410 | TCF7L2_6 | 10_114824473_G_A | 0.868 | 1.062 | 0.113 | 0.000 | 0.000 | 0.049 | 0.000 | 0.226 | 0.000 | 0.163 |
| rs6585206 | TCF7L2_7 | 10_114859251_A_G | 0.691 | 0.590 | 0.000 | 0.126 | 0.000 | 0.423 | 0.000 | 0.094 | 0.000 | 0.121 |
| rs10885414 | TCF7L2_8 | 10_114861304_A_G | 0.409 | 1.166 | 0.000 | 0.146 | 0.000 | 0.414 | 0.000 | 0.200 | 0.000 | 0.006 |
| rs4918796 | TCF7L2_9 | 10_114880342_T_C | 0.622 | 0.103 | 0.116 | 0.055 | 0.000 | 0.161 | 0.071 | 0.108 | 0.040 | 0.178 |
| rs290483 | TCF7L2_10 | 10_114915214_G_T | 0.411 | 0.629 | 0.080 | 0.127 | 0.000 | 0.206 | 0.395 | 0.149 | 0.000 | 0.000 |
| rs7895525 | CDC123_1 | 10_12250620_T_C | 0.795 | 0.480 | 0.934 | 0.043 | 0.000 | 0.312 | 0.000 | 0.002 | 0.011 | 0.097 |
| rs10906115 | CDC123_2 | 10_12314997_A_G | 0.000 | 0.169 | 0.362 | 0.021 | 0.000 | 0.135 | 0.141 | 0.011 | 0.556 | 0.000 |
| rs11200594 | PLEKHA1 | 10_124139393_C_T | 1.067 | 0.000 | 0.259 | 0.162 | 0.009 | 0.461 | 0.000 | 0.052 | 0.005 | 0.000 |
| rs2616055 | NEUROG3_1 | 10_71317835_G_A | 0.118 | 0.465 | 0.000 | 0.004 | 0.114 | 0.268 | 0.114 | 0.063 | 0.236 | 0.000 |
| rs177045 | NEUROG3_2 | 10_71321279_A_G | 0.500 | 0.000 | 0.891 | 0.056 | 0.149 | 0.045 | 0.251 | 0.000 | 0.106 | 0.071 |
| rs2812533 | FAM241B_1 | 10_71452285_T_C | 1.132 | 0.207 | 0.071 | 0.180 | 0.000 | 0.112 | 0.000 | 0.057 | 0.013 | 0.031 |
| rs2642588 | FAM241B_2 | 10_71466578_T_G | 0.164 | 0.000 | 0.417 | 0.109 | 0.000 | 0.266 | 0.067 | 0.092 | 0.029 | 0.085 |
| rs697239 | ZMIZ1_1 | 10_80947438_T_C | 0.028 | 0.201 | 0.000 | 0.000 | 0.404 | 0.590 | 0.020 | 0.009 | 0.126 | 0.148 |
| rs10509406 | ZMIZ1_2 | 10_80959517_T_C | 0.497 | 0.000 | 0.588 | 0.015 | 0.498 | 0.150 | 0.000 | 0.001 | 0.041 | 0.173 |
| rs11202627 | PTEN | 10_89769340_C_T | 0.000 | 0.961 | 0.000 | 0.100 | 0.487 | 0.630 | 0.077 | 0.003 | 0.754 | 0.000 |
| rs7094274 | CPEB3 | 10_93945476_C_T | 1.457 | 0.201 | 0.018 | 0.075 | 0.268 | 0.143 | 0.000 | 0.000 | 0.051 | 0.279 |
| rs2209972 | MARK2P9 | 10_94179028_C_T | 1.119 | 0.000 | 0.085 | 0.000 | 0.161 | 0.308 | 0.150 | 0.170 | 0.000 | 0.000 |
| rs5015480 | HHEX | 10_94465559_C_T | 2.918 | 0.000 | 0.000 | 0.000 | 0.114 | 0.359 | 0.030 | 0.002 | 0.000 | 0.022 |
| rs10893829 | ETS1_1 | 11_128042575_T_C | 0.326 | 0.056 | 0.157 | 0.000 | 0.044 | 0.393 | 0.203 | 0.059 | 0.077 | 0.029 |
| rs10750397 | ETS1_2 | 11_128234144_A_G | 0.274 | 0.174 | 0.000 | 0.074 | 0.011 | 0.234 | 0.262 | 0.142 | 0.072 | 0.022 |
| rs11819995 | ETS1_3 | 11_128389391_C_T | 0.043 | 0.772 | 0.000 | 0.124 | 0.168 | 0.000 | 0.105 | 0.022 | 0.140 | 0.000 |
| rs757110 | ABCC8 | 11_17418477_C_A | 0.987 | 0.097 | 0.861 | 0.041 | 0.061 | 0.121 | 0.235 | 0.071 | 0.059 | 0.180 |
| rs11600952 | IGF2 | 11_2101283_G_A | 1.489 | 0.000 | 0.056 | 0.339 | 0.000 | 0.419 | 0.000 | 0.096 | 0.000 | 0.000 |
| rs7111341 | TH | 11_2213166_C_T | 0.038 | 0.164 | 0.005 | 0.053 | 0.389 | 0.618 | 0.103 | 0.000 | 0.278 | 0.127 |
| rs231349 | KCNQ1_1 | 11_2672821_C_T | 0.023 | 0.046 | 0.120 | 0.046 | 0.130 | 0.248 | 0.153 | 0.000 | 0.532 | 0.117 |
| rs231362 | KCNQ1_2 | 11_2691471_A_G | 0.081 | 0.756 | 0.483 | 0.060 | 0.000 | 0.369 | 0.158 | 0.000 | 0.323 | 0.036 |
| rs2283222 | KCNQ1_3 | 11_2755601_T_C | 0.002 | 0.000 | 0.944 | 0.000 | 0.000 | 0.332 | 0.037 | 0.000 | 0.250 | 0.069 |
| rs2074197 | KCNQ1_4 | 11_2825279_C_T | 0.411 | 0.661 | 0.227 | 0.132 | 0.000 | 0.178 | 0.123 | 0.000 | 0.052 | 0.000 |
| rs163177 | KCNQ1_5 | 11_2838413_T_C | 1.158 | 0.641 | 0.084 | 0.000 | 0.000 | 0.439 | 0.157 | 0.000 | 0.000 | 0.096 |
| rs4923543 | METTL15 | 11_28534898_G_A | 0.013 | 0.000 | 1.068 | 0.066 | 0.111 | 0.223 | 0.355 | 0.078 | 0.310 | 0.019 |
| rs504034 | PDHX | 11_34997562_T_C | 0.000 | 0.399 | 0.022 | 0.000 | 0.367 | 0.262 | 0.000 | 0.000 | 0.763 | 0.033 |
| rs1061810 | HSD17B12 | 11_43877934_C_A | 1.290 | 0.000 | 0.000 | 0.000 | 1.189 | 0.226 | 0.000 | 0.000 | 0.000 | 0.201 |
| rs12805422 | SLC35C1 | 11_45839889_G_A | 2.031 | 0.285 | 0.461 | 0.000 | 0.000 | 0.000 | 0.269 | 0.000 | 0.000 | 0.000 |
| rs7124681 | CELF1 | 11_47529947_C_A | 0.000 | 1.484 | 0.000 | 0.295 | 1.659 | 0.328 | 0.133 | 0.309 | 0.102 | 0.000 |
| rs694243 | FRMD8 | 11_65172197_C_T | 0.000 | 0.369 | 0.041 | 0.116 | 0.070 | 0.484 | 0.277 | 0.264 | 0.579 | 0.368 |
| rs947791 | SCYL1 | 11_65302893_G_A | 0.000 | 0.000 | 0.043 | 0.000 | 0.589 | 0.259 | 0.301 | 0.223 | 1.345 | 0.420 |
| rs3814707 | OVOL1 | 11_65560785_G_A | 0.499 | 0.000 | 0.000 | 0.168 | 0.554 | 1.113 | 0.070 | 0.029 | 0.001 | 0.500 |
| rs3918298 | CCND1 | 11_69463273_G_A | 0.729 | 0.189 | 0.491 | 0.048 | 0.063 | 0.139 | 0.300 | 0.190 | 0.000 | 0.000 |
| rs11603334 | ARAP1 | 11_72432985_G_A | 0.501 | 0.000 | 5.705 | 0.000 | 0.000 | 0.000 | 0.000 | 0.016 | 0.000 | 0.000 |
| rs663015 | STARD10 | 11_72499035_T_C | 0.000 | 0.000 | 3.824 | 0.072 | 0.118 | 0.000 | 0.162 | 0.000 | 0.318 | 0.000 |
| rs1387153 | MTNR1B_1 | 11_92673828_C_T | 4.659 | 1.614 | 0.000 | 0.000 | 0.086 | 0.000 | 0.000 | 0.000 | 0.000 | 0.097 |
| rs9971402 | MTNR1B_2 | 11_92756551_C_A | 1.318 | 0.443 | 0.009 | 0.019 | 0.141 | 0.006 | 0.063 | 0.000 | 0.000 | 0.118 |
| rs1900385 | SLC36A4 | 11_93014343_T_G | 0.523 | 0.189 | 0.249 | 0.000 | 0.206 | 0.000 | 0.103 | 0.079 | 0.000 | 0.071 |
| rs3764002 | WSCD2 | 12_108618630_C_T | 1.928 | 0.000 | 0.000 | 0.000 | 0.595 | 0.464 | 0.000 | 0.038 | 0.000 | 0.029 |
| rs2241737 | RFC5 | 12_118454833_C_G | 0.000 | 0.051 | 0.039 | 0.055 | 0.216 | 0.253 | 0.066 | 0.055 | 0.309 | 0.046 |
| rs550349 | SPPL3_1 | 12_121236160_A_C | 1.543 | 0.000 | 0.000 | 0.220 | 0.000 | 0.049 | 1.136 | 0.000 | 0.013 | 0.471 |
| rs11065299 | SPPL3_2 | 12_121297815_G_A | 0.978 | 0.159 | 0.380 | 0.086 | 0.000 | 0.214 | 0.871 | 0.008 | 0.000 | 0.284 |
| rs1169302 | HNF1A | 12_121432302_T_G | 1.197 | 0.000 | 0.517 | 0.000 | 0.000 | 0.000 | 2.144 | 0.000 | 0.000 | 0.426 |
| rs7298521 | P2RX7_1 | 12_121541831_C_T | 0.617 | 0.001 | 0.732 | 0.059 | 0.000 | 0.072 | 0.150 | 0.000 | 0.014 | 0.179 |
| rs208302 | P2RX7_2 | 12_121602760_G_A | 0.723 | 0.000 | 0.287 | 0.165 | 0.358 | 0.266 | 0.313 | 0.001 | 0.000 | 0.158 |
| rs11057276 | SBNO1 | 12_123820397_T_C | 0.799 | 0.314 | 0.000 | 0.189 | 0.092 | 0.671 | 0.263 | 0.285 | 0.083 | 0.000 |
| rs11837287 | ZNF664 | 12_124470059_C_T | 0.000 | 0.080 | 0.000 | 0.000 | 0.000 | 2.284 | 0.000 | 0.058 | 1.211 | 0.015 |
| rs863750 | FAM101A | 12_124505444_C_T | 0.000 | 0.000 | 0.000 | 0.000 | 0.000 | 2.121 | 0.000 | 0.096 | 0.870 | 0.139 |
| rs1882297 | FBRSL1 | 12_133069134_G_A | 0.851 | 0.765 | 0.108 | 0.050 | 0.094 | 0.136 | 0.008 | 0.182 | 0.000 | 0.000 |
| rs2343872 | SSPN_1 | 12_26398707_C_T | 0.537 | 0.000 | 0.000 | 0.045 | 0.322 | 0.756 | 0.189 | 0.055 | 0.021 | 0.000 |
| rs1872992 | SSPN_2 | 12_26457190_A_G | 0.623 | 0.377 | 0.000 | 0.062 | 0.250 | 1.497 | 0.001 | 0.380 | 0.000 | 0.000 |
| rs10492373 | KLHL42 | 12_27959998_G_A | 0.019 | 0.644 | 0.104 | 0.000 | 0.068 | 0.131 | 0.085 | 0.136 | 0.283 | 0.273 |
| rs7963651 | CCND2_1 | 12_4297831_C_A | 0.104 | 0.385 | 0.013 | 0.000 | 0.348 | 0.161 | 0.005 | 0.024 | 0.031 | 0.064 |
| rs11063069 | CCND2_2 | 12_4374373_A_G | 0.000 | 0.157 | 1.073 | 0.109 | 0.000 | 0.549 | 0.086 | 0.043 | 0.150 | 0.117 |
| rs3217795 | CCND2_3 | 12_4386064_A_G | 0.531 | 0.405 | 0.441 | 0.000 | 0.151 | 0.000 | 0.352 | 0.134 | 0.000 | 0.000 |
| rs3217862 | CCND2_4 | 12_4399087_T_G | 0.008 | 0.186 | 0.283 | 0.092 | 0.202 | 0.296 | 0.056 | 0.056 | 0.188 | 0.207 |
| rs12299509 | CCND2_5 | 12_4406281_A_G | 0.025 | 0.250 | 0.144 | 0.133 | 0.040 | 0.050 | 0.203 | 0.232 | 0.226 | 0.253 |
| rs10849090 | DYRK4 | 12_4669954_A_G | 0.332 | 0.107 | 0.105 | 0.000 | 0.000 | 0.381 | 0.116 | 0.789 | 0.125 | 0.000 |
| rs17101712 | RPSAP52_1 | 12_66166746_T_A | 0.455 | 0.424 | 0.015 | 0.115 | 0.115 | 0.268 | 0.081 | 0.049 | 0.055 | 0.145 |
| rs2612075 | RPSAP52_2 | 12_66209646_C_T | 0.174 | 0.144 | 0.255 | 0.040 | 0.106 | 0.449 | 0.126 | 0.099 | 0.268 | 0.306 |
| rs1042725 | HMGA2 | 12_66358347_C_T | 0.291 | 0.000 | 0.573 | 0.659 | 0.051 | 0.906 | 0.000 | 0.100 | 0.215 | 0.356 |
| rs1051334 | TSPAN8 | 12_71523134_A_C | 0.970 | 0.084 | 0.035 | 0.002 | 0.026 | 0.378 | 0.089 | 0.061 | 0.000 | 0.180 |
| rs2197973 | USP44 | 12_95928560_C_T | 0.381 | 0.485 | 0.000 | 0.232 | 0.661 | 0.137 | 0.000 | 0.167 | 0.085 | 0.272 |
| rs1592519 | RMST | 12_97847018_C_T | 0.263 | 0.544 | 0.624 | 0.005 | 0.048 | 0.041 | 0.141 | 0.060 | 0.205 | 0.000 |
| rs9555581 | MYO16 | 13_109944192_C_T | 0.000 | 0.000 | 0.772 | 0.000 | 0.302 | 0.468 | 0.000 | 0.000 | 0.064 | 0.010 |
| rs10507349 | RNF6 | 13_26781528_G_A | 0.849 | 0.326 | 0.000 | 0.163 | 0.000 | 0.369 | 0.024 | 0.035 | 0.000 | 0.069 |
| rs1045411 | HMGB1 | 13_31033232_C_T | 0.317 | 0.171 | 0.000 | 0.063 | 0.798 | 0.659 | 0.000 | 0.025 | 0.000 | 0.000 |
| rs576674 | KL | 13_33554302_G_A | 1.221 | 0.771 | 0.082 | 0.000 | 0.037 | 0.000 | 0.214 | 0.107 | 0.000 | 0.023 |
| rs9316500 | DLEU1 | 13_51094114_T_G | 0.111 | 0.145 | 0.445 | 0.018 | 0.240 | 0.051 | 0.677 | 0.049 | 0.109 | 0.000 |
| rs7988007 | PCDH17 | 13_58988271_A_C | 0.000 | 0.000 | 0.000 | 0.000 | 0.572 | 0.124 | 0.000 | 0.000 | 1.367 | 0.098 |
| rs1359790 | SPRY2 | 13_80717156_G_A | 0.020 | 0.523 | 0.934 | 0.145 | 0.062 | 0.050 | 0.000 | 0.031 | 0.908 | 0.144 |
| rs1028613 | MARK3 | 14_103888324_G_A | 0.000 | 0.000 | 0.011 | 0.172 | 0.882 | 0.254 | 0.000 | 0.187 | 0.651 | 0.000 |
| rs7145828 | AKAP6 | 14_33303298_A_G | 0.645 | 0.458 | 0.000 | 0.029 | 0.946 | 0.092 | 0.000 | 0.031 | 0.000 | 0.076 |
| rs8017808 | CLEC14A | 14_38848419_G_T | 0.820 | 0.423 | 0.000 | 0.000 | 0.297 | 0.547 | 0.000 | 0.093 | 0.000 | 0.000 |
| rs10150332 | NRXN3 | 14_79936964_T_C | 0.000 | 0.000 | 0.000 | 0.160 | 1.468 | 0.522 | 0.127 | 0.000 | 0.000 | 0.000 |
| rs1742546 | CCDC88C | 14_91883499_G_A | 0.437 | 0.198 | 0.281 | 0.000 | 0.132 | 0.130 | 0.225 | 0.122 | 0.000 | 0.215 |
| rs8032939 | RASGRP1 | 15_38834033_T_C | 0.452 | 0.140 | 0.658 | 0.028 | 0.398 | 0.000 | 0.053 | 0.097 | 0.239 | 0.048 |
| rs11637681 | BMF | 15_40387971_A_G | 0.303 | 0.057 | 0.008 | 0.183 | 0.020 | 0.895 | 0.016 | 0.110 | 0.348 | 0.323 |
| rs2289739 | LTK | 15_41801512_G_T | 0.000 | 0.052 | 0.175 | 0.000 | 0.358 | 0.613 | 0.000 | 0.029 | 0.108 | 0.063 |
| rs2456530 | ONECUT1 | 15_53091553_C_T | 0.000 | 0.063 | 0.000 | 0.000 | 0.477 | 0.249 | 0.000 | 0.000 | 1.210 | 0.000 |
| rs12440878 | TCF12 | 15_57535215_G_A | 0.033 | 0.141 | 0.000 | 0.000 | 0.551 | 0.180 | 0.058 | 0.037 | 0.193 | 0.072 |
| rs7163757 | C2CD4A | 15_62391608_C_T | 2.780 | 1.708 | 0.000 | 0.401 | 0.000 | 0.000 | 0.450 | 0.000 | 0.000 | 0.000 |
| rs7178762 | USP3 | 15_63871292_C_T | 0.234 | 0.249 | 0.000 | 0.083 | 0.571 | 0.658 | 0.141 | 0.015 | 0.000 | 0.354 |
| rs4776970 | MAP2K5 | 15_68080886_A_T | 0.000 | 0.041 | 0.012 | 0.006 | 1.160 | 0.000 | 0.000 | 0.000 | 0.559 | 0.000 |
| rs7166281 | IMP3 | 15_75928192_T_G | 0.214 | 0.604 | 0.171 | 0.144 | 0.124 | 0.319 | 0.190 | 0.052 | 0.796 | 0.232 |
| rs4886508 | PSTPIP1 | 15_77307910_C_T | 0.000 | 0.154 | 0.036 | 0.151 | 0.177 | 0.303 | 0.000 | 0.000 | 1.547 | 0.000 |
| rs1005752 | HMG20A | 15_77818128_C_A | 0.000 | 0.589 | 0.020 | 0.111 | 0.441 | 0.518 | 0.000 | 0.000 | 0.573 | 0.005 |
| rs17242083 | ARPIN | 15_90449476_C_T | 1.261 | 0.441 | 0.000 | 0.204 | 0.020 | 0.123 | 0.000 | 0.043 | 0.003 | 0.082 |
| rs2290203 | PRC1 | 15_91512067_G_A | 1.300 | 0.170 | 0.169 | 0.000 | 0.116 | 0.119 | 0.493 | 0.097 | 0.167 | 0.000 |
| rs7189927 | ATP2A1 | 16_28913787_T_C | 0.000 | 0.470 | 0.000 | 0.165 | 1.910 | 0.131 | 0.209 | 0.178 | 0.896 | 0.217 |
| rs11248914 | FAM234A | 16_293562_T_C | 1.164 | 0.318 | 0.206 | 0.013 | 0.129 | 0.144 | 0.000 | 0.130 | 0.353 | 0.253 |
| rs12325539 | DOC2A | 16_30033633_T_C | 0.131 | 0.612 | 0.000 | 0.158 | 1.113 | 0.361 | 0.201 | 0.394 | 0.000 | 0.287 |
| rs12445430 | NLRC3 | 16_3613126_C_T | 0.709 | 0.183 | 0.000 | 0.000 | 0.758 | 0.335 | 0.054 | 0.011 | 0.000 | 0.215 |
| rs3095571 | AKTIP | 16_53544993_G_A | 0.905 | 0.004 | 0.084 | 0.022 | 0.137 | 0.736 | 0.000 | 0.045 | 0.000 | 0.000 |
| rs1421085 | FTO_1 | 16_53800954_T_C | 0.090 | 0.024 | 0.010 | 0.000 | 4.456 | 0.000 | 0.000 | 0.000 | 0.000 | 0.236 |
| rs16952522 | FTO_2 | 16_53807498_C_G | 0.055 | 0.000 | 0.109 | 0.000 | 1.074 | 0.371 | 0.000 | 0.000 | 0.405 | 0.000 |
| rs6499646 | FTO_3 | 16_53843533_T_C | 0.007 | 0.000 | 0.064 | 0.000 | 0.896 | 0.143 | 0.000 | 0.000 | 0.647 | 0.000 |
| rs17218700 | FTO_4 | 16_53844579_G_A | 0.000 | 0.497 | 0.000 | 0.002 | 0.987 | 0.078 | 0.000 | 0.021 | 0.082 | 0.082 |
| rs6499653 | FTO_5 | 16_53877592_T_C | 0.010 | 0.311 | 0.003 | 0.000 | 1.295 | 0.067 | 0.000 | 0.000 | 0.017 | 0.000 |
| rs889398 | NFAT5 | 16_69556715_C_T | 0.052 | 0.154 | 0.028 | 0.105 | 1.284 | 0.644 | 0.000 | 0.000 | 0.223 | 0.165 |
| rs7202844 | CTRB1 | 16_75247391_A_G | 1.433 | 0.464 | 0.000 | 0.153 | 0.223 | 0.279 | 0.042 | 0.016 | 0.000 | 0.043 |
| rs292597 | CMIP_1 | 16_81534790_T_C | 0.000 | 0.000 | 0.410 | 0.000 | 0.000 | 2.127 | 0.000 | 0.000 | 0.522 | 0.063 |
| rs8063007 | CMIP_2 | 16_81541587_C_A | 0.414 | 0.280 | 0.115 | 0.121 | 0.000 | 0.797 | 0.016 | 0.112 | 0.000 | 0.037 |
| rs4925109 | RAI1 | 17_17661802_A_G | 0.000 | 0.000 | 0.785 | 0.103 | 0.006 | 1.213 | 0.000 | 0.036 | 0.512 | 0.504 |
| rs1108511 | SREBF1 | 17_17726469_T_C | 0.000 | 0.000 | 0.168 | 0.080 | 0.231 | 0.553 | 0.000 | 0.075 | 1.024 | 0.275 |
| rs10962 | HNF1B_1 | 17_36046451_G_C | 0.423 | 0.299 | 0.217 | 0.000 | 0.029 | 0.228 | 0.284 | 0.000 | 0.028 | 0.139 |
| rs3110641 | HNF1B_2 | 17_36047417_A_G | 0.269 | 0.442 | 0.145 | 0.000 | 0.000 | 0.200 | 0.268 | 0.000 | 0.000 | 0.086 |
| rs2189301 | HNF1B_3 | 17_36063685_G_A | 0.000 | 0.093 | 0.494 | 0.095 | 0.226 | 0.134 | 0.097 | 0.089 | 0.183 | 0.000 |
| rs4239217 | HNF1B_4 | 17_36098987_G_A | 1.076 | 0.176 | 0.709 | 0.000 | 0.000 | 0.295 | 0.003 | 0.090 | 0.000 | 0.016 |
| rs12453022 | ATP2A3_1 | 17_3840055_C_T | 0.005 | 0.000 | 1.011 | 0.123 | 0.067 | 0.459 | 0.000 | 0.052 | 0.049 | 0.000 |
| rs3826482 | ATP2A3_2 | 17_3860356_A_T | 0.437 | 0.036 | 0.174 | 0.023 | 0.000 | 0.573 | 0.021 | 0.188 | 0.000 | 0.000 |
| rs4790581 | CYB5D2 | 17_4066657_T_C | 0.027 | 0.103 | 0.007 | 0.000 | 0.434 | 0.177 | 0.099 | 0.043 | 0.897 | 0.048 |
| rs646123 | MLX | 17_40720632_G_A | 0.289 | 0.000 | 0.222 | 0.000 | 0.572 | 0.942 | 0.000 | 0.045 | 0.000 | 0.318 |
| rs9906944 | IGF2BP1 | 17_47091420_C_T | 0.000 | 0.266 | 0.000 | 0.069 | 1.017 | 0.571 | 0.000 | 0.000 | 0.375 | 0.181 |
| rs9899520 | BPTF | 17_65957568_A_G | 0.064 | 0.146 | 0.000 | 0.000 | 0.877 | 0.925 | 0.000 | 0.098 | 0.000 | 0.000 |
| rs727428 | SHBG | 17_7537792_T_C | 0.486 | 0.000 | 0.236 | 0.160 | 0.000 | 0.690 | 0.000 | 0.012 | 0.000 | 7.236 |
| rs7219033 | GLP2R | 17_9787958_G_A | 1.850 | 0.000 | 0.000 | 0.000 | 0.478 | 0.088 | 0.000 | 0.000 | 0.000 | 0.000 |
| rs7505951 | CELF4 | 18_36313311_G_A | 0.198 | 0.814 | 0.000 | 0.000 | 0.772 | 0.220 | 0.000 | 0.000 | 0.000 | 0.136 |
| rs17594301 | TCF4 | 18_53048885_T_C | 0.016 | 0.422 | 0.120 | 0.109 | 0.000 | 0.258 | 0.103 | 0.020 | 0.827 | 0.069 |
| rs17684074 | WDR7 | 18_54675384_G_C | 1.058 | 0.253 | 0.000 | 0.039 | 0.159 | 0.164 | 0.000 | 0.130 | 0.000 | 0.286 |
| rs9957145 | GRP | 18_56876228_G_A | 0.012 | 0.183 | 0.004 | 0.001 | 0.953 | 0.034 | 0.000 | 0.000 | 0.601 | 0.064 |
| rs2168711 | MC4R_1 | 18_57848531_T_C | 0.066 | 0.000 | 0.372 | 0.000 | 2.593 | 0.335 | 0.000 | 0.000 | 0.000 | 0.346 |
| rs9675376 | MC4R_2 | 18_57969244_G_A | 0.000 | 0.047 | 0.000 | 0.000 | 1.959 | 0.384 | 0.000 | 0.000 | 0.000 | 0.049 |
| rs9947450 | MC4R_3 | 18_58052665_C_T | 0.649 | 0.000 | 0.000 | 0.000 | 1.259 | 0.302 | 0.000 | 0.000 | 0.000 | 0.000 |
| rs12454712 | BCL2 | 18_60845884_T_C | 0.000 | 0.309 | 0.000 | 0.073 | 0.000 | 2.007 | 0.000 | 0.009 | 0.292 | 0.002 |
| rs7240767 | LAMA1 | 18_7070642_T_C | 0.005 | 0.150 | 0.226 | 0.005 | 0.078 | 0.001 | 0.232 | 0.121 | 0.205 | 0.006 |
| rs1049481 | CALR | 19_13054781_G_T | 0.027 | 0.064 | 0.106 | 0.166 | 0.130 | 0.482 | 0.150 | 0.085 | 0.511 | 0.187 |
| rs10401969 | SUGP1_{—} | 19_19407718_T_C | 0.000 | 0.084 | 0.000 | 0.214 | 0.365 | 1.084 | 1.209 | 1.643 | 0.063 | 0.000 |
| | TM6SF2 | | | | | | | | | | | |
| rs11667352 | PEPD_1 | 19_33918423_A_G | 0.000 | 0.249 | 0.166 | 0.005 | 0.069 | 1.273 | 0.145 | 0.000 | 0.202 | 0.015 |
| rs7258937 | PEPD_2 | 19_33938800_C_T | 0.142 | 0.347 | 0.009 | 0.000 | 0.023 | 1.208 | 0.000 | 0.000 | 0.077 | 0.269 |
| rs2075650 | TOMM40 | 19_45395619_A_G | 0.000 | 0.018 | 0.000 | 0.093 | 3.463 | 0.000 | 1.489 | 0.444 | 0.152 | 0.254 |
| rs10408179 | EML2 | 19_46157004_T_C | 1.503 | 0.567 | 0.019 | 0.086 | 0.264 | 0.534 | 0.000 | 0.000 | 0.116 | 0.000 |
| rs11671664 | GIPR | 19_46172278_G_A | 1.561 | 0.966 | 0.000 | 0.100 | 0.000 | 0.405 | 0.000 | 0.169 | 0.000 | 0.000 |
| rs2075142 | SNRPD2 | 19_46191021_A_G | 0.129 | 0.028 | 0.120 | 0.076 | 0.631 | 0.292 | 0.162 | 0.058 | 0.091 | 0.000 |
| rs3810291 | ZC3H4 | 19_47569003_G_A | 0.618 | 0.774 | 0.000 | 0.000 | 1.105 | 0.193 | 0.044 | 0.572 | 0.000 | 0.395 |
| rs17175860 | INSR | 19_7235146_A_G | 0.185 | 0.000 | 0.095 | 0.118 | 0.020 | 1.026 | 0.000 | 0.000 | 0.048 | 0.613 |
| rs4804833 | MAP2K7 | 19_7970635_A_G | 0.000 | 0.525 | 0.000 | 0.000 | 0.260 | 0.778 | 0.174 | 0.000 | 1.448 | 0.166 |
| rs813393 | NKX2-2 | 20_21451139_G_A | 0.608 | 0.144 | 0.271 | 0.000 | 0.490 | 0.104 | 0.029 | 0.000 | 0.000 | 0.027 |
| rs2065703 | CDK5RAP1 | 20_31966698_C_T | 0.164 | 0.346 | 0.123 | 0.265 | 0.073 | 1.106 | 0.000 | 0.164 | 0.075 | 0.264 |
| rs736953 | CHMP4B | 20_32415089_G_T | 0.878 | 0.686 | 0.000 | 0.044 | 0.001 | 0.205 | 0.088 | 0.025 | 0.000 | 0.000 |
| rs6120487 | RALY | 20_32622608_A_G | 0.294 | 0.017 | 0.451 | 0.020 | 0.412 | 0.940 | 0.001 | 0.031 | 0.000 | 0.282 |
| rs2144908 | HNF4A_1 | 20_42985717_G_A | 0.799 | 0.000 | 0.992 | 0.141 | 0.000 | 0.000 | 0.255 | 0.023 | 0.055 | 0.081 |
| rs1800961 | HNF4A_2 | 20_43042364_C_T | 0.161 | 0.000 | 0.459 | 0.000 | 0.067 | 0.979 | 1.107 | 0.261 | 0.000 | 0.126 |
| rs1412956 | EYA2_1 | 20_45557017_A_G | 0.506 | 0.072 | 0.006 | 0.071 | 0.000 | 1.449 | 0.000 | 0.016 | 0.000 | 0.150 |
| rs6063048 | EYA2_2 | 20_45598564_G_A | 0.309 | 0.581 | 0.000 | 0.172 | 0.073 | 1.285 | 0.000 | 0.049 | 0.000 | 0.146 |
| rs6012876 | CEBPB | 20_48830265_T_C | 0.516 | 0.837 | 0.124 | 0.058 | 0.000 | 0.105 | 0.108 | 0.301 | 0.002 | 0.003 |
| rs17733217 | TSHZ2 | 20_51222704_A_G | 0.039 | 0.000 | 0.077 | 0.000 | 1.282 | 0.526 | 0.000 | 0.000 | 0.127 | 0.097 |
| rs6064709 | GNAS | 20_57379850_G_A | 0.011 | 0.305 | 0.349 | 0.000 | 0.257 | 0.087 | 0.099 | 0.000 | 0.285 | 0.092 |
| rs2023681 | LIF | 22_30599562_A_G | 0.566 | 0.075 | 0.151 | 0.000 | 0.382 | 0.960 | 0.000 | 0.059 | 0.087 | 0.000 |
| rs2006867 | YWHAH | 22_32384307_C_T | 0.712 | 0.326 | 0.016 | 0.099 | 0.185 | 0.393 | 0.261 | 0.035 | 0.053 | 0.090 |
| rs5758223 | EP300 | 22_41489920_G_A | 0.020 | 0.240 | 0.055 | 0.000 | 0.668 | 0.073 | 0.106 | 0.026 | 0.672 | 0.000 |
| rs738408 | PNPLA3 | 22_44324730_C_T | 0.000 | 0.000 | 0.000 | 0.000 | 0.166 | 1.059 | 1.385 | 0.343 | 0.589 | 0.000 |
| rs713669 | IL17REL | 22_50451771_C_T | 0.759 | 0.000 | 0.448 | 0.000 | 0.305 | 0.205 | 0.069 | 0.402 | 0.000 | 0.186 |
| rs12484907 | MOV10L1 | 22_50588313_G_A | 0.177 | 0.409 | 0.053 | 0.000 | 0.057 | 0.312 | 0.278 | 0.184 | 0.024 | 0.089 |

TABLE 6
Cluster weights for traits for T2D clustering

| | | | Pro- | | | Lipo- | | ALP_{—} | | |
|---|---|---|---|---|---|---|---|---|---|---|
| | Beta- | Beta- | insu- | Lipo- | Obe- | dys- | Liver/ | neg- | | |
| trait | cell1 | cell2 | lin | proteinA | sity | trophy | Lipid | ative | HyperInsulinSecretion | SHBG |
| MAGIC_dv1.2hrG.ZN_pos | 1.721 | 3.152 | 0.955 | 0.000 | 1.093 | 1.486 | 0.000 | 0.021 | 0.000 | 0.687 |
| ADIPOGen_{—} | 0.329 | 0.419 | 0.276 | 0.000 | 0.319 | 0.000 | 0.622 | 0.952 | 0.338 | 0.000 |
| dv1.Adiponectin.ZN_pos | | | | | | | | | | |
| BFpercent_{—} | 0.169 | 0.032 | 0.000 | 0.000 | 3.056 | 0.000 | 0.000 | 0.037 | 0.042 | 0.000 |
| dv1.BFP.ZN_pos | | | | | | | | | | |
| GiantUKBB_eu_{—} | 0.042 | 0.000 | 0.000 | 0.000 | 4.299 | 0.000 | 0.000 | 0.000 | 0.000 | 0.000 |
| dv1.BMI.ZN_pos | | | | | | | | | | |
| MAGIC_dv1.FG.ZN_pos | 1.666 | 3.543 | 1.463 | 0.000 | 0.305 | 0.068 | 1.180 | 0.053 | 1.056 | 0.301 |
| MAGIC_dv2.FI.ZN_pos | 0.000 | 0.000 | 0.000 | 0.000 | 1.574 | 2.091 | 0.786 | 0.030 | 0.360 | 0.000 |
| MAGIC_eu_dv6.HBA1C.ZN_pos | 1.278 | 2.032 | 1.025 | 0.000 | 0.598 | 0.387 | 0.021 | 0.252 | 0.583 | 0.436 |
| GiantUKBB_eu_{—} | 0.174 | 0.451 | 0.059 | 0.000 | 0.569 | 0.290 | 0.864 | 0.007 | 0.000 | 0.144 |
| dv1.HEIGHT.ZN_pos | | | | | | | | | | |
| GIANT_eu_females_{—} | 0.120 | 0.130 | 0.145 | 0.000 | 0.503 | 0.061 | 0.237 | 0.180 | 0.197 | 0.064 |
| dv5.HIPCadjBMI.ZN_pos | | | | | | | | | | |
| GIANT_eu_males_{—} | 0.117 | 0.379 | 0.218 | 0.000 | 0.530 | 0.081 | 0.510 | 0.002 | 0.065 | 0.068 |
| dv5.HIPCadjBMI.ZN_pos | | | | | | | | | | |
| MAGIC_dv1.HOMAB.ZN_pos | 0.000 | 0.000 | 0.000 | 0.000 | 1.173 | 1.443 | 0.062 | 0.000 | 0.000 | 0.000 |
| MAGIC_dv1.HOMAIR.ZN_pos | 0.000 | 0.208 | 0.080 | 0.000 | 1.614 | 1.860 | 0.856 | 0.000 | 0.091 | 0.000 |
| MAGIC_{—} | 0.174 | 0.691 | 0.538 | 0.052 | 0.623 | 0.000 | 0.380 | 0.000 | 0.631 | 0.000 |
| dv4.ISIadjAgeSexBMI.ZN_pos | | | | | | | | | | |
| Leptin_eu_dv1.LEP.ZN_pos | 0.500 | 0.000 | 0.258 | 0.370 | 0.676 | 0.524 | 1.058 | 1.008 | 0.686 | 0.000 |
| Leptin_eu_{—} | 0.284 | 0.064 | 0.066 | 0.201 | 2.760 | 0.212 | 1.051 | 0.662 | 0.318 | 0.006 |
| dv1.LEPadjBMI.ZN_pos | | | | | | | | | | |
| MAGIC_dv1.PI.ZN_pos | 0.415 | 6.911 | 0.000 | 1.119 | 1.357 | 0.531 | 0.000 | 0.562 | 0.000 | 0.000 |
| GIANT_eu_females_{—} | 0.158 | 0.000 | 0.000 | 0.000 | 3.727 | 0.338 | 0.000 | 0.000 | 0.003 | 0.000 |
| dv5.WAIST.ZN_pos | | | | | | | | | | |
| GIANT_eu_males_{—} | 0.030 | 0.000 | 0.009 | 0.000 | 3.852 | 0.000 | 0.140 | 0.000 | 0.000 | 0.000 |
| dv5.WAIST.ZN_pos | | | | | | | | | | |
| GIANT_eu_females_{—} | 0.373 | 0.000 | 0.000 | 0.208 | 0.510 | 2.178 | 0.017 | 0.010 | 0.218 | 0.037 |
| dv5.WAISTadjBMI.ZN_pos | | | | | | | | | | |
| GIANT_eu_males_{—} | 0.294 | 0.072 | 0.171 | 0.000 | 0.869 | 0.187 | 0.242 | 0.095 | 0.366 | 0.041 |
| dv5.WAISTadjBMI.ZN_pos | | | | | | | | | | |
| GIANT_eu_females_{—} | 0.323 | 0.000 | 0.000 | 0.110 | 1.828 | 2.548 | 0.000 | 0.000 | 0.071 | 0.000 |
| dv5.WHR.ZN_pos | | | | | | | | | | |
| GIANT_eu_males_{—} | 0.302 | 0.000 | 0.049 | 0.005 | 2.768 | 0.258 | 0.000 | 0.240 | 0.277 | 0.000 |
| dv5.WHR.ZN_pos | | | | | | | | | | |
| GIANT_eu_females_{—} | 0.476 | 0.058 | 0.000 | 0.324 | 0.181 | 3.318 | 0.000 | 0.008 | 0.191 | 0.000 |
| dv5.WHRadjBMI.ZN_pos | | | | | | | | | | |
| GIANT_eu_males_{—} | 0.547 | 0.000 | 0.191 | 0.000 | 0.646 | 0.763 | 0.000 | 0.339 | 0.627 | 0.165 |
| dv5.WHRadjBMI.ZN_pos | | | | | | | | | | |
| White.blood.cell . . . leukocyte . . . | 0.134 | 0.000 | 0.132 | 0.228 | 0.927 | 1.106 | 0.000 | 0.000 | 0.893 | 0.132 |
| count_irnt_both.ZN_pos | | | | | | | | | | |
| Red.blood.cell . . . erythrocyte . . . | 0.173 | 0.010 | 0.000 | 0.000 | 0.521 | 1.195 | 0.463 | 0.000 | 0.686 | 0.000 |
| count_irnt_both.ZN_pos | | | | | | | | | | |
| Haemoglobin.concentration_{—} | 0.000 | 0.000 | 0.083 | 0.000 | 0.262 | 1.262 | 0.970 | 0.000 | 0.226 | 0.000 |
| irnt_both.ZN_pos | | | | | | | | | | |
| Mean.corpuscular.volume_{—} | 0.097 | 0.020 | 0.427 | 0.000 | 0.168 | 0.389 | 0.899 | 0.047 | 0.000 | 0.000 |
| irnt_both.ZN_pos | | | | | | | | | | |
| Red.blood.cell . . . erythrocyte . . . | 0.208 | 0.000 | 0.000 | 0.002 | 1.156 | 0.000 | 1.010 | 0.259 | 0.849 | 0.130 |
| distribution.width_{—} | | | | | | | | | | |
| irnt_both.ZN_pos | | | | | | | | | | |
| Platelet.count_irnt_both.ZN_pos | 0.223 | 0.448 | 0.116 | 0.321 | 0.175 | 0.188 | 0.000 | 0.044 | 0.563 | 0.387 |
| Mean.platelet . . . thrombocyte . . . | 0.240 | 0.049 | 0.000 | 0.000 | 0.661 | 0.184 | 0.673 | 0.286 | 0.931 | 0.000 |
| volume_irnt_both.ZN_pos | | | | | | | | | | |
| Platelet.distribution.width_{—} | 0.249 | 0.272 | 0.054 | 0.000 | 0.300 | 0.976 | 0.237 | 0.000 | 0.807 | 0.000 |
| irnt_both.ZN_pos | | | | | | | | | | |
| Lymphocyte.count_{—} | 0.185 | 0.000 | 0.057 | 0.239 | 0.562 | 1.213 | 0.000 | 0.000 | 1.062 | 0.048 |
| irnt_both.ZN_pos | | | | | | | | | | |
| Monocyte.count_{—} | 0.191 | 0.000 | 0.061 | 0.000 | 0.613 | 0.580 | 0.428 | 0.000 | 0.314 | 0.363 |
| irnt_both.ZN_pos | | | | | | | | | | |
| Eosinophill.count_{—} | 0.149 | 0.000 | 0.098 | 0.134 | 0.710 | 0.613 | 0.000 | 0.029 | 0.766 | 0.000 |
| irnt_both.ZN_pos | | | | | | | | | | |
| Basophill.count_{—} | 0.109 | 0.024 | 0.067 | 0.031 | 0.248 | 0.197 | 0.000 | 0.000 | 0.355 | 0.034 |
| irnt_both.ZN_pos | | | | | | | | | | |
| Mean.reticulocyte.volume_{—} | 0.110 | 0.000 | 0.239 | 0.000 | 0.382 | 0.203 | 1.416 | 0.688 | 0.218 | 0.001 |
| irnt_both.ZN_pos | | | | | | | | | | |
| High.light.scatter.reticulocyte.count | 0.305 | 0.000 | 0.134 | 0.072 | 1.187 | 2.055 | 0.005 | 0.000 | 0.388 | 0.000 |
| irnt both.ZN_pos | | | | | | | | | | |
| Albumin . . . quantile._{—} | 0.132 | 0.610 | 0.043 | 0.434 | 0.000 | 1.687 | 0.000 | 0.065 | 0.000 | 0.263 |
| irnt_both.ZN_pos | | | | | | | | | | |
| Alkaline.phosphatase . . . quantile._{—} | 0.387 | 0.000 | 0.033 | 0.033 | 0.810 | 0.164 | 0.796 | 0.000 | 1.559 | 0.107 |
| irnt_both.ZN_pos | | | | | | | | | | |
| Alanine.aminotransferase . . . | 0.000 | 0.000 | 0.271 | 0.272 | 1.202 | 2.313 | 0.600 | 0.797 | 0.471 | 0.091 |
| quantile._irnt_both.ZN_pos | | | | | | | | | | |
| Apoliprotein.B . . . | 0.023 | 0.000 | 0.000 | 0.512 | 0.000 | 1.175 | 0.000 | 0.498 | 0.076 | 0.294 |
| quantile._irnt_both.ZN_pos | | | | | | | | | | |
| Aspartate.aminotransferase . . . | 0.000 | 0.000 | 0.082 | 0.000 | 0.536 | 1.576 | 0.590 | 0.028 | 0.721 | 0.000 |
| quantile._irnt_both.ZN_pos | | | | | | | | | | |
| Direct.bilirubin . . . | 0.091 | 0.138 | 0.094 | 0.000 | 0.204 | 0.308 | 0.806 | 0.156 | 0.000 | 0.000 |
| quantile._irnt_both.ZN_pos | | | | | | | | | | |
| Urea . . . quantile._{—} | 0.353 | 0.061 | 0.180 | 0.104 | 0.783 | 0.042 | 0.131 | 0.326 | 0.266 | 0.343 |
| irnt_both.ZN_pos | | | | | | | | | | |
| Calcium . . . quantile._{—} | 0.327 | 0.483 | 0.181 | 0.335 | 0.004 | 1.124 | 0.003 | 0.071 | 0.030 | 0.058 |
| irnt_both.ZN_pos | | | | | | | | | | |
| C.reactive.protein . . . | 0.000 | 0.000 | 0.200 | 0.101 | 3.170 | 0.177 | 0.294 | 0.757 | 0.252 | 0.086 |
| quantile._irnt_both.ZN_pos | | | | | | | | | | |
| Gamma.glutamyltransferase . . . | 0.088 | 0.379 | 0.133 | 0.256 | 0.869 | 1.717 | 0.000 | 0.772 | 0.284 | 0.000 |
| quantile._irnt_both.ZN_pos | | | | | | | | | | |
| Glucose . . . quantile._{—} | 0.368 | 3.699 | 1.347 | 0.000 | 0.292 | 0.391 | 1.075 | 0.515 | 0.564 | 0.401 |
| irnt_both.ZN_pos | | | | | | | | | | |
| HDL.cholesterol . . . | 0.103 | 0.065 | 0.148 | 0.008 | 0.355 | 0.000 | 0.384 | 0.384 | 0.000 | 0.000 |
| quantile._irnt_both.ZN_pos | | | | | | | | | | |
| IGF.1 . . . quantile._{—} | 0.307 | 0.201 | 0.120 | 1.267 | 0.000 | 0.970 | 1.706 | 0.325 | 0.085 | 0.106 |
| irnt_both.ZN_pos | | | | | | | | | | |
| Lipoprotein.A . . . | 0.000 | 0.000 | 0.000 | 7.240 | 0.000 | 0.000 | 0.000 | 0.000 | 0.000 | 0.000 |
| quantile._irnt_both.ZN_pos | | | | | | | | | | |
| Phosphate . . . | 0.214 | 0.139 | 0.041 | 0.056 | 0.057 | 0.634 | 0.045 | 0.363 | 0.301 | 0.054 |
| quantile._irnt_both.ZN_pos | | | | | | | | | | |
| SHBG . . . quantile._{—} | 0.000 | 0.011 | 0.000 | 0.000 | 0.000 | 0.000 | 2.138 | 0.042 | 0.000 | 0.000 |
| irnt_both.ZN_pos | | | | | | | | | | |
| Total.bilirubin . . . | 0.040 | 0.122 | 0.133 | 0.000 | 0.000 | 0.484 | 0.420 | 0.099 | 0.012 | 0.000 |
| quantile._irnt_both.ZN_pos | | | | | | | | | | |
| Testosterone . . . | 0.290 | 0.115 | 0.131 | 0.087 | 0.360 | 0.136 | 0.928 | 0.000 | 0.494 | 0.000 |
| quantile._irnt_female.ZN_pos | | | | | | | | | | |
| Total.protein . . . | 0.126 | 0.277 | 0.061 | 0.415 | 0.154 | 1.117 | 0.000 | 0.471 | 0.328 | 0.216 |
| quantile._irnt_both.ZN_pos | | | | | | | | | | |
| Triglycerides . . . | 0.244 | 0.174 | 0.029 | 0.179 | 0.478 | 4.066 | 0.000 | 0.000 | 0.000 | 0.164 |
| quantile._irnt_both.ZN_pos | | | | | | | | | | |
| Urate . . . | 0.278 | 0.123 | 0.070 | 0.440 | 1.377 | 1.294 | 0.000 | 0.024 | 0.002 | 0.139 |
| quantile._irnt_both.ZN_pos | | | | | | | | | | |
| Vitamin.D . . . | 0.000 | 0.091 | 0.182 | 0.056 | 0.130 | 0.078 | 0.663 | 0.134 | 0.160 | 0.000 |
| quantile._irnt_both.ZN_pos | | | | | | | | | | |
| Diastolic.blood.pressure . . . | 0.190 | 0.000 | 0.092 | 0.056 | 0.990 | 0.506 | 0.054 | 0.000 | 0.513 | 0.000 |
| automated.reading_irnt_{—} | | | | | | | | | | |
| both.ZN_pos | | | | | | | | | | |
| Systolic.blood.pressure . . . | 0.312 | 0.218 | 0.135 | 0.140 | 0.730 | 0.818 | 0.000 | 0.015 | 0.398 | 0.047 |
| automated.reading_irnt_{—} | | | | | | | | | | |
| both.ZN_pos | | | | | | | | | | |
| MAGIC dv5.CIR.ZN pos | 0.000 | 0.000 | 0.000 | 0.000 | 0.102 | 1.517 | 0.000 | 0.000 | 5.081 | 0.000 |
| MAGIC_dv5.DI.ZN_pos | 0.000 | 0.183 | 0.000 | 0.065 | 0.751 | 0.000 | 0.173 | 0.000 | 5.644 | 0.000 |
| MAGIC_dv1.2hrG.ZN_neg | 0.249 | 0.050 | 0.232 | 0.378 | 0.157 | 0.025 | 1.831 | 0.000 | 0.870 | 0.000 |
| ADIPOGen_{—} | 0.502 | 0.000 | 0.596 | 0.244 | 0.197 | 4.236 | 0.055 | 0.012 | 0.511 | 0.000 |
| dv1.Adiponectin.ZN_neg | | | | | | | | | | |
| BFpercent_dv1.BFP.ZN_neg | 0.042 | 0.466 | 0.598 | 0.435 | 0.000 | 1.713 | 0.000 | 0.000 | 0.000 | 0.516 |
| GiantUKBB_eu_{—} | 0.155 | 0.473 | 0.297 | 0.309 | 0.000 | 0.786 | 0.008 | 0.000 | 0.000 | 0.076 |
| dv1.BMI.ZN_neg | | | | | | | | | | |
| MAGIC_dv1.FG.ZN_neg | 0.166 | 0.000 | 0.103 | 0.056 | 0.098 | 0.187 | 0.000 | 0.007 | 0.170 | 0.000 |
| MAGIC_dv2.FI.ZN_neg | 0.511 | 1.166 | 0.789 | 0.381 | 0.000 | 0.000 | 0.000 | 0.000 | 0.197 | 0.000 |
| MAGIC_eu_dv6.HBA1C.ZN_neg | 0.012 | 0.000 | 0.026 | 0.011 | 0.032 | 0.114 | 0.007 | 0.019 | 0.103 | 0.000 |
| GiantUKBB_eu_{—} | 0.461 | 0.000 | 0.497 | 1.339 | 0.386 | 0.928 | 0.000 | 0.325 | 0.684 | 0.019 |
| dv1.HEIGHT.ZN_neg | | | | | | | | | | |
| GIANT_eu_females_{—} | 0.347 | 0.280 | 0.296 | 0.264 | 0.000 | 1.984 | 0.000 | 0.025 | 0.199 | 0.000 |
| dv5.HIPCadjBMI.ZN_neg | | | | | | | | | | |
| GIANT_eu_males_{—} | 0.528 | 0.000 | 0.272 | 0.698 | 0.000 | 1.397 | 0.000 | 0.383 | 0.911 | 0.020 |
| dv5.HIPCadjBMI.ZN_neg | | | | | | | | | | |
| MAGIC_dv1.HOMAB.ZN_neg | 1.259 | 2.405 | 1.498 | 0.333 | 0.000 | 0.000 | 0.000 | 0.000 | 0.386 | 0.000 |
| MAGIC_dv1.HOMAIR.ZN_neg | 0.492 | 0.519 | 0.744 | 0.294 | 0.000 | 0.000 | 0.000 | 0.027 | 0.309 | 0.000 |
| MAGIC | 0.552 | 0.644 | 0.481 | 0.000 | 0.402 | 4.052 | 0.000 | 0.704 | 0.000 | 0.000 |
| dv4.ISIadjAgeSexBMI.ZN neg | | | | | | | | | | |
| Leptin_eu_dv1.LEP.ZN_neg | 0.139 | 0.656 | 0.476 | 0.000 | 0.021 | 1.278 | 0.000 | 0.000 | 0.050 | 0.037 |
| Leptin_eu_{—} | 0.138 | 0.544 | 0.557 | 0.000 | 0.000 | 1.335 | 0.000 | 0.005 | 0.000 | 0.374 |
| dv1.LEPadjBMI.ZN_neg | | | | | | | | | | |
| MAGIC_dv1.PI.ZN_neg | 0.000 | 0.000 | 8.206 | 0.000 | 0.116 | 0.000 | 0.000 | 0.000 | 0.000 | 0.156 |
| GIANT_eu_females_{—} | 0.112 | 0.575 | 0.708 | 0.142 | 0.000 | 0.303 | 0.026 | 0.050 | 0.014 | 0.159 |
| dv5.WAIST.ZN_neg | | | | | | | | | | |
| GIANT_eu_males_{—} | 0.165 | 0.879 | 0.605 | 0.587 | 0.000 | 1.208 | 0.000 | 0.000 | 0.000 | 0.257 |
| dv5.WAIST.ZN_neg | | | | | | | | | | |
| GIANT_eu_females_{—} | 0.138 | 0.161 | 0.433 | 0.084 | 0.050 | 0.077 | 0.183 | 0.097 | 0.217 | 0.000 |
| dv5.WAISTadjBMI.ZN_neg | | | | | | | | | | |
| GIANT_eu_males_{—} | 0.287 | 0.306 | 0.272 | 0.438 | 0.000 | 0.993 | 0.048 | 0.085 | 0.653 | 0.000 |
| dv5.WAISTadjBMI.ZN_neg | | | | | | | | | | |
| GIANT_eu_females_{—} | 0.070 | 0.181 | 0.413 | 0.000 | 0.000 | 0.109 | 0.264 | 0.101 | 0.048 | 0.022 |
| dv5.WHR.ZN_neg | | | | | | | | | | |
| GIANT_eu_males_{—} | 0.073 | 0.633 | 0.384 | 0.202 | 0.000 | 0.634 | 0.135 | 0.007 | 0.070 | 0.087 |
| dv5.WHR.ZN_neg | | | | | | | | | | |
| GIANT_eu_females_{—} | 0.068 | 0.132 | 0.263 | 0.007 | 0.168 | 0.025 | 0.342 | 0.167 | 0.108 | 0.007 |
| dv5.WHRadjBMI.ZN_neg | | | | | | | | | | |
| GIANT_eu_males_{—} | 0.190 | 0.316 | 0.211 | 0.000 | 0.065 | 0.439 | 0.256 | 0.021 | 0.282 | 0.068 |
| dv5.WHRadjBMI.ZN_neg | | | | | | | | | | |
| White.blood.cell . . . leukocyte . . . | 0.149 | 0.221 | 0.034 | 0.000 | 0.000 | 0.000 | 1.216 | 0.980 | 0.273 | 0.041 |
| count_irnt_both.ZN_neg | | | | | | | | | | |
| Red.blood.cell . . . erythrocyte . . . | 0.222 | 0.354 | 0.373 | 0.102 | 0.054 | 0.000 | 0.181 | 1.193 | 0.100 | 0.170 |
| count_irnt_both.ZN_neg | | | | | | | | | | |
| Haemoglobin.concentration_{—} | 0.195 | 0.499 | 0.216 | 0.209 | 0.046 | 0.000 | 0.000 | 1.426 | 0.394 | 0.403 |
| irnt_both.ZN_neg | | | | | | | | | | |
| Mean.corpuscular.volume_{—} | 0.393 | 0.255 | 0.005 | 0.243 | 0.437 | 0.708 | 0.000 | 0.086 | 0.952 | 0.118 |
| irnt_both.ZN_neg | | | | | | | | | | |
| Red.blood.cell . . . erythrocyte . . . | 0.175 | 0.278 | 0.232 | 0.090 | 0.000 | 0.956 | 0.143 | 0.010 | 0.047 | 0.409 |
| distribution.width_irnt_both.ZN_neg | | | | | | | | | | |
| Platelet.count_irnt_both.ZN_neg | 0.155 | 0.253 | 0.064 | 0.000 | 0.271 | 0.376 | 1.676 | 0.104 | 0.489 | 0.038 |
| Mean.platelet . . . thrombocyte . . . | 0.192 | 0.085 | 0.486 | 0.043 | 0.000 | 0.685 | 0.013 | 0.030 | 0.363 | 0.051 |
| volume_irnt_both.ZN_neg | | | | | | | | | | |
| Platelet.distribution.width_{—} | 0.100 | 0.000 | 0.300 | 0.069 | 0.165 | 0.000 | 0.183 | 0.576 | 0.370 | 0.453 |
| irnt_both.ZN_neg | | | | | | | | | | |
| Lymphocyte.count_irnt_both.ZN_neg | 0.104 | 0.295 | 0.083 | 0.000 | 0.000 | 0.046 | 0.925 | 0.591 | 0.264 | 0.040 |
| Monocyte.count_irnt_both.ZN_neg | 0.122 | 0.359 | 0.153 | 0.351 | 0.000 | 0.000 | 0.139 | 1.487 | 0.390 | 0.066 |
| Eosinophill.count_irnt_both.ZN_neg | 0.261 | 0.243 | 0.105 | 0.000 | 0.000 | 0.177 | 0.188 | 0.555 | 0.203 | 0.300 |
| Basophill.count_irnt_both.ZN_neg | 0.200 | 0.165 | 0.141 | 0.019 | 0.027 | 0.089 | 0.147 | 0.132 | 0.226 | 0.009 |
| Mean.reticulocyte.volume_irnt_{—} | 0.255 | 0.335 | 0.139 | 0.127 | 0.000 | 1.068 | 0.000 | 0.000 | 0.643 | 0.018 |
| both.ZN_neg | | | | | | | | | | |
| High.light.scatter.reticulocyte.count_{—} | 0.238 | 0.000 | 0.081 | 0.010 | 0.000 | 0.000 | 0.771 | 0.454 | 0.194 | 0.145 |
| irnt_both.ZN_neg | | | | | | | | | | |
| Albumin . . . quantile._{—} | 0.000 | 0.000 | 0.312 | 0.000 | 0.666 | 0.000 | 1.774 | 0.009 | 0.453 | 0.000 |
| irnt_both.ZN_neg | | | | | | | | | | |
| Alkaline.phosphatase . . . quantile._{—} | 0.000 | 0.000 | 0.000 | 0.000 | 0.000 | 0.177 | 0.078 | 7.994 | 0.000 | 0.102 |
| irnt_both.ZN_neg | | | | | | | | | | |
| Alanine.aminotransferase . . . | 0.174 | 0.212 | 0.077 | 0.000 | 0.000 | 0.000 | 0.230 | 0.066 | 0.112 | 0.000 |
| quantile._irnt_both.ZN_neg | | | | | | | | | | |
| Apoliprotein.B . . . | 0.000 | 0.087 | 0.000 | 0.000 | 1.667 | 0.000 | 2.394 | 0.315 | 0.000 | 0.000 |
| quantile._irnt_both.ZN_neg | | | | | | | | | | |
| Aspartate.aminotransferase . . . | 0.157 | 0.514 | 0.210 | 0.000 | 0.000 | 0.000 | 0.643 | 1.117 | 0.219 | 0.134 |
| quantile._irnt_both.ZN_neg | | | | | | | | | | |
| Direct.bilirubin . . . | 0.149 | 0.023 | 0.118 | 0.431 | 0.177 | 0.289 | 0.000 | 0.534 | 0.272 | 0.497 |
| quantile._irnt_both.ZN_neg | | | | | | | | | | |
| Urea . . . | 0.072 | 0.159 | 0.071 | 0.000 | 0.044 | 0.256 | 0.774 | 0.038 | 0.177 | 0.000 |
| quantile.irnt both.ZN neg | | | | | | | | | | |
| Calcium . . . quantile._{—} | 0.000 | 0.000 | 0.187 | 0.000 | 0.254 | 0.000 | 1.563 | 0.325 | 0.450 | 0.035 |
| irnt_both.ZN_neg | | | | | | | | | | |
| C.reactive.protein . . . | 0.439 | 0.045 | 0.000 | 0.000 | 0.000 | 0.157 | 3.335 | 0.000 | 0.093 | 0.214 |
| quantile._irnt_both.ZN_neg | | | | | | | | | | |
| Gamma.glutamyltransferase . . . | 0.264 | 0.000 | 0.017 | 0.000 | 0.000 | 0.000 | 2.576 | 0.000 | 0.000 | 0.070 |
| quantile._irnt_both.ZN_neg | | | | | | | | | | |
| Glucose . . . quantile._{—} | 0.216 | 0.012 | 0.000 | 0.022 | 0.000 | 0.002 | 0.000 | 0.000 | 0.023 | 0.000 |
| irnt_both.ZN_neg | | | | | | | | | | |
| HDL.cholesterol . . . quantile._{—} | 0.000 | 0.089 | 0.000 | 0.000 | 1.275 | 3.697 | 0.356 | 0.000 | 0.480 | 0.234 |
| irnt_both.ZN_neg | | | | | | | | | | |
| IGF.1 . . . quantile._{—} | 0.226 | 0.205 | 0.191 | 0.000 | 0.910 | 0.000 | 0.163 | 0.039 | 0.190 | 0.519 |
| irnt_both.ZN_neg | | | | | | | | | | |
| Lipoprotein.A . . . | 0.133 | 0.154 | 0.062 | 0.000 | 0.112 | 0.452 | 0.001 | 0.152 | 0.193 | 0.000 |
| quantile._irnt_both.ZN_neg | | | | | | | | | | |
| Phosphate . . . quantile._{—} | 0.139 | 0.069 | 0.149 | 0.020 | 0.342 | 0.238 | 0.359 | 0.016 | 0.303 | 0.000 |
| irnt_both.ZN_neg | | | | | | | | | | |
| SHBG . . . | 0.395 | 0.083 | 0.034 | 0.694 | 0.809 | 1.980 | 0.000 | 0.000 | 0.284 | 7.704 |
| quantile._irnt_both.ZN_neg | | | | | | | | | | |
| Total.bilirubin . . . | 0.167 | 0.085 | 0.137 | 0.368 | 0.495 | 0.121 | 0.063 | 0.531 | 0.416 | 0.545 |
| quantile._irnt_both.ZN_neg | | | | | | | | | | |
| Testosterone . . . | 0.212 | 0.166 | 0.286 | 0.180 | 0.000 | 0.787 | 0.000 | 0.372 | 0.123 | 0.443 |
| quantile._irnt_female.ZN_neg | | | | | | | | | | |
| Total.protein . . . | 0.160 | 0.000 | 0.145 | 0.007 | 0.061 | 0.000 | 1.563 | 0.184 | 0.663 | 0.106 |
| quantile._irnt_both.ZN_neg | | | | | | | | | | |
| Triglycerides . . . | 0.000 | 0.000 | 0.000 | 0.000 | 0.098 | 0.000 | 3.047 | 0.871 | 0.000 | 0.000 |
| quantile._irnt_both.ZN_neg | | | | | | | | | | |
| Urate . . . | 0.069 | 0.207 | 0.106 | 0.013 | 0.000 | 0.022 | 1.856 | 0.047 | 0.094 | 0.000 |
| quantile._irnt_both.ZN_neg | | | | | | | | | | |
| Vitamin.D . . . | 0.304 | 0.081 | 0.071 | 0.000 | 0.500 | 0.456 | 0.046 | 0.165 | 0.238 | 0.288 |
| quantile._irnt_both.ZN_neg | | | | | | | | | | |
| Diastolic.blood.pressure . . . | 0.219 | 0.341 | 0.299 | 0.169 | 0.000 | 0.053 | 0.041 | 0.553 | 0.175 | 0.069 |
| automated.reading_{—} | | | | | | | | | | |
| irnt_both.ZN_neg | | | | | | | | | | |
| Systolic.blood.pressure . . . | 0.175 | 0.075 | 0.241 | 0.003 | 0.018 | 0.000 | 0.218 | 0.032 | 0.074 | 0.113 |
| automated.reading_{—} | | | | | | | | | | |
| irnt_both.ZN_neg | | | | | | | | | | |
| MAGIC_dv5.CIR.ZN_neg | 9.582 | 0.456 | 0.668 | 0.000 | 0.077 | 0.000 | 0.000 | 0.054 | 0.000 | 0.000 |
| MAGIC_dv5.DI.ZN_neg | 9.016 | 0.000 | 0.000 | 0.000 | 0.000 | 0.979 | 0.537 | 0.146 | 0.000 | 0.254 |

TABLE 7
T2D cluster GWAS pPS associations with traits used in clustering

| | | | |
|---|---|---|---|
| Beta-cell 1 (N = 66) | Beta-cell 2 (N = 27) | Proinsulin (N = 18) | Lipoprotein A (N = 1) |

| | beta | Pvalue | beta | Pvalue | beta | Pvalue | beta | Pvalue |
|---|---|---|---|---|---|---|---|---|
| Glycemic traits | | | | | | | | |
| 2-hour glucose | 0.0253 | 3.33E−17 | 0.0324 | 2.66E−12 | 0.0118 | 2.71E−02 | −0.0153 | 4.35E−01 |
| Corrected insulin response | −0.0585 | 2.51E−78 | −0.0364 | 1.14E−13 | −0.0119 | 3.89E−02 | −0.0003 | 9.86E−01 |
| Disposition index | −0.0525 | 7.36E−63 | −0.0242 | 8.98E−07 | −0.0091 | 1.15E−01 | 0.0029 | 8.85E−01 |
| Fasting glucose | 0.0077 | 4.64E−41 | 0.0179 | 6.67E−88 | 0.0105 | 4.83E−24 | 0.0056 | 1.30E−01 |
| Fasting Insulin | −0.0022 | 4.18E−06 | −0.0041 | 4.73E−08 | −0.0029 | 1.07E−03 | −0.0070 | 2.87E−02 |
| Fasting proinsulin | 0.0057 | 1.97E−07 | 0.0236 | 3.42E−42 | −0.0253 | 3.51E−36 | 0.0180 | 9.09E−03 |
| HbA1c | 0.0048 | 4.49E−74 | 0.0081 | 2.43E−88 | 0.0054 | 2.29E−29 | 0.0000 | 1.00E+00 |
| HOMA-B | −0.0052 | 7.21E−25 | −0.0113 | 7.33E−45 | −0.0069 | 9.45E−14 | −0.0080 | 1.53E−02 |
| HOMA-IR | −0.0014 | 2.37E−02 | −0.0011 | 2.60E−01 | −0.0006 | 5.95E−01 | −0.0044 | 2.71E−01 |
| Modified Stumvoll insulin sensitivity | −0.0051 | 7.63E−04 | −0.0002 | 9.17E−01 | 0.0018 | 5.23E−01 | 0.0027 | 7.87E−01 |
| index adjAgeSexBMI | | | | | | | | |
| Random glucose | 0.0099 | 1.71E−161 | 0.0233 | 5.20E−373 | 0.0164 | 6.98E−126 | 0.0057 | 2.31E−02 |
| Non-glycemic traits - anthropometric | | | | | | | | |
| BMI | −0.0008 | 8.24E−04 | −0.0023 | 5.43E−09 | −0.0021 | 3.56E−06 | −0.0110 | 6.20E−12 |
| Bodyfat percent | −0.0014 | 5.96E−02 | −0.0028 | 1.58E−02 | −0.0048 | 4.86E−04 | −0.0189 | 3.98E−05 |
| Diastolic blood pressure | −0.0005 | 1.86E−01 | −0.0022 | 3.22E−05 | −0.0008 | 2.14E−01 | −0.0055 | 2.03E−02 |
| Height | −0.0014 | 2.63E−11 | 0.0010 | 3.63E−03 | −0.0017 | 2.40E−05 | −0.0202 | 3.42E−47 |
| Hip circumference adjBMI Female | −0.0040 | 6.85E−08 | −0.0040 | 4.42E−04 | −0.0046 | 8.33E−04 | −0.0051 | 2.57E−01 |
| Hip circumference adjBMI Male | −0.0037 | 2.25E−05 | −0.0003 | 8.10E−01 | −0.0015 | 3.57E−01 | −0.0140 | 6.05E−03 |
| Systolic blood pressure | 0.0018 | 3.47E−08 | 0.0013 | 1.32E−02 | 0.0016 | 1.20E−02 | 0.0029 | 1.95E−01 |
| Waist circumference adjBMI Female | 0.0030 | 3.30E−05 | 0.0011 | 3.01E−01 | −0.0013 | 3.21E−01 | 0.0130 | 2.50E−03 |
| Waist circumference adjBMI Male | −0.0012 | 1.43E−01 | −0.0018 | 1.42E−01 | 0.0006 | 6.73E−01 | −0.0092 | 5.53E−02 |
| Waist circumference Female | −0.0002 | 7.83E−01 | −0.0035 | 1.42E−03 | −0.0058 | 1.43E−05 | −0.0046 | 2.96E−01 |
| Waist circumference Male | −0.0033 | 3.94E−05 | −0.0055 | 6.58E−06 | −0.0046 | 1.53E−03 | −0.0180 | 1.77E−04 |
| Waist-to-hip ratio adjBMI Female | 0.0054 | 1.35E−13 | 0.0043 | 1.58E−04 | 0.0020 | 1.46E−01 | 0.0160 | 3.77E−04 |
| Waist-to-hip ratio adjBMI Male | 0.0023 | 7.24E−03 | −0.0005 | 7.09E−01 | 0.0028 | 6.84E−02 | −0.0012 | 8.10E−01 |
| Waist-to-hip ratio Female | 0.0041 | 1.68E−08 | 0.0014 | 2.05E−01 | −0.0005 | 6.78E−01 | 0.0091 | 3.86E−02 |
| Waist-to-hip ratio Male | 0.0007 | 3.96E−01 | −0.0026 | 4.04E−02 | −0.0010 | 5.24E−01 | −0.0077 | 1.16E−01 |
| Non-glycemic traits - lab measures | | | | | | | | |
| Adiponectin | −0.0017 | 1.51E−02 | −0.0008 | 4.84E−01 | −0.0022 | 7.72E−02 | −0.0065 | 1.47E−01 |
| Alanine aminotransferase | 0.0023 | 2.35E−12 | −0.0008 | 1.24E−01 | 0.0022 | 3.57E−04 | 0.0099 | 1.11E−05 |
| Albumin | 0.0027 | 7.70E−14 | 0.0036 | 9.24E−11 | 0.0000 | 9.54E−01 | 0.0159 | 1.23E−10 |
| Alkaline phosphotase | −0.0034 | 1.47E−23 | −0.0018 | 5.11E−04 | 0.0023 | 2.88E−04 | 0.0015 | 5.30E−01 |
| Apolipoprotein B | −0.0004 | 2.99E−01 | −0.0014 | 1.05E−02 | −0.0007 | 2.96E−01 | 0.0182 | 2.37E−14 |
| Aspartate aminotransferase | −0.0014 | 5.96E−05 | −0.0046 | 1.55E−18 | −0.0014 | 3.01E−02 | −0.0010 | 6.50E−01 |
| Basophill count | −0.0009 | 3.47E−03 | −0.0009 | 4.57E−02 | −0.0005 | 3.73E−01 | −0.0009 | 6.61E−01 |
| Calcium | 0.0025 | 1.56E−11 | 0.0030 | 1.64E−07 | 0.0010 | 1.28E−01 | 0.0123 | 7.17E−07 |
| C-reactive protein | −0.0038 | 1.33E−27 | −0.0015 | 5.63E−03 | 0.0010 | 1.51E−01 | 0.0000 | 9.87E−01 |
| Direct bilirubin | 0.0000 | 9.38E−01 | −0.0002 | 6.74E−01 | 0.0015 | 3.03E−02 | −0.0120 | 1.69E−06 |
| Eosinophill count | −0.0009 | 5.48E−04 | −0.0006 | 1.15E−01 | −0.0004 | 3.59E−01 | 0.0008 | 6.31E−01 |
| Gamma-glutamyl transferase | 0.0000 | 9.14E−01 | 0.0003 | 5.82E−01 | 0.0020 | 1.14E−03 | 0.0084 | 1.60E−04 |
| HDL | −0.0026 | 1.19E−14 | −0.0025 | 1.69E−06 | −0.0012 | 6.60E−02 | −0.0074 | 1.16E−03 |
| Hemoglobin concentration | −0.0010 | 4.87E−04 | −0.0023 | 7.17E−08 | −0.0006 | 2.62E−01 | −0.0059 | 1.93E−03 |
| High light scatter reticulocyte count | 0.0019 | 1.33E−07 | 0.0016 | 2.71E−03 | 0.0017 | 9.04E−03 | 0.0047 | 5.16E−02 |
| IGF-1 | 0.0018 | 2.09E−07 | 0.0012 | 2.37E−02 | 0.0006 | 3.69E−01 | 0.0330 | 1.37E−45 |
| Leptin | 0.0014 | 6.24E−02 | −0.0021 | 8.12E−02 | 0.0016 | 2.48E−01 | 0.0047 | 3.57E−01 |
| Leptin adjBMI | 0.0009 | 3.84E−01 | −0.0014 | 3.49E−01 | −0.0019 | 2.77E−01 | 0.0029 | 6.60E−01 |
| Lipoprotein A | −0.0004 | 2.63E−01 | −0.0010 | 9.98E−02 | −0.0002 | 7.77E−01 | 0.2288 | 4.06E−1586 |
| Lymphocyte count | 0.0010 | 2.98E−03 | 0.0006 | 2.54E−01 | 0.0001 | 8.85E−01 | 0.0046 | 5.27E−02 |
| Mean corpuscular volume | −0.0021 | 1.05E−09 | −0.0013 | 1.33E−02 | 0.0001 | 8.57E−01 | −0.0076 | 1.43E−03 |
| Mean platelet volume | 0.0004 | 2.22E−01 | 0.0004 | 4.73E−01 | −0.0014 | 4.12E−02 | −0.0009 | 7.15E−01 |
| Mean reticulocyte volume | −0.0010 | 5.32E−03 | −0.0021 | 1.64E−04 | −0.0009 | 1.54E−01 | −0.0057 | 1.67E−02 |
| Monocyte count | −0.0004 | 2.05E−01 | −0.0018 | 4.53E−04 | −0.0007 | 2.65E−01 | −0.0110 | 1.98E−06 |
| Phosphate | 0.0002 | 5.91E−01 | 0.0011 | 5.42E−02 | −0.0009 | 1.83E−01 | 0.0019 | 4.46E−01 |
| Platelet count | −0.0007 | 4.64E−02 | 0.0022 | 1.85E−05 | −0.0013 | 3.57E−02 | 0.0075 | 1.29E−03 |
| Platelet distribution width | 0.0014 | 4.15E−05 | 0.0017 | 1.72E−03 | 0.0004 | 5.25E−01 | 0.0033 | 1.64E−01 |
| Red blood cell count | −0.0002 | 5.00E−01 | −0.0017 | 1.91E−04 | −0.0010 | 7.50E−02 | −0.0031 | 1.33E−01 |
| Red blood cell distribution width | −0.0006 | 9.56E−02 | −0.0029 | 7.95E−08 | −0.0024 | 2.31E−04 | −0.0047 | 4.73E−02 |
| Sex hormone-binding globulin | −0.0052 | 4.83E−54 | −0.0038 | 1.74E−13 | −0.0037 | 6.12E−09 | −0.0321 | 2.43E−45 |
| Testosterone Female | −0.0001 | 8.45E−01 | 0.0002 | 8.50E−01 | 0.0004 | 6.66E−01 | −0.0086 | 1.58E−02 |
| Total bilirubin | −0.0006 | 8.09E−02 | −0.0010 | 4.57E−02 | 0.0012 | 5.16E−02 | −0.0093 | 6.05E−05 |
| Total protein | 0.0011 | 2.77E−03 | 0.0005 | 3.97E−01 | 0.0006 | 3.95E−01 | 0.0134 | 1.04E−07 |
| Triglycerides | 0.0039 | 3.53E−30 | 0.0037 | 3.74E−12 | 0.0026 | 4.83E−05 | 0.0114 | 8.73E−07 |
| Urate | 0.0003 | 3.52E−01 | −0.0007 | 1.16E−01 | 0.0007 | 1.89E−01 | 0.0129 | 2.22E−10 |
| Urea | 0.0019 | 1.72E−08 | 0.0010 | 4.56E−02 | 0.0014 | 2.92E−02 | 0.0033 | 1.47E−01 |
| Vitamin D | −0.0017 | 2.27E−06 | 0.0002 | 7.77E−01 | 0.0004 | 5.22E−01 | 0.0014 | 5.78E−01 |
| White blood cell count | 0.0000 | 9.23E−01 | 0.0010 | 6.36E−02 | 0.0014 | 3.26E−02 | 0.0025 | 2.98E−01 |

| | Obesity | | Lipodystrophy | | Liver/Lipid | | ALP negative | |
|---|---|---|---|---|---|---|---|---|
| | (N = 37) | | (N = 54) | | (N = 12) | | (N = 4) | |
| | beta | Pvalue | beta | Pvalue | beta | Pvalue | beta | Pvalue |
| Glycemic traits | | | | | | | | |
| 2-hour glucose | 0.0101 | 5.23E−03 | 0.0173 | 1.11E−08 | −0.0006 | 9.36E−01 | −0.0349 | 6.51E−03 |
| Corrected insulin response | 0.0003 | 9.43E−01 | 0.0099 | 2.26E−03 | −0.0040 | 5.99E−01 | −0.0123 | 3.81E−01 |
| Disposition index | 0.0038 | 3.23E−01 | −0.0073 | 2.40E−02 | −0.0020 | 7.96E−01 | −0.0297 | 3.50E−02 |
| Fasting glucose | 0.0028 | 8.15E−05 | 0.0026 | 1.06E−05 | 0.0077 | 3.02E−08 | 0.0146 | 1.17E−09 |
| Fasting Insulin | 0.0056 | 5.12E−21 | 0.0069 | 8.85E−43 | 0.0037 | 1.89E−03 | 0.0073 | 2.79E−04 |
| Fasting proinsulin | 0.0066 | 1.51E−06 | 0.0037 | 9.40E−04 | 0.0028 | 3.06E−01 | 0.0145 | 1.86E−03 |
| HbA1c | 0.0026 | 7.38E−15 | 0.0023 | 1.74E−16 | 0.0026 | 3.72E−05 | 0.0041 | 1.35E−04 |
| HOMA-B | 0.0038 | 1.13E−09 | 0.0045 | 5.00E−17 | −0.0006 | 6.39E−01 | −0.0001 | 9.70E−01 |
| HOMA-IR | 0.0065 | 1.11E−17 | 0.0069 | 9.08E−27 | 0.0037 | 1.28E−02 | 0.0083 | 1.45E−03 |
| Modified Stumvoll insulin sensitivity | −0.0006 | 7.56E−01 | −0.0207 | 5.26E−38 | −0.0037 | 3.26E−01 | −0.0039 | 5.33E−01 |
| index adjAgeSexBMI | | | | | | | | |
| Random glucose | 0.0058 | 2.39E−35 | 0.0049 | 2.87E−36 | 0.0136 | 3.32E−51 | 0.0194 | 1.09E−42 |
| Non-glycemic traits - anthropometric | | | | | | | | |
| BMI | 0.0259 | 5.41E−1464 | −0.0040 | 5.63E−50 | 0.0030 | 9.60E−06 | 0.0022 | 2.45E−02 |
| Bodyfat percent | 0.0187 | 4.36E−86 | −0.0097 | 1.35E−35 | 0.0024 | 1.89E−01 | 0.0019 | 5.35E−01 |
| Diastolic blood pressure | 0.0058 | 5.00E−39 | 0.0030 | 4.26E−16 | −0.0007 | 3.95E−01 | −0.0012 | 3.75E−01 |
| Height | 0.0007 | 7.00E−03 | −0.0040 | 1.47E−68 | 0.0004 | 4.64E−01 | 0.0020 | 2.04E−02 |
| Hip circumference adjBMI Female | 0.0016 | 9.40E−02 | −0.0124 | 7.72E−58 | −0.0010 | 5.83E−01 | 0.0012 | 6.98E−01 |
| Hip circumference adjBMI Male | 0.0019 | 8.29E−02 | −0.0091 | 1.46E−24 | −0.0011 | 5.92E−01 | −0.0014 | 6.96E−01 |
| Systolic blood pressure | 0.0045 | 5.09E−27 | 0.0051 | 9.44E−48 | −0.0001 | 8.99E−01 | 0.0008 | 5.22E−01 |
| Waist circumference adjBMI Female | 0.0041 | 7.04E−06 | 0.0125 | 1.81E−63 | 0.0017 | 3.00E−01 | −0.0022 | 4.34E−01 |
| Waist circumference adjBMI Male | 0.0051 | 7.56E−07 | −0.0052 | 5.05E−10 | −0.0013 | 4.72E−01 | 0.0043 | 1.89E−01 |
| Waist circumference Female | 0.0244 | 3.86E−156 | 0.0013 | 7.87E−02 | 0.0023 | 1.81E−01 | −0.0011 | 7.08E−01 |
| Waist circumference Male | 0.0258 | 1.05E−140 | −0.0074 | 3.36E−19 | 0.0011 | 5.57E−01 | 0.0070 | 2.92E−02 |
| Waist-to-hip ratio adjBMI Female | 0.0027 | 3.38E−03 | 0.0197 | 1.84E−148 | 0.0025 | 1.49E−01 | −0.0033 | 2.55E−01 |
| Waist-to-hip ratio adjBMI Malc | 0.0040 | 1.94E−04 | 0.0028 | 1.22E−03 | 0.0007 | 7.35E−01 | 0.0061 | 7.46E−02 |
| Waist-to-hip ratio Female | 0.0133 | 4.16E−48 | 0.0152 | 4.56E−93 | 0.0031 | 6.89E−02 | −0.0025 | 3.86E−01 |
| Waist-to-hip ratio Male | 0.0181 | 1.10E−67 | −0.0011 | 1.88E−01 | 0.0021 | 2.61E−01 | 0.0077 | 1.86E−02 |
| Non-glycemic traits - lab measures | | | | | | | | |
| Adiponectin | −0.0015 | 7.26E−02 | −0.0128 | 1.38E−73 | −0.0005 | 7.80E−01 | 0.0119 | 3.04E−05 |
| Alanine aminotransferase | 0.0083 | 3.27E−86 | 0.0142 | 3.29E−357 | 0.0169 | 2.88E−95 | 0.0123 | 6.03E−22 |
| Albumin | −0.0052 | 1.09E−29 | 0.0097 | 7.05E−138 | −0.0118 | 1.18E−39 | −0.0129 | 3.49E−20 |
| Alkaline phosphotase | 0.0061 | 2.85E−44 | −0.0003 | 4.83E−01 | 0.0034 | 7.46E−05 | −0.1073 | 1.97E−1431 |
| Apolipoprotein B | −0.0044 | 8.41E−23 | 0.0053 | 2.19E−46 | −0.0261 | 6.67E−200 | −0.0190 | 9.05E−45 |
| Aspartate aminotransferase | 0.0038 | 2.29E−18 | 0.0107 | 2.79E−194 | 0.0033 | 6.69E−05 | −0.0106 | 5.79E−16 |
| Basophill count | 0.0005 | 1.46E−01 | 0.0010 | 1.64E−03 | −0.0028 | 1.15E−04 | −0.0010 | 3.94E−01 |
| Calcium | −0.0017 | 1.74E−04 | 0.0070 | 4.52E−72 | −0.0115 | 4.17E−37 | −0.0151 | 1.20E−26 |
| C-reactive protein | 0.0154 | 5.63E−257 | 0.0020 | 2.01E−07 | −0.0185 | 1.34E−99 | −0.0115 | 4.43E−17 |
| Direct bilirubin | −0.0008 | 9.85E−02 | −0.0006 | 1.60E−01 | 0.0054 | 3.21E−09 | 0.0062 | 1.46E−05 |
| Eosinophill count | 0.0032 | 3.17E−22 | 0.0025 | 5.66E−19 | −0.0036 | 1.87E−08 | −0.0013 | 2.12E−01 |
| Gamma-glutamyl transferase | 0.0064 | 9.73E−53 | 0.0105 | 5.62E−199 | −0.0236 | 3.09E−186 | −0.0047 | 1.84E−04 |
| HDL | −0.0110 | 1.87E−145 | −0.0174 | 2.91E−513 | −0.0053 | 1.85E−10 | 0.0043 | 9.22E−04 |
| Hemoglobin concentration | 0.0012 | 1.11E−03 | 0.0052 | 3.52E−67 | 0.0025 | 3.17E−04 | −0.0027 | 1.12E−02 |
| High light scatter reticulocyte count | 0.0078 | 4.66E−68 | 0.0128 | 6.84E−256 | −0.0093 | 7.30E−27 | −0.0104 | 1.95E−14 |
| IGF-1 | −0.0043 | 1.77E−22 | 0.0057 | 7.62E−55 | 0.0053 | 3.39E−10 | 0.0235 | 2.17E−70 |
| Leptin | 0.0021 | 2.92E−02 | −0.0012 | 1.33E−01 | 0.0058 | 1.81E−03 | 0.0174 | 2.06E−08 |
| Leptin adjBMI | 0.0129 | 3.24E−26 | −0.0026 | 1.15E−02 | 0.0100 | 3.09E−05 | 0.0181 | 6.39E−06 |
| Lipoprotein A | 0.0002 | 6.26E−01 | −0.0018 | 1.53E−05 | 0.0008 | 3.99E−01 | −0.0001 | 9.73E−01 |
| Lymphocyte count | 0.0047 | 1.57E−25 | 0.0084 | 4.62E−111 | −0.0151 | 9.82E−68 | −0.0112 | 1.41E−16 |
| Mean corpuscular volume | −0.0022 | 1.02E−06 | −0.0019 | 5.21E−07 | 0.0059 | 1.25E−11 | 0.0020 | 1.37E−01 |
| Mean platelet volume | 0.0040 | 7.16E−19 | 0.0001 | 8.93E−01 | 0.0096 | 4.83E−28 | 0.0081 | 3.34E−09 |
| Mean reticulocyte volume | 0.0019 | 1.46E−05 | −0.0037 | 5.43E−23 | 0.0169 | 1.48E−83 | 0.0135 | 7.11E−23 |
| Monocyte count | 0.0016 | 2.12E−04 | 0.0044 | 5.62E−33 | −0.0066 | 4.35E−15 | −0.0065 | 9.82E−07 |
| Phosphate | −0.0010 | 2.29E−02 | 0.0015 | 5.94E−05 | −0.0025 | 4.56E−03 | 0.0004 | 7.87E−01 |
| Platelet count | −0.0019 | 7.57E−06 | −0.0010 | 6.48E−03 | −0.0159 | 6.53E−79 | −0.0100 | 2.90E−14 |
| Platelet distribution width | 0.0027 | 2.18E−09 | 0.0054 | 3.38E−48 | 0.0010 | 2.52E−01 | −0.0088 | 6.33E−11 |
| Red blood cell count | 0.0027 | 4.09E−12 | 0.0051 | 3.23E−56 | 0.0010 | 1.60E−01 | −0.0038 | 1.27E−03 |
| Red blood cell distribution width | 0.0070 | 1.21E−55 | −0.0042 | 1.96E−29 | 0.0113 | 4.14E−39 | 0.0041 | 2.37E−03 |
| Sex hormone-binding globulin | −0.0093 | 3.56E−106 | −0.0139 | 3.04E−333 | 0.0043 | 2.39E−07 | 0.0136 | 8.75E−26 |
| Testosterone Female | 0.0018 | 7.44E−03 | −0.0029 | 2.30E−07 | 0.0065 | 5.44E−07 | 0.0070 | 5.50E−04 |
| Total bilirubin | −0.0031 | 4.98E−13 | 0.0004 | 3.23E−01 | −0.0019 | 2.20E−02 | 0.0015 | 2.56E−01 |
| Total protein | 0.0002 | 7.08E−01 | 0.0069 | 1.84E−68 | −0.0164 | 4.67E−72 | −0.0054 | 1.87E−04 |
| Triglycerides | 0.0065 | 1.28E−50 | 0.0194 | 5.78E−618 | −0.0235 | 2.72E−170 | −0.0444 | 4.49E−247 |
| Urate | 0.0078 | 6.50E−92 | 0.0068 | 3.95E−100 | −0.0089 | 5.78E−33 | −0.0129 | 1.25E−28 |
| Urea | 0.0040 | 2.19E−20 | 0.0000 | 9.68E−01 | 0.0001 | 9.52E−01 | −0.0060 | 4.74E−06 |
| Vitamin D | −0.0036 | 4.10E−15 | −0.0021 | 6.54E−08 | 0.0056 | 4.39E−10 | 0.0086 | 8.55E−10 |
| White blood cell count | 0.0063 | 8.15E−45 | 0.0084 | 2.41E−110 | −0.0174 | 1.25E−89 | −0.0152 | 5.57E−29 |

| | Hyper Insulin | | | | All Cluster | |
|---|---|---|---|---|---|---|
| | Secretion (N = 32) | | SHBG (N = 1) | | SNPs (N = 211) | |
| | beta | Pvalue | beta | Pvalue | beta | Pvalue |
| Glycemic traits | | | | | | |
| 2-hour glucose | −0.0002 | 9.48E−01 | 0.0327 | 1.02E−01 | 0.0151 | 1.91E−25 |
| Corrected insulin response | 0.0317 | 9.49E−15 | −0.0170 | 4.40E−01 | −0.0122 | 1.72E−15 |
| Disposition index | 0.0316 | 2.02E−14 | −0.0260 | 2.37E−01 | −0.0129 | 6.51E−17 |
| Fasting glucose | 0.0040 | 6.37E−08 | 0.0060 | 1.34E−01 | 0.0065 | 2.91E−119 |
| Fasting Insulin | 0.0020 | 2.05E−03 | 0.0031 | 3.62E−01 | 0.0018 | 9.71E−14 |
| Fasting proinsulin | 0.0043 | 2.65E−03 | −0.0072 | 3.37E−01 | 0.0047 | 2.36E−18 |
| HbA1c | 0.0028 | 7.80E−16 | 0.0060 | 1.59E−03 | 0.0039 | 1.20E−195 |
| HOMA-R | −0.0006 | 3.73E−01 | −0.0015 | 6.68E−01 | −0.0014 | 9.14E−09 |
| HOMA-IR | 0.0016 | 4.95E−02 | 0.0026 | 5.45E−01 | 0.0026 | 2.85E−17 |
| Modified Stumvoll insulin sensitivity | −0.0019 | 3.48E−01 | −0.0004 | 9.71E−01 | −0.0062 | 1.02E−16 |
| index adjAgeSexBMI | | | | | | |
| Random glucose | 0.0072 | 3.76E−48 | 0.0081 | 1.27E−03 | 0.0100 | 4.16E−648 |
| Non-glycemic traits - anthropometric | | | | | | |
| BMI | 0.0039 | 2.58E−29 | −0.0057 | 1.54E−03 | 0.0032 | 5.46E−140 |
| Bodyfat percent | −0.0002 | 8.46E−01 | −0.0226 | 7.34E−05 | −0.0005 | 2.20E−01 |
| Diastolic blood pressure | 0.0023 | 8.68E−07 | −0.0019 | 4.39E−01 | 0.0014 | 3.03E−15 |
| Height | −0.0019 | 5.36E−10 | 0.0011 | 4.63E−01 | −0.0015 | 1.46E−42 |
| Hip circumference adjBMI Female | −0.0028 | 5.53E−03 | −0.0046 | 4.20E−01 | −0.0048 | 4.33E−38 |
| Hip circumference adjBMI Male | −0.0045 | 2.00E−04 | −0.0045 | 5.20E−01 | −0.0036 | 4.36E−17 |
| Systolic blood pressure | 0.0028 | 5.91E−10 | −0.0035 | 1.18E−01 | 0.0029 | 6.58E−69 |
| Waist circumference adjBMI Female | 0.0063 | 8.64E−11 | 0.0110 | 4.55E−02 | 0.0052 | 4.36E−49 |
| Waist circumference adjBMI Male | −0.0012 | 3.09E−01 | 0.0026 | 6.94E−01 | −0.0011 | 8.39E−03 |
| Waist circumference Female | 0.0046 | 3.52E−06 | −0.0041 | 4.64E−01 | 0.0038 | 1.99E−26 |
| Waist circumference Male | −0.0001 | 9.05E−01 | −0.0130 | 4.55E−02 | 0.0004 | 3.08E−01 |
| Waist-to-hip ratio adjBMI Female | 0.0078 | 5.35E−15 | 0.0130 | 1.81E−02 | 0.0080 | 2.54E−109 |
| Waist-to-hip ratio adjBMI Male | 0.0025 | 3.04E−02 | 0.0093 | 1.71E−01 | 0.0024 | 5.89E−09 |
| Waist-to-hip ratio Female | 0.0076 | 8.15E−15 | 0.0083 | 1.38E−01 | 0.0078 | 6.91E−106 |
| Waist-to-hip ratio Male | 0.0019 | 9.45E−02 | −0.0032 | 6.28E−01 | 0.0027 | 2.59E−11 |
| Non-glycemic traits - lab measures | | | | | | |
| Adiponectin | −0.0033 | 1.86E−04 | −0.0054 | 2.57E−01 | −0.0042 | 3.15E−35 |
| Alanine aminotransferase | 0.0054 | 3.71E−34 | 0.0058 | 9.84E−03 | 0.0067 | 5.73E−367 |
| Albumin | −0.0026 | 1.56E−07 | 0.0092 | 2.00E−04 | 0.0015 | 1.10E−15 |
| Alkaline phosphotase | 0.0054 | 4.09E−31 | −0.0064 | 5.93E−03 | −0.0010 | 2.56E−08 |
| Apolipoprotein B | 0.0011 | 1.56E−02 | 0.0123 | 2.42E−07 | −0.0008 | 1.23E−06 |
| Aspartate aminotransferase | 0.0022 | 8.52E−07 | −0.0052 | 2.44E−02 | 0.0022 | 2.86E−40 |
| Basophill count | 0.0003 | 4.29E−01 | 0.0017 | 4.13E−01 | −0.0001 | 3.87E−01 |
| Calcium | −0.0016 | 8.48E−04 | 0.0015 | 5.40E−01 | 0.0014 | 2.77E−14 |
| C-reactive protein | 0.0048 | 3.71E−24 | −0.0024 | 3.22E−01 | 0.0014 | 2.07E−16 |
| Direct bilirubin | −0.0027 | 8.09E−08 | −0.0129 | 2.78E−07 | −0.0003 | 6.25E−02 |
| Eosinophill count | 0.0030 | 2.52E−17 | −0.0061 | 6.00E−04 | 0.0009 | 1.24E−12 |
| Gamma-glutamyl transferase | 0.0039 | 1.18E−18 | 0.0007 | 7.66E−01 | 0.0030 | 2.32E−74 |
| HDL | −0.0095 | 2.92E−98 | −0.0114 | 6.20E−07 | −0.0080 | 1.28E−499 |
| Hemoglobin concentration | −0.0010 | 6.93E−03 | −0.0058 | 2.26E−03 | 0.0006 | 5.66E−06 |
| High light scatter reticulocyte count | 0.0034 | 1.40E−12 | −0.0038 | 1.16E−01 | 0.0047 | 1.02E−155 |
| IGF-1 | 0.0019 | 3.84E−05 | −0.0170 | 4.12E−13 | 0.0022 | 1.87E−36 |
| Leptin | 0.0025 | 1.30E−02 | −0.0022 | 6.89E−01 | 0.0012 | 1.87E−03 |
| Leptin adjBMI | 0.0037 | 3.64E−03 | −0.0131 | 6.50E−02 | 0.0025 | 1.94E−07 |
| Lipoprotein A | −0.0013 | 1.32E−02 | 0.0025 | 3.57E−01 | 0.0005 | 8.18E−03 |
| Lymphocyte count | 0.0050 | 3.43E−26 | 0.0034 | 1.61E−01 | 0.0028 | 7.62E−58 |
| Mean corpuscular volume | −0.0056 | 1.06E−32 | −0.0008 | 7.48E−01 | −0.0019 | 6.30E−29 |
| Mean platelet volume | 0.0039 | 2.79E−16 | −0.0032 | 1.85E−01 | 0.0017 | 2.60E−22 |
| Mean reticulocyte volume | −0.0023 | 9.72E−07 | −0.0025 | 3.06E−01 | −0.0005 | 5.21E−03 |
| Monocyte count | 0.0009 | 6.08E−02 | 0.0128 | 4.23E−08 | 0.0006 | 4.46E−04 |
| Phosphate | −0.0002 | 6.17E−01 | 0.0010 | 6.94E−01 | 0.0002 | 3.76E−01 |
| Platelet count | −0.0020 | 1.52E−05 | 0.0121 | 1.89E−07 | −0.0015 | 3.76E−19 |
| Platelet distribution width | 0.0042 | 4.39E−19 | −0.0124 | 1.78E−07 | 0.0026 | 1.49E−49 |
| Red blood cell count | 0.0028 | 7.09E−12 | −0.0026 | 2.03E−01 | 0.0015 | 8.37E−25 |
| Red blood cell distribution width | 0.0053 | 2.22E−29 | −0.0150 | 3.40E−10 | 0.0007 | 8.34E−05 |
| Sex hormone-binding globulin | −0.0093 | 5.14E−95 | −0.2041 | 1.20E−1784 | −0.0086 | 4.13E−577 |
| Testosterone Female | 0.0008 | 2.33E−01 | −0.0167 | 3.14E−06 | 0.0000 | 9.31E−01 |
| Total bilirubin | −0.0034 | 1.08E−13 | −0.0118 | 3.33E−07 | −0.0012 | 1.25E−12 |
| Total protein | −0.0019 | 1.05E−04 | 0.0093 | 2.44E−04 | 0.0010 | 5.65E−08 |
| Triglycerides | 0.0070 | 2.77E−52 | 0.0103 | 9.62E−06 | 0.0062 | 7.87E−286 |
| Urate | 0.0026 | 5.50E−11 | −0.0012 | 5.71E−01 | 0.0026 | 2.60E−66 |
| Urea | 0.0000 | 9.14E−01 | 0.0065 | 4.63E−03 | 0.0013 | 1.21E−13 |
| Vitamin D | 0.0000 | 9.94E−01 | −0.0084 | 6.46E−04 | −0.0011 | 4.69E−09 |
| White blood cell count | 0.0052 | 1.85E−28 | 0.0062 | 9.93E−03 | 0.0028 | 9.62E−58 |

TABLE 8
Epigenomics analysis results for T2D clusters

| | | | | |
|---|---|---|---|---|
| cluster | annotation | obs_cppa | p | fdr |

a) Parker annotations

| | | | | |
|---|---|---|---|---|
| ALP_neg | Adipose | 0.063759287 | 0.2393 | 0.855482 |
| ALP_neg | AnteriorCaudate | 0.016470182 | 0.4033 | 0.855482 |
| ALP_neg | CD34-PB | 2.42E−06 | 0.8907 | 0.9005 |
| ALP_neg | CingulateGyrus | 0.066021485 | 0.1781 | 0.855482 |
| ALP_neg | ColonicMucosa | 0.065970848 | 0.1824 | 0.855482 |
| ALP_neg | DuodenumMucosa | 0.063761797 | 0.2279 | 0.855482 |
| ALP_neg | ES-HUES6 | 8.85E−05 | 0.5949 | 0.9005 |
| ALP_neg | ES-HUES64 | 1.51E−05 | 0.7645 | 0.9005 |
| ALP_neg | H1 | 8.15E−06 | 0.8484 | 0.9005 |
| ALP_neg | HMEC | 0.002153999 | 0.4859 | 0.855482 |
| ALP_neg | HSMM | 1.49E−05 | 0.7659 | 0.9005 |
| ALP_neg | HippocampusMiddle | 0.004662596 | 0.4528 | 0.855482 |
| ALP_neg | Huvec | 2.06E−06 | 0.9005 | 0.9005 |
| ALP_neg | InferiorTemporalLobe | 7.93E−05 | 0.6421 | 0.9005 |
| ALP_neg | Islets | 0.01653397 | 0.38 | 0.855482 |
| ALP_neg | Liver | 0.372004848 | 0.0001 | 0.0028 |
| ALP_neg | MidFrontalLobe | 0.059542938 | 0.2834 | 0.855482 |
| ALP_neg | NHEK | 0.002148464 | 0.5045 | 0.855482 |
| ALP_neg | NHLF | 8.67E−06 | 0.8322 | 0.9005 |
| ALP_neg | RectalMucosa | 0.080296569 | 0.0898 | 0.855482 |
| ALP_neg | RectalSmoothMuscle | 0.00213708 | 0.5194 | 0.855482 |
| ALP_neg | SkeletalMuscle | 0.063755953 | 0.2331 | 0.855482 |
| ALP_neg | StomachSmoothMuscle | 0.052311054 | 0.3036 | 0.855482 |
| ALP_neg | SubstantiaNigra | 0.01646963 | 0.4023 | 0.855482 |
| ALP_neg | hASC-t1 | 0.016469157 | 0.4148 | 0.855482 |
| ALP_neg | hASC-t2 | 8.17E−06 | 0.8504 | 0.9005 |
| ALP_neg | hASC-t3 | 8.34E−06 | 0.8363 | 0.9005 |
| ALP_neg | hASC-t4 | 8.31E−06 | 0.8382 | 0.9005 |
| Beta-cell1 | Adipose | 0.189978471 | 0.0009 | 0.0084 |
| Beta-cell1 | AnteriorCaudate | 0.099889556 | 0.3348 | 0.9995 |
| Beta-cell1 | CD34-PB | 0.024165971 | 0.9964 | 0.9995 |
| Beta-cell1 | CingulateGyrus | 0.078489947 | 0.6255 | 0.9995 |
| Beta-cell1 | ColonicMucosa | 0.046827142 | 0.9469 | 0.9995 |
| Beta-cell1 | DuodenumMucosa | 0.069090174 | 0.7541 | 0.9995 |
| Beta-cell1 | ES-HUES6 | 0.054599805 | 0.9017 | 0.9995 |
| Beta-cell1 | ES-HUES64 | 0.017709999 | 0.9995 | 0.9995 |
| Beta-cell1 | H1 | 0.048071265 | 0.9428 | 0.9995 |
| Beta-cell1 | HMEC | 0.148782722 | 0.0235 | 0.1316 |
| Beta-cell1 | HSMM | 0.137103382 | 0.0504 | 0.2352 |
| Beta-cell1 | HippocampusMiddle | 0.19287662 | 0.0006 | 0.0084 |
| Beta-cell1 | Huvec | 0.05723626 | 0.8808 | 0.9995 |
| Beta-cell1 | InferiorTemporalLobe | 0.097840669 | 0.3583 | 0.9995 |
| Beta-cell1 | Islets | 0.221930928 | 0.0001 | 0.0028 |
| Beta-cell1 | Liver | 0.07300073 | 0.6997 | 0.9995 |
| Beta-cell1 | MidFrontalLobe | 0.114134073 | 0.1875 | 0.75 |
| Beta-cell1 | NHEK | 0.179493213 | 0.0025 | 0.0175 |
| Beta-cell1 | NHLF | 0.061184883 | 0.8421 | 0.9995 |
| Beta-cell1 | RectalMucosa | 0.051538361 | 0.9145 | 0.9995 |
| Beta-cell1 | RectalSmoothMuscle | 0.022236108 | 0.9978 | 0.9995 |
| Beta-cell1 | SkeletalMuscle | 0.108737714 | 0.2353 | 0.82355 |
| Beta-cell1 | StomachSmoothMuscle | 0.055825096 | 0.8879 | 0.9995 |
| Beta-cell1 | SubstantiaNigra | 0.066641837 | 0.7741 | 0.9995 |
| Beta-cell1 | hASC-t1 | 0.072835563 | 0.7103 | 0.9995 |
| Beta-cell1 | hASC-t2 | 0.05884822 | 0.859 | 0.9995 |
| Beta-cell1 | hASC-t3 | 0.07549253 | 0.6716 | 0.9995 |
| Beta-cell1 | hASC-t4 | 0.073319612 | 0.7103 | 0.9995 |
| Beta-cell2 | Adipose | 0.460515498 | 0 | 0 |
| Beta-cell2 | AnteriorCaudate | 0.116806527 | 0.561 | 0.9982 |
| Beta-cell2 | CD34-PB | 0.025303673 | 0.9895 | 0.9982 |
| Beta-cell2 | CingulateGyrus | 0.095824019 | 0.7024 | 0.9982 |
| Beta-cell2 | ColonicMucosa | 0.01775238 | 0.9958 | 0.9982 |
| Beta-cell2 | DuodenumMucosa | 0.067808981 | 0.8781 | 0.9982 |
| Beta-cell2 | ES-HUES6 | 0.044458159 | 0.9605 | 0.9982 |
| Beta-cell2 | ES-HUES64 | 0.024147566 | 0.9877 | 0.9982 |
| Beta-cell2 | H1 | 0.012358578 | 0.9982 | 0.9982 |
| Beta-cell2 | HMEC | 0.357275716 | 0 | 0 |
| Beta-cell2 | HSMM | 0.097588187 | 0.6917 | 0.9982 |
| Beta-cell2 | HippocampusMiddle | 0.459406204 | 0 | 0 |
| Beta-cell2 | Huvec | 0.038697558 | 0.9733 | 0.9982 |
| Beta-cell2 | InferiorTemporalLobe | 0.107558373 | 0.6296 | 0.9982 |
| Beta-cell2 | Islets | 0.290724244 | 0.0041 | 0.02296 |
| Beta-cell2 | Liver | 0.164298608 | 0.2425 | 0.9982 |
| Beta-cell2 | MidFrontalLobe | 0.083120311 | 0.7998 | 0.9982 |
| Beta-cell2 | NHEK | 0.359775178 | 0.0001 | 0.0007 |
| Beta-cell2 | NHLF | 0.076825638 | 0.8305 | 0.9982 |
| Beta-cell2 | RectalMucosa | 0.036475015 | 0.9712 | 0.9982 |
| Beta-cell2 | RectalSmoothMuscle | 0.030291495 | 0.9823 | 0.9982 |
| Beta-cell2 | SkeletalMuscle | 0.123189436 | 0.5144 | 0.9982 |
| Beta-cell2 | StomachSmoothMuscle | 0.090919444 | 0.7412 | 0.9982 |
| Beta-cell2 | SubstantiaNigra | 0.088434934 | 0.748 | 0.9982 |
| Beta-cell2 | hASC-t1 | 0.051517738 | 0.94 | 0.9982 |
| Beta-cell2 | hASC-t2 | 0.081190535 | 0.8015 | 0.9982 |
| Beta-cell2 | hASC-t3 | 0.082833922 | 0.7978 | 0.9982 |
| Beta-cell2 | hASC-t4 | 0.110128046 | 0.6069 | 0.9982 |
| Lipodystrophy | Adipose | 0.36704256 | 0 | 0 |
| Lipodystrophy | AnteriorCaudate | 0.101799207 | 0.8078 | 0.9996 |
| Lipodystrophy | CD34-PB | 0.090867706 | 0.9079 | 0.9996 |
| Lipodystrophy | CingulateGyrus | 0.104499356 | 0.7845 | 0.9996 |
| Lipodystrophy | ColonicMucosa | 0.112343795 | 0.6822 | 0.9996 |
| Lipodystrophy | DuodenumMucosa | 0.084437814 | 0.9443 | 0.9996 |
| Lipodystrophy | ES-HUES6 | 0.061902841 | 0.9945 | 0.9996 |
| Lipodystrophy | ES-HUES64 | 0.060058973 | 0.997 | 0.9996 |
| Lipodystrophy | H1 | 0.048465971 | 0.9996 | 0.9996 |
| Lipodystrophy | HMEC | 0.143815289 | 0.2799 | 0.78372 |
| Lipodystrophy | HSMM | 0.119060527 | 0.6063 | 0.9996 |
| Lipodystrophy | HippocampusMiddle | 0.144824177 | 0.2711 | 0.78372 |
| Lipodystrophy | Huvec | 0.088285616 | 0.9193 | 0.9996 |
| Lipodystrophy | InferiorTemporalLobe | 0.12908288 | 0.4604 | 0.9996 |
| Lipodystrophy | Islets | 0.12222718 | 0.5571 | 0.9996 |
| Lipodystrophy | Liver | 0.177285035 | 0.0544 | 0.35392 |
| Lipodystrophy | MidFrontalLobe | 0.113584787 | 0.6813 | 0.9996 |
| Lipodystrophy | NHEK | 0.127241883 | 0.497 | 0.9996 |
| Lipodystrophy | NHLF | 0.183420047 | 0.0375 | 0.35 |
| Lipodystrophy | RectalMucosa | 0.170616849 | 0.0759 | 0.3542 |
| Lipodystrophy | RectalSmoothMuscle | 0.08131007 | 0.9588 | 0.9996 |
| Lipodystrophy | SkeletalMuscle | 0.175712356 | 0.0632 | 0.35392 |
| Lipodystrophy | StomachSmoothMuscle | 0.095734747 | 0.8736 | 0.9996 |
| Lipodystrophy | SubstantiaNigra | 0.091040828 | 0.9028 | 0.9996 |
| Lipodystrophy | hASC-t1 | 0.185240471 | 0.0352 | 0.35 |
| Lipodystrophy | hASC-t2 | 0.103555378 | 0.7982 | 0.9996 |
| Lipodystrophy | hASC-t3 | 0.155642478 | 0.1709 | 0.6836 |
| Lipodystrophy | hASC-t4 | 0.15072315 | 0.2215 | 0.77525 |
| LipoproteinA | Adipose | 0.209811162 | 0.1834 | 0.97664 |
| LipoproteinA | AnteriorCaudate | 3.05E−06 | 0.8544 | 0.9968 |
| LipoproteinA | CD34-PB | 0 | 1 | 1 |
| LipoproteinA | CingulateGyrus | 3.05E−06 | 0.8213 | 0.9968 |
| LipoproteinA | ColonicMucosa | 0.227092884 | 0.1411 | 0.97664 |
| LipoproteinA | DuodenumMucosa | 0.00034653 | 0.5713 | 0.985473 |
| LipoproteinA | ES-HUES6 | 0.00017464 | 0.6059 | 0.985473 |
| LipoproteinA | ES-HUES64 | 1.76E−06 | 0.893 | 1 |
| LipoproteinA | H1 | 0.036267091 | 0.4647 | 0.981 |
| LipoproteinA | HMEC | 0.071520039 | 0.3488 | 0.97664 |
| LipoproteinA | HSMM | 1.30E−05 | 0.6401 | 0.985473 |
| LipoproteinA | HippocampusMiddle | 4.60E−06 | 0.7515 | 0.985473 |
| LipoproteinA | Huvec | 4.78E−06 | 0.7202 | 0.985473 |
| LipoproteinA | InferiorTemporalLobe | 0 | 1 | 1 |
| LipoproteinA | Islets | 3.10E−06 | 0.7743 | 0.985473 |
| LipoproteinA | Liver | 0.612296987 | 0.0346 | 0.9688 |
| LipoproteinA | MidFrontalLobe | 1.63E−06 | 0.9333 | 1 |
| LipoproteinA | NHEK | 0.103550581 | 0.322 | 0.97664 |
| LipoproteinA | NHLF | 7.99E−06 | 0.6781 | 0.985473 |
| LipoproteinA | RectalMucosa | 0.036444093 | 0.4329 | 0.981 |
| LipoproteinA | RectalSmoothMuscle | 0.048266524 | 0.3905 | 0.981 |
| LipoproteinA | SkeletalMuscle | 0.23295792 | 0.113 | 0.97664 |
| LipoproteinA | StomachSmoothMuscle | 0.189331062 | 0.2144 | 0.97664 |
| LipoproteinA | SubstantiaNigra | 0.002182016 | 0.5491 | 0.985473 |
| LipoproteinA | hASC-t1 | 0.333134803 | 0.0703 | 0.97664 |
| LipoproteinA | hASC-t2 | 0.139462111 | 0.2972 | 0.97664 |
| LipoproteinA | hASC-t3 | 0.185034012 | 0.2492 | 0.97664 |
| LipoproteinA | hASC-t4 | 0.003039613 | 0.4905 | 0.981 |
| LiverLipid | Adipose | 0.101856485 | 0.0716 | 0.800904 |
| LiverLipid | AnteriorCaudate | 0.01868031 | 0.5252 | 0.800904 |
| LiverLipid | CD34-PB | 0.012828332 | 0.6327 | 0.800904 |
| LiverLipid | CingulateGyrus | 0.017627269 | 0.5562 | 0.800904 |
| LiverLipid | ColonicMucosa | 0.019510696 | 0.5007 | 0.800904 |
| LiverLipid | DuodenumMucosa | 0.02735888 | 0.3853 | 0.800904 |
| LiverLipid | ES-HUES6 | 0.024864013 | 0.4085 | 0.800904 |
| LiverLipid | ES-HUES64 | 0.026719964 | 0.3967 | 0.800904 |
| LiverLipid | H1 | 0.011192991 | 0.6698 | 0.800904 |
| LiverLipid | HMEC | 0.011121508 | 0.6615 | 0.800904 |
| LiverLipid | HSMM | 0.017035079 | 0.5748 | 0.800904 |
| LiverLipid | HippocampusMiddle | 0.03701319 | 0.2955 | 0.800904 |
| LiverLipid | Huvec | 0.005425565 | 0.7723 | 0.800904 |
| LiverLipid | InferiorTemporalLobe | 0.019483658 | 0.4983 | 0.800904 |
| LiverLipid | Islets | 0.088523004 | 0.1295 | 0.800904 |
| LiverLipid | Liver | 0.374739344 | 0 | 0 |
| LiverLipid | MidFrontalLobe | 0.018951322 | 0.507 | 0.800904 |
| LiverLipid | NHEK | 0.020566008 | 0.478 | 0.800904 |
| LiverLipid | NHLF | 0.007714648 | 0.7312 | 0.800904 |
| LiverLipid | RectalMucosa | 0.021436992 | 0.4598 | 0.800904 |
| LiverLipid | RectalSmoothMuscle | 0.023293369 | 0.4421 | 0.800904 |
| LiverLipid | SkeletalMuscle | 0.005979181 | 0.7565 | 0.800904 |
| LiverLipid | StomachSmoothMuscle | 0.003051958 | 0.8555 | 0.8555 |
| LiverLipid | SubstantiaNigra | 0.020517984 | 0.481 | 0.800904 |
| LiverLipid | hASC-t1 | 0.008374124 | 0.703 | 0.800904 |
| LiverLipid | hASC-t2 | 0.008094487 | 0.7153 | 0.800904 |
| LiverLipid | hASC-t3 | 0.007405989 | 0.734 | 0.800904 |
| LiverLipid | hASC-t4 | 0.009605639 | 0.6835 | 0.800904 |
| Obesity | Adipose | 0.107547268 | 0.0516 | 0.1827 |
| Obesity | AnteriorCaudate | 0.057829049 | 0.6669 | 0.9999 |
| Obesity | CD34-PB | 0.023801189 | 0.9911 | 0.9999 |
| Obesity | CingulateGyrus | 0.045489548 | 0.8528 | 0.9999 |
| Obesity | ColonicMucosa | 0.017995963 | 0.999 | 0.9999 |
| Obesity | DuodenumMucosa | 0.048208151 | 0.8221 | 0.9999 |
| Obesity | ES-HUES6 | 0.049779955 | 0.7992 | 0.9999 |
| Obesity | ES-HUES64 | 0.043398569 | 0.8775 | 0.9999 |
| Obesity | H1 | 0.013669654 | 0.9999 | 0.9999 |
| Obesity | HMEC | 0.117003876 | 0.0233 | 0.147467 |
| Obesity | HSMM | 0.107108524 | 0.0522 | 0.1827 |
| Obesity | HippocampusMiddle | 0.045434865 | 0.8569 | 0.9999 |
| Obesity | Huvec | 0.055585939 | 0.7028 | 0.9999 |
| Obesity | InferiorTemporalLobe | 0.049805065 | 0.8004 | 0.9999 |
| Obesity | Islets | 0.054000869 | 0.7255 | 0.9999 |
| Obesity | Liver | 0.070215302 | 0.4424 | 0.9999 |
| Obesity | MidFrontalLobe | 0.043413765 | 0.877 | 0.9999 |
| Obesity | NHEK | 0.130305151 | 0.008 | 0.112 |
| Obesity | NHLF | 0.089328501 | 0.1769 | 0.49532 |
| Obesity | RectalMucosa | 0.036110728 | 0.9407 | 0.9999 |
| Obesity | RectalSmoothMuscle | 0.03855732 | 0.9246 | 0.9999 |
| Obesity | SkeletalMuscle | 0.095313606 | 0.1235 | 0.384222 |
| Obesity | StomachSmoothMuscle | 0.026778168 | 0.9884 | 0.9999 |
| Obesity | SubstantiaNigra | 0.078500265 | 0.3087 | 0.785782 |
| Obesity | hASC-t1 | 0.114288447 | 0.0316 | 0.147467 |
| Obesity | hASC-t2 | 0.119721032 | 0.0212 | 0.147467 |
| Obesity | hASC-t3 | 0.131738871 | 0.0061 | 0.112 |
| Obesity | hASC-t4 | 0.114770479 | 0.0308 | 0.147467 |
| Proinsulin | Adipose | 0.137738796 | 0.4936 | 0.97216 |
| Proinsulin | AnteriorCaudate | 0.322787066 | 0.0113 | 0.1232 |
| Proinsulin | CD34-PB | 0.132143779 | 0.5208 | 0.97216 |
| Proinsulin | CingulateGyrus | 0.237964737 | 0.095 | 0.665 |
| Proinsulin | ColonicMucosa | 0.160186717 | 0.3709 | 0.877567 |
| Proinsulin | DuodenumMucosa | 0.176109066 | 0.2973 | 0.877567 |
| Proinsulin | ES-HUES6 | 0.157997293 | 0.3761 | 0.877567 |
| Proinsulin | ES-HUES64 | 0.062318411 | 0.8817 | 0.9772 |
| Proinsulin | H1 | 0.032037833 | 0.9523 | 0.9772 |
| Proinsulin | HMEC | 0.08359951 | 0.7768 | 0.9772 |
| Proinsulin | HSMM | 0.113647866 | 0.6335 | 0.9772 |
| Proinsulin | HippocampusMiddle | 0.321492655 | 0.0132 | 0.1232 |
| Proinsulin | Huvec | 0.025596082 | 0.9656 | 0.9772 |
| Proinsulin | InferiorTemporalLobe | 0.207658279 | 0.1741 | 0.812467 |
| Proinsulin | Islets | 0.414598719 | 0.0006 | 0.0168 |
| Proinsulin | Liver | 0.193403386 | 0.2271 | 0.8232 |
| Proinsulin | MidFrontalLobe | 0.189992112 | 0.2352 | 0.8232 |
| Proinsulin | NHEK | 0.075337169 | 0.8244 | 0.9772 |
| Proinsulin | NHLF | 0.081285417 | 0.7921 | 0.9772 |
| Proinsulin | RectalMucosa | 0.166686948 | 0.3336 | 0.877567 |
| Proinsulin | RectalSmoothMuscle | 0.067003625 | 0.8616 | 0.9772 |
| Proinsulin | SkeletalMuscle | 0.092094999 | 0.737 | 0.9772 |
| Proinsulin | StomachSmoothMuscle | 0.225841832 | 0.1249 | 0.69944 |
| Proinsulin | SubstantiaNigra | 0.150208198 | 0.4147 | 0.8932 |
| Proinsulin | hASC-t1 | 0.039293015 | 0.9375 | 0.9772 |
| Proinsulin | hASC-t2 | 0.039022932 | 0.9379 | 0.9772 |
| Proinsulin | hASC-t3 | 0.021132073 | 0.9772 | 0.9772 |
| Proinsulin | hASC-t4 | 0.037259677 | 0.9408 | 0.9772 |
| SHBG | Adipose | 0.039473134 | 0.785 | 0.999091 |
| SHBG | AnteriorCaudate | 0.371630572 | 0.0699 | 0.9786 |
| SHBG | CD34-PB | 0.042014278 | 0.6746 | 0.994147 |
| SHBG | CingulateGyrus | 0.374312243 | 0.0345 | 0.966 |
| SHBG | ColonicMucosa | 0.039555678 | 0.7496 | 0.999091 |
| SHBG | DuodenumMucosa | 0.201767171 | 0.2861 | 0.989644 |
| SHBG | ES-HUES6 | 0.117397795 | 0.3655 | 0.989644 |
| SHBG | ES-HUES64 | 0.024128501 | 0.9659 | 1 |
| SHBG | H1 | 0.058608946 | 0.39 | 0.989644 |
| SHBG | HMEC | 0.02714065 | 0.9335 | 1 |
| SHBG | HSMM | 0.045516679 | 0.5715 | 0.989644 |
| SHBG | HippocampusMiddle | 0.224696038 | 0.2476 | 0.989644 |
| SHBG | Huvec | 0.040809682 | 0.7125 | 0.9975 |
| SHBG | InferiorTemporalLobe | 0.035739411 | 0.8277 | 1 |
| SHBG | Islets | 0.168260812 | 0.3301 | 0.989644 |
| SHBG | Liver | 0.333777984 | 0.106 | 0.987 |
| SHBG | MidFrontalLobe | 0.241529001 | 0.1808 | 0.989644 |
| SHBG | NHEK | 0.033956901 | 0.8948 | 1 |
| SHBG | NHLF | 0.04898335 | 0.5017 | 0.989644 |
| SHBG | RectalMucosa | 0.051481021 | 0.4596 | 0.989644 |
| SHBG | RectalSmoothMuscle | 0.044075888 | 0.604 | 0.989644 |
| SHBG | SkeletalMuscle | 0.035343679 | 0.858 | 1 |
| SHBG | StomachSmoothMuscle | 0.255467612 | 0.141 | 0.987 |
| SHBG | SubstantiaNigra | 0.225469069 | 0.2122 | 0.989644 |
| SHBG | hASC-t1 | 0.048255582 | 0.5303 | 0.989644 |
| SHBG | hASC-t2 | 0.04231833 | 0.6362 | 0.989644 |
| SHBG | hASC-t3 | 0.020813211 | 1 | 1 |
| SHBG | hASC-t4 | 0.051570854 | 0.4278 | 0.989644 |
| Hyper Insulin Secretion | Adipose | 0.200965634 | 0.085 | 0.603292 |
| Hyper Insulin Secretion | AnteriorCaudate | 0.172167695 | 0.2452 | 0.603292 |
| Hyper Insulin Secretion | CD34-PB | 0.1688846 | 0.2616 | 0.603292 |
| Hyper Insulin Secretion | CingulateGyrus | 0.168696081 | 0.2663 | 0.603292 |
| Hyper Insulin Secretion | ColonicMucosa | 0.16753301 | 0.2801 | 0.603292 |
| Hyper Insulin Secretion | DuodenumMucosa | 0.151449378 | 0.4266 | 0.74655 |
| Hyper Insulin Secretion | ES-HUES6 | 0.098165995 | 0.9234 | 0.9923 |
| Hyper Insulin Secretion | ES-HUES64 | 0.106760013 | 0.8663 | 0.9923 |
| Hyper Insulin Secretion | H1 | 0.070469045 | 0.9923 | 0.9923 |
| Hyper Insulin Secretion | HMEC | 0.097663299 | 0.921 | 0.9923 |
| Hyper Insulin Secretion | HSMM | 0.143664685 | 0.5042 | 0.830447 |
| Hyper Insulin Secretion | HippocampusMiddle | 0.174737159 | 0.2181 | 0.603292 |
| Hyper Insulin Secretion | Huvec | 0.095842517 | 0.9296 | 0.9923 |
| Hyper Insulin Secretion | InferiorTemporalLobe | 0.181563188 | 0.1778 | 0.603292 |
| Hyper Insulin Secretion | Islets | 0.187618732 | 0.1395 | 0.603292 |
| Hyper Insulin Secretion | Liver | 0.211917113 | 0.0483 | 0.603292 |
| Hyper Insulin Secretion | MidFrontalLobe | 0.1764188 | 0.2059 | 0.603292 |
| Hyper Insulin Secretion | NHEK | 0.123024859 | 0.7256 | 0.9923 |
| Hyper Insulin Secretion | NHLF | 0.162777412 | 0.324 | 0.648 |
| Hyper Insulin Secretion | RectalMucosa | 0.156979623 | 0.3737 | 0.697573 |
| Hyper Insulin Secretion | RectalSmoothMuscle | 0.120255776 | 0.7583 | 0.9923 |
| Hyper Insulin Secretion | SkeletalMuscle | 0.203116686 | 0.0781 | 0.603292 |
| Hyper Insulin Secretion | StomachSmoothMuscle | 0.179539128 | 0.189 | 0.603292 |
| Hyper Insulin Secretion | SubstantiaNigra | 0.188524765 | 0.1294 | 0.603292 |
| Hyper Insulin Secretion | hASC-t1 | 0.126267661 | 0.6997 | 0.9923 |
| Hyper Insulin Secretion | hASC-t2 | 0.089921558 | 0.9548 | 0.9923 |
| Hyper Insulin Secretion | hASC-t3 | 0.081243246 | 0.9768 | 0.9923 |
| Hyper Insulin Secretion | hASC-t4 | 0.115852537 | 0.791 | 0.9923 |

b) HEA islet annotations

| | | | | |
|---|---|---|---|---|
| ALP_neg | GCGhi_alpha | 0.027362 | 0.0884 | 0.6188 |
| ALP_neg | GCGlo_alpha | 7.81E−05 | 0.3751 | 0.741767 |
| ALP_neg | INShi_beta | 0.027362 | 0.0746 | 0.6188 |
| ALP_neg | INSlo_beta | 7.84E−05 | 0.3138 | 0.741767 |
| ALP_neg | SSThi_delta | 7.15E−05 | 0.4781 | 0.741767 |
| ALP_neg | SSTlo_delta | 7.00E−06 | 0.6358 | 0.741767 |
| ALP_neg | acinar | 7.16E−05 | 0.4297 | 0.741767 |
| ALP_neg | activated_stellate | 7.11E−05 | 0.5995 | 0.741767 |
| ALP_neg | ductal | 7.85E−05 | 0.3 | 0.741767 |
| ALP_neg | endothelial | 7.12E−05 | 0.5677 | 0.741767 |
| ALP_neg | gamma | 7.84E−05 | 0.3194 | 0.741767 |
| ALP_neg | pancreatic CD8 T | 1.10E−08 | 0.9534 | 0.9534 |
| ALP_neg | pancreatic_macrophage | 6.74E−08 | 0.8755 | 0.942846 |
| ALP_neg | quiescent_stellate | 7.15E−05 | 0.5004 | 0.741767 |
| Beta-cell1 | GCGhi_alpha | 0.073119 | 0.0319 | 0.2233 |
| Beta-cell1 | GCGlo_alpha | 0.041266 | 0.4042 | 0.943133 |
| Beta-cell1 | INShi_beta | 0.11923 | 0.0001 | 0.0014 |
| Beta-cell1 | INSlo_beta | 0.052364 | 0.2079 | 0.72765 |
| Beta-cell1 | SSThi_delta | 0.065395 | 0.0684 | 0.3192 |
| Beta-cell1 | SSTlo_delta | 0.009377 | 0.9892 | 1 |
| Beta-cell1 | acinar | 0.044841 | 0.3279 | 0.91812 |
| Beta-cell1 | activated_stellate | 0.013311 | 0.9591 | 1 |
| Beta-cell1 | ductal | 0.032969 | 0.5962 | 1 |
| Beta-cell1 | endothelial | 0.021683 | 0.8479 | 1 |
| Beta-cell1 | gamma | 0.010864 | 0.9802 | 1 |
| Beta-cell1 | pancreatic_CD8_T | 0.002564 | 1 | 1 |
| Beta-cell1 | pancreatic_macrophage | 0.022242 | 0.8323 | 1 |
| Beta-cell1 | quiescent_stellate | 0.012461 | 0.9657 | 1 |
| Beta-cell2 | GCGhi_alpha | 0.092096 | 0.0703 | 0.328067 |
| Beta-cell2 | GCGlo_alpha | 0.0642 | 0.2657 | 0.74396 |
| Beta-cell2 | INShi_beta | 0.108989 | 0.0254 | 0.1778 |
| Beta-cell2 | INSlo_beta | 0.11721 | 0.0129 | 0.1778 |
| Beta-cell2 | SSThi_delta | 0.067507 | 0.2391 | 0.74396 |
| Beta-cell2 | SSTlo_delta | 0.023371 | 0.8654 | 1 |
| Beta-cell2 | acinar | 0.05118 | 0.4471 | 1 |
| Beta-cell2 | activated_stellate | 0.024793 | 0.8388 | 1 |
| Beta-cell2 | ductal | 0.031056 | 0.7562 | 1 |
| Beta-cell2 | endothelial | 0.010624 | 0.9828 | 1 |
| Beta-cell2 | gamma | 0.039029 | 0.6429 | 1 |
| Beta-cell2 | pancreatic_CD8_T | 6.89E−06 | 1 | 1 |
| Beta-cell2 | pancreatic_macrophage | 0.007274 | 0.9935 | 1 |
| Beta-cell2 | quiescent_stellate | 0.022109 | 0.8864 | 1 |
| Lipodystrophy | GCGhi_alpha | 0.053889 | 0.2251 | 0.5838 |
| Lipodystrophy | GCGlo_alpha | 0.074263 | 0.0417 | 0.470867 |
| Lipodystrophy | INShi_beta | 0.057532 | 0.172 | 0.5838 |
| Lipodystrophy | INSlo_beta | 0.043415 | 0.4259 | 0.785925 |
| Lipodystrophy | SSThi_delta | 0.028055 | 0.7717 | 0.982164 |
| Lipodystrophy | SSTlo_delta | 0.020588 | 0.9087 | 1 |
| Lipodystrophy | acinar | 0.031075 | 0.7124 | 0.982164 |
| Lipodystrophy | activated_stellate | 0.064625 | 0.1007 | 0.470867 |
| Lipodystrophy | ductal | 0.042063 | 0.4491 | 0.785925 |
| Lipodystrophy | endothelial | 0.064867 | 0.1009 | 0.470867 |
| Lipodystrophy | gamma | 0.011808 | 0.9924 | 1 |
| Lipodystrophy | pancreatic_CD8_T | 0.005 | 1 | 1 |
| Lipodystrophy | pancreatic_macrophage | 0.035623 | 0.6092 | 0.947644 |
| Lipodystrophy | quiescent_stellate | 0.052287 | 0.2502 | 0.5838 |
| LipoproteinA | GCGhi_alpha | 0.057229 | 0.1375 | 0.81564 |
| LipoproteinA | GCGlo_alpha | 3.14E−06 | 0.5795 | 0.99344 |
| LipoproteinA | INShi_beta | 0.037366 | 0.2913 | 0.81564 |
| LipoproteinA | INSlo_beta | 0.044099 | 0.2815 | 0.81564 |
| LipoproteinA | SSThi_delta | 3.11E−06 | 0.6448 | 0.99344 |
| LipoproteinA | SSTlo_delta | 3.03E−06 | 0.928 | 0.999385 |
| LipoproteinA | acinar | 0.137578 | 0.0688 | 0.81564 |
| LipoproteinA | activated_stellate | 4.65E−06 | 0.5015 | 0.99344 |
| LipoproteinA | ductal | 0.047564 | 0.2136 | 0.81564 |
| LipoproteinA | endothelial | 3.10E−06 | 0.7096 | 0.99344 |
| LipoproteinA | gamma | 3.04E−06 | 0.7848 | 0.99785 |
| LipoproteinA | pancreatic_CD8_T | 3.03E−06 | 1 | 1 |
| LipoproteinA | pancreatic_macrophage | 3.04E−06 | 0.8553 | 0.99785 |
| LipoproteinA | quiescent_stellate | 0.034099 | 0.36 | 0.84 |
| LiverLipid | GCGhi_alpha | 0.019152 | 0.0071 | 0.0994 |
| LiverLipid | GCGlo_alpha | 0.003242 | 0.3685 | 0.8884 |
| LiverLipid | INShi_beta | 0.012639 | 0.0389 | 0.2723 |
| LiverLipid | INSlo_beta | 0.002296 | 0.5023 | 0.8884 |
| LiverLipid | SSThi_delta | 0.001468 | 0.6802 | 0.8884 |
| LiverLipid | SSTlo_delta | 0.00039 | 0.8884 | 0.8884 |
| LiverLipid | acinar | 0.001119 | 0.7385 | 0.8884 |
| LiverLipid | activated_stellate | 0.003844 | 0.3065 | 0.8884 |
| LiverLipid | ductal | 0.002012 | 0.5574 | 0.8884 |
| LiverLipid | endothelial | 0.001117 | 0.7358 | 0.8884 |
| LiverLipid | gamma | 0.000548 | 0.8584 | 0.8884 |
| LiverLipid | pancreatic_CD8_T | 0.000397 | 0.8848 | 0.8884 |
| LiverLipid | pancreatic_macrophage | 0.0037 | 0.3291 | 0.8884 |
| LiverLipid | quiescent_stellate | 0.003051 | 0.3888 | 0.8884 |
| Obesity | GCGhi_alpha | 0.036827 | 0.0126 | 0.1295 |
| Obesity | GCGlo_alpha | 0.019106 | 0.2592 | 0.72576 |
| Obesity | INShi_beta | 0.035545 | 0.0185 | 0.1295 |
| Obesity | INSlo_beta | 0.023574 | 0.1376 | 0.4816 |
| Obesity | SSThi_delta | 0.007578 | 0.9172 | 0.9999 |
| Obesity | SSTlo_delta | 0.011722 | 0.6828 | 0.9999 |
| Obesity | acinar | 0.030261 | 0.0497 | 0.231933 |
| Obesity | activated_stellate | 0.015803 | 0.4103 | 0.957367 |
| Obesity | ductal | 0.008172 | 0.8897 | 0.9999 |
| Obesity | endothelial | 0.013641 | 0.5484 | 0.9999 |
| Obesity | gamma | 0.004783 | 0.9889 | 0.9999 |
| Obesity | pancreatic_CD8_T | 0.001795 | 0.9999 | 0.9999 |
| Obesity | pancreatic_macrophage | 0.003264 | 0.9977 | 0.9999 |
| Obesity | quiescent_stellate | 0.008911 | 0.8568 | 0.9999 |
| Proinsulin | GCGhi_alpha | 0.085108 | 0.3205 | 0.828489 |
| Proinsulin | GCGlo_alpha | 0.070661 | 0.4529 | 0.828489 |
| Proinsulin | INShi_beta | 0.174349 | 0.0423 | 0.5922 |
| Proinsulin | INSlo_beta | 0.117381 | 0.2004 | 0.828489 |
| Proinsulin | SSThi_delta | 0.073843 | 0.4084 | 0.828489 |
| Proinsulin | SSTlo_delta | 0.065468 | 0.5326 | 0.828489 |
| Proinsulin | acinar | 0.077479 | 0.3651 | 0.828489 |
| Proinsulin | activated_stellate | 0.006764 | 0.9708 | 0.9986 |
| Proinsulin | ductal | 0.069862 | 0.4653 | 0.828489 |
| Proinsulin | endothelial | 0.008232 | 0.9562 | 0.9986 |
| Proinsulin | gamma | 0.068186 | 0.4913 | 0.828489 |
| Proinsulin | pancreatic_CD8_T | 0.000831 | 0.9986 | 0.9986 |
| Proinsulin | pancreatic_macrophage | 0.00112 | 0.9968 | 0.9986 |
| Proinsulin | quiescent_stellate | 0.013039 | 0.8989 | 0.9986 |
| SIIBG | GCGhi_alpha | 0.082519 | 0.0672 | 0.9408 |
| SHBG | GCGlo_alpha | 0.071577 | 0.2159 | 0.9856 |
| SHBG | INShi_beta | 0.082503 | 0.1463 | 0.9856 |
| SHBG | INSlo_beta | 0.014065 | 0.5648 | 0.9884 |
| SHBG | SSThi_delta | 0.013821 | 0.7836 | 0.997309 |
| SHBG | SSTlo_delta | 0.002528 | 0.8563 | 0.999017 |
| SHBG | acinar | 0.021437 | 0.3546 | 0.9884 |
| SHBG | activated_stellate | 0.013833 | 0.7182 | 0.997309 |
| SHBG | ductal | 0.020883 | 0.427 | 0.9884 |
| SHBG | endothelial | 0.069333 | 0.2816 | 0.9856 |
| SHBG | gamma | 0.002377 | 0.9291 | 1 |
| SHBG | pancreatic_CD8_T | 0.001291 | 1 | 1 |
| SHBG | pancreatic_macrophage | 0.013876 | 0.6487 | 0.997309 |
| SHBG | quiescent_stellate | 0.01974 | 0.5033 | 0.9884 |
| Hyper Insulin Secretion | GCGhi_alpha | 0.113853 | 0.0131 | 0.1834 |
| Hyper Insulin Secretion | GCGlo_alpha | 0.07875 | 0.148 | 0.64372 |
| Hyper Insulin Secretion | INShi_beta | 0.100366 | 0.0372 | 0.2604 |
| Hyper Insulin Secretion | INSlo_beta | 0.071129 | 0.2299 | 0.64372 |
| Hyper Insulin Secretion | SSThi_delta | 0.05734 | 0.3997 | 0.7994 |
| Hyper Insulin Secretion | SSTlo_delta | 0.03187 | 0.8308 | 0.969267 |
| Hyper Insulin Secretion | acinar | 0.071039 | 0.2234 | 0.64372 |
| Hyper Insulin Secretion | activated_stellate | 0.043014 | 0.661 | 0.969267 |
| Hyper Insulin Secretion | ductal | 0.064016 | 0.3073 | 0.717033 |
| Hyper Insulin Secretion | endothelial | 0.03559 | 0.7747 | 0.969267 |
| Hyper Insulin Secretion | gamma | 0.015693 | 0.9786 | 0.9985 |
| Hyper Insulin Secretion | pancreatic_CD8_T | 0.008411 | 0.9985 | 0.9985 |
| Hyper Insulin Secretion | pancreatic_macrophage | 0.031965 | 0.8275 | 0.969267 |
| Hyper Insulin Secretion | quiescent_stellate | 0.038272 | 0.7332 | 0.969267 |

TABLE 9
Individual-level cluster pPS associations with traits in MGB Biobank (T2D clusters)
Unadjusted for T2D status

| | Lipoprotein A | | | | Triglycerides** | | | | White Blood Cell | |
|---|---|---|---|---|---|---|---|---|---|---|
| | (N = 181) | | BMI** (N = 23,274) | | (N = 16,059) | | HDL (N = 15,895) | | (N = 22,549) | |
| cluster | beta | p-value | beta | p-value | beta | p-value | beta | p-value | beta | p-value |
| Betacell1 | −0.9935 | 7.89E−01 | −0.0054 | 3.28E−05 | 0.0046 | 2.28E−01 | 0.0791 | 5.71E−01 | −0.0229 | 2.78E−01 |
| Betacell2 | 0.7618 | 8.46E−01 | −0.0043 | 9.71E−04 | 0.0013 | 7.40E−01 | −0.0641 | 6.48E−01 | −0.0225 | 2.92E−01 |
| Proinsulin | −9.1512 | 1.52E−02 | −0.0015 | 2.59E−01 | 0.0045 | 2.43E−01 | 0.1413 | 3.15E−01 | −0.0280 | 1.87E−01 |
| LipoproteinA | 18.7610 | 5.95E−07 | 0.0000 | 9.85E−01 | 0.0015 | 6.93E−01 | 0.0318 | 8.20E−01 | 0.0089 | 6.76E−01 |
| Obesity | −1.5036 | 6.98E−01 | 0.0182 | 9.63E−45 | 0.0089 | 2.06E−02 | −0.6239 | 8.46E−06 | 0.0586 | 5.64E−03 |
| Lipodystrophy | −1.7627 | 6.49E−01 | −0.0035 | 7.16E−03 | 0.0490 | 5.40E−37 | −1.8789 | 5.60E−41 | 0.0361 | 8.89E−02 |
| LiverLipid | −5.1847 | 1.93E−01 | 0.0000 | 9.95E−01 | −0.0363 | 4.32E−20 | 0.0695 | 6.29E−01 | −0.0393 | 7.13E−02 |
| ALP_neg | 0.3905 | 9.20E−01 | −0.0012 | 3.71E−01 | −0.0139 | 3.49E−04 | 0.3050 | 3.06E−02 | −0.0502 | 1.78E−02 |
| Hyper_Insulin_Secretion | 6.2941 | 1.15E−01 | 0.0012 | 3.55E−01 | 0.0110 | 5.38E−03 | −0.6738 | 2.52E−06 | 0.0294 | 1.75E−01 |
| SHBG | −9.5278 | 1.84E−02 | −0.0018 | 1.60E−01 | 0.0056 | 1.47E−01 | −0.0586 | 6.74E−01 | 0.0117 | 5.80E−01 |
| Combined pPS | 4.6919 | 2.04E−01 | −0.0007 | 6.18E−01 | 0.0228 | 3.49E−09 | −1.0619 | 3.38E−14 | −0.0032 | 8.79E−01 |

Adjusted for T2D status

| | Lipoprotein A | | | | Triglycerides** | | | | White Blood Cell | |
|---|---|---|---|---|---|---|---|---|---|---|
| | (N = 181) | | BMI** (N = 23,274) | | (N = 16,059) | | HDL (N = 15,895) | | (N = 22,549) | |
| cluster | beta | p-value | beta | p-value | beta | p-value | beta | p-value | beta | p-value |
| Betacell1 | −1.0539 | 7.80E−01 | −0.0071 | 4.22E−08 | −0.0014 | 7.10E−01 | 0.3182 | 2.17E−02 | −0.0339 | 1.15E−01 |
| Betacell2 | 0.9376 | 8.13E−01 | −0.0060 | 3.76E−06 | −0.0043 | 2.67E−01 | 0.1312 | 3.46E−01 | −0.0349 | 1.08E−01 |
| Proinsulin | −8.9656 | 1.88E−02 | −0.0022 | 8.72E−02 | 0.0013 | 7.34E−01 | 0.2626 | 5.99E−02 | −0.0328 | 1.30E−01 |
| LipoproteinA | 18.8075 | 6.64E−07 | −0.0005 | 6.96E−01 | 0.0008 | 8.40E−01 | 0.0755 | 5.87E−01 | 0.0070 | 7.47E−01 |
| Obesity | −1.3204 | 7.36E−01 | 0.0166 | 5.36E−38 | 0.0060 | 1.19E−01 | −0.4932 | 3.90E−04 | 0.0502 | 2.00E−02 |
| Lipodystrophy | −1.6380 | 6.91E−01 | −0.0048 | 2.06E−04 | 0.0453 | 4.38E−32 | −1.6818 | 1.31E−33 | 0.0252 | 2.44E−01 |
| LiverLipid | −4.9228 | 2.24E−01 | −0.0005 | 6.91E−01 | −0.0367 | 9.70E−21 | 0.0897 | 5.29E−01 | −0.0437 | 4.82E−02 |
| ALP_neg | 0.1193 | 9.76E−01 | −0.0015 | 2.37E−01 | −0.0157 | 4.98E−05 | 0.3025 | 3.07E−02 | −0.0568 | 8.53E−03 |
| Hyper_Insulin_Secretion | 6.4342 | 1.14E−01 | 0.0006 | 6.69E−01 | 0.0091 | 2.05E−02 | −0.5978 | 2.56E−05 | 0.0219 | 3.22E−01 |
| SHBG | −9.5265 | 1.98E−02 | −0.0019 | 1.44E−01 | 0.0039 | 3.08E−01 | 0.0038 | 9.78E−01 | 0.0111 | 6.04E−01 |
| Combined pPS | 5.2420 | 1.68E−01 | −0.0044 | 6.71E−04 | 0.0117 | 2.46E−03 | −0.6262 | 7.66E−06 | −0.0291 | 1.82E−01 |

T2D only

| | Lipoprotein A | | | | Triglycerides** | | | | White Blood Cell | |
|---|---|---|---|---|---|---|---|---|---|---|
| | (N = 14) | | BMI** (N = 1,893) | | (N = 1,675) | | HDL (N = 1,675) | | (N = 1,900) | |
| cluster | beta | p-value | beta | p-value | beta | p-value | beta | p-value | beta | p-value |
| Betacell1 | 29.169 | 8.24E−01 | −0.0168 | 1.24E−04 | −0.0178 | 1.31E−01 | 1.0643 | 9.95E−04 | −0.0366 | 7.00E−01 |
| Betacell2 | −16.498 | 6.10E−01 | −0.0142 | 1.15E−03 | 0.0011 | 9.27E−01 | 0.2339 | 4.69E−01 | 0.0180 | 8.53E−01 |
| Proinsulin | −28.733 | 5.81E−01 | −0.0019 | 6.66E−01 | −0.0065 | 5.80E−01 | 0.5078 | 1.16E−01 | −0.0816 | 3.96E−01 |
| LipoproteinA | 18.286 | 2.27E−01 | −0.0078 | 7.40E−02 | −0.0050 | 6.67E−01 | 0.4888 | 1.27E−01 | −0.0678 | 4.80E−01 |
| Obesity | 4.764 | 8.20E−01 | 0.0174 | 6.43E−05 | −0.0078 | 5.03E−01 | −0.4950 | 1.23E−01 | 0.1920 | 4.44E−02 |
| Lipodystrophy | −40.195 | 5.14E−01 | −0.0184 | 2.57E−05 | 0.0401 | 5.92E−04 | −1.5710 | 9.24E−07 | −0.3076 | 1.39E−03 |
| LiverLipid | 7.672 | 7.88E−01 | −0.0029 | 5.17E−01 | −0.0681 | 1.19E−08 | −0.1571 | 6.35E−01 | −0.1800 | 6.98E−02 |
| ALP_neg | −12.959 | 4.75E−01 | −0.0089 | 4.22E−02 | −0.0430 | 2.55E−04 | 0.4975 | 1.24E−01 | −0.1010 | 2.99E−01 |
| Hyper_Insulin_Secretion | 3.145 | 9.62E−01 | −0.0085 | 5.59E−02 | 0.0050 | 6.78E−01 | −0.3002 | 3.59E−01 | −0.0830 | 3.96E−01 |
| SHBG | 44.245 | 5.79E−01 | −0.0028 | 5.16E−01 | −0.0097 | 4.06E−01 | 0.4582 | 1.52E−01 | 0.0406 | 6.70E−01 |
| Combined pPS | 4.644 | 8.91E−01 | −0.0204 | 3.30E−06 | −0.0144 | 2.21E−01 | −0.0044 | 9.89E−01 | −0.2525 | 9.18E−03 |

Unadjusted for T2D status

| | C-reactive protein | | | | | | | | | |
|---|---|---|---|---|---|---|---|---|---|---|
| | (N = 7,316) | | ALP (N = 20,663) | | SHBG (N = 752) | | HbA1c (N = 13,571) | | ALT (N = 20,733) | |
| cluster | beta | p-value | beta | p-value | beta | p-value | beta | p-value | beta | p-value |
| Betacell1 | 0.1744 | 7.30E−01 | −0.6242 | 4.08E−02 | −1.1087 | 4.12E−01 | 0.0714 | 5.74E−18 | −0.2577 | 2.59E−01 |
| Betacell2 | 1.0495 | 3.98E−02 | −0.6876 | 2.61E−02 | 0.9455 | 4.88E−01 | 0.0667 | 7.71E−16 | −0.2168 | 3.49E−01 |
| Proinsulin | 0.1343 | 7.92E−01 | 0.0264 | 9.31E−01 | −1.6342 | 2.61E−01 | 0.0327 | 8.21E−05 | −0.0465 | 8.39E−01 |
| LipoproteinA | −0.2512 | 6.19E−01 | −0.2020 | 5.09E−01 | 0.2429 | 8.58E−01 | 0.0069 | 4.04E−01 | −0.4486 | 5.00E−02 |
| Obesity | 0.7849 | 1.17E−01 | 0.4836 | 1.15E−01 | 0.5379 | 6.91E−01 | 0.0386 | 3.06E−06 | −0.0304 | 8.94E−01 |
| Lipodystrophy | 0.5846 | 2.53E−01 | −0.5389 | 7.99E−02 | 0.3493 | 7.97E−01 | 0.0717 | 5.31E−18 | 0.6925 | 2.61E−03 |
| LiverLipid | −0.7475 | 1.52E−01 | −0.4432 | 1.58E−01 | 1.7450 | 2.07E−01 | 0.0206 | 1.56E−02 | −0.1354 | 5.65E−01 |
| ALP_neg | 0.4072 | 4.21E−01 | −3.0657 | 1.51E−23 | 1.5455 | 2.51E−01 | 0.0285 | 6.47E−04 | 0.3040 | 1.85E−01 |
| Hyper_Insulin_Secretion | −0.2322 | 6.52E−01 | 0.4787 | 1.28E−01 | −1.4202 | 3.09E−01 | 0.0378 | 7.95E−06 | 0.1804 | 4.43E−01 |
| SHBG | 0.1771 | 7.27E−01 | −0.6962 | 2.27E−02 | −5.1183 | 1.45E−04 | 0.0051 | 5.34E−01 | 0.1014 | 6.57E−01 |
| Combined pPS | 0.9390 | 6.47E−02 | −0.4792 | 1.20E−01 | −0.8492 | 5.40E−01 | 0.1398 | 1.20E−64 | 0.0347 | 8.80E−01 |

Adjusted for T2D status

| | C-reactive protein | | | | | | | | | |
|---|---|---|---|---|---|---|---|---|---|---|
| | (N = 7,316) | | ALP (N = 20,663) | | SHBG (N = 752) | | HbA1c (N = 13,571) | | ALT (N = 20,733) | |
| cluster | beta | p-value | beta | p-value | beta | p-value | beta | p-value | beta | p-value |
| Betacell1 | 0.0395 | 9.38E−01 | −0.7685 | 1.23E−02 | −0.6302 | 6.45E−01 | 0.0402 | 2.46E−09 | −0.2858 | 2.21E−01 |
| Betacell2 | 0.8934 | 8.20E−02 | −0.7973 | 1.02E−02 | 1.1704 | 3.93E−01 | 0.0304 | 6.68E−06 | −0.2896 | 2.20E−01 |
| Proinsulin | −0.1026 | 8.41E−01 | 0.0302 | 9.22E−01 | −1.7887 | 2.27E−01 | 0.0178 | 8.56E−03 | −0.0712 | 7.61E−01 |
| LipoproteinA | −0.2596 | 6.10E−01 | −0.2967 | 3.34E−01 | 0.2420 | 8.60E−01 | 0.0033 | 6.20E−01 | −0.4720 | 4.32E−02 |
| Obesity | 0.8011 | 1.12E−01 | 0.4067 | 1.87E−01 | 0.8697 | 5.23E−01 | 0.0113 | 9.35E−02 | −0.0318 | 8.92E−01 |
| Lipodystrophy | 0.3608 | 4.82E−01 | −0.6698 | 3.04E−02 | 0.3261 | 8.11E−01 | 0.0353 | 1.82E−07 | 0.6493 | 5.74E−03 |
| LiverLipid | −0.8585 | 1.01E−01 | −0.4791 | 1.29E−01 | 1.5924 | 2.53E−01 | 0.0073 | 2.95E−01 | −0.1738 | 4.68E−01 |
| ALP_neg | 0.4900 | 3.36E−01 | −3.0620 | 3.00E−23 | 1.3981 | 3.04E−01 | 0.0222 | 1.09E−03 | 0.3304 | 1.59E−01 |
| Hyper_Insulin_Secretion | −0.3110 | 5.48E−01 | 0.4218 | 1.82E−01 | −1.7364 | 2.18E−01 | 0.0180 | 8.77E−03 | 0.1519 | 5.26E−01 |
| SHBG | 0.3654 | 4.73E−01 | −0.5540 | 7.11E−02 | −4.8131 | 3.92E−04 | 0.0047 | 4.86E−01 | 0.1032 | 6.58E−01 |
| Combined pPS | 0.4668 | 3.63E−01 | −0.7306 | 1.90E−02 | −0.4723 | 7.36E−01 | 0.0680 | 1.08E−23 | −0.0492 | 8.35E−01 |

T2D only

| | C-reactive protein | | | | | | | | | |
|---|---|---|---|---|---|---|---|---|---|---|
| | (N = 759) | | ALP (N = 1,848) | | SHBG (N = 80) | | HbA1c (N = 1,804) | | ALT (N = 1,862) | |
| cluster | beta | p-value | beta | p-value | beta | p-value | beta | p-value | beta | p-value |
| Betacell1 | −0.5639 | 7.82E−01 | −1.7295 | 1.26E−01 | 0.5176 | 8.66E−01 | 0.0442 | 1.29E−01 | −0.4785 | 7.42E−01 |
| Betacell2 | 2.8334 | 1.67E−01 | −1.9823 | 8.47E−02 | 0.6111 | 8.26E−01 | 0.0260 | 3.76E−01 | −1.7487 | 2.35E−01 |
| Proinsulin | −4.9142 | 1.56E−02 | 0.3148 | 7.83E−01 | −2.3584 | 4.07E−01 | 0.0437 | 1.33E−01 | 0.4269 | 7.71E−01 |
| LipoproteinA | 1.7175 | 3.98E−01 | −0.5422 | 6.35E−01 | 6.5776 | 2.14E−02 | 0.0039 | 8.94E−01 | −0.1000 | 9.46E−01 |
| Obesity | 4.3062 | 2.62E−02 | 0.2665 | 8.14E−01 | −0.1440 | 9.60E−01 | 0.0112 | 7.00E−01 | −0.3484 | 8.11E−01 |
| Lipodystrophy | 0.2759 | 8.95E−01 | −1.5560 | 1.76E−01 | −1.1874 | 6.96E−01 | 0.0645 | 2.70E−02 | −0.2239 | 8.79E−01 |
| LiverLipid | −5.6415 | 7.29E−03 | −1.5655 | 1.83E−01 | 0.3539 | 9.15E−01 | −0.0259 | 3.91E−01 | −3.7557 | 1.27E−02 |
| ALP_neg | 0.0002 | 1.00E+00 | −3.7197 | 1.26E−03 | 2.5014 | 4.31E−01 | 0.0044 | 8.80E−01 | 0.4690 | 7.51E−01 |
| Hyper_Insulin_Secretion | 1.8535 | 3.59E−01 | 0.9057 | 4.37E−01 | 2.7862 | 3.90E−01 | 0.0035 | 9.07E−01 | −0.4491 | 7.63E−01 |
| SHBG | −0.4363 | 8.30E−01 | −1.7392 | 1.25E−01 | −3.6478 | 2.43E−01 | 0.0321 | 2.68E−01 | 0.0444 | 9.76E−01 |
| Combined pPS | 1.3917 | 5.04E−01 | −2.6720 | 2.03E−02 | 1.8241 | 5.26E−01 | 0.0719 | 1.42E−02 | −0.6763 | 6.47E−01 |

Unadjusted for T2D status

| | | | | | | | Hemoglobin | | | |
|---|---|---|---|---|---|---|---|---|---|---|
| | AST (N = 20,703) | | Calcium (N = 22,801) | | GGT (N = 1,134) | | (N = 22,526) | | Albumin (N = 20,940) | |
| cluster | beta | p-value | beta | p-value | beta | p-value | beta | p-value | beta | p-value |
| Betacell1 | −0.3769 | 1.22E−01 | 0.0074 | 1.27E−02 | −2.9172 | 6.58E−01 | −0.0091 | 4.37E−01 | 0.0068 | 2.67E−02 |
| Betacell2 | −0.2671 | 2.79E−01 | 0.0042 | 1.61E−01 | −4.6974 | 4.68E−01 | −0.0051 | 6.69E−01 | 0.0001 | 9.64E−01 |
| Proinsulin | −0.0974 | 6.91E−01 | −0.0026 | 3.79E−01 | −2.4619 | 7.07E−01 | −0.0074 | 5.31E−01 | 0.0021 | 4.96E−01 |
| LipoproteinA | −0.5664 | 2.05E−02 | 0.0016 | 5.93E−01 | −7.7736 | 2.46E−01 | −0.0118 | 3.14E−01 | 0.0055 | 7.22E−02 |
| Obesity | −0.0165 | 9.46E−01 | −0.0062 | 3.71E−02 | 5.5351 | 3.94E−01 | −0.0376 | 1.40E−03 | −0.0153 | 6.66E−07 |
| Lipodystrophy | 0.3722 | 1.30E−01 | 0.0047 | 1.13E−01 | −0.0196 | 9.98E−01 | −0.0153 | 1.95E−01 | 0.0036 | 2.40E−01 |
| LiverLipid | −0.0927 | 7.12E−01 | −0.0078 | 1.01E−02 | −7.1955 | 2.78E−01 | 0.0234 | 5.30E−02 | −0.0066 | 3.76E−02 |
| ALP_neg | 0.2413 | 3.25E−01 | −0.0037 | 2.12E−01 | −0.7959 | 9.05E−01 | 0.0030 | 8.00E−01 | −0.0007 | 8.25E−01 |
| Hyper_Insulin_Secretion | 0.0269 | 9.15E−01 | −0.0022 | 4.68E−01 | −7.7414 | 2.50E−01 | −0.0167 | 1.67E−01 | 0.0014 | 6.69E−01 |
| SHBG | −0.2230 | 3.61E−01 | 0.0026 | 3.80E−01 | 2.1312 | 7.52E−01 | 0.0147 | 2.08E−01 | 0.0035 | 2.52E−01 |
| Combined pPS | −0.2017 | 4.12E−01 | 0.0045 | 1.27E−01 | −8.3925 | 2.21E−01 | −0.0249 | 3.46E−02 | 0.0014 | 6.56E−01 |

Adjusted for T2D status

| | | | | | | | Hemoglobin | | | |
|---|---|---|---|---|---|---|---|---|---|---|
| | AST (N = 20,703) | | Calcium (N = 22,801) | | GGT (N = 1,134) | | (N = 22,526) | | Albumin (N = 20,940) | |
| cluster | beta | p-value | beta | p-value | beta | p-value | beta | p-value | beta | p-value |
| Betacell1 | −0.3930 | 1.15E−01 | 0.0072 | 1.64E−02 | −1.1848 | 8.58E−01 | 0.0017 | 8.88E−01 | 0.0088 | 4.70E−03 |
| Betacell2 | −0.2995 | 2.35E−01 | 0.0040 | 1.87E−01 | −2.9422 | 6.50E−01 | 0.0104 | 3.80E−01 | 0.0024 | 4.42E−01 |
| Proinsulin | −0.0927 | 7.11E−01 | −0.0029 | 3.33E−01 | −1.1930 | 8.57E−01 | −0.0002 | 9.84E−01 | 0.0029 | 3.57E−01 |
| LipoproteinA | −0.6136 | 1.40E−02 | 0.0010 | 7.39E−01 | −5.7911 | 3.94E−01 | −0.0103 | 3.80E−01 | 0.0056 | 6.93E−02 |
| Obesity | 0.0034 | 9.89E−01 | −0.0070 | 1.97E−02 | 6.8557 | 2.94E−01 | −0.0266 | 2.42E−02 | −0.0140 | 6.29E−06 |
| Lipodystrophy | 0.3563 | 1.56E−01 | 0.0048 | 1.09E−01 | 0.7586 | 9.11E−01 | −0.0029 | 8.09E−01 | 0.0062 | 4.83E−02 |
| LiverLipid | −0.1044 | 6.84E−01 | −0.0083 | 6.76E−03 | −5.6436 | 3.98E−01 | 0.0260 | 3.15E−02 | −0.0063 | 4.65E−02 |
| ALP_neg | 0.2620 | 2.96E−01 | −0.0031 | 3.08E−01 | 0.1781 | 9.79E−01 | 0.0073 | 5.35E−01 | 0.0008 | 7.88E−01 |
| Hyper_Insulin_Secretion | 0.0147 | 9.54E−01 | −0.0016 | 6.05E−01 | −4.4104 | 5.15E−01 | −0.0074 | 5.42E−01 | 0.0028 | 3.79E−01 |
| SHBG | −0.2125 | 3.94E−01 | 0.0018 | 5.54E−01 | 3.6680 | 5.89E−01 | 0.0128 | 2.75E−01 | 0.0031 | 3.16E−01 |
| Combined pPS | −0.2216 | 3.81E−01 | 0.0038 | 2.03E−01 | −5.2212 | 4.50E−01 | 0.0049 | 6.83E−01 | 0.0058 | 6.62E−02 |

T2D only

| | AST (N = 1,860) | | Calcium (N = 1,930) | | GGT (N = 156) | | Hemoglobin (N = 1,895) | | Albumin (N = 1,863) | |
|---|---|---|---|---|---|---|---|---|---|---|
| cluster | beta | p-value | beta | p-value | beta | p-value | beta | p-value | beta | p-value |
| Betacell1 | −0.1792 | 8.75E−01 | −0.0032 | 7.60E−01 | −7.902 | 6.53E−01 | 0.0265 | 5.56E−01 | 0.0193 | 8.62E−02 |
| Betacell2 | −2.1161 | 6.61E−02 | −0.0078 | 4.67E−01 | −25.238 | 1.22E−01 | −0.0027 | 9.52E−01 | 0.0121 | 2.91E−01 |
| Proinsulin | −0.3450 | 7.63E−01 | 0.0091 | 3.95E−01 | −18.190 | 2.59E−01 | 0.0303 | 5.04E−01 | 0.0150 | 1.84E−01 |
| LipoproteinA | −0.3052 | 7.90E−01 | −0.0036 | 7.32E−01 | −21.045 | 2.05E−01 | 0.0310 | 4.93E−01 | 0.0184 | 1.05E−01 |
| Obesity | 0.4747 | 6.77E−01 | −0.0178 | 9.37E−02 | 21.081 | 1.70E−01 | −0.0655 | 1.47E−01 | −0.0200 | 7.62E−02 |
| Lipodystrophy | 0.1644 | 8.86E−01 | 0.0082 | 4.43E−01 | 30.408 | 8.90E−02 | 0.0315 | 4.90E−01 | 0.0205 | 7.25E−02 |
| LiverLipid | −3.2806 | 5.42E−03 | 0.0092 | 4.04E−01 | −3.909 | 8.20E−01 | 0.0869 | 6.40E−02 | 0.0293 | 1.23E−02 |
| ALP_neg | −0.1072 | 9.26E−01 | 0.0177 | 9.99E−02 | 19.280 | 2.34E−01 | −0.0064 | 8.90E−01 | −0.0027 | 8.11E−01 |
| Hyper_Insulin_Secretion | −0.2914 | 8.03E−01 | −0.0113 | 2.96E−01 | −3.668 | 8.36E−01 | −0.0444 | 3.36E−01 | 0.0004 | 9.70E−01 |
| SHBG | −0.2545 | 8.23E−01 | 0.0067 | 5.25E−01 | 28.878 | 6.87E−02 | 0.0428 | 3.43E−01 | 0.0158 | 1.62E−01 |
| Combined pPS | −0.7178 | 5.34E−01 | −0.0031 | 7.70E−01 | 15.146 | 3.97E−01 | 0.0498 | 2.77E−01 | 0.0235 | 3.99E−02 |

Unadjusted for T2D status

| | Lymphocytes | | Mean Corpuscular | | | | Mean Platelet Volume | | | |
|---|---|---|---|---|---|---|---|---|---|---|
| | (N = 19,834) | | Volume (N = 21,567) | | Monocytes (N = 19,833) | | (N = 22,334) | | Platelet (N = 22,556) | |
| cluster | beta | p-value | beta | p-value | beta | p-value | beta | p-value | beta | p-value |
| Betacell1 | 0.0017 | 9.15E−01 | −0.0580 | 8.20E−02 | −0.0026 | 1.60E−01 | 0.0107 | 1.03E−01 | −1.0711 | 1.34E−02 |
| Betacell2 | 0.0119 | 4.45E−01 | −0.0164 | 6.27E−01 | −0.0054 | 3.40E−03 | 0.0095 | 1.51E−01 | −0.7579 | 8.34E−02 |
| Proinsulin | −0.0050 | 7.48E−01 | 0.0155 | 6.43E−01 | −0.0006 | 7.32E−01 | −0.0158 | 1.70E−02 | 0.5089 | 2.43E−01 |
| LipoproteinA | 0.0030 | 8.45E−01 | −0.0523 | 1.18E−01 | −0.0028 | 1.25E−01 | −0.0026 | 6.94E−01 | 0.3537 | 4.16E−01 |
| Obesity | −0.0005 | 9.74E−01 | −0.0501 | 1.34E−01 | 0.0014 | 4.35E−01 | 0.0083 | 2.08E−01 | −0.2728 | 5.30E−01 |
| Lipodystrophy | 0.0300 | 5.33E−02 | −0.1442 | 1.62E−05 | 0.0044 | 1.63E−02 | 0.0086 | 1.94E−01 | −1.3051 | 2.69E−03 |
| LiverLipid | −0.0127 | 4.24E−01 | −0.0270 | 4.32E−01 | 0.0013 | 4.73E−01 | 0.0167 | 1.34E−02 | −1.4887 | 8.59E−04 |
| ALP_neg | −0.0299 | 5.36E−02 | −0.0784 | 1.92E−02 | −0.0032 | 8.21E−02 | 0.0051 | 4.40E−01 | −0.5523 | 2.04E−01 |
| Hyper_Insulin_Secretion | 0.0126 | 4.27E−01 | −0.0927 | 6.80E−03 | 0.0034 | 7.15E−02 | 0.0249 | 2.22E−04 | −0.0368 | 9.34E−01 |
| SHBG | 0.0115 | 4.58E−01 | 0.0693 | 3.75E−02 | 0.0007 | 6.95E−01 | −0.0057 | 3.87E−01 | 0.2408 | 5.78E−01 |
| Combined pPS | 0.0147 | 3.44E−01 | −0.1189 | 3.92E−04 | −0.0008 | 6.64E−01 | 0.0133 | 4.46E−02 | −1.3290 | 2.30E−03 |

Adjusted for T2D status

| | Lymphocytes | | Mean Corpuscular | | | | Mean Platelet | | | |
|---|---|---|---|---|---|---|---|---|---|---|
| | (N = 19,834) | | Volume (N = 21,567) | | Monocytes (N = 19,833) | | Volume (N = 22,334) | | Platelet (N = 22,556) | |
| cluster | beta | p-value | beta | p-value | beta | p-value | beta | p-value | beta | p-value |
| Betacell1 | 0.0014 | 9.31E−01 | −0.0295 | 3.79E−01 | −0.0029 | 1.16E−01 | 0.0071 | 2.88E−01 | −1.0884 | 1.30E−02 |
| Betacell2 | 0.0119 | 4.59E−01 | 0.0016 | 9.62E−01 | −0.0064 | 5.75E−04 | 0.0054 | 4.20E−01 | −0.7138 | 1.07E−01 |
| Proinsulin | −0.0049 | 7.59E−01 | 0.0294 | 3.82E−01 | −0.0009 | 6.26E−01 | −0.0177 | 8.10E−03 | 0.5518 | 2.10E−01 |
| LipoproteinA | 0.0021 | 8.93E−01 | −0.0420 | 2.11E−01 | −0.0031 | 9.61E−02 | −0.0034 | 6.06E−01 | 0.3918 | 3.72E−01 |
| Obesity | −0.0009 | 9.55E−01 | −0.0339 | 3.14E−01 | 0.0004 | 8.14E−01 | 0.0064 | 3.41E−01 | −0.2509 | 5.68E−01 |
| Lipodystrophy | 0.0292 | 6.66E−02 | −0.1176 | 4.70E−04 | 0.0037 | 4.45E−02 | 0.0073 | 2.76E−01 | −1.3112 | 2.85E−03 |
| LiverLipid | −0.0142 | 3.85E−01 | −0.0272 | 4.30E−01 | 0.0015 | 4.40E−01 | 0.0164 | 1.69E−02 | −1.4583 | 1.21E−03 |
| ALP_neg | −0.0292 | 6.63E−02 | −0.0799 | 1.76E−02 | −0.0037 | 4.41E−02 | 0.0048 | 4.73E−01 | −0.5855 | 1.83E−01 |
| Hyper_Insulin_Secretion | 0.0114 | 4.83E−01 | −0.0854 | 1.31E−02 | 0.0025 | 1.79E−01 | 0.0231 | 7.27E−04 | 0.0093 | 9.84E−01 |
| SHBG | 0.0098 | 5.34E−01 | 0.0722 | 3.08E−02 | 0.0008 | 6.60E−01 | −0.0054 | 4.14E−01 | 0.3054 | 4.85E−01 |
| Combined pPS | 0.0131 | 4.15E−01 | −0.0702 | 3.84E−02 | −0.0025 | 1.75E−01 | 0.0068 | 3.12E−01 | −1.3003 | 3.34E−03 |

T2D only

| | Lymphocytes | | Mean Corpuscular | | | | Mean Platelet Volume | | | |
|---|---|---|---|---|---|---|---|---|---|---|
| | (N = 1,771) | | Volume (N = 1,902) | | Monocytes (N = 1,771) | | (N = 1,887) | | Platelet (1,899) | |
| cluster | beta | p-value | beta | p-value | beta | p-value | beta | p-value | beta | p-value |
| Betacell1 | −0.0544 | 2.79E−01 | −0.0434 | 7.22E−01 | −0.0052 | 3.50E−01 | 0.0063 | 7.62E−01 | 0.4798 | 7.54E−01 |
| Betacell2 | 0.0656 | 2.04E−01 | 0.0126 | 9.19E−01 | −0.0023 | 6.93E−01 | −0.0138 | 5.15E−01 | 1.6915 | 2.78E−01 |
| Proinsulin | 0.0127 | 8.04E−01 | 0.0489 | 6.91E−01 | −0.0033 | 5.57E−01 | −0.0031 | 8.82E−01 | 0.7877 | 6.10E−01 |
| LipoproteinA | −0.0790 | 1.22E−01 | −0.2363 | 5.44E−02 | −0.0120 | 3.33E−02 | 0.0099 | 6.36E−01 | 2.2223 | 1.50E−01 |
| Obesity | 0.0112 | 8.26E−01 | −0.2279 | 6.29E−02 | 0.0127 | 2.39E−02 | 0.0193 | 3.55E−01 | 0.2919 | 8.49E−01 |
| Lipodystrophy | 0.0097 | 8.49E−01 | −0.1177 | 3.40E−01 | −0.0051 | 3.72E−01 | 0.0125 | 5.53E−01 | −1.4750 | 3.41E−01 |
| LiverLipid | −0.0306 | 5.62E−01 | −0.1593 | 2.11E−01 | 0.0052 | 3.71E−01 | 0.0039 | 8.58E−01 | −0.6165 | 7.00E−01 |
| ALP_neg | −0.0274 | 5.96E−01 | −0.1562 | 2.10E−01 | 0.0055 | 3.36E−01 | 0.0135 | 5.25E−01 | −0.0950 | 9.52E−01 |
| Hyper_Insulin_Secretion | 0.0228 | 6.60E−01 | −0.0121 | 9.23E−01 | 0.0026 | 6.53E−01 | 0.0367 | 8.58E−02 | −0.9970 | 5.26E−01 |
| SHBG | 0.0297 | 5.61E−01 | 0.1252 | 3.06E−01 | −0.0062 | 2.73E−01 | −0.0318 | 1.27E−01 | 1.2133 | 4.29E−01 |
| Combined pPS | −0.0150 | 7.72E−01 | −0.1185 | 3.40E−01 | −0.0001 | 9.91E−01 | 0.0153 | 4.68E−01 | 0.3213 | 8.37E−01 |

Unadjusted for T2D status

| | Red Blood Cell (N = 21,523) | | Total Protein (N = 20,813) | | Urate (N = 4,202) | | IGF-1 (N = 484) | |
|---|---|---|---|---|---|---|---|---|
| cluster | beta | p-value | beta | p-value | beta | p-value | beta | p-value |
| Betacell1 | −0.00013 | 5.75E−01 | −0.0003 | 9.30E−01 | −0.0053 | 8.57E−01 | −0.0090 | 9.98E−01 |
| Betacell2 | −0.00022 | 3.60E−01 | −0.0029 | 4.41E−01 | 0.0091 | 7.65E−01 | −4.4465 | 2.04E−01 |
| Proinsulin | 0.00007 | 7.70E−01 | −0.0005 | 8.84E−01 | −0.0415 | 1.61E−01 | 0.3476 | 9.27E−01 |
| LipoproteinA | 0.00000 | 9.91E−01 | −0.0007 | 8.52E−01 | 0.0280 | 3.45E−01 | −1.9482 | 5.87E−01 |
| Obesity | 0.00035 | 1.47E−01 | −0.0036 | 3.39E−01 | 0.0495 | 9.27E−02 | −0.0588 | 9.86E−01 |
| Lipodystrophy | −0.00001 | 9.51E−01 | 0.0053 | 1.59E−01 | 0.0194 | 5.18E−01 | 2.5921 | 4.82E−01 |
| LiverLipid | 0.00005 | 8.31E−01 | −0.0076 | 4.59E−02 | −0.0520 | 8.71E−02 | 3.9667 | 2.87E−01 |
| ALP_neg | 0.00020 | 4.08E−01 | −0.0005 | 8.89E−01 | 0.0202 | 5.05E−01 | 1.6189 | 6.55E−01 |
| Hyper_Insulin_Secretion | −0.00005 | 8.48E−01 | 0.0047 | 2.17E−01 | 0.0189 | 5.32E−01 | 4.5923 | 2.12E−01 |
| SHBG | −0.00042 | 8.27E−02 | 0.0043 | 2.42E−01 | −0.0082 | 7.82E−01 | 7.1376 | 4.33E−02 |
| Combined pPS | −0.00014 | 5.51E−01 | 0.0017 | 6.47E−01 | −0.0063 | 8.32E−01 | 1.0440 | 7.70E−01 |

Adjusted for T2D status

| | Red Blood Cell (N = 21,523) | | Total Protein (N = 20,813) | | Urate (N = 4,202) | | IGF-1 (N = 484) | |
|---|---|---|---|---|---|---|---|---|
| cluster | beta | p-value | beta | p-value | beta | p-value | beta | p-value |
| Betacell1 | −0.00017 | 4.91E−01 | 0.0005 | 9.03E−01 | −0.0119 | 6.89E−01 | −0.5456 | 8.91E−01 |
| Betacell2 | −0.00026 | 2.86E−01 | −0.0019 | 6.26E−01 | −0.0022 | 9.44E−01 | −3.8078 | 2.96E−01 |
| Proinsulin | 0.00004 | 8.81E−01 | 0.0000 | 9.98E−01 | −0.0454 | 1.28E−01 | 0.1966 | 9.60E−01 |
| LipoproteinA | −0.00001 | 9.80E−01 | −0.0014 | 7.12E−01 | 0.0244 | 4.15E−01 | −2.4561 | 5.06E−01 |
| Obesity | 0.00035 | 1.56E−01 | −0.0040 | 2.91E−01 | 0.0393 | 1.86E−01 | 0.5219 | 8.83E−01 |
| Lipodystrophy | −0.00007 | 7.70E−01 | 0.0061 | 1.06E−01 | 0.0008 | 9.80E−01 | 2.9737 | 4.30E−01 |
| LiverLipid | 0.00003 | 9.06E−01 | −0.0077 | 4.54E−02 | −0.0507 | 9.68E−02 | 4.3267 | 2.55E−01 |
| ALP_neg | 0.00021 | 3.90E−01 | −0.0002 | 9.54E−01 | 0.0050 | 8.71E−01 | 1.6756 | 6.58E−01 |
| Hyper_Insulin_Secretion | −0.00007 | 7.66E−01 | 0.0047 | 2.23E−01 | 0.0185 | 5.45E−01 | 4.2962 | 2.56E−01 |
| SHBG | −0.00040 | 1.02E−01 | 0.0038 | 3.11E−01 | −0.0093 | 7.54E−01 | 8.3387 | 2.15E−02 |
| Combined pPS | −0.00024 | 3.33E−01 | 0.0026 | 4.99E−01 | −0.0338 | 2.64E−01 | 1.8490 | 6.18E−01 |

T2D only

| | Red Blood Cell (N = 1,844) | | Total Protein (N = 1,854) | | Urate (N = 570) | | IGF-1 (N = 32) | |
|---|---|---|---|---|---|---|---|---|
| cluster | beta | p-value | beta | p-value | beta | p-value | beta | p-value |
| Betacell1 | −0.0014 | 3.39E−01 | −0.0074 | 5.74E−01 | −0.0666 | 4.62E−01 | −22.743 | 2.19E−01 |
| Betacell2 | −0.0006 | 6.86E−01 | −0.0140 | 2.98E−01 | −0.0206 | 8.27E−01 | −16.461 | 1.10E−01 |
| Proinsulin | 0.0004 | 7.94E−01 | 0.0042 | 7.51E−01 | −0.0430 | 6.50E−01 | −9.261 | 5.03E−01 |
| LipoproteinA | 0.0024 | 1.01E−01 | 0.0060 | 6.50E−01 | −0.0545 | 5.44E−01 | 15.965 | 7.94E−02 |
| Obesity | 0.0003 | 8.40E−01 | −0.0181 | 1.70E−01 | 0.1139 | 2.12E−01 | −4.706 | 6.28E−01 |
| Lipodystrophy | −0.0020 | 1.75E−01 | 0.0050 | 7.08E−01 | 0.0184 | 8.37E−01 | −7.134 | 5.16E−01 |
| LiverLipid | −0.0013 | 4.09E−01 | 0.0070 | 6.11E−01 | −0.1112 | 2.48E−01 | 15.053 | 2.10E−01 |
| ALP_neg | 0.0015 | 3.02E−01 | −0.0125 | 3.54E−01 | −0.0638 | 4.95E−01 | 8.129 | 5.90E−01 |
| Hyper_Insulin_Secretion | −0.0022 | 1.41E−01 | 0.0102 | 4.50E−01 | −0.0737 | 3.90E−01 | −3.559 | 7.95E−01 |
| SHBG | −0.0021 | 1.49E−01 | 0.0024 | 8.57E−01 | 0.0758 | 4.01E−01 | 9.935 | 4.18E−01 |
| Combined pPS | −0.0020 | 1.69E−01 | −0.0134 | 3.21E−01 | −0.0658 | 4.75E−01 | −15.147 | 1.87E−01 |

* beta estimates represent change of traits per SD of pPS
**BMI and Triglycerides were log-transformed
bold indicates defining cluster traits

TABLE 10
T2D cluster GWAS pPS associations with clinical outcomes

| | | CAD | | CKD | | eGFR | |
|---|---|---|---|---|---|---|---|
| cluster | N_SNP | beta | pvalue | beta | pvalue | beta | pvalue |
| Beta-cell1 | 66 | 0.0094 | 9.88E−15 | 0.0006 | 6.04E−01 | 0.00011 | 1.78E−02 |
| Beta-cell2 | 27 | 0.0049 | 1.01E−02 | −0.0005 | 8.00E−01 | 0.00005 | 4.84E−01 |
| Proinsulin | 18 | 0.0104 | 5.62E−06 | −0.0009 | 6.66E−01 | 0.00005 | 5.37E−01 |
| LipoproteinA | 1 | 0.0330 | 4.69E−05 | 0.0042 | 5.95E−01 | 0.00020 | 5.05E−01 |
| Obesity | 37 | 0.0138 | 4.87E−19 | 0.0074 | 1.05E−06 | −0.00009 | 9.46E−02 |
| Lipodystrophy | 54 | 0.0167 | 6.05E−38 | 0.0027 | 3.31E−02 | −0.00012 | 1.09E−02 |
| Liver/Lipid | 12 | −0.0064 | 4.13E−02 | 0.0140 | 1.73E−06 | −0.00091 | 1.01E−16 |
| ALP_neg | 4 | 0.0045 | 3.75E−01 | 0.0121 | 1.04E−02 | −0.00140 | 6.12E−15 |
| Hyper Insulin Secretion | 32 | 0.0113 | 9.36E−12 | 0.0012 | 4.41E−01 | 0.00016 | 5.95E−03 |
| SHBG | 1 | 0.0143 | 9.10E−02 | 0.0071 | 3.69E−01 | −0.00120 | 6.33E−05 |
| allSNPs | 252 | 0.0111 | 1.70E−73 | 0.0027 | 3.61E−06 | −0.00005 | 3.01E−02 |

| | Hypertension | | Ischemic Stroke | | Neuropathy in T2D | |
|---|---|---|---|---|---|---|
| cluster | beta | pvalue | beta | pvalue | beta | pvalue |
| Beta-cell1 | 0.0122 | 1.04E−09 | 0.0074 | 5.43E−09 | 0.0329 | 2.19E−04 |
| Beta-cell2 | 0.0076 | 1.92E−02 | 0.0084 | 3.39E−05 | −0.0021 | 8.86E−01 |
| Proinsulin | 0.0191 | 2.62E−07 | 0.0083 | 4.12E−04 | 0.0183 | 2.68E−01 |
| LipoproteinA | −0.0093 | 4.91E−01 | −0.0069 | 4.22E−01 | 0.0053 | 9.30E−01 |
| Obesity | 0.0235 | 2.50E−19 | 0.0092 | 1.09E−08 | 0.0321 | 5.87E−03 |
| Lipodystrophy | 0.0165 | 2.08E−14 | 0.0067 | 6.32E−07 | 0.0391 | 4.66E−05 |
| Liver/Lipid | −0.0041 | 4.39E−01 | −0.0055 | 1.31E−01 | 0.0595 | 1.07E−02 |
| ALP_neg | −0.0157 | 4.16E−02 | 0.0084 | 1.21E−01 | 0.0586 | 8.89E−02 |
| Hyper Insulin Secretion | 0.0188 | 4.83E−11 | 0.0039 | 2.78E−02 | 0.0553 | 1.26E−05 |
| SHBG | 0.0071 | 6.09E−01 | 0.0076 | 3.77E−01 | 0.1183 | 5.48E−02 |
| allSNPs | 0.0145 | 3.48E−46 | 0.0068 | 3.76E−26 | 0.0342 | 4.15E−14 |

* note:
study GWAS references in Table 6.
* highlighted: pval < 8.3 × 10 − 4 [0.05/(10*6)]

TABLE 11
Individual-level cluster pPS associations with clinical outcomes in MGB Biobank (T2D clusters)
Unadjusted for T2D status

| | CAD | | CKD | | | | Hypertension | |
|---|---|---|---|---|---|---|---|---|
| | (N_total = 17,167/ | | (N_total = 21,837/ | | eGFR | | (N_total = 20,949/ | |
| | N_case = 2,938) | | N_case = 10,421) | | (N_total = 22,876) | | N_case = 9,868) | |
| cluster | OR | p-value | OR | p-value | beta | p-value | OR | p-value |
| Betacell1 | 1.007 | 1.15E−02 | 1.002 | 3.03E−01 | −0.0021 | 8.68E−01 | 1.001 | 4.75E−01 |
| Betacell2 | 1.000 | 9.22E−01 | 1.001 | 6.39E−01 | −0.0146 | 4.26E−01 | 1.002 | 5.47E−01 |
| Proinsulin | 0.995 | 3.09E−01 | 1.008 | 8.88E−03 | −0.0114 | 5.97E−01 | 1.005 | 1.46E−01 |
| LipoproteinA | 1.012 | 5.70E−03 | 0.997 | 3.18E−01 | 0.0346 | 7.85E−02 | 1.003 | 2.90E−01 |
| Obesity | 1.005 | 1.24E−01 | 1.002 | 3.57E−01 | −0.0052 | 7.48E−01 | 1.014 | 3.11E−08 |
| Lipodystrophy | 1.015 | 6.24E−07 | 1.010 | 7.08E−07 | −0.0140 | 3.16E−01 | 1.011 | 6.46E−07 |
| LiverLipid | 0.989 | 6.50E−03 | 1.005 | 7.76E−02 | −0.0653 | 6.18E−04 | 1.000 | 9.41E−01 |
| ALP_neg | 0.997 | 4.44E−01 | 0.999 | 8.48E−01 | −0.0116 | 5.30E−01 | 0.999 | 7.76E−01 |
| Hyper_Insulin_Secretion | 1.014 | 3.96E−03 | 1.003 | 4.34E−01 | −0.0086 | 7.07E−01 | 1.003 | 3.19E−01 |
| SHBG | 0.990 | 1.63E−02 | 0.997 | 3.63E−01 | −0.0314 | 1.25E−01 | 0.997 | 3.01E−01 |
| Combined pPS | 1.153 | 1.20E−04 | 1.083 | 1.72E−03 | −0.1428 | 4.13E−01 | 1.142 | 5.99E−07 |

* beta/OR estimates represent change of traits per unit of pPS
* highlighted: pval < 0.05/(10*4) = 1.2 × 10 − 3
Adjusted for T2D status

| | CAD | | CKD | | | | Hypertension | |
|---|---|---|---|---|---|---|---|---|
| | (N_total = 17,167/ | | (N_total = 21,837/ | | eGFR | | (N_total = 20,949/ | |
| | N_case = 2,938) | | N_case = 10,421) | | (N_total = 22,876) | | N_case = 9,868) | |
| cluster | OR | p-value | OR | p-value | beta | p-value | OR | p-value |
| Betacell1 | 1.003 | 2.28E−01 | 0.999 | 5.47E−01 | 0.0063 | 6.24E−01 | 0.996 | 5.63E−02 |
| Betacell2 | 0.994 | 1.22E−01 | 0.997 | 2.92E−01 | −0.0042 | 8.21E−01 | 0.994 | 3.44E−02 |
| Proinsulin | 0.992 | 8.70E−02 | 1.007 | 4.58E−02 | −0.0054 | 8.02E−01 | 1.002 | 5.41E−01 |
| LipoproteinA | 1.011 | 1.55E−02 | 0.997 | 3.30E−01 | 0.0356 | 7.18E−02 | 1.002 | 5.46E−01 |
| Obesity | 1.002 | 5.71E−01 | 1.000 | 8.70E−01 | 0.0043 | 7.88E−01 | 1.010 | 7.51E−05 |
| Lipodystrophy | 1.011 | 3.38E−04 | 1.007 | 5.34E−04 | −0.0085 | 5.42E−01 | 1.006 | 7.48E−03 |
| LiverLipid | 0.987 | 1.60E−03 | 1.005 | 7.56E−02 | −0.0701 | 2.42E−04 | 0.998 | 4.97E−01 |
| ALP_neg | 0.996 | 3.46E−01 | 0.999 | 6.18E−01 | −0.0080 | 6.65E−01 | 0.999 | 6.58E−01 |
| Hyper_Insulin_Secretion | 1.009 | 7.09E−02 | 1.000 | 9.96E−01 | 0.0016 | 9.43E−01 | 0.999 | 7.55E−01 |
| SHBG | 0.989 | 1.65E−02 | 0.997 | 2.90E−01 | −0.0328 | 1.11E−01 | 0.996 | 2.72E−01 |
| Combined pPS | 1.019 | 6.26E−01 | 1.006 | 8.31E−01 | 0.0791 | 6.54E−01 | 0.992 | 7.80E−01 |

* beta/OR estimates represent change of traits per unit of pPS
* highlighted: pval < 0.00125 [=0.05/(10*4)]

T2D cases

| | CAD | | CKD | | | | Hypertension | |
|---|---|---|---|---|---|---|---|---|
| | (N_total = 1,626/ | | (N_total = 1,944/ | | eGFR | | (N_total = 1,988/ | |
| | N_case = 759) | | N_case = 1,585) | | (N_total = 1,938) | | N_case = 1,903) | |
| cluster | OR | p-value | OR | p-value | beta | p-value | OR | p-value |
| Betacell1 | 1.007 | 3.15E−01 | 0.988 | 1.17E−01 | 0.0695 | 2.54E−01 | 0.980 | 1.41E−01 |
| Betacell2 | 0.999 | 9.40E−01 | 0.984 | 1.23E−01 | 0.0172 | 8.40E−01 | 0.989 | 5.87E−01 |
| Proinsulin | 0.991 | 4.23E−01 | 0.999 | 9.60E−01 | 0.0453 | 6.60E−01 | 0.977 | 3.33E−01 |
| LipoproteinA | 0.998 | 8.01E−01 | 0.985 | 2.05E−01 | 0.0684 | 4.64E−01 | 0.998 | 9.23E−01 |
| Obesity | 1.002 | 8.31E−01 | 0.984 | 8.08E−02 | −0.0079 | 9.12E−01 | 1.005 | 7.51E−01 |
| Lipodystrophy | 1.010 | 1.52E−01 | 1.012 | 1.41E−01 | −0.0028 | 9.67E−01 | 1.003 | 8.53E−01 |
| LiverLipid | 0.975 | 1.01E−02 | 0.997 | 7.93E−01 | −0.0866 | 3.41E−01 | 0.989 | 6.12E−01 |
| ALP neg | 1.008 | 3.96E−01 | 1.004 | 6.89E−01 | −0.1051 | 2.38E−01 | 0.970 | 1.19E−01 |
| Hyper_Insulin_Secretion | 1.003 | 8.11E−01 | 0.992 | 5.69E−01 | −0.0440 | 6.85E−01 | 0.990 | 6.88E−01 |
| SHBG | 0.978 | 2.87E−02 | 0.996 | 7.60E−01 | −0.2455 | 1.22E−02 | 0.986 | 5.05E−01 |
| Combined pPS | 1.099 | 2.86E−01 | 0.810 | 4.49E−02 | 0.7016 | 4.05E−01 | 0.773 | 1.77E−01 |

* beta/OR estimates represent change of traits per unit of pPS
* highlighted: pval < 0.05/(10*4)

T2D controls

| | CAD | | CKD | | | | Hypertension | |
|---|---|---|---|---|---|---|---|---|
| | (N_total = 15,011/ | | (N_total = 19,299/ | | eGFR | | (N_total = 22,305/ | |
| | N_case = 2,018) | | N_case = 8,435) | | (N_total = 20,356) | | N_case = 7,474) | |
| cluster | OR | p-value | OR | p-value | beta | p-value | OR | p-value |
| Betacell1 | 1.002 | 5.31E−01 | 1.000 | 8.19E−01 | 0.0016 | 9.02E−01 | 0.996 | 7.94E−02 |
| Bctacell2 | 0.992 | 6.50E−02 | 0.998 | 4.53E−01 | −0.0055 | 7.69E−01 | 0.994 | 3.64E−02 |
| Proinsulin | 0.992 | 1.28E−01 | 1.007 | 4.33E−02 | −0.0094 | 6.70E−01 | 1.003 | 4.58E−01 |
| LipoproteinA | 1.014 | 3.37E−03 | 0.998 | 5.09E−01 | 0.0330 | 1.01E−01 | 1.002 | 5.20E−01 |
| Obesity | 1.002 | 5.74E−01 | 1.002 | 5.22E−01 | 0.0061 | 7.11E−01 | 1.011 | 6.42E−05 |
| Lipodystrophy | 1.011 | 2.09E−03 | 1.007 | 1.78E−03 | −0.0094 | 5.07E−01 | 1.006 | 8.35E−03 |
| LiverLipid | 0.990 | 2.63E−02 | 1.006 | 6.12E−02 | −0.0678 | 5.00E−04 | 0.998 | 5.45E−01 |
| ALP_neg | 0.993 | 1.31E−01 | 0.998 | 5.15E−01 | −0.0028 | 8.84E−01 | 0.999 | 8.24E−01 |
| Hyper_Insulin_Secretion | 1.011 | 5.37E−02 | 1.000 | 9.14E−01 | 0.0040 | 8.64E−01 | 0.999 | 8.06E−01 |
| SHBG | 0.992 | 1.18E−01 | 0.997 | 3.11E−01 | −0.0180 | 3.89E−01 | 0.997 | 3.00E−01 |
| Combined pPS | 0.993 | 8.77E−01 | 1.018 | 5.11E−01 | 0.0336 | 8.52E−01 | 0.997 | 9.16E−01 |

* beta/OR estimates represent change of traits per unit of pPS
* highlighted: pval < 0.05/(10*4)

TABLE 12
Cluster weights for CAD Loci

| | | | Lipoprotein | | Blood | ALP | HDL |
|---|---|---|---|---|---|---|---|
| rsID | locus | VAR_ID_hg19 | A | Cholesterol | markers | negative | negative |
| rs11806567 | MORN1 | 1_2251982_C_T | 0.059925 | 0.092801 | 0.503756 | 0 | 0.163983 |
| rs16823332 | ACTRT2 | 1_2920158_C_T | 0.055089 | 0.052943 | 0.416367 | 0.197671 | 0 |
| rs2493291 | PRDM16 | 1_3329384_C_T | 0.002026 | 0.029645 | 0.492939 | 0.15594 | 0 |
| rs4072980 | SF3A3 | 1_38456106_G_A | 0.002004 | 0.081361 | 0.722925 | 0.077301 | 0 |
| rs11206510 | PCSK9 | 1_55496039_T_C | 0.063767 | 1.115963 | 0.087682 | 0.227436 | 0.05946 |
| rs11206803 | PLPP3_1 | 1_56877509_C_T | 0.01434 | 0.077228 | 0.495692 | 0.042043 | 0 |
| rs6674131 | PLPP3_2 | 1_56991294_C_T | 0.006873 | 0 | 0.536598 | 0.072725 | 0.187193 |
| rs2404715 | PLPP3_3 | 1_57008778_C_T | 0.141063 | 0.061315 | 0.846102 | 0.059611 | 0.024101 |
| rs602633 | PSRC1 | 1_109821511_T_G | 0.21676 | 3.694977 | 0.166939 | 0 | 0.757253 |
| rs10858079 | NGF | 1_115899071_A_G | 0.006454 | 0.010791 | 0.249078 | 0.127395 | 0.002164 |
| rs2048558 | MCL1 | 1_150571730_A_G | 0.057555 | 0 | 0.879712 | 0.33515 | 0.043114 |
| rs3790514 | TDRKH | 1_151744168_T_C | 0.105302 | 0.090611 | 0.393487 | 0 | 0.041455 |
| rs4453032 | IL6R | 1_154414086_A_G | 0.082443 | 0 | 1.047633 | 0.545171 | 0.282879 |
| rs7517394 | ATP1B1 | 1_169093762_A_G | 0.00788 | 0 | 0.373757 | 0.095818 | 0.149817 |
| rs6700559 | DDX59 | 1_200646073_C_T | 0.044007 | 0.04171 | 0.393811 | 0.285575 | 0 |
| rs2820315 | LMOD1 | 1_201872264_C_T | 0.037299 | 0 | 0.784245 | 0.033422 | 0.332239 |
| rs12138216 | HHAT | 1_210480361_G_A | 0.142358 | 0 | 0.505037 | 0.059118 | 0.219526 |
| rs3002142 | MIA3 | 1_222788062_C_T | 0.045642 | 0.00474 | 0.432525 | 0.17499 | 0.09661 |
| rs6683071 | FAM177B | 1_222923351_A_G | 0.00327 | 0.054631 | 0.472849 | 0.060296 | 0.361245 |
| rs12710727 | TTC32_1 | 2_19935033_T_G | 0.000278 | 0 | 0.421124 | 0.105431 | 0.139972 |
| rs16986953 | TTC32_2 | 2_19942473_G_A | 0.023211 | 0.027149 | 0.486015 | 0.02957 | 0.038194 |
| rs515135 | APOB | 2_21286057_T_C | 0.164551 | 2.981289 | 0.074136 | 0.039692 | 0.138849 |
| rs6544713 | ABCG8_1 | 2_44073881_T_C | 0.077433 | 1.286412 | 0.207027 | 0.328097 | 0.058183 |
| rs4953023 | ABCG8_2 | 2_44074000_G_A | 0.006043 | 1.289126 | 0.202845 | 0.444517 | 0.055438 |
| rs582384 | PRKCE | 2_45896437_C_A | 0.076565 | 0.046147 | 0.544967 | 0.095032 | 0 |
| rs1446669 | MAT2A | 2_85762209_T_C | 0.010294 | 0.021411 | 0.387682 | 0.297769 | 0 |
| rs6740731 | ZEB2 | 2_145270592_G_A | 0.004591 | 0.067501 | 0.562469 | 0.263796 | 0.063941 |
| rs2252641 | TEX41 | 2_145801461_T_C | 0.057097 | 0.074556 | 0.20626 | 0.489667 | 0 |
| rs7583960 | FIGN | 2_164962632_T_C | 0.002516 | 0.07662 | 0.476505 | 0.18656 | 0 |
| rs1541853 | NBEAL1 | 2_203835177_A_C | 0.003446 | 0.071707 | 0.562363 | 0.730183 | 0.173961 |
| rs1250240 | FN1 | 2_216295014_A_G | 0.001015 | 0.049972 | 0.76536 | 0.085015 | 0 |
| rs1035672 | TNS1_1 | 2_218675199_G_A | 0.007299 | 0.077814 | 0.565004 | 0.331172 | 0 |
| rs3791944 | TNS1_2 | 2_218743211_A_T | 0.103402 | 0.010295 | 0.352458 | 0.213426 | 0.015393 |
| rs1801251 | GIGYF2 | 2_233633460_G_A | 0.13988 | 0.051318 | 0.577271 | 0.286679 | 0 |
| rs11677932 | COL6A3 | 2_238223955_G_A | 0.01029 | 0.060166 | 0.243899 | 0.076415 | 0.067092 |
| rs744892 | FGD5_1 | 3_14871766_G_A | 0.051258 | 0.040136 | 0.436029 | 0.063036 | 0.103884 |
| rs2131570 | FGD5_2 | 3_14906734_C_T | 0.020878 | 0 | 0.749738 | 0 | 0.146243 |
| rs7617773 | CDC25A | 3_48193515_C_T | 0 | 0.05278 | 0.860881 | 0.069297 | 0.105063 |
| rs3172494 | IP6K2 | 3_48731487_G_T | 0.043523 | 0 | 0.896581 | 0.201404 | 0 |
| rs17282057 | UMPS | 3_124446077_T_C | 0.009934 | 0 | 0.483499 | 0.273049 | 0.170775 |
| rs483465 | PCCB | 3_136047977_A_G | 0.036317 | 0.265383 | 1.909554 | 0.076471 | 0.78268 |
| rs1199338 | MRAS | 3_138087467_A_C | 0.078506 | 1.25E−08 | 0.709855 | 0.019872 | 0.242265 |
| rs357504 | ARHGEF26 | 3_153956857_T_G | 0.023701 | 0 | 0.465139 | 0.176317 | 0.324021 |
| rs4561781 | CCNL1 | 3_156852943_A_G | 0.007153 | 0.026618 | 0.681127 | 0.224697 | 0 |
| rs12897 | FNDC3B | 3_172115902_G_A | 0.064011 | 0.127852 | 0.522894 | 0.236244 | 0.076207 |
| rs781663 | REST | 4_57781754_A_G | 0.017287 | 0.132263 | 0.521016 | 0.502101 | 0.175981 |
| rs13106227 | SHROOM3 | 4_77418681_G_A | 0.015268 | 0.240965 | 0.669976 | 0.175316 | 0 |
| rs1458038 | FGF5 | 4_81164723_C_T | 0.047418 | 0.042621 | 1.009404 | 0 | 0 |
| rs11099493 | RASGEF1B | 4_82587050_A_G | 0.075746 | 0.077351 | 0.277531 | 0.18982 | 0 |
| rs1434538 | BMPR1B | 4_96066672_C_T | 0.113413 | 0.063329 | 0.714219 | 0.093064 | 0.296506 |
| rs6820115 | PDE5A | 4_120398182_T_C | 0.011528 | 0 | 0.711646 | 0.145761 | 0 |
| rs13134800 | MAD2L1 | 4_120900282_T_C | 0.015193 | 0.039525 | 0.422946 | 0.10522 | 0 |
| rs1429141 | EDNRA_1 | 4_148288067_T_C | 0.005002 | 0.048544 | 0.258219 | 0.173815 | 0.051861 |
| rs6825620 | EDNRA_2 | 4_148381253_G_A | 0.000337 | 0.093044 | 0.309766 | 0.172599 | 0.054385 |
| rs6842241 | EDNRA_3 | 4_148400819_C_A | 0.073431 | 0 | 0.509718 | 0.157785 | 0 |
| rs2880099 | MAP9 | 4_156432368_A_C | 0.008229 | 0.047994 | 0.392318 | 0 | 0 |
| rs3796581 | GUCY1A3 | 4_156642884_A_G | 0.038075 | 0 | 0.793347 | 2.11E−09 | 0 |
| rs7696431 | PALLD | 4_169687725_T_G | 0.023737 | 0.065146 | 0.436872 | 0.120279 | 0.027212 |
| rs17328863 | SEMA5A | 5_9543031_C_T | 0.026979 | 0 | 0.196827 | 0.115175 | 0.274703 |
| rs3936511 | C5orf67 | 5_55860781_A_G | 0.025532 | 0.335864 | 1.286186 | 0.43861 | 1.360142 |
| rs273909 | SLC22A4 | 5_131667353_A_G | 0.021211 | 0.196672 | 0.904488 | 0.243463 | 0.042334 |
| rs246604 | ARHGAP26 | 5_142516458_G_A | 0 | 0 | 0.43646 | 0.021246 | 0.230998 |
| rs9349379 | PHACTR1_1 | 6_12903957_A_G | 0.019421 | 0.075928 | 0.228289 | 0.311557 | 0 |
| rs4711939 | PHACTR1_2 | 6_13222877_T_C | 0.082651 | 0.024801 | 0.384374 | 0.055453 | 0 |
| rs6456497 | HDGFL1 | 6_22603559_G_A | 0.006873 | 0.051631 | 0.363547 | 0.173029 | 0.017857 |
| rs3869098 | MUC22 | 6_31002452_A_G | 0.048613 | 0 | 1.779023 | 0.027698 | 0.145085 |
| rs2857605 | NFKBIL1 | 6_31524851_C_T | 0.055361 | 0.145906 | 1.465654 | 0.433581 | 0.101039 |
| rs2072633 | CFB | 6_31919578_A_G | 0.101012 | 0.135393 | 2.715495 | 0 | 0.063768 |
| rs2857103 | TAP2 | 6_32791299_C_A | 0.033422 | 0.461135 | 2.061795 | 0 | 0.145554 |
| rs2814985 | ILRUN | 6_34548296_C_T | 0.006497 | 0 | 0.843171 | 0.605783 | 0.499701 |
| rs1334894 | FKBP5 | 6_35615130_C_T | 0.131458 | 0 | 0.797449 | 0.155278 | 0.170464 |
| rs1321309 | CDKN1A | 6_36638636_G_A | 0.032816 | 0.071392 | 0.682904 | 0.041141 | 0.342657 |
| rs733701 | KCNK5_1 | 6_39171862_C_T | 0.133376 | 0.009993 | 0.403688 | 0.119609 | 0.115087 |
| rs10947789 | KCNK5_2 | 6_39174922_T_C | 0.126734 | 0 | 0.628492 | 0.116902 | 0.263294 |
| rs6905288 | VEGFA | 6_43758873_G_A | 0.022339 | 0.1348 | 1.583637 | 0 | 1.587898 |
| rs13219670 | PRIM2_1 | 6_57143175_A_G | 0.026122 | 0.046923 | 0.500648 | 0.039566 | 0 |
| rs9367716 | PRIM2_2 | 6_57160572_G_T | 0.010198 | 0.033892 | 0.421343 | 0.17655 | 1.11E−05 |
| rs9351814 | OGFRL1 | 6_72193707_A_C | 0.007678 | 0.011605 | 0.417785 | 0.261147 | 0.08095 |
| rs4613862 | FAM46A | 6_82612271_A_C | 0.047267 | 0.036422 | 0.191678 | 0.388507 | 0 |
| rs1591805 | CENPW | 6_126717064_A_G | 0 | 0.358195 | 1.479683 | 0.033942 | 0.302348 |
| rs969282 | TARID | 6_134205092_G_A | 0.009128 | 0.049014 | 0.365916 | 0.189194 | 0 |
| rs2627230 | SLC2A12 | 6_134379869_A_T | 0.044057 | 0.013379 | 0.229078 | 0.153364 | 0 |
| rs17080091 | PLEKHG1 | 6_150997401_C_T | 0.074014 | 0.083819 | 0.621405 | 0.168207 | 0 |
| rs686168 | SOD2 | 6_159951508_C_T | 1.032554 | 0.133041 | 0.29518 | 0.029629 | 0 |
| rs9456496 | IGF2R_1 | 6_160443390_A_G | 1.221281 | 0.297878 | 0.178499 | 0.089115 | 0 |
| rs688359 | IGF2R_2 | 6_160465291_G_A | 2.819323 | 0 | 0.253303 | 0.166315 | 0 |
| rs2297362 | IGF2R_3 | 6_160499565_T_C | 1.716963 | 4.48E−10 | 0.194908 | 0.092775 | 0 |
| rs9347386 | SLC22A1 | 6_160571499_A_G | 2.305535 | 0.55209 | 0.204189 | 0.029535 | 0 |
| rs3903279 | SLC22A2 | 6_160625033_T_G | 1.224651 | 0.292821 | 0.279706 | 0.120628 | 0 |
| rs512077 | SLC22A3_1 | 6_160774928_A_G | 1.2309 | 0.197465 | 0.702832 | 0.023449 | 0.019709 |
| rs2292334 | SLC22A3_2 | 6_160858188_G_A | 5.54078 | 0.219827 | 0.689338 | 1.66E−10 | 0.104724 |
| rs3127596 | LPA_1 | 6_160953035_A_G | 5.406823 | 0.384384 | 0.615387 | 0 | 0.491863 |
| rs12175867 | LPA_2 | 6_161019138_T_C | 3.784191 | 0.310475 | 0.033339 | 0.045274 | 0 |
| rs5014650 | LPA_3 | 6_161099503_G_A | 1.08069 | 0.101183 | 0.360748 | 0.276011 | 0 |
| rs2315065 | PLG_1 | 6_161108144_C_A | 12.33878 | 0.334955 | 0 | 0 | 0 |
| rs6455698 | PLG_2 | 6_161108803_G_T | 4.053381 | 0.338069 | 0.147276 | 0.129583 | 0 |
| rs783147 | PLG_3 | 6_161137990_G_A | 4.848889 | 0 | 0.226874 | 0 | 0.190001 |
| rs3798936 | AGPAT4 | 6_161646714_G_A | 0.189037 | 0.292009 | 0.487268 | 0.081682 | 0 |
| rs10267593 | MAD1L1 | 7_1937261_G_A | 0 | 0 | 0.636065 | 0.056713 | 0.067004 |
| rs3828944 | DAGLB | 7_6487156_T_C | 0.000993 | 0 | 0.408877 | 0.40271 | 0.497238 |
| rs2107595 | HDAC9 | 7_19049388_G_A | 0.024883 | 0 | 0.540981 | 0.028673 | 0 |
| rs2107732 | CCM2 | 7_45077978_G_A | 0.072515 | 0 | 0.4361 | 0.217386 | 0.091851 |
| rs7778250 | BCAP29 | 7_107224565_C_A | 0.083646 | 0.312572 | 0.533353 | 0.062968 | 0.014017 |
| rs975722 | CFTR | 7_117332914_A_G | 0.081575 | 0 | 0.443439 | 0.116575 | 0.256522 |
| rs11556924 | ZC3HC1 | 7_129663496_C_T | 0.035004 | 0 | 1.246952 | 0.06211 | 0.331684 |
| rs10237377 | PARP12 | 7_139757136_G_T | 0 | 0 | 0.715971 | 0.05532 | 0.20944 |
| rs6997340 | NAT2 | 8_18286997_T_C | 0.073961 | 0.258098 | 0.302693 | 0.446337 | 0.390625 |
| rs3289 | LPL_1 | 8_19823192_T_C | 0.001808 | 0.090928 | 0.30436 | 0 | 2.516136 |
| rs17091891 | LPL_2 | 8_19843171_T_C | 0 | 0.016767 | 0.594592 | 0 | 4.968011 |
| rs11204085 | SLC18A1 | 8_19940796_T_C | 0 | 0.106485 | 0.340475 | 0.010784 | 2.669215 |
| rs7819541 | BMP1 | 8_22042151_G_T | 0.005975 | 0.054049 | 0.238725 | 0.102363 | 0.00427 |
| rs10093110 | ZFPM2 | 8_106565414_G_A | 0 | 0 | 0.966319 | 0.099381 | 0.052233 |
| rs2001844 | TRIB1_1 | 8_126478745_A_G | 0 | 1.55063 | 1.448484 | 7.21E−08 | 2.832115 |
| rs7012891 | TRIB1_2 | 8_126514676_T_C | 0 | 0.538725 | 0.979791 | 0 | 1.485395 |
| rs1869204 | MTAP_1 | 9_21690958_T_G | 0.003464 | 0.029735 | 0.337964 | 0.116835 | 0.051856 |
| rs896655 | MTAP_2 | 9_21706571_A_G | 0.002874 | 0.049536 | 0.38236 | 0.087089 | 0 |
| rs10965139 | MTAP_3 | 9_21797186_G_A | 0.082526 | 0.042728 | 0.360198 | 0.097965 | 0.278762 |
| rs11515 | CDKN2A_1 | 9_21968199_C_G | 0.005273 | 0.063002 | 0.618477 | 0.117174 | 0.089496 |
| rs3731239 | CDKN2A_2 | 9_21974218_A_G | 0.008352 | 0.091365 | 0.469696 | 0.127971 | 0.030715 |
| rs12352425 | CDKN2B_1 | 9_22042086_G_A | 0.07092 | 0.038844 | 0.432124 | 0.23982 | 0.109621 |
| rs10811650 | CDKN2B_2 | 9_22067593_A_G | 0.03573 | 0.07604 | 0.361174 | 0.140315 | 0.011559 |
| rs7045889 | CDKN2B_3 | 9_22133251_G_A | 0.008179 | 0.074946 | 0.531845 | 0.151264 | 0.033694 |
| rs1967604 | KLF4 | 9_110530324_A_G | 0.021703 | 0.13491 | 0.470343 | 0.323035 | 0.020517 |
| rs885150 | DAB2IP | 9_124420173_T_C | 0.041867 | 0.094193 | 0.410708 | 0.092572 | 0.002696 |
| rs649129 | ABO | 9_136154304_C_T | 0 | 0.838256 | 0 | 8.699242 | 0 |
| rs7916879 | CDC123 | 10_12300790_G_A | 0.073282 | 0.061277 | 0.497216 | 0.104335 | 0.005687 |
| rs2478835 | KIAA1462 | 10_30317949_C_T | 0.10277 | 0.02821 | 0.560013 | 0.183403 | 0.139542 |
| rs10793514 | ZNF32 | 10_44496971_T_C | 0.053033 | 0.057517 | 0.259307 | 0.202498 | 0.004516 |
| rs677465 | C10orf142 | 10_44732917_G_A | 0.003013 | 0 | 0.226699 | 0.142919 | 0.066302 |
| rs12260962 | TSPAN14 | 10_82262826_C_T | 0.038265 | 0.01556 | 0.321558 | 0.053017 | 0.014052 |
| rs2246942 | LIPA | 10_91004886_A_G | 0.135975 | 0.036646 | 0.595056 | 0.267922 | 0.083181 |
| rs3740390 | AS3MT | 10_104638480_C_ | 0.008019 | 0.110936 | 1.20188 | 0 | 0 |
| | | T | | | | | |
| rs4918072 | STN1 | 10_105693644_G_ | 0.011918 | 0.078095 | 0.432162 | 0.267379 | 0 |
| | | A | | | | | |
| rs4752700 | HTRA1 | 10_124237612_A_ | 0.045951 | 0.078535 | 0.523174 | 0 | 0.02551 |
| | | G | | | | | |
| rs93139 | SWAP70 | 11_9759608_T_C | 0.017712 | 0.009283 | 0.984987 | 0 | 0.241508 |
| rs11042937 | CTR9 | 11_10745394_G_T | 0.052655 | 0.034691 | 0.300587 | 0.021004 | 0.098853 |
| rs2403662 | ARNTL | 11_13302822_A_G | 0.011134 | 0 | 0.961343 | 0.261797 | 0.285729 |
| rs7116641 | HSD17B12 | 11_43696917_T_G | 0 | 0 | 0.771238 | 0.075033 | 0.470014 |
| rs644740 | OVOL1 | 11_65561468_C_T | 0.009141 | 0 | 1.30958 | 0 | 0.635034 |
| rs590121 | SERPINH1_ | 11_75274150_G_T | 0.031432 | 0 | 0.372914 | 0.035627 | 0.025759 |
| | 1 | | | | | | |
| rs606452 | SERPINH1_ | 11_75276178_A_C | 0.003264 | 0.081191 | 1.014908 | 0.022423 | 0 |
| | 2 | | | | | | |
| rs607562 | ARHGAP42 | 11_100624538_T_ | 0.074891 | 0 | 1.106434 | 0 | 0.020519 |
| | | G | | | | | |
| rs11226029 | PDGFD | 11_103693627_G_ | 0 | 0.053893 | 0.358062 | 0.185466 | 0.008995 |
| | | A | | | | | |
| rs7127880 | SLN | 11_107600570_C_ | 0.005986 | 0.119296 | 0.183123 | 0.221798 | 0 |
| | | T | | | | | |
| rs662799 | APOA5 | 11_116663707_G_ | 0 | 0.460582 | 0.308262 | 0.200876 | 3.902471 |
| | | A | | | | | |
| rs2728620 | PDE3A_1 | 12_20157483_G_A | 0.053217 | 0.019576 | 0.396613 | 0.093101 | 0.019024 |
| rs11044977 | PDE3A_2 | 12_20158160_C_T | 0.026437 | 0 | 0.352904 | 0.102402 | 0.046428 |
| rs11170824 | SMUG1 | 12_54531481_G_A | 0.002975 | 0.178377 | 0.49556 | 0.056087 | 0 |
| rs11172113 | LRP1 | 12_57527283_T_C | 0.095352 | 0.058916 | 0.560147 | 0.323307 | 0.012482 |
| rs4760278 | R3HDM2 | 12_57771153_C_A | 0.133734 | 0.510933 | 1.44667 | 0.274627 | 0.862876 |
| rs2681472 | ATP2B1 | 12_90008959_A_G | 0.013127 | 0.071628 | 0.519659 | 0.129912 | 0 |
| rs11107896 | FGD6 | 12_95502303_G_A | 0.036274 | 0.024165 | 0.39923 | 0.122054 | 0.051858 |
| rs653178 | ATXN2 | 12_112007756_C_ | 0 | 0 | 4.484212 | 0 | 0.30534 |
| | | T | | | | | |
| rs12422941 | NAA25 | 12_112537738_T_ | 0 | 0 | 1.91413 | 0 | 0.413427 |
| | | C | | | | | |
| rs1183910 | IINF1A | 12_121420807_G_ | 0.060509 | 1.471167 | 1.422909 | 0 | 0 |
| | | A | | | | | |
| rs11057830 | SCARB1_1 | 12_125307053_G_ | 0.0029 | 0.366161 | 0.534122 | 0 | 0.507216 |
| | | A | | | | | |
| rs10773111 | SCARB1_2 | 12_125332955_T_ | 0 | 0.033978 | 0.418552 | 0.093176 | 0.402053 |
| | | C | | | | | |
| rs9319428 | FLT1 | 13_28973621_G_A | 0.012006 | 0.057234 | 0.669192 | 0.043915 | 0.147022 |
| rs7328733 | N4BP2L2 | 13_33126737_A_C | 0 | 0 | 1.084848 | 0.195856 | 0.113651 |
| rs11617955 | COL4A1_1 | 13_110818102_T_ | 0.001767 | 0.003267 | 0.533735 | 0 | 0.092043 |
| | | A | | | | | |
| rs9521636 | COL4A1_2 | 13_110834312_T_ | 0.085854 | 0 | 0.843082 | 1.07E−05 | 0.007205 |
| | | C | | | | | |
| rs693831 | COL4A1_3 | 13_110898375_T_ | 0.027811 | 0.06853 | 0.397359 | 0.189734 | 0 |
| | | C | | | | | |
| rs9521678 | COL4A1_4 | 13_110916118_T_ | 0.008474 | 0.120355 | 0.464135 | 0.053878 | 0.115493 |
| | | C | | | | | |
| rs3809346 | COL4A2_1 | 13_110960943_G_ | 0.001851 | 0.011581 | 0.314411 | 0.129255 | 0.076606 |
| | | A | | | | | |
| rs9515203 | COL4A2_2 | 13_111049623_T_ | 0.002377 | 0.038977 | 0.37402 | 0.324824 | 0 |
| | | C | | | | | |
| rs7333991 | COL4A2_3 | 13_111108436_T_ | 0.055987 | 0.013587 | 0.433946 | 0 | 0.189025 |
| | | C | | | | | |
| rs1317507 | MCF2L | 13_113631780_A_ | 0.033864 | 0.011456 | 0.598736 | 0.036603 | 0 |
| | | C | | | | | |
| rs2145598 | ARID4A | 14_58794001_G_A | 0 | 0.0085 | 0.993456 | 0.017776 | 0.167783 |
| rs175057 | MLH3 | 14_75489632_C_T | 0.001769 | 0.044513 | 0.439262 | 0.591507 | 0.119539 |
| rs2146238 | CYP46A1 | 14_100172729_T_ | 0.00711 | 0 | 0.315212 | 0.183764 | 0.003735 |
| | | G | | | | | |
| rs3783324 | EML1 | 14_100350017_T_ | 0 | 0.047114 | 0.504542 | 0 | 0.333216 |
| | | C | | | | | |
| rs6494488 | RBPMS2 | 15_65024204_G_A | 0.011251 | 0.166471 | 0.693707 | 0.112205 | 0.155369 |
| rs17293632 | SMAD3 | 15_67442596_C_T | 0.008866 | 0.03162 | 0.504336 | 0.167809 | 0.034098 |
| rs2277547 | ADAMTS7 | 15_79082431_A_G | 0.0102 | 0.056682 | 0.612762 | 0.102187 | 0 |
| rs7173743 | MORF4L1 | 15_79141784_T_C | 0.002837 | 0.027538 | 0.504152 | 0.107626 | 0.072659 |
| rs730657 | ABHD2 | 15_89566578_T_C | 0 | 0.008399 | 0.340033 | 0.264524 | 0.010035 |
| rs17514846 | FURIN | 15_91416550_C_A | 0.002 | 0.043478 | 1.010004 | 0 | 0 |
| rs10520790 | NR2F2 | 15_96151040_G_T | 0.03339 | 0.03961 | 0.252146 | 0.269114 | 0 |
| rs3764261 | CETP | 16_56993324_C_A | 0.449421 | 0.407495 | 0 | 0 | 6.516109 |
| rs12325142 | DHX38 | 16_72138112_G_T | 0.087353 | 0.469562 | 0.532783 | 0 | 0.11347 |
| rs4887824 | CFDP1 | 16_75441613_T_C | 0.014164 | 0.021942 | 0.684164 | 0.091815 | 0 |
| rs7199941 | PLCG2 | 16_81906423_A_G | 0.084601 | 0.128366 | 0.301095 | 0.308973 | 0 |
| rs7500448 | CDH13 | 16_83045790_A_G | 0 | 0 | 0.732858 | 0.045321 | 0.040622 |
| rs7209460 | SMG6_1 | 17_2048713_C_T | 0.10917 | 0.014873 | 0.61308 | 0.617166 | 0.172242 |
| rs16951962 | SMG6_2 | 17_2060686_A_G | 0.011708 | 0 | 0.478773 | 0.066361 | 0.041736 |
| rs8081803 | PAFAH1B1 | 17_2565009_T_C | 0 | 0.015019 | 0.546836 | 0.126506 | 0.182956 |
| rs12936587 | RAI1 | 17_17543722_G_A | 0.14532 | 0.169371 | 1.105292 | 0.046517 | 0.117784 |
| rs7220401 | ANKRD13B | 17_27935688_T_C | 0.005987 | 0 | 0.839695 | 0.113276 | 0.274134 |
| rs11080166 | UTP6 | 17_30028469_C_A | 0.000487 | 0 | 0.355785 | 0.05153 | 0.103207 |
| rs2074166 | KAT2A | 17_40267255_T_C | 0.186292 | 0.103381 | 0.460268 | 0.226476 | 0.176665 |
| rs17608766 | GOSR2 | 17_45013271_T_C | 0 | 0.038249 | 0.809636 | 0.122798 | 0 |
| rs1523136 | IGF2BP1 | 17_47144532_T_C | 0.00247 | 0 | 1.073203 | 0.014863 | 0.295506 |
| rs16948048 | ZNF652 | 17_47440466_A_G | 0.033499 | 0 | 1.782712 | 0.110261 | 0.288136 |
| rs7502499 | PHB | 17_47490102_G_A | 0.018046 | 0.003413 | 1.088502 | 1.60E−08 | 0.144168 |
| rs2159373 | BCAS3 | 17_59237499_A_G | 0.016948 | 0.018042 | 0.947252 | 0.229734 | 0.176633 |
| rs2812 | PECAM1 | 17_62401118_T_C | 0.049984 | 0.0188 | 0.7191 | 0.039041 | 0 |
| rs736839 | SMAD7 | 18_46528065_C_T | 0.017418 | 0.015049 | 0.421522 | 0.323951 | 0 |
| rs7238063 | ACAA2 | 18_47250165_C_T | 0.031283 | 0 | 0.409643 | 0.369652 | 0.024905 |
| rs538656 | MC4R | 18_57850422_G_T | 0.007504 | 0 | 1.305734 | 0.204629 | 0.813725 |
| rs13465 | ILF3 | 19_10802792_A_G | 0.070003 | 1.230874 | 0.523846 | 0 | 0.147126 |
| rs2304087 | SMARCA4 | 19_11107133_T_C | 0.009442 | 0.923816 | 0.311163 | 0.068623 | 0.212139 |
| rs6511720 | LDLR_1 | 19_11202306_G_T | 0.29423 | 3.354427 | 0.113452 | 0 | 0.037973 |
| rs5930 | LDLR_2 | 19_11224265_A_G | 0.025921 | 0.944547 | 0.208844 | 0.089446 | 0.261558 |
| rs17616620 | KANK2 | 19_11293642_G_A | 0.086358 | 0.194541 | 0.940386 | 0 | 0.705856 |
| rs318720 | EPOR | 19_11497104_T_G | 0.0024 | 0.036029 | 0.349838 | 0.12744 | 0.014439 |
| rs7251815 | MAP1S | 19_17844942_G_T | 0.064937 | 0.045971 | 0.618927 | 0.006024 | 0.093187 |
| rs12459996 | AXL | 19_41756906_G_T | 0.009191 | 0.025761 | 0.621896 | 0.192421 | 0.018779 |
| rs12984160 | TGFB1 | 19_41815713_A_G | 0.001585 | 0 | 0.492278 | 0.044478 | 0.182345 |
| rs11083616 | B9D2 | 19_41865643_G_A | 0.022858 | 0.001666 | 0.50858 | 0.095126 | 0.058387 |
| rs6859 | NECTIN2_1 | 19_45382034_A_G | 0.119851 | 3.199112 | 0.061169 | 0 | 0.482136 |
| rs7254892 | NECTIN2_2 | 19_45389596_G_A | 0.778427 | 6.10116 | 0 | 0.001465 | 0.462148 |
| rs405509 | APOE | 19_45408836_T_G | 0.2434 | 3.875854 | 0.187746 | 0 | 0.55045 |
| rs12721109 | APOC4 | 19_45447221_G_A | 0.428896 | 4.299327 | 0 | 0.094342 | 0.415879 |
| rs11672894 | MARK4 | 19_45801951_C_T | 0.046804 | 0 | 1.135005 | 0.581672 | 0.283302 |
| rs2341097 | SIX5 | 19_46268902_C_T | 0.003852 | 0.071143 | 0.735645 | 0.110011 | 0.323351 |
| rs1415771 | EDEM2 | 20_33734493_G_A | 0.005653 | 0 | 0.705333 | 0.577221 | 0.497775 |
| rs867186 | PROCR | 20_33764554_A_G | 0.078441 | 0.02948 | 0.545867 | 0.165016 | 0.113269 |
| rs3827066 | ZNF335 | 20_44586023_C_T | 0.007534 | 0.053436 | 0.501401 | 0.040702 | 0.004044 |
| rs236721 | ZNF831 | 20_57705485_T_C | 0 | 0 | 0.732815 | 0.093428 | 0.195037 |
| rs2832227 | MAP3K7CL | 21_30533076_A_G | 0.003038 | 0.075534 | 0.462682 | 0.137253 | 0.01519 |
| rs9305545 | KCNE2 | 21_35595821_A_G | 0.061792 | 0.031481 | 0.73155 | 0.087033 | 0 |
| rs5760293 | SPECC1L | 22_24662171_T_G | 0.051923 | 0.052072 | 0.441804 | 0.065234 | 0.194553 |

TABLE 13
Cluster weights for traits for CAD clustering

| | Lipoprotein | | Blood | ALP | HDL |
|---|---|---|---|---|---|
| trait | A | Cholesterol | markers | negative | negative |
| ADIPOGen_dv1.Adiponectin.ZN_pos | 0.26292 | 0.277232 | 0.489096 | 1.185717 | 0.03517 |
| BFpercent_dv1.BFP.ZN_pos | 0.002603 | 0 | 0.894408 | 0.328144 | 0 |
| GiantUKBB_eu_dv1.BMI.ZN_pos | 0.015491 | 0 | 0.850668 | 0.178845 | 0.116588 |
| MAGIC_eu_dv6.HBA1C.ZN_pos | 0.070445 | 0.072317 | 0.680786 | 0.626936 | 0.177532 |
| GiantUKBB_eu_dv1.HEIGHT.ZN_pos | 0.054718 | 0 | 0.706094 | 0.334199 | 0.158571 |
| GIANT_eu_females_dv5.HIPC.ZN_pos | 0.013382 | 0 | 0.685324 | 0.375201 | 0.118133 |
| GIANT_eu_males_dv5.HIPC.ZN_pos | 0.040709 | 0 | 0.63804 | 0.179166 | 0.231798 |
| GIANT_eu_females_dv5.HIPCadjBMI.ZN_pos | 0.208484 | 0 | 0.436694 | 0.296955 | 0.082803 |
| GIANT_eu_males_dv5.HIPCadjBMI.ZN_pos | 0.035691 | 0.015149 | 0.524924 | 0.215502 | 0.219758 |
| HRgene_dv1.HR.ZN_pos | 0.102151 | 0.00793 | 0.680784 | 0.030904 | 0.369967 |
| GIANT_eu_females_dv5.WAIST.ZN_pos | 0.023235 | 0 | 0.891569 | 0.089433 | 0.187576 |
| GIANT_eu_males_dv5.WAIST.ZN_pos | 0.014005 | 0 | 0.66188 | 0.123644 | 0.223152 |
| GIANT_eu_females_dv5.WAISTadjBMI.ZN_pos | 0.256073 | 0 | 1.059415 | 0.05911 | 0.288401 |
| GIANT_eu_males_dv5.WAISTadjBMI.ZN_pos | 0 | 0 | 0.584796 | 0.256596 | 0.127989 |
| GIANT_eu_females_dv5.WHRadjBMI.ZN_pos | 0.148079 | 0.187644 | 1.225989 | 0 | 0.421219 |
| GIANT_eu_males_dv5.WHRadjBMI.ZN_pos | 0.013468 | 3.96E−05 | 0.924927 | 0.683735 | 0.245814 |
| Haemoglobin.concentration_irnt_both.ZN_pos | 0 | 0 | 1.911406 | 0 | 0.016745 |
| Mean.corpuscular.volume_irnt_both.ZN_pos | 0 | 0.225407 | 0.630836 | 0.394849 | 0.066654 |
| Red.blood.cell . . . erythrocyte . . . | 0.03106 | 0.065909 | 0.630417 | 1.07721 | 0 |
| distribution.width_irnt_both.ZN_pos | | | | | |
| Platelet.count_irnt_both.ZN_pos | 0 | 0 | 2.239701 | 0.092648 | 0 |
| Mean.platelet . . . thrombocyte . . . volume_irnt_both.ZN_pos | 0.076362 | 0.174889 | 0.762979 | 0.436164 | 0.354535 |
| Platelet.distribution.width_irnt_both.ZN_pos | 0 | 0 | 0.911155 | 0.067355 | 1.33248 |
| Lymphocyte.count_irnt_both.ZN_pos | 0 | 2.24E−06 | 2.645523 | 0 | 0.025527 |
| Monocyte.count_irnt_both.ZN_pos | 0.010457 | 0.022283 | 1.811531 | 0 | 0 |
| Neutrophill.count_irnt_both.ZN_pos | 0 | 0 | 1.913515 | 0 | 0 |
| Eosinophill.count_irnt_both.ZN_pos | 0 | 0.178602 | 2.409169 | 0 | 0 |
| Basophill.count_irnt_both.ZN_pos | 0 | 0.141942 | 0.701372 | 0.009436 | 0 |
| Mean.reticulocyte.volume_irnt_both.ZN_pos | 0 | 0.028395 | 0.477214 | 0.978933 | 0 |
| Mean.sphered.cell.volume_irnt_both.ZN_pos | 0 | 0.051387 | 0.376279 | 0.858757 | 0 |
| High.light.scatter.reticulocyte.count_irnt_both.ZN_pos | 0 | 0.530386 | 2.622228 | 0 | 0.68218 |
| Albumin . . . quantile._irnt_both.ZN_pos | 0.061739 | 0.440154 | 0.807169 | 0.018933 | 0.256276 |
| Alkaline.phosphatase . . . quantile._irnt_both.ZN_pos | 0 | 0.518503 | 1.685699 | 0 | 0.775481 |
| Alanine.aminotransferase . . . quantile._irnt_both.ZN_pos | 0.060229 | 0.131556 | 1.772487 | 0.397788 | 0.584102 |
| Apoliprotein.B . . . quantile._irnt_both.ZN_pos | 0.78054 | 10.64567 | 0 | 0.107395 | 1.605279 |
| Aspartate.aminotransferase . . . quantile._irnt_both.ZN_pos | 0 | 1.94E−08 | 1.479066 | 0 | 0.777022 |
| Direct.bilirubin . . . quantile._irnt_both.ZN_pos | 0 | 0 | 1.023717 | 0.063799 | 0 |
| Urea . . . quantile._irnt_both.ZN_pos | 0.040903 | 0.097951 | 0.779935 | 0.570154 | 0.028258 |
| Calcium . . . quantile._irnt_both.ZN_pos | 0.061345 | 0.172744 | 1.051758 | 1.41E−07 | 0.046611 |
| C.reactive.protein . . . quantile._irnt_both.ZN_pos | 0 | 0 | 1.479889 | 0.973656 | 0.259606 |
| Gamma.glutamyltransferase . . . quantile._irnt_both.ZN_pos | 0.019033 | 0.177854 | 1.20968 | 0.949927 | 0.475918 |
| Glucose . . . quantile._irnt_both.ZN_pos | 0.052396 | 0.098264 | 0.464039 | 0.802501 | 0.287808 |
| HDL.cholesterol . . . quantile._irnt_both.ZN_pos | 0.258897 | 0.096428 | 0.469308 | 0.693414 | 0 |
| IGF.1 . . . quantile._irnt_both.ZN_pos | 0.440639 | 0.408361 | 1.297481 | 0.271519 | 0.008077 |
| Lipoprotein.A . . . quantile._irnt_both.ZN_pos | 16.9884 | 0.000357 | 0 | 0 | 1.75E−05 |
| Phosphate . . . quantile._irnt_both.ZN_pos | 0 | 0.155653 | 0.953155 | 0.024883 | 0 |
| SHBG . . . quantile._irnt_both.ZN_pos | 0.070404 | 0.141701 | 0.325519 | 0.102448 | 0 |
| Total.bilirubin . . . quantile._irnt_both.ZN_pos | 0 | 0.177351 | 1.036479 | 0.030518 | 0.299593 |
| Testosterone . . . quantile._irnt_female.ZN_pos | 0.00133 | 0.197459 | 0.806747 | 0.102665 | 0 |
| Testosterone . . . quantile._irnt_male.ZN_pos | 0.03051 | 0 | 0.476447 | 0.065654 | 0.108541 |
| Total.protein . . . quantile._irnt_both.ZN_pos | 0.093372 | 0.395134 | 1.384559 | 0.366367 | 0 |
| Triglycerides . . . quantile._irnt_both.ZN_pos | 0 | 0 | 1.227129 | 0 | 5.805899 |
| Urate . . . quantile._irnt_both.ZN_pos | 0.103599 | 0 | 1.773449 | 0.044725 | 0.260107 |
| Vitamin.D . . . quantile._irnt_both.ZN_pos | 0.010365 | 0.004309 | 0.37936 | 0.106125 | 0.297074 |
| Diastolic.blood.pressure . . . | 0 | 0 | 2.367671 | 0 | 0 |
| automated.reading_irnt_both.ZN_pos | | | | | |
| Systolic.blood.pressure . . . | 0 | 0.09509 | 2.30948 | 0.070857 | 0 |
| automated.reading_irnt_both.ZN_pos | | | | | |
| ADIPOGen_dv1.Adiponectin.ZN_neg | 0 | 0.273196 | 1.819608 | 0.083918 | 0.874962 |
| BFpercent_dv1.BFP.ZN_neg | 0.384426 | 0.759974 | 0.75765 | 0.022026 | 0.379532 |
| GiantUKBB_eu_dv1.BMI.ZN_neg | 0.10318 | 0.459491 | 0.58116 | 5.95E−06 | 0.041796 |
| MAGIC_eu_dv6.HBA1C.ZN_neg | 0.065935 | 0.115555 | 0.872886 | 0.028936 | 0.111993 |
| GiantUKBB_eu_dv1.HEIGHT.ZN_neg | 0.166106 | 0.565475 | 2.164537 | 0.195603 | 0 |
| GIANT_eu_females_dv5.HIPC.ZN_neg | 0.12339 | 0.812694 | 1.016738 | 0 | 0.14111 |
| GIANT_eu_males_dv5.HIPC.ZN_neg | 0.14159 | 0.57226 | 1.45861 | 0.407909 | 0.02218 |
| GIANT_eu_females_dv5.HIPCadjBMI.ZN_neg | 0.025625 | 0.582165 | 1.015994 | 0 | 0.275234 |
| GIANT_eu_males_dv5.HIPCadjBMI.ZN_neg | 0.10314 | 0.196637 | 1.681662 | 0.45665 | 0.063815 |
| HRgene_dv1.HR.ZN_neg | 0.141645 | 0.213237 | 0.610453 | 0.464979 | 0.020417 |
| GIANT_eu_females_dv5.WAIST.ZN_neg | 0.072685 | 0.676563 | 0.627723 | 0.152382 | 0.026714 |
| GIANT_eu_males_dv5.WAIST.ZN_neg | 0.187381 | 0.697734 | 0.963342 | 0.081624 | 0 |
| GIANT_eu_females_dv5.WAISTadjBMI.ZN_neg | 0 | 0.251967 | 0.510298 | 0.508256 | 4.22E−05 |
| GIANT_eu_males_dv5.WAISTadjBMI.ZN_neg | 0.116688 | 0.491159 | 1.117123 | 0.041993 | 0 |
| GIANT_eu_females_dv5.WHRadjBMI.ZN_neg | 0.006618 | 0.023761 | 0.411663 | 0.590947 | 1.23E−08 |
| GIANT_eu_males_dv5.WHRadjBMI.ZN_neg | 0.033232 | 0.276085 | 0.504952 | 0.116794 | 0.093481 |
| Haemoglobin.concentration_irnt_both.ZN_neg | 0.143183 | 0.347393 | 0.38529 | 2.005495 | 0 |
| Mean.corpuscular.volume_irnt_both.ZN_neg | 0.099598 | 0.066313 | 1.032213 | 0.025992 | 0.219325 |
| Red.blood.cell . . . erythrocyte . . . | 0 | 1.172678 | 0.751185 | 0 | 1.19253 |
| distribution.width_irnt_both.ZN_neg | | | | | |
| Platelet.count_irnt_both.ZN_neg | 0.193578 | 0.331519 | 0.986109 | 0.338916 | 0.378652 |
| Mean.platelet . . . thrombocyte . . . volume_irnt_both.ZN_neg | 0 | 0.222516 | 1.066147 | 0 | 0 |
| Platelet.distribution.width_irnt_both.ZN_neg | 0.127937 | 0.251255 | 0.935004 | 0.715871 | 0 |
| Lymphocyte.count_irnt_both.ZN_neg | 0.052424 | 0.243517 | 0.527167 | 0.434663 | 0 |
| Monocyte.count_irnt_both.ZN_neg | 0.125685 | 0.189228 | 0.613164 | 1.357662 | 0.177805 |
| Neutrophill.count_irnt_both.ZN_neg | 0 | 0.206768 | 0.375924 | 0.955549 | 0.217955 |
| Eosinophill.count_irnt_both.ZN_neg | 0.031932 | 0.085776 | 0.397758 | 0.770405 | 0.263969 |
| Basophill.count_irnt_both.ZN_neg | 0.022978 | 0.094699 | 0.329291 | 0.107407 | 0.111909 |
| Mean.reticulocyte.volume_irnt_both.ZN_neg | 0.155125 | 0.577033 | 0.963712 | 0 | 0.88801 |
| Mean.sphered.cell.volume_irnt_both.ZN_neg | 0.099464 | 0.634579 | 1.486793 | 0 | 0.767173 |
| High.light.scatter.reticulocyte.count_irnt_both.ZN_neg | 0.050212 | 0.076037 | 0.234965 | 0.551511 | 0 |
| Albumin . . . quantile._irnt_both.ZN_neg | 0.373328 | 0.041923 | 0.903898 | 0.357231 | 0 |
| Alkaline.phosphatase . . . quantile._irnt_both.ZN_neg | 0 | 0.25258 | 0 | 7.793546 | 0 |
| Alanine.aminotransferase . . . quantile._irnt_both.ZN_neg | 0 | 0.591091 | 0.235524 | 0.137032 | 0 |
| Apoliprotein.B . . . quantile._irnt_both.ZN_neg | 0 | 0 | 0.954137 | 0.147482 | 0 |
| Aspartate.aminotransferase . . . quantile._irnt_both.ZN_neg | 0.018786 | 0.421198 | 0.289677 | 1.306801 | 0 |
| Direct.bilirubin . . . quantile._irnt_both.ZN_neg | 0.195753 | 1.33065 | 0.570196 | 0.534546 | 0.22944 |
| Urea . . . quantile._irnt_both.ZN_neg | 3.08E−07 | 0.201855 | 0.658365 | 0.077844 | 0.009988 |
| Calcium . . . quantile._irnt_both.ZN_neg | 0.169403 | 0.105635 | 0.347125 | 0.496839 | 0.150024 |
| C.reactive.protein . . . quantile._irnt_both.ZN_neg | 0 | 2.26866 | 0.730176 | 0 | 0 |
| Gamma.glutamyltransferase . . . quantile._irnt_both.ZN_neg | 0.042573 | 0.521093 | 0.782552 | 0.052586 | 0 |
| Glucose . . . quantile._irnt_both.ZN_neg | 0.015716 | 0.105053 | 0.484832 | 0.022577 | 0 |
| HDL.cholesterol . . . quantile._irnt_both.ZN_neg | 0.097379 | 0.384189 | 0.139053 | 0.004398 | 8.590624 |
| IGF.1 . . . quantile._irnt_both.ZN_neg | 0.051976 | 0.265975 | 0.829015 | 0.027442 | 0.082583 |
| Lipoprotein.A . . . quantile._irnt_both.ZN_neg | 0 | 0 | 0.419275 | 0.158814 | 0.181215 |
| Phosphate . . . quantile._irnt_both.ZN_neg | 0.071427 | 0.042505 | 0.497293 | 0.089619 | 0.122969 |
| SHBG . . . quantile._irnt_both.ZN_neg | 0.495557 | 0.117028 | 2.333344 | 0.195658 | 0.215873 |
| Total.bilirubin . . . quantile._irnt_both.ZN_neg | 0.112317 | 0.086897 | 0.774485 | 0.596668 | 0.001286 |
| Testosterone . . . quantile._irnt_female.ZN_neg | 0.105643 | 0.092739 | 0.818897 | 0.209218 | 0.331535 |
| Testosterone . . . quantile._irnt_male.ZN_neg | 0.339557 | 0.320743 | 1.653654 | 0.240659 | 0.267952 |
| Total.protein . . . quantile._irnt_both.ZN_neg | 0.040067 | 3.54E−05 | 0.642861 | 0.194208 | 0.074005 |
| Triglycerides . . . quantile._irnt_both.ZN_neg | 0.578618 | 1.123703 | 0.216234 | 0.558519 | 0 |
| Urate . . . quantile._irnt_both.ZN_neg | 0.028833 | 0.376992 | 0.403272 | 0.022104 | 0 |
| Vitamin.D . . . quantile._irnt_both.ZN_neg | 0.061465 | 0.692901 | 0.34187 | 0.273068 | 0.242229 |
| Diastolic.blood.pressure . . . | 0.136507 | 0.391983 | 0.377491 | 0.597398 | 0 |
| automated.reading_irnt_both.ZN_neg | | | | | |
| Systolic.blood.pressure . . . | 0.129402 | 0.015266 | 0.232933 | 0.123455 | 0.002232 |
| automated.reading_irnt_both.ZN_neg | | | | | |

TABLE 14
Cluster weights for CKD Loci

| | | | Blood | Reduced | Lipoprotein | Beta-cell |
|---|---|---|---|---|---|---|
| rsID | locus | VAR_ID_hg19 | markers | hematopoiesis | A | opposite |
| rs284277 | CASZ1 | 1_10790797_C_A | 0.262039 | 0.745249 | 0.044751 | 0.35284 |
| rs2849025 | ZNF436 | 1_23693422_T_C | 0.063356 | 0.911594 | 0.022404 | 0.376456 |
| rs12741781 | SDCCAG8 | 1_243428152_T_G | 0.452179 | 0.771199 | 0.096725 | 0.115704 |
| rs966003 | SPTBN1 | 2_54884870_C_T | 0.27874 | 0.493622 | 0.041056 | 0.526471 |
| rs10206899 | ALMS1P1 | 2_73900900_T_C | 0.423214 | 0.524435 | 0.014571 | 0.593661 |
| rs1015754 | PSD4 | 2_113967990_T_C | 0.448118 | 0.997477 | 0 | 0.017363 |
| rs1078441 | TFCP2L1 | 2_121989071_A_C | 0.086175 | 0.569627 | 0.045972 | 1.177887 |
| rs12987817 | LRP2_1 | 2_170093296_A_G | 0.158322 | 0.449965 | 0.021233 | 0.010531 |
| rs1668134 | LRP2_2 | 2_170206062_T_A | 0.107487 | 0.624893 | 0.024227 | 0 |
| rs6723109 | IIOXD1 | 2_177081547_A_G | 0.222634 | 0.744307 | 0.04772 | 0.168271 |
| rs16844862 | CPS1 | 2_211587631_C_T | 0.384623 | 1.492596 | 0 | 0.000677 |
| rs2712169 | TNP1 | 2_217671349_G_A | 0.395735 | 0.87953 | 0.057376 | 0.224057 |
| rs7648352 | GADL1 | 3_30777944_A_G | 0.166752 | 0.487599 | 0.095772 | 0.260208 |
| rs12107150 | TFDP2 | 3_141823766_G_A | 0.040135 | 0.977799 | 0.148395 | 0 |
| rs12629364 | KNG1 | 3_186428182_C_A | 0.28179 | 0.644036 | 0.116183 | 0.046735 |
| rs16874072 | PPARGC1A | 4_23743450_T_C | 0.416265 | 0.706966 | 0.047292 | 6.12E−10 |
| rs1876535 | SHROOM3 | 4_77296600_T_C | 0.331797 | 0.725764 | 0.015587 | 0.164449 |
| rs1458038 | FGF5 | 4_81164723_C_T | 0.012614 | 1.41509 | 0.089031 | 0.031108 |
| rs6880621 | RAI14 | 5_34504382_G_A | 0.573243 | 0.562438 | 2.65E−05 | 0.272425 |
| rs700237 | C9 | 5_39368176_A_G | 0.040892 | 0.752973 | 0.006715 | 0.428634 |
| rs12654812 | SLC34A1 | 5_176794191_G_A | 1.180764 | 0.852946 | 0.031692 | 0.062461 |
| rs6929219 | CDKAL1 | 6_21214200_T_C | 0.104735 | 0.657309 | 0.008608 | 0.074455 |
| rs2736157 | PRRC2A | 6_31600820_A_G | 0 | 3.875908 | 0 | 0.348247 |
| rs9268853 | HLA-DRB9 | 6_32429643_T_C | 2.594727 | 0.662547 | 0.125283 | 0 |
| rs1058768 | TINAG | 6_54186147_T_C | 0.112027 | 0.529726 | 0.056223 | 0.085403 |
| rs7740107 | L3MBTL3 | 6_130374461_T_A | 1.333837 | 1.336834 | 0.078821 | 0.336422 |
| rs10945656 | SLC22A2 | 6_160635886_G_A | 0 | 7.94E−06 | 8.949837 | 0 |
| rs1949783 | UNCX | 7_1303439_C_T | 0.091698 | 0.831205 | 0.101425 | 0.168062 |
| rs3956705 | AVL9 | 7_32953295_C_T | 0.280363 | 1.072412 | 0.022844 | 0.012148 |
| rs6958520 | KCTD7 | 7_66151453_T_C | 0.411667 | 0.782855 | 0.060495 | 0.048325 |
| rs11761707 | PTPN12 | 7_77238184_A_G | 0 | 1.235003 | 0.062601 | 0 |
| rs6947064 | PRKAG2 | 7_151397343_A_G | 0.179571 | 1.35152 | 0.257846 | 0.254976 |
| rs1405809 | RNF32 | 7_156266712_G_A | 0.031952 | 0.420314 | 0.177507 | 0.697673 |
| rs10109414 | STC1 | 8_23751151_C_T | 0.25844 | 0.957276 | 0.082395 | 0.169665 |
| rs1556751 | PIP5K1B | 9_71433212_G_A | 0.318315 | 0.599598 | 0.007494 | 0.286761 |
| rs1044261 | IDI2 | 10_1065710_C_T | 0.051143 | 0.949671 | 0.077682 | 0.063708 |
| rs963837 | MPPED2 | 11_30749090_T_C | 0.494222 | 1.134633 | 0 | 0.055124 |
| rs7103648 | RAPSN | 11_47461783_A_G | 0.339586 | 0 | 0 | 6.472114 |
| rs1109906 | OR4A1P | 11_18284033_C_T | 0.198907 | 0.337865 | 0.080932 | 0.775264 |
| rs11227279 | KRT8P26 | 11_65495211_G_A | 1.296588 | 0.959603 | 0.150161 | 0.300666 |
| rs4980598 | MYEOV | 11_69231796_T_C | 0.56313 | 0.649567 | 0.114891 | 0.170517 |
| rs2256187 | USP35 | 11_77919768_G_A | 0.27583 | 0.882899 | 0.044115 | 0.269168 |
| rs1472687 | SORL1 | 11_121584560_G_A | 0.016839 | 0.817505 | 0.036015 | 0.80445 |
| rs7139311 | RERG | 12_15266796_C_T | 0.368071 | 0.260417 | 0.049473 | 0.395543 |
| rs710750 | LRRC10 | 12_70007775_T_C | 0.005783 | 0.230319 | 0.123834 | 1.141821 |
| rs4766578 | ATXN2 | 12_111904371_T_A | 7.420266 | 0 | 0 | 0.483846 |
| rs1594810 | C15orf53 | 15_39225365_C_A | 0.201044 | 0.524352 | 0.127384 | 0.179218 |
| rs7180259 | C15orf54 | 15_39302724_C_A | 0.564241 | 0.504866 | 0 | 0.781782 |
| rs9920634 | GATM | 15_45596546_G_T | 0.221093 | 0.775574 | 5.42E−05 | 0 |
| rs674582 | SQRDL | 15_45936589_G_A | 0.049342 | 0.315931 | 0.045614 | 0.363209 |
| rs11070991 | WDR72_1 | 15_53856688_C_A | 0.163206 | 0.327897 | 3.51E−10 | 0.703651 |
| rs690428 | WDR72_2 | 15_53950578_A_C | 0 | 0.944798 | 0.049359 | 0 |
| rs1378942 | CSK | 15_75077367_C_A | 0.085537 | 1.54425 | 0.039469 | 0.259118 |
| rs2593280 | UBE2Q2 | 15_76150965_G_A | 0.107895 | 0.995927 | 0.025204 | 0.541557 |
| rs1391470 | C15orf27 | 15_76429785_C_T | 0.323572 | 0.414733 | 0 | 0.695344 |
| rs12708631 | UMOD_1 | 16_20365697_T_A | 0.17879 | 0.355551 | 0.057879 | 0.046598 |
| rs13334633 | UMOD_2 | 16_20393959_G_T | 0.052025 | 0.573048 | 0 | 0.85164 |
| rs11864909 | PDILT | 16_20400839_C_T | 0.10843 | 1.2674 | 0.061325 | 0 |
| rs2453580 | SLC47A1 | 17_19438321_T_C | 0.243019 | 0.522896 | 0.029594 | 0.286984 |
| rs2291285 | MY019 | 17_34890362_G_A | 0.328076 | 1.403175 | 7.75E−06 | 0.037371 |
| rs9916302 | FBXL20 | 17_37499949_T_C | 0.792805 | 1.204703 | 0.097533 | 0 |
| rs1312351 | SKAP1 | 17_46377892_T_C | 0.539262 | 0.567364 | 0 | 0.378112 |
| rs9895661 | BCAS3 | 17_59456589_C_T | 0.05176 | 1.708284 | 0 | 0.053561 |
| rs12460876 | SLC7A9 | 19_33356891_T_C | 0.060113 | 0.988645 | 0.035713 | 0.262828 |
| rs6048699 | NXT1 | 20_23285903_C_T | 0.157654 | 0.424871 | 0.079703 | 0.088288 |
| rs13044242 | CST4 | 20_23699253_G_C | 0.077205 | 0.496756 | 0 | 0.021344 |
| rs2983539 | GGTLC1 | 20_23930543_C_A | 0.03281 | 0.59068 | 0.025908 | 0 |
| rs764597 | PIGU | 20_33161225_G_A | 0.901211 | 1.073046 | 0.128622 | 0.508963 |
| rs1328757 | PCK1 | 20_56135199_C_T | 0.672969 | 1.215166 | 0.085005 | 0 |
| rs4809393 | TCEA2 | 20_62701092_A_C | 0.525628 | 0.990624 | 0.061499 | 0.094371 |
| rs2823139 | NRIP1 | 21_16576783_G_A | 0.222364 | 1.245705 | 0.229056 | 0.189613 |

TABLE 15
Cluster weights for traits for CKD clustering

| | Blood | Reduced | Lipoprotein | Beta-cell |
|---|---|---|---|---|
| trait | markers | hematopoiesis | A | opposite |
| ADIPOGen_dv1.Adiponectin.ZN_pos | 0 | 1.105888 | 0.009824 | 0.361897 |
| BFpercent_dv1.BFP.ZN_pos | 0.007313 | 0.746353 | 0.012559 | 0.032295 |
| GiantUKBB_eu_dv1.BMI.ZN_pos | 0 | 0.68117 | 0 | 0.011824 |
| MAGIC_dv1.FG.ZN_pos | 0.302855 | 0.655672 | 0.486875 | 0.056952 |
| MAGIC_eu_dv6.HBA1C.ZN_pos | 0.030157 | 0.655582 | 0.003227 | 0.070783 |
| GiantUKBB_eu_dv1.HEIGHT.ZN_pos | 0.129841 | 0.844592 | 0.0284 | 0.215886 |
| GIANT_eu_males_dv5.HIPC.ZN_pos | 0 | 0.8642 | 0.203454 | 0.103485 |
| GIANT_eu_males_dv5.HIPCadjBMI.ZN_pos | 0.052248 | 0.79916 | 0.241713 | 0.08687 |
| MAGIC_dv1.PI.ZN_pos | 0.42406 | 2.356471 | 0.107549 | 0 |
| GIANT_eu_females_dv5.WAIST.ZN_pos | 0.112273 | 0.94159 | 0.064703 | 0 |
| GIANT_eu_males_dv5.WAIST.ZN_pos | 0.0174 | 0.717196 | 0.006346 | 0.090634 |
| GIANT_eu_females_dv5.WAISTadjBMI.ZN_pos | 0.35948 | 0.775532 | 0.456036 | 0 |
| GIANT_eu_females_dv5.WHRadjBMI.ZN_pos | 0.453242 | 0.874961 | 0.210674 | 0.166317 |
| GIANT_eu_males_dv5.WHRadjBMI.ZN_pos | 0.393165 | 0.67675 | 0.062011 | 0.165892 |
| White.blood.cell . . . leukocyte . . . count_irnt_both.ZN_pos | 3.129565 | 8.00E−09 | 0.008498 | 0 |
| Haemoglobin.concentration_irnt_both.ZN_pos | 2.72812 | 0.432637 | 0 | 0 |
| Mean.corpuscular.volume_irnt_both.ZN_pos | 0.520377 | 0.421958 | 0 | 0.523752 |
| Red.blood.cell . . . erythrocyte . . . | 0 | 1.277579 | 0 | 0 |
| distribution.width_irnt_both.ZN_pos | | | | |
| Platelet.count_irnt_both.ZN_pos | 3.455546 | 0 | 0 | 0 |
| Mean.platelet . . . thrombocyte . . . volume_irnt_both.ZN_pos | 0.035164 | 1.163021 | 0.02691 | 0.005733 |
| Platelet.distribution.width_irnt_both.ZN_pos | 0.727109 | 0.708975 | 0.027462 | 0.609635 |
| Eosinophill.count_irnt_both.ZN_pos | 3.754564 | 0 | 0 | 0.290324 |
| Basophill.count_irnt_both.ZN_pos | 0.766742 | 0.111214 | 0.00427 | 0.004918 |
| Nucleated.red.blood.cell.count_irnt_both.ZN_pos | 2.05E−06 | 0.425811 | 0.002012 | 0.178598 |
| Reticulocyte.count_irnt_both.ZN_pos | 2.483526 | 0.012531 | 0.009714 | 0.06889 |
| Mean.reticulocyte.volume_irnt_both.ZN_pos | 0 | 1.16558 | 0 | 0.279789 |
| Mean.sphered.cell.volume_irnt_both.ZN_pos | 0 | 0.898007 | 0 | 0.298021 |
| Albumin . . . quantile._irnt_both.ZN_pos | 0.024141 | 0.458626 | 0.342316 | 0.792467 |
| Alkaline.phosphatase . . . quantile._irnt_both.ZN_pos | 1.119224 | 0.327081 | 0.100342 | 0.102291 |
| Alanine.aminotransferase . . . quantile._irnt_both.ZN_pos | 1.266412 | 0.327507 | 0.13512 | 0.38412 |
| Aspartate.aminotransferase . . . quantile._irnt_both.ZN_pos | 1.08579 | 0.183167 | 0.011633 | 0.847333 |
| Urea . . . quantile._irnt_both.ZN_pos | 0.189175 | 1.641991 | 0.061309 | 0.831635 |
| Calcium . . . quantile._irnt_both.ZN_pos | 0.355142 | 0.75368 | 0.299316 | 0.621247 |
| Cholesterol . . . quantile._irnt_both.ZN_pos | 0.039408 | 0.548811 | 0.649572 | 0.132481 |
| C.reactive.protein . . . quantile._irnt_both.ZN_pos | 0.66526 | 0.608574 | 0.016965 | 0.267685 |
| Gamma.glutamyltransferase . . . quantile._irnt_both.ZN_pos | 0.328802 | 0.5232 | 0.118299 | 0.641104 |
| Glucose . . . quantile._irnt_both.ZN_pos | 0.024171 | 0.630249 | 0 | 0.028432 |
| HDL.cholesterol . . . quantile._irnt_both.ZN_pos | 0.123562 | 0.383393 | 0.007729 | 0.11318 |
| IGF.1 . . . quantile._irnt_both.ZN_pos | 0.099089 | 0.472343 | 0.858916 | 0.13376 |
| Lipoprotein.A . . . quantile._irnt_both.ZN_pos | 0 | 0 | 8.784624 | 0 |
| Phosphate . . . quantile._irnt_both.ZN_pos | 0.925666 | 0.360839 | 0.004259 | 0.057615 |
| SHBG . . . quantile._irnt_both.ZN_pos | 0.071195 | 0.504554 | 0.005231 | 0.061085 |
| Total.bilirubin . . . quantile._irnt_both.ZN_pos | 1.371437 | 0.215658 | 0 | 0.141753 |
| Testosterone . . . quantile._irnt_female.ZN_pos | 0.188628 | 0.557479 | 0.009535 | 0.313155 |
| Testosterone . . . quantile._irnt_male.ZN_pos | 0.484059 | 0.277252 | 0.004381 | 0.091749 |
| Total.protein . . . quantile._irnt_both.ZN_pos | 0.748148 | 0.037593 | 0.287943 | 0.69745 |
| Triglycerides . . . quantile._irnt_both.ZN_pos | 0.774115 | 0.668014 | 0.234567 | 0.139448 |
| Urate . . . quantile._irnt_both.ZN_pos | 1.39348 | 1.641738 | 0.489912 | 0.602928 |
| Vitamin.D . . . quantile._irnt_both.ZN_pos | 0.058409 | 0.488456 | 0.163445 | 0.391625 |
| Diastolic.blood.pressure . . . | 1.560755 | 0.205464 | 0.01954 | 0.629942 |
| automated.reading_irnt_both.ZN_pos | | | | |
| Systolic.blood.pressure . . . | 0.82153 | 0.365011 | 0.018999 | 0.683587 |
| automated.reading_irnt_both.ZN_pos | | | | |
| ADIPOGen_dv1.Adiponectin.ZN_neg | 0.382437 | 1.367265 | 0.278276 | 0.04457 |
| BFpercent_dv1.BFP.ZN_neg | 0.295525 | 0.559415 | 0.304863 | 0.477248 |
| GiantUKBB_eu_dv1.BMI.ZN_neg | 0.040299 | 0.226442 | 0.22829 | 0.580019 |
| MAGIC_dv1.FG.ZN_neg | 0.037034 | 0.417124 | 0.016751 | 1.088916 |
| MAGIC_eu_dv6.HBA1C.ZN_neg | 0.796641 | 0.157705 | 0.041592 | 0.394369 |
| GiantUKBB_eu_dv1.HEIGHT.ZN_neg | 0.28814 | 0.64331 | 0.173835 | 0.007244 |
| GIANT_eu_males_dv5.HIPC.ZN_neg | 0.853523 | 0.380341 | 0.007455 | 0.894328 |
| GIANT_eu_males_dv5.HIPCadjBMI.ZN_neg | 0.578576 | 0.587767 | 0.016995 | 0.300425 |
| MAGIC_dv1.PI.ZN_neg | 0.384789 | 0 | 0 | 6.331012 |
| GIANT_eu_females_dv5.WAIST.ZN_neg | 0.297364 | 0.169552 | 0.007696 | 0.567722 |
| GIANT_eu_males_dv5.WAIST.ZN_neg | 0.589465 | 0.517997 | 0.101789 | 0.773423 |
| GIANT_eu_females_dv5.WAISTadjBMI.ZN_neg | 0 | 0.58095 | 0.006376 | 0.287444 |
| GIANT_eu_females_dv5.WHRadjBMI.ZN_neg | 0 | 0.706578 | 0.003222 | 0.050794 |
| GIANT_eu_males_dv5.WHRadjBMI.ZN_neg | 0 | 1.012061 | 0.002685 | 0.103675 |
| White.blood.cell . . . leukocyte . . . count_irnt_both.ZN_neg | 0 | 1.511877 | 0.057402 | 0.11785 |
| Haemoglobin.concentration_irnt_both.ZN_neg | 0 | 2.107566 | 0.238882 | 0.300814 |
| Mean.corpuscular.volume_irnt_both.ZN_neg | 0.145368 | 0.551576 | 0.250186 | 0 |
| Red.blood.cell . . . erythrocyte . . . | 0.509977 | 0.636175 | 0.01455 | 0.476039 |
| distribution.width_irnt_both.ZN_neg | | | | |
| Platelet.count_irnt_both.ZN_neg | 0 | 1.097056 | 0.018878 | 0.01589 |
| Mean.platelet . . . thrombocyte . . . volume_irnt_both.ZN_neg | 0.311411 | 0.311414 | 0.011938 | 0.465085 |
| Platelet.distribution.width_irnt_both.ZN_neg | 0.034908 | 0.445293 | 0.001001 | 0.04095 |
| Eosinophill.count_irnt_both.ZN_neg | 0 | 0.954391 | 0.056056 | 0 |
| Basophill.count_irnt_both.ZN_neg | 0 | 0.593853 | 0.007324 | 0.099653 |
| Nucleated.red.blood.cell.count_irnt_both.ZN_neg | 0.026229 | 0.172779 | 0.002132 | 0.044091 |
| Reticulocyte.count_irnt_both.ZN_neg | 0 | 1.143351 | 0 | 0.013814 |
| Mean.reticulocyte.volume_irnt_both.ZN_neg | 0.294993 | 0.669823 | 0.270058 | 0 |
| Mean.sphered.cell.volume_irnt_both.ZN_neg | 1.020638 | 0.450778 | 0.200955 | 0 |
| Albumin . . . quantile._irnt_both.ZN_neg | 0.743511 | 0.607999 | 0 | 0.009338 |
| Alkaline.phosphatase . . . quantile._irnt_both.ZN_neg | 0 | 0.731422 | 0.013186 | 0.039227 |
| Alanine.aminotransferase . . . quantile._irnt_both.ZN_neg | 0 | 0.751712 | 0.004737 | 0.034259 |
| Aspartate.aminotransferase . . . quantile._irnt_both.ZN_neg | 0 | 1.245084 | 0.029984 | 0.031954 |
| Urea . . . quantile._irnt_both.ZN_neg | 0.288708 | 1.175512 | 0.008844 | 0.127131 |
| Calcium . . . quantile._irnt_both.ZN_neg | 0.00291 | 1.298992 | 0 | 0 |
| Cholesterol . . . quantile._irnt_both.ZN_neg | 1.043174 | 0.564289 | 0 | 0 |
| C.reactive.protein . . . quantile._irnt_both.ZN_neg | 0 | 0.713619 | 0.127171 | 0.120418 |
| Gamma.glutamyltransferase . . . quantile._irnt_both.ZN_neg | 0 | 0.939174 | 0 | 0 |
| Glucose . . . quantile._irnt_both.ZN_neg | 0.246005 | 0.135532 | 0.036161 | 0.743917 |
| HDL.cholesterol . . . quantile._irnt_both.ZN_neg | 1.050702 | 1.02236 | 0.197789 | 0 |
| IGF.1 . . . quantile._irnt_both.ZN_neg | 0.289632 | 1.204491 | 0 | 0.664273 |
| Lipoprotein.A . . . quantile._irnt_both.ZN_neg | 0.154588 | 0.280427 | 0 | 0.022923 |
| Phosphate . . . quantile._irnt_both.ZN_neg | 0.270463 | 0.955434 | 0.003418 | 0.11104 |
| SHBG . . . quantile._irnt_both.ZN_neg | 0.34278 | 0.470191 | 0.381031 | 0.34375 |
| Total.bilirubin . . . quantile._irnt_both.ZN_neg | 0 | 0.801804 | 0.17247 | 0.025958 |
| Testosterone . . . quantile._irnt_female.ZN_neg | 0.465528 | 0.576326 | 0.002779 | 0.040863 |
| Testosterone . . . quantile._irnt_male.ZN_neg | 0.163238 | 0.666457 | 0.383498 | 0.431608 |
| Total.protein . . . quantile._irnt_both.ZN_neg | 0 | 1.397219 | 0 | 0 |
| Triglycerides . . . quantile._irnt_both.ZN_neg | 0 | 0.768437 | 0 | 0.032372 |
| Urate . . . quantile._irnt_both.ZN_neg | 0.220802 | 0.903966 | 0 | 0.122611 |
| Vitamin.D . . . quantile._irnt_both.ZN_neg | 0.136521 | 0.434081 | 0 | 0 |
| Diastolic.blood.pressure . . . | 0 | 1.14119 | 0.181538 | 0 |
| automated.reading_irnt_both.ZN_neg | | | | |
| Systolic.blood.pressure . . . | 0 | 0.926435 | 0.080083 | 0 |
| automated.reading_irnt_both.ZN_neg | | | | |

TABLE 16
Overview of T2D clusters
Physiological impact, key traits, key loci, suspected
mechanism for each cluster. The numbers in parentheses
next to cluster names indicate the numbers of top-weighted
variants in each of the clusters.

| | Physiological | | | | |
|---|---|---|---|---|---|
| Cluster | impact | Key traits | Key loci | Suspected mechanism | Note |
| Beta-cell 1 (66) | Insulin | CIR (−), DI (−) | MTNR1B, CDKAL1, HHEX, | Beta cell | Beta Cell cluster from |
| | deficiency | | C2CD4A, ANK1, ST6GAL1, | function, glucose | Udler et al. 2018 divided |
| | | | SLC35C1, SLC30A8 | homeostasis | into 2 clusters |
| Beta-cell 2 (27) | | fasting proinsulin adj FI | TCF7L2, SLC30A8, | Beta cell | Beta Cell cluster from |
| | | (+), HOMA-B (−), | ADCY5, GCK, DGKB, | function, insulin | Udler et al. 2018 divided |
| | | fasting insulin (−) | C2CD4A, MTNR1B | processing | into 2 clusters |
| Proinsulin (18) | | fasting proinsulin adj FI | ARAP1, STARD10 | Insulin synthesis | replicated Proinsulin cluster |
| | | (−), HOMA-B (−) | | | from Udler et al. 2018 |
| Lipoprotein A (1) | | lipoprotein A (+) | SLC22A3/LPA | Lipoprotein A metabolism | new cluster in this study |
| Obesity (37) | Insulin | BMI (+), waistC (+), % | FTO, MC4R | Obesity-mediated insulin | replicated Obesity cluster |
| | resistance | body fat (+), CRP (+) | | resistance | from Udler et al. 2018 |
| Lipodystrophy | | adiponectin (−), | IRS, KLF14, PPARG | Fat distribution-mediated | replicated Lipodystrophy |
| (54) | | ISI (−), HDL (−) | | insulin resistance | cluster from Udler |
| | | | | | et al. 2018 |
| Liver/Lipid (12) | | CRP (−), TG (−), | GCKR, HNF1A, PPP1R3B, | Liver/Lipid metabolism | replicated Liver/Lipid |
| | | GGT (−) | TOMM40, PNPLA3 | | cluster from Udler |
| | | | | | et al. 2018 |
| ALP negative (4) | | ALP (−) | ABO | Alkaline phosphatase | new cluster in this study |
| | | | | activity levels | |
| Hyper Insulin | | DI (+), CIR (+) | PPP1R3B, CNTN2, DTNB, | Insulin secretion, | new cluster in this study |
| Secretion (32) | | | SREBF1, TNF | inflammation | |
| SHBG (1) | | SHBG (−) | SHBG | SHBG metabolism | new cluster in this study |

### Example 2—Genetically-Anchored Molecular Signature for Defining Mechanistic Disease Pathways

Current understanding of mechanistic pathways leading to T2D is limited but is critical for much-needed advances in T2D management and prevention. T2D causes significant global health and economic burden,¹⁰ which has been exacerbated in the midst of the COVID-19 pandemic, where T2D is a major risk factor for worse prognonsis.¹¹ Despite multiple therapeutic options available for T2D, remission or cure is rarely achieved.^{12,13} Furthermore, T2D is well-appreciated to be a heterogeneous disease,¹⁴ which is attributable in part to its diagnosis hinged on a non-specific endpoint of hyperglycemia;¹⁵ as many different mechanistic pathways lead to hyperglycemia (e.g. defects in insulin production, secretion, or action), it is generally not clear for a given patient with T2D precisely why the disease has developed, and individualizing patient management remains a challenge.¹⁶ Understanding the mechanistic pathways leading to disease offers a means to i) subtype disease, ii) identify those at highest risk of subtypes, iii) develop targeted therapeutics tailored to each T2D subtype, all advancing T2D management to a precision medicine framework.

Genetic data can help guide “precision medicine” by elucidating disease pathways, but making sense of T2D's complex genetics is challenging, and existing discoveries have lacked clinical or pharmacological translation. In monogenic diabetes, which represents ~0.4% of all diabetes cases, ¹⁷ precision medicine already exists: the underlying gene mutation defines a severely disrupted biological pathway causing diabetes and provides insight into patients' clinical course and management. For example, people with diabetes due to HNF1A and HNF4A mutations suffer from dysfunctional beta cell insulin secretion, and are highly sensitive to sulfonylurea treatment, which promotes insulin secretion.¹⁸⁻²⁰ While most people with T2D will not carry rare monogenic mutations, Applicants and others have shown that individuals with T2D carry more rare variants in monogenic diabetes genes than would be expected by chance.^{21,22} Indeed, the genetic contribution to T2D is exceedingly complex with more than 500 distinct common genetic variants associated with disease,²³ most of which are located outside of protein-coding DNA. How common genetic variation leads to T2D is not fully understood, but it is suspected that thousands of genes contribute to regulatory networks that modify disease risk.²⁴ Since single common genetic variants associated with T2D typically have very small risk estimates, there have been efforts to aggregate risk across thousands of variants, including those not reaching genome-wide significance.² These polygenic scores capture composite risk of T2D and can identify the top 5% of score carriers as having a 4.5-fold increased risk of type 2 diabetes compared to the rest of the population;^{9,25} however since this risk captures many different disease pathways, the driving disease mechanism(s) in people with the highest full T2D polygenic scores is not known. Limited knowledge of T2D genetic mechanisms precludes advances in both disease subtyping and drug development.

T2D has a strong genetic basis, yielding 100's of genetic risk loci, yet clinical translation has lagged.² Polygenic scores combine thousands of genetic variants identified in genome-wide association studies (GWAS) and can stratify people at highest risk of T2D, even before traditional biomarkers become elevated; however, traditional scores capture risk across a conglomerate of disease pathways, precluding elucidation of disease mechanism. Additionally, their predictive ability is currently limited,² though Applicants have recently demonstrated that incorporation of rare genetic variation improves predictive power.^{3,4}

Pathway-specific “partitioned polygenic scores” (pPS) capture a person's genetic risk for a given pathway of disease pathogenesis,⁵ offering exciting potential for clinical translation, but require further enhancement. These pPS can define subtypes of T2D^{5,6} with distinct cellular profiles,⁷ and, as described here, can guide identification of novel drugs. However, existing pPS for T2D have only involved common variants reaching genome-wide significance,^{5,8,9} and could therefore have improved predictive ability by including additional common and rare genetic variants relevant to T2D. This example further describes these polygenic T2D pathways how they can be anchored to specific gene signatures that can identify their key regulatory genes and drug targets.

### Expanded pPS

Applicants provide an improved genetic T2D subtyping that can implicate new T2D genes via enhanced T2D pPS that incorporate additional common and rare variation. Applicants generate enhanced T2D pPS that 1a) include common variants reaching sub-genome-wide significance using summary statistics from multiple published T2D GWAS and 1b) incorporate rare variation using exome and whole genome sequence data (WGS). 1c) The updated pPS can be tested for robust associations with clinical phenotypes in participants from the Mass General Brigham Biobank, the All of Us Research Program, and NHLBI's TOPMed WGS study. An expanded T2D pPS can place hundreds of genetic loci into pathways, including rare and common variants, implicating new T2D loci, and helping to elucidate T2D pathways and subtypes. An enhanced pPS can facilitate identification of polygenic T2D pathways, T2D genetic subtypes, and people at highest risk for these subtypes.

Applicants can elucidate the transcriptional regulation of T2D genetic pathways by generating gene expression signatures associated with the pPS. To demonstrate the transcriptional program of each pPS, gene-expression signatures can be developed in ~60 tissues using the Genotype-Tissue Expression (GTEx) project and additional muscle, adipose, liver, and pancreatic islet tissue expression datasets. Applicants can a) generate expression signatures associated with each pPS and b) perform pathway and transcription factor enrichment analyses based on canonical gene sets and cistrome datasets. A pPS is associated with robust expression signatures that are significantly enriched for functional programs. Novel T2D pPS gene-expression signatures can help characterize T2D pathways. These expression signatures can elucidate regulatory networks of T2D pathways.

Applicants can utilize gene expression signatures from T2D pPS to identify key regulatory genes and drugs. The process-specific gene expression signatures can be mapped to perturbations from 7,494 genes and 19,811 small molecules in the Broad Institute's Connectivity Map (CMap) to identify key regulatory genes and small molecules acting on the pathways. The expression signatures derived from pPS map to pathways that can be modified by specific gene or compound perturbations. In addition to connecting known T2D genes and drugs to genetic pathways, Applicants can identify potential drug targets and therapies. Applicants further describe a novel method for identification of T2D druggable genes and therapeutics.

Improved understanding of the pathways causing T2D can enable patient stratification and discovery of perturbations targeting specific pathways, potentially modifying pathological processes. The novel approach for enhanced disease subtyping and drug discovery can generate robust candidates for follow-up dedicated experimentation, is also applicable to other complex genetic diseases, and can provide a framework for high-throughput identification of novel therapies.

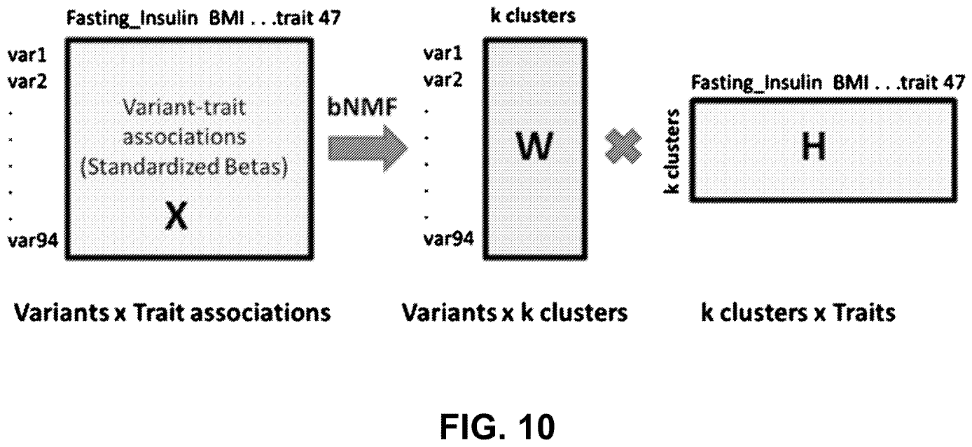

Assigning T2D genetic risk variants into disease pathways is challenging because the majority of signals fall within non-protein coding regions of the genome and have unknown function.⁸ Applicants have recently developed a novel method to move from associated genetic signal to mechanistic pathway.⁵ Applicants leverage published GWAS summary statistics and a soft-clustering algorithm called Bayesian Non-negative Matrix Factorization (bNMF)²⁶ (FIG. 10) to group T2D genetic variants together that share a similar pattern of associations across T2D-related traits. The underlying assumption is that genetic variants that are associated with multiple phenotypes in a consistent manner are likely part of a shared pathway. In the initial work, 94 T2D genetic variants were clustered using 47 traits into five robust groupings, representing readily interpretable mechanistic pathways of T2D⁵. Two clusters signified reduced pancreatic beta-cell function, differing from each other by high vs. low proinsulin levels, representing defective insulin processing vs defective insulin synthesis, respectively (labeled “beta-cell,” “proinsulin”). The three other clusters represented mechanisms of insulin resistance: obesity-mediated (“obesity”), “lipodystrophy-like” fat distribution (“lipodystrophy”), and disrupted liver lipid metabolism (“liver/lipid”). A parallel, independent clustering effort also converged on similar clusters,⁸ further validating these five clusters. Additionally, the clusters were enriched for active DNA regulatory elements in tissues consistent with suspected mechanism (e.g., liver/lipid cluster was significantly enriched for regulatory elements in liver cells, P≤0.001).5 Using the genetic clusters, T2D “partitioned polygenic scores” (pPS) can be calculated, which capture a person's genetic risk for a given disease pathway.⁵ By identifying people with the highest scores, it is possible to generate putative T2D subtypes. For example, people with the top 10% scores for lipodystrophy pPS had significantly lower body mass index (BMI) and high-density lipoprotein cholesterol (HDL), but higher triglycerides and fasting c-peptide levels compared to others with T2D. T2D pPS are also differentially associated with other metabolic conditions, such high blood pressure, kidney disease, and coronary artery disease.^{5,6}

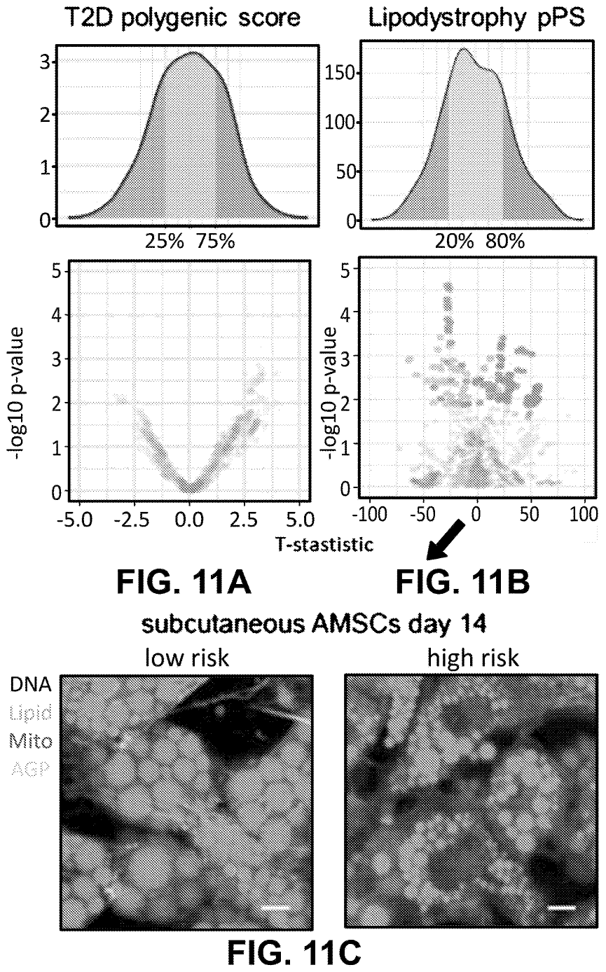

Additionally, it has recently been demonstrated that the T2D pPS can capture molecular and cellular phenotypes.⁷ In vitro differentiated adipose-derived mesenchymal stem cells from subcutaneous (SC) fat biopsies in 26 donors were profiled based on morphological and cellular features. The individuals were first stratified by their full T2D polygenic score, but no differences in image-based morphological and cellular features were notable (FIG. 11a)⁷. In contrast, when stratification was performed using the lipodystrophy T2D pPS, marked differences in profiles were evident: lipocytes in those with high lipodystrophy pPS had increased mitochondrial and actin cytoskeleton informative features vs low lipodystrophy pPS. The differences were visible in ground truth images, indicating increased mitochondrial activity and decreased lipid accumulation in high vs low pPS groups (FIGS. 11b and c)⁷. Furthermore, the high lipodystrophy cellular profile shared a morphological signature with genes causing monogenic lipodystrophy. These findings support that the polygenic lipodystrophy pathway converges on the monogenic pathway, and that the T2D pPS provide insight into T2D biology. Further strategies are described herein to enhance the pPS in order to augment T2D subtyping and drug discovery.

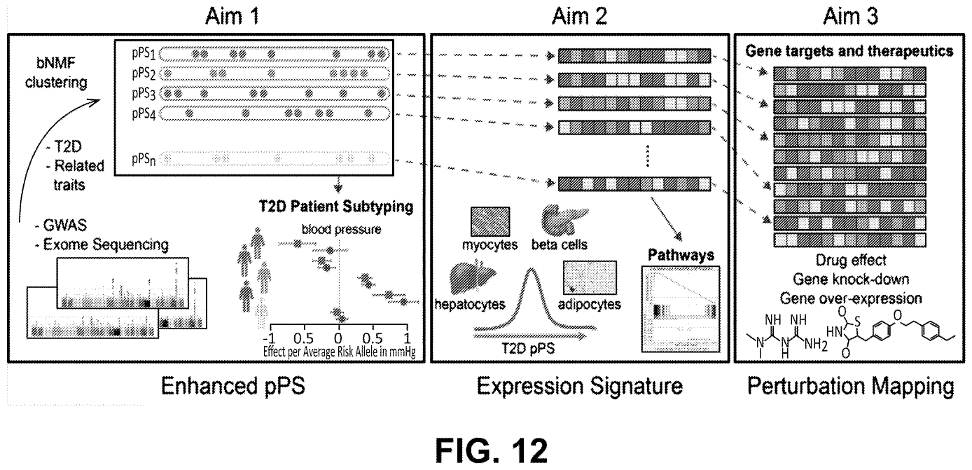

This example shows that enhancing T2D pPS can i) improve T2D genetic subtyping, ii) characterize T2D pathways including their gene expression signatures, and iii) identify key T2D regulatory genes and new therapeutics (FIG. 12). This concept can be applied to any disease that a polygenic risk score can be generated for and where a molecular profile can be provided.

Applicants provide for an improved genetic T2D subtyping and implicate new T2D genes via expanded T2D pPS that incorporate additional common and rare variation. Existing pPS for T2D have been useful for identifying disease pathways and subtypes but contain only a small subset of relevant genetic variants, limiting clinical translation. The enhanced pPS for T2D provides stronger instruments for subtyping T2D and implicates new T2D genes and pathways. The expanded T2D pPS places hundreds of known and novel genetic loci into pathways, including rare and common variants, and through increased power these pPS better define T2D pathways and subtypes. Applicants i) use published summary statistics from multiple GWAS as well as exome sequencing studies to construct enhanced pPS and then ii) validate the scores in large cohorts and biobanks that are richly phenotyped with relevant clinical and genetic data. Applicants can compare the associations with clinical phenotypes and the enhanced T2D pPS to those with currently published pPS to show objective improvement in prediction. The genetic instruments are more informative for drug development and better predictors of clinical outcomes than existing polygenic scores. This addresses the heterogeneity in clinical characteristics seen in patients and allows for the development of pathway-driven therapeutic approaches. The enhanced T2D pPS can define T2D pathways and subtypes, ones that are novel, and identify new putative T2D loci.

The T2D process-specific scores can be expanded by incorporating more common variants, including those reaching sub-genome-wide significance for T2D. To facilitate incorporating additional genetic loci at scale, Applicants have developed a semi-automated high-throughput pipeline to perform genetic clustering (code available on GitHub²⁹). Using this pipeline, Applicants have aggregated summary statistics from 13 T2D studies in the Accelerating Medicines Partnership Common Metabolic Disease Knowledge Portal (AMP-CMDKP),³⁰ from which Applicants extracted 21,666 variants associated with T2D reaching genome-wide significance (P≤5×10⁻⁸). After performing stringent linkage disequilibrium (LD)-pruning (r²<0.1) and optimizing variants for inclusion in the variant-trait association matrix (removing those with >80% missingness or multi-allelic or ambiguous alleles), 336 variants remained, representing independent T2D risk loci. For these 336 variants, Applicants extracted summary statistics for 75 traits related to T2D from GWAS studies of glycemic, anthropometric, and laboratory traits as well as vital signs, all housed either in the AMP-CMDKP³⁰ or in a publicly available analysis of UK Biobank (UKB) data.³¹ Of the 75 traits, 64 were significantly associated with at least one T2D variant at Bonferroni-corrected P<0.05/75=0.00066 and included in the clustering matrix. Soft clustering using bNMF (FIG. 10) was performed using the resulting 336 by 64 variant-trait association matrix and converged optimally on ten clusters. The ten T2D clusters captured the previously published five clusters (with beta-cell separating into two clusters) as well as additional novel clusters. Notably, newer versions of the clusters had more than twice the number of variants

TABLE 17
Demonstrated improvement in enhancing cluster
size by increasing starting set of variants

N variants

| | Original | Preliminary |
|---|---|---|
| | clusters | results |
| | (using 94 T2D | (using 336 T2D |
| Cluster | variants) | variants) |
| Beta cell | 30 | Beta Cell 1: 66 |
| | | Beta Cell 2: 27 |
| Proinsulin | 7 | 18 |
| Obesity | 5 | 37 |
| Lipodystrophy | 20 | 54 |
| Liver/Lipid | 5 | 12 |

To obtain GWAS summary statistics for T2D and related phenotypes used in generating the clustering matrix, Applicants can access publicly available data in dbGaP, UKB, and AMP-CMDKP³⁰. Applicants take a similar approach to previous work⁵, but expand to include additional GWAS datasets (e.g., recent transethnic met-analyses^{23,32}) and permit a relaxed significance threshold for inclusion of T2D variants at multiple Pvalue thresholds (e.g., P<10⁻⁴, 10⁻⁶, 5×10⁻⁸). The expected counts for T2D variants from 14 T2D GWAS studies filtered at various Pvalue thresholds are shown in Table 18. Applicants can perform clustering of these variants using summary statistics from GWAS of at least 75 T2D-related traits. The high-throughput pipeline²⁹ can generate the variant-trait association input matrix and perform bNMF clustering (FIG. 2). The Bayesian implementation of this approach has the advantage of letting the data determine the optimal number of clusters.

TABLE 18
Expected counts of T2D variants for
generation of enhanced T2D pPS

| | N SNPs from | | |
|---|---|---|---|
| | 14 T2D studies | N SNPs | N SNPs |
| | meeting | after LD | after all |
| Pvalue | Pvalue | pruning | filtering |
| threshold | threshold | r² < 0.1 | steps |
| 5 × 10⁻⁸ | 40,993 | 1,021 | 551 |
| 5 × 10⁻⁷ | 63,155 | 1,725 | 805 |
| 5 × 10⁻⁶ | 93,043 | 3,175 | 1,285 |

The T2D process-specific scores can be expanded by combining low frequency and rare variation with common variation into both full and partitioned polygenic scores. Applicants have developed a framework for incorporating rare and common genetic variation and applied to it glycated hemoglobin (HbA1c)-based T2D diagnosis.³ This same method, as described herein, can be extended to full and partitioned T2D polygenic scores.

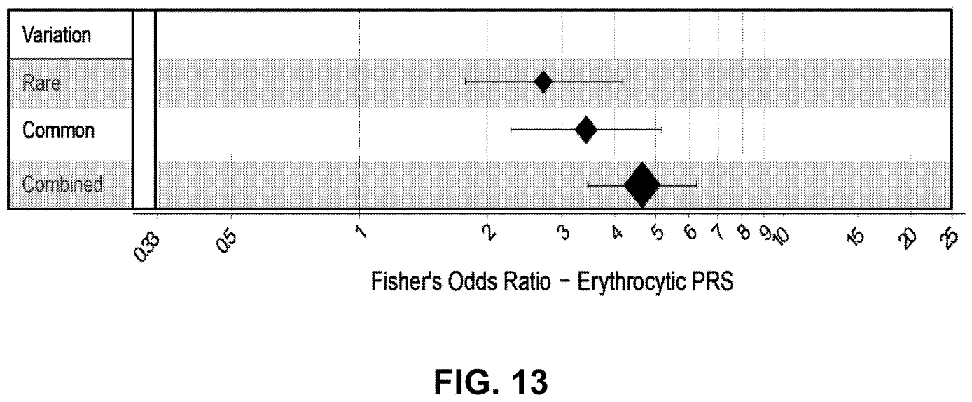

HbA1c values are commonly used to diagnose T2D but can be confounded by erythrocytic (non-glycemic) factors. Therefore, modeling a person's erythrocytic (non-glycemic) genetic factors can improve accuracy of the HbA1c's glycemic prediction.” Building on a previously developed common variant score,³³ Applicants developed a composite rare-common polygenic score to capture a person's HbA1c level, taking into account these erythrocytic (non-glycemic) factors. The main challenge in extending polygenic scores to include rare variation is that many rare variants observed in an individual are too rare to produce accurate effect size estimates (βv) in reasonable “discovery” cohorts. In previous work, Applicants resolved this issue by assigning rare variants to a set of pre-defined groups g, for each of which the needed parameters are constant and estimable at the time the model is built. Applicants then defined a rare variant polygenic score as

$${y}_{i}^{r} = {\sum }_{g} {\sum }_{v ∈ g} {β}_{g} ( {d}_{iv} - ( E ( {d}_{v} ) ) ) = {\sum }_{g} {β}_{g} {\sum }_{v ∈ g} ( {d}_{iv} - ( F ( {d}_{v} ) ) ) = {\sum }_{g} {β}_{g} ( {d}_{ig} - ( E ( {d}_{g} ) ) )$$

where β_{g} is the estimated effect derived for variants within group g, d_{ig} is the combined allelic dosage for all variants in the group, and E(d_{g}) is the combined minor allele frequency of variants in g. Applicants applied this methodology to 200K individuals with exome data in the UKB as a discovery cohort. For all genes in the exome, Applicants defined a ranked set of seven variant groups (or “masks”)³⁴ and calculated the aggregate association between each mask and HbA1c using burden tests, producing seven values of β_{g} for each gene. Burden tests were adjusted for age, sex, and ten principal components (PC's). Applicants included in the risk score genes that achieved P<0.05 for at least one mask and also had evidence from mouse models as impacting erythrocyte function.³⁵ To obtain an integrated rare-common variant polygenic score, Applicants re-calculate the gene-level associations conditional upon nearby common variant associations, modified the rare variant score using the conditional results, and created a combined score as the sum of the common variant and conditional rare variant scores. Applicants then applied the scores to a validation cohort of 42K exomes from the AMP-T2D-GENES study.³⁴ Applicants evaluated score accuracy by identifying individuals who had reported HbA1c <6.5% and were not on diabetes medication but, after accounting for the effects of the score, would be “reclassified” as a T2D case according to their genetically adjusted HbA1c. Applicants then assessed whether the “reclassified” T2D cases were “true” cases, defined as T2D diagnosed also based non-HbA1c measurements. Applicants calculated an odds ratio comparing the proportion of reclassified “true” cases to the proportion expected by chance. Applicants calculated these statistics for the rare variant score, the common variant score, and the combined score. Compared to the exclusively common variant score, the combined score (a) reclassified 50% more true T2D cases and (b) reclassified as controls a similar ratio of true T2D cases (FIG. 13).

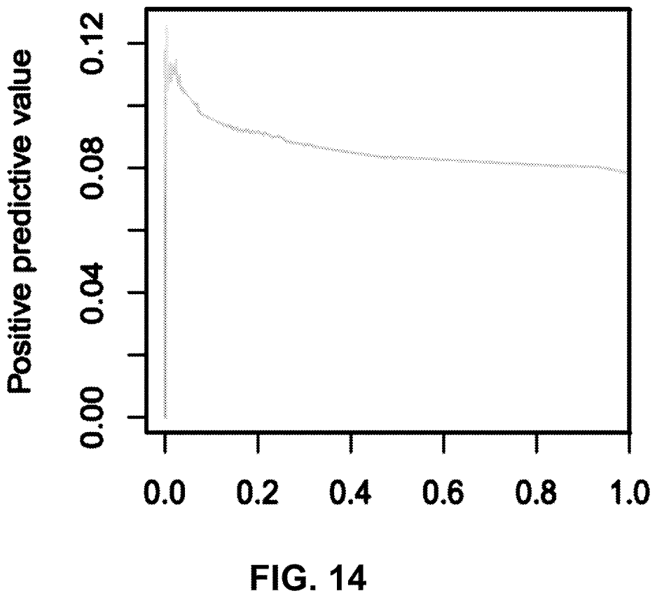

Preliminary application of this method to T2D indicated that a combined rare-common variant score could significantly predict T2D risk. Applicants computed T2D burden statistics for all genes in the AMP-T2D-GENES exomes. Applicants generated a rare variant T2D score using the top five genes with burden tests associated with T2D at P<10⁻⁴ for at least one mask in AMP-T2D-GENES. Applicants then applied this rare variant score to 200K individuals with exome data in the UKB. The results (FIG. 14) show a statistically significant ability of the rare variant score to predict T2D (P=1.5×10⁻¹⁹). These findings support that the T2D rare variant score would contribute significantly when combined with the T2D common polygenic score.⁹

The following studies can be training datasets^{4,34} to develop rare variant polygenic scores. Phenotypes available in the datasets are in Table 19.

TABLE 19
Traits available in datasets with exome sequence

| Trait category | AMP-T2D-GENES | UK Biobank |
|---|---|---|
| Glycemic | T2D, HbA1c, Fasting | T2D, HbA1c, Fasting |
| | Glucose, 2 hr Glucose, | Glucose, Random |
| | Fasting Insulin, 2 hour | Glucose |
| | Insulin, Fasting c- | |
| | peptide, 2 hr c-peptide | |
| Other serum | Total cholesterol, LDL, | Total cholesterol, |
| laboratory | HDL, Triglycerides, | LDL, HDL, |
| values | creatine, Leptin, | Triglycerides, Liver |
| | Adiponectin | Function tests, |
| | | Complete blood |
| | | counts, CRP, urate, |
| | | SHBG, IGF-1, 20 + |
| | | additional labs |
| Vital signs and | Height, Weight, Hip | Height, Weight, Hip |
| anthropometric | circumference, Waist | circumference, Waist |
| measures | circumference, SBP, | circumference, SBP, |
| | DBP | DBP, HR, percent |
| | | body fat |

AMP-T2D-GENES³⁴: This cohort consists of 20,791 T2D cases and 24,440 controls from multiple distinct multi-ancestry studies for which there is exome sequence and phenotype data. Analysis of the data has been approved by the Mass General Brigham (MGB) institutional review board (IRB) (protocol #2017P000445/PHS).

UKB³⁶: This prospective cohort has ~500K recruited individuals from the United Kingdom, with genotype, phenotype, and linked EHR data. The analyses can utilize exome data generated for 450K participants. Analysis of the data was approved by the MGB IRB under UK Biobank application 27892.

Applicants can first generate a rare variant polygenic score for T2D (full score), using the method described above. Applicants can derive T2D gene sets using 1) the top T2D gene burden results in the training datasets (e.g., those with P<0.05) and 2) knock out mouse models of hyperglycemia from the Mouse Genome Database.³⁵ Applicants use these genes to generate the rare variant score for T2D and then combine it with the common polygenic T2D score,⁹ as described, to generate the combined rare-common score.

Applicants can then extend the methodology to generate a rare variant T2D pPS. The best performing T2D gene set for the full-T2D rare variant polygenic score can provide the starting set of genes. T2D pPS are defined by a set of weights

$${w}_{j}^{p}$$

across traits j=1 . . . t. For example, the common variant “lipodystrophy” pPS has as strongly weighted traits: reduced BMI, increased fasting insulin adjusted for BMI, increased serum triglycerides, and reduced HDL cholesterol.⁵ For process p Applicants can first generate process-specific effect sizes

$${\hat{β}}_{i}^{p}$$

and standard errors

$${σ}_{i}^{p}$$

for each mask through a weighted sum of the trait-specific effect sizes

$${\hat{β}}_{i}^{p}$$

and standard errors

$${σ}_{i}^{j}$$

$${\hat{β}}_{i}^{p} = {\sum }_{j = 1}^{t} {w}_{j}^{p} {\hat{β}}_{i}^{j} ; {( {σ}_{i}^{p} )}^{2} = {\sum }_{j = 1}^{t} {( {w}_{j}^{p} )}^{2} {( {σ}_{i}^{j} )}^{2} ; {Z}_{i}^{p} = \frac{{\hat{β}}_{i}^{p}}{{σ}_{i}^{p}}$$

and then use these values analogously to those for any other trait. This weighted sum can allow for quantifying each gene's membership into the various T2D clusters (defined by common variants) based on each gene's burden testing result for the set of traits. Using this approach, Applicants can develop rare variant T2D pPS using whole exome sequence data from AMP-T2D-GENES³⁴ and the UKB.^{36,37} Within each study and for each mask, Applicants can conduct burden tests for all available traits included in the T2D common pPS (Table 19). The vast number of traits in the UKB can complement the glycemic traits available in AMP-T2D-GENES, together providing substantial representation of the traits highly weighted in the common variant T2D pPS. Applicants can meta-analyze results across AMP-T2D-GENES and UKB for each mask and each trait and apply the equation above to produce gene-level weights for each T2D pPS. As the genes have weights for each pPS, Applicants can determine the optimal threshold for including genes in each score by ranking them by

$${Z}_{i}^{p}$$

and using cross-validation. The rare variant T2D score for each pPS can then be added to the common variant score to generate a combined rare-common pPS, which can be validated in independent datasets.

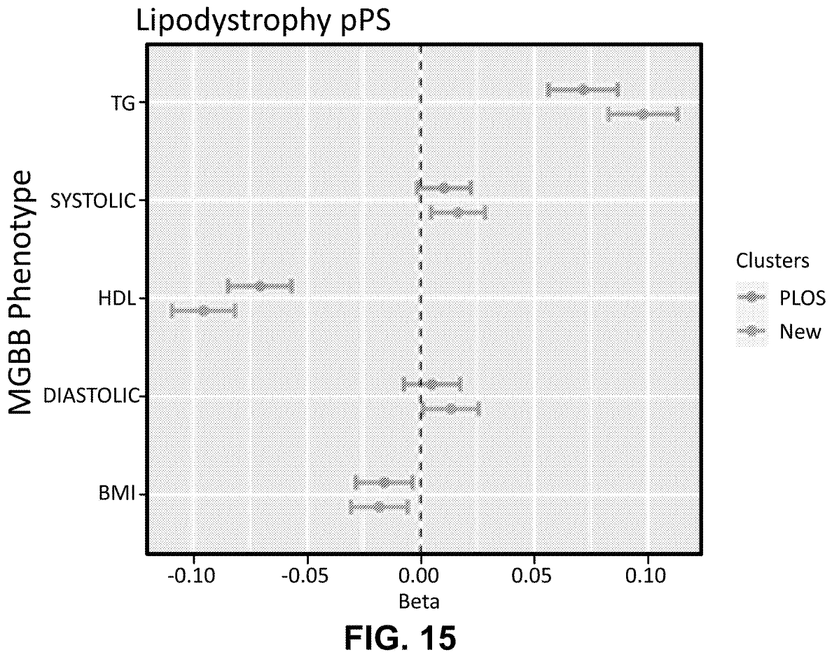

Applicants can assess the clinical impact of enhanced T2D pPS in independent studies: MGB Biobank, TOPMed, and All of Us. Applicants previously demonstrated that five T2D pPS derived from clustering 94 T2D variants were associated with clinical phenotypes and outcomes in individual-level datasets,^{5,6} but that these associations had modest effect sizes. Applicants can improve the predictive ability of pPS. To demonstrate feasibility, Applicants analyzed the updated T2D pPS generated (Table 17) in 33,745 individuals in the Mass General Brigham Biobank (MGBB) and compared results to the original five T2D pPS. The results for the lipodystrophy pPS are representative, demonstrating that the updated pPS had more marked associations with relevant clinical features (FIG. 15). These findings indicate that the enhanced lipodystrophy pPS is a stronger genetic instrument for downstream analyses. Applicants expect even larger improvements with additional variation incorporated.

Applicants can construct pPS in three datasets that are independent from the datasets used to derive the pPS: MGB Biobank (MGBB), TOPMed, and All of Us (Table 20).

MGB Biobank (MGBB)³⁸: The MGBB has genotyped >53,000 consented patients seen at hospitals and clinics across the MGB healthcare system. Phenotypic clinical data are available via patient EHR. Analyses are covered by IRB 2016P001018. Quality control (QC) of the genetic data and imputation to the TOPMed Reference Panel³⁹ have already been performed.

All of Us Research Program⁴⁰: This NIH-funded research program has developed a large and diverse cohort (anticipated N=1,000,000; current Registered Tier Dataset v5 N=329,050) with accompanying clinical data including laboratory values (v5 Sample with EHR data N=214,206).

Trans-Omics for Precision Medicine (TOPMed)³⁹: This cohort sponsored by the National Heart, Lung, and Blood Institute (NHLBI) consists of ~180K participants from >85 studies, including biobanks and cohorts. Data is released in freezes, with currently WGS and harmonized phenotype data available for 81,642 (Freeze 9). As Freeze 10 sequencing has completed and is undergoing QC, the sample size will increase imminently.

To characterize clinical features of potential genetic subtypes of T2D, Applicants can generate the T2D pPS in individuals from independent validation cohorts, MGBB,³⁸ TOPMed,³⁹ and All of Us⁴⁰ (Table 20). The common variant pPS can be generated using a standard weighted method where by the number of risk alleles (0, 1, or 2) at each SNP are summed and then multiplied by their cluster weight, then multiplied by the number of SNPs in the genetic risk score and divided by the sum of all the weights.⁴¹ Rare variant pPS can be added to the corresponding common variant pPS in the studies with WGS to generate rare-common pPS. To test the association between each pPS and each clinical phenotype (Table 21), Applicants can use regression models (logistic for dichotomous outcomes, linear for continuous traits). Analyses can be adjusted for age, sex, reported race/ethnicity, and the top ten ancestry-specific PC's. T2D-adjusted and T2D-restricted analyses can be performed as sensitivity analyses. A Bonferroni level of significance can be used to reduce reporting false positive associations in the context of multiple testing. To assess improvement between enhanced pPS that represent one of the five previously published pathways,⁵ Applicants can test for stronger associations with expected clinical phenotypes (Table 22), demonstrated by non-overlapping 95% confidence intervals of regression beta estimates.

TABLE 20
Study characteristics

| | MGBB | All of Us* | TOPMed |
|---|---|---|---|
| N total | 53,316 | 329,050 | 81,642 |
| % Male | 45% | 39% | 41% |
| % European | 84% | 48% | 49% |
| ancestry | | | |
| N with T2D | 4,355 | 40,040 | 20,737 |
| Genetic data | SNP Array | WGS | WGS |

*All of Us data reflects Tier Dataset v5; genomic data expected March 2022

TABLE 21
Individual-level data available in the studies
Clinical Data

| | |
|---|---|
| Vital signs | DBP, SBP, heart rate, weight, height, body-mass index |
| Measures of renal function | Serum creatinine, estimated glomerular filtration rate, urine |
| (serum and urine studies) | microalbumin |
| Measures of liver function | aspartate aminotransferase (AST), alanine aminotransferase |
| (serum studies) | (ALT), alkaline phosphatase |
| Lipid traits (serum studies) | Total cholesterol, LDL cholesterol, HDL cholesterol, |
| | Triglycerides |
| Glycemic traits (serum traits) | Glucose (random or fasting), hemoglobin A1c |
| Medications (used as either | Including diabetes, blood pressure, and cholesterol |
| outcomes or to | medications |
| adjust other measures) | |
| Other disease diagnoses: | Diagnosis of: Coronary artery disease (CAD), chronic kidney |
| | disease (CKD), ischemic stroke |

TABLE 22
Examples of expected associations in high
vs low score carriers for published pPS^{5, 6}

| | Clinical phenotype associations |
|---|---|
| T2D pPS | in individual-level data |
| Beta Cell | Increased SBP, CAD risk |
| | Decreased DBP |
| Proinsulin | Decreased DBP |
| Obesity | Increased BMI, SBP, DBP, HTN |
| Lipodystrophy | Increased Triglycerides, SBP, |
| | DBP, HTN, CAD risk |
| | Decreased HDL, BMI |
| Liver/Lipid | Increased creatinine, CKD |
| | Decreased Triglycerides, eGFR, |
| | CAD |

This can generate a robust clinical characterization of new enhanced pPS derived from augmented T2D genetic clusters. Applicants also expect to uncover new putative T2D loci that have T2D associations below the standard GWAS threshold, but their coordinated associations with other traits can increase the likelihood that they are genuine T2D loci.

Elucidating the Transcriptional Regulation of T2D Genetic Pathways by Identifying Gene Expression Signatures Associated with pPS.

Current understanding of the complex genetic regulatory networks contributing to T2D pathophysiology is very limited, but critical for exploiting disease pathways for clinical and pharmacological translation. Applicants can identify T2D pathway-specific gene expression signatures that capture transcriptional regulation related to each T2D pPS. Applicants hypothesized that T2D pPS is associated with gene expression sets that are robust and are significantly enriched for relevant functional programs and transcription factor binding sites. Applicants can i) develop gene-expression signatures in the Genotype-Tissue Expression (GTEx) project as well as in adipose, pancreatic islets (using TIGER study dataset), muscle, and hepatic tissue b) perform pathway and transcription factor enrichment using analyses of canonical gene sets and cistrome datasets. The gene-expression signatures and analysis provide improved understanding of genetic transcriptional programs for pathways of T2D that can be utilized for drug development. The T2D pathway-specific gene expression signatures can define transcriptional programs for T2D pathways, characterized by enrichment of canonical gene sets and transcription factor binding sites.

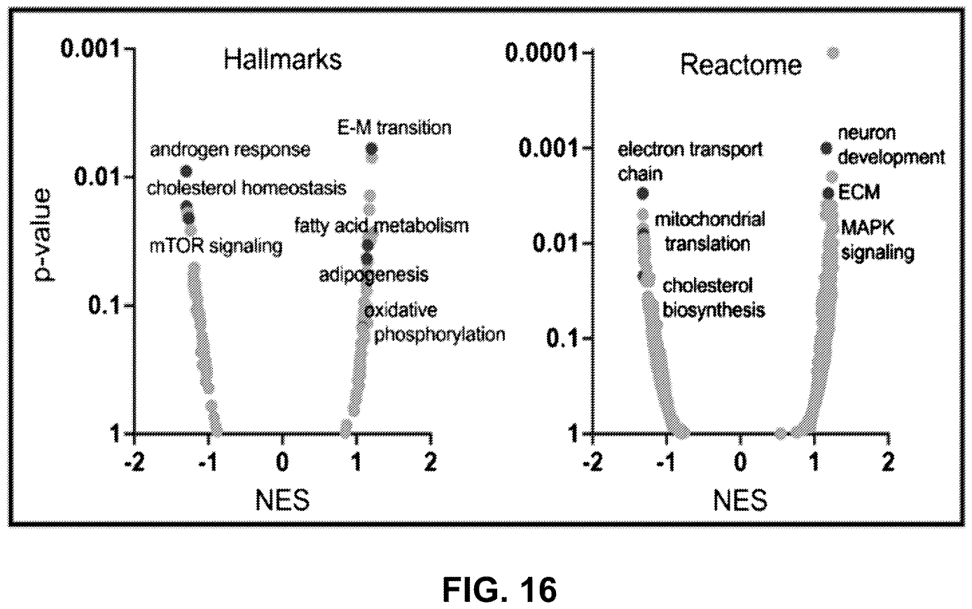

Applicants have used published T2D pPS⁵ to generate gene expression signatures in data from SC adipocytes derived from 26 donors from Munich Obesity BioBank (MOBB, dataset described below).⁷ Applicants constructed the “beta-cell” and “lipodystrophy” pPS in all individuals, focusing on these two pPS since they have the most genetic variants of any cluster (30 and 20, respectively). Applicants ran linear regression models with each pPS for each tissue transcript, adjusting for age, sex, and batch. The resulting vector of 1000's of transcript expression changes per increase in pPS defines the gene expression signature for each pPS. Gene set enrichment analysis of the beta cell pPS expression signature had top findings related to gene expression in early pancreas (in BioPlanet 2019, Elsevier Pathway Collection, Reactome 2016, all P<0.05, Q<0.3). This finding was remarkable given that expression data came from such a small dataset (26 samples) and from SC adipose tissue, since the primary active tissue for the beta-cell pPS is expected to be pancreas.⁵ In contrast, the lipodystrophy pPS expression profile in the same 26 SC adipose samples was nominally enriched for different processes (P<0.05, but Q<0.1), including electron transport chain and mitochondrial translation (FIG. 16). These findings were consistent with image-based lipocyte profiling performed in the high vs low lipodystrophy pPS in the same samples, which strongly converged on features of mitochondrial hyperactivity⁷ (FIG. 11b,c). Both the beta-cell and lipodystrophy pPS were composed of T2D risk increasing alleles, yet they demonstrated markedly different gene expression signatures and enrichment patterns, highlighting the benefit of analyzing pPS rather than the full T2D polygenic score. These results can benefit from using the enhanced pPS and additional tissue expression datasets.

Applicants can further generate robust gene expression signatures for each T2D pPS. Applicants can utilize multiple datasets with tissue expression and genetic data (Table 23):

The Genotype-Tissue Expression (GTEx)⁴² is a publicly available resource of 49 non-disease tissues collected from up to 838 human deceased donors. Expression data were generated with Illumina TruSeq RNA sequencing and WGS with Illumina HiSeq. Covariates for analysis include the top 5 genotyping PCs, sex, sequencing platform, sequencing protocol (PCR-based or PCR-free), and Probabilistic Estimation of Expression Residuals (PEER)⁴³ factors. Data are available via dbGAP (phs000424.v9).

Munich Obesity BioBank (MOBB)⁴⁴ contains SC and visceral fat biopsies from 26 and 31 people, respectively, taken while undergoing abdominal laparoscopic surgery. Each participant gave written informed consent, and the study protocol was approved by the ethics committee of the Technical University of Munich (Study 5716/13). Genotype data was generated with the Illumina Infinium Global Screening Array BeadChip with imputation performed using the Haplotype Consortium Reference (HRC) Panel reference panel.⁴⁵ RNA-seq data was generated from primary human adipose derived mesenchymal stem cells (AMSCs) at differentiation days 0, 3, 8, and 14. QC of the genotype and RNA-seq data has been described in detail.⁷ Covariates for analysis include the top 5 genotyping PCs, age, sex, BMI, and batch.

Translational Human Pancreatic Islet Genotype Tissue-Expression Resource (TIGER)⁴⁶ has generated a large eQTL resource for pancreatic islets obtained from >500 organ donators. Recent analysis of 404 islet samples identified 35 novel T2D effector genes including some driven by low-frequency variants.⁴⁶ Genotype and expression data platforms varied by contributing study.

The Finland-United States Investigation of NIDDM Genetics (FUSION) Tissue Biopsy Study (FUSION)⁴⁷ involved 271 individuals with variable levels of glucose tolerance: 97 normal glucose tolerance (NGT), 71 impaired glucose tolerance (IGT), 36 impaired fasting glucose (IFG), and 67 newly diagnosed T2D. Skeletal muscle biopsies from the 271 individuals and abdominal SC adipose tissue from a subset of 251 are available in dbGaP(phs001048). Expression data was generated with Illumina TruSeq RNA sequencing and genotyping on the Illumina Omni2.5M array with imputation using the HRC reference panel.⁴⁵

Phenotyping Lipid traits in iPS derived hepatocytes Study (PhLiPS)⁴⁸ focuses on individuals free of cardiovascular or lipoprotein metabolism disorders in the community served by the Hospital of the University of Pennsylvania. Data from 89 individuals has been made available on dbGAP (Accession: phs001341.v1.p1), including RNA-seq data from induced pluripotent stem (iPS) cell-derived hepatocytes using HiSeq 2000. The gene expression data is paired to participant genotype data using Infinium Human CoreExome-24 BeadChip.

TABLE 23
Datasets for generation of pPS gene expression signatures

| | N | | Age | | | | |
|---|---|---|---|---|---|---|---|
| Study | individuals | Tissue(s) | (mean) | % Male | % White | SNP array | WGS |
| GTEx | 838 | 54 including: Subcutaneous adipose, | 53 | 67% | 85% | ✓ | ✓ |
| | | visceral adipose, pancreas, liver, | | | | | |
| | | skeletal muscle | | | | | |
| TIGER | 404 | Pancreatic islets | 56 | 53% | 100% | ✓ | |
| MOBB | 26 | Subcutaneous adipose | 44 | 25% | 100% | ✓ | |
| | 31 | Visceral adipose | | | | | |
| FUSION | 271 | Skeletal muscle | 60 | 55% | 100% | ✓ | |
| | 251 | Subcutaneous adipose | | | | | |
| PhLIPS | 89 | IPS derived hepatocytes | 32 | 44% | 55% | ✓ | |

WGS: whole genome sequence

In each dataset, Applicants can calculate T2D pPS, both published⁵ and enhanced (see above). Since the cohorts used different tissue isolation protocols, genotyping arrays, and RNA sequencing technology, Applicants can perform QC separately for each (as done in TIGER⁴⁶). Genotype imputation using multiple reference panels, including the TOPMed³⁹ can be performed to obtain the most comprehensive set of genetic variation. The pPS involving rare and common variation, as well as expanded pPS, can be generated in the GTEx dataset since it has WGS. Likewise, QC can be performed on each expression data set separately following the protocols developed for each cohort. Although the downstream analysis can be adjusted for different cell types, weighted normalization methods can be used to address expression variances across different cell types. RNA-seq data can be aligned with STAR⁴⁹ to the newest GENCODE⁵⁰ reference database. Further PC-based QC steps can be considered to address covariate effects.

For each cohort and tissue dataset, Applicants can run linear regression models with each pPS and every transcript as an outcome, adjusting for age, sex, the top five PC's, and study-specific covariates including PEER factors⁴³ when appropriate, and producing an effect estimate (beta), Pvalue, and Qvalue. The regression beta represents the change in transcript level per change in the pPS. The analysis therefore produces a vector of 1,000's of gene expression changes per SD change in the pPS. This vector represents a pPS expression signature for each tissue (or expression quantitative trait score, “eQTS”⁵¹). Applicants can meta-analyze expression signatures for a given pPS across shared tissues (e.g., SC adipose in MOBB and GTEx). Since Applicants can generate expression signatures in multiple tissues, to determine which signature(s) to carry forward, Applicants can prioritize pPS signatures with the largest magnitude of regression beta values, indicating largest mean expression changes.

When sample size allows, Applicants can perform i) sex-stratified analyses to understand if gene expression profiles differ in men and women and ii) analysis of European-only ancestry since the pPS were derived in datasets of predominantly European ancestry due to published data availability and methodological constraints.

Power Calculation: Applicants estimate having at least 80% power to detect at a 70% change in single transcript expression per SD increase in pPS in the smallest dataset (N=26) at P<0.05. Of note, the downstream analyses generally consider a multi-gene expression signature rather than a single gene effect.

Applicants can further identify functional programs related to each T2D process specific genetic cluster. After identification of one or more robust gene expression signatures for each pPS, Applicants can perform in silico functional characterization of the gene expression signatures. Applicants can test for enrichment of established get set libraries including Gene Ontologies (GO),⁵²,” Reactome,¹⁴ BioPlanet,” KEGG,⁵⁶ and using establish gene-set enrichment tools including GSEA,⁵⁷ the Ingenuity Pathway Analysis Tool,⁵⁸ and Gene-List Network Enrichment Analysis (GeLiNEA).⁵⁹ Applicants can also assess the pPS gene expression signatures for enrichment of transcriptional regulators using the “epigenetic Landscape In Silico deletion Analysis” (Lisa).⁶⁰ Lisa incorporates chromatin profile data and transcription factor/chromatin regulator ChIP-seq datasets from human and mouse studies to assess for enrichment of transcription factor and chromatin regulator binding sites across the top genes in an expression signature. Additionally, Applicants can use the Ensembl Variant Effect Predictor (VEP)⁶¹ to assess for pathway interactions between genes. For all enrichment analyses, Applicants can focus on results reaching significance after accounting for multiple testing using a Qvalue<0.1 threshold and expect all real biology to be captured by more than one tool.

In addition to characterizing the biological pathways underlying each T2D pPS, the enrichment tools can also help prioritize tissue signatures to move forward to analyses to identify key regulatory genes and drugs and improve the ability to capture true signal vs noise; since several of the unenhanced T2D pPS relate to pathways that are well-established (e.g. abnormal adipose differentiation, obesity, beta-cell function and have expected enrichment results (as seen herein), capturing expected enrichment signals can provide positive controls for the integrity of the analysis process and prioritize which tissue expression signatures are chosen for downstream analyses of each pPS.

Given that FUSION includes tissues samples for individuals in different stages of diabetes development (normal glycemia, pre-diabetes, and newly diagnosed T2D), Applicants can compare pPS expression signatures developed in each of the three groups using Adjusted Rank Index (ARI), Fowlkes-Mallows Index, and Equivalence Mismatch Distance. Applicants can identify which genes are shared and distinct across the three groups.

Gene expression signatures for each T2D pPS can define pathway-specific transcriptional programs described by enrichment of canonical gene sets and transcription factor binding sites.

In datasets involving whole tissues, there is heterogeneity of cell types and possibly artifacts of tissue collection (e.g., effects of death for GTEx).⁶² Applicants can follow best practice analysis procedures developed by GTEx,⁴² including controlling for covariates. If Applicants find that expression profiles differ greatly between matched tissue types in different datasets, Applicants can perform analyses on the subset of top genes with shared directionally in the expression signatures, thus removing genes with likely study-specific artifact. If a given pPS does not have a robust expression signature across cell types and tissues from the same organ system, Applicants can generate a new hybrid signature including the genes with highest (e.g., >60%) representation across all tissues. If gene expression signatures are not enriched for functional programs, they can be deprioritized in subsequent analyses.

Applicants can further use the gene expression signatures from T2D pPS to identify key regulatory genes and drugs. The gene expression signatures associated with pathway-specific genetic risk can be used to discover novel T2D therapeutic targets. Applicants demonstrate herein the feasibility of using gene expression signatures of T2D pPS and CMap to identify key regulatory genes and relevant disease drugs. Applicants show that expression signatures of T2D pPS can be either matching or opposing to those of relevant pathway-acting perturbagens, and they can be used to identify genes and drugs acting on each pathway. Applicants a) start with a defined set of known T2D regulatory genes and medications and demonstrate enrichment of shared expression signatures with the T2D pPS, and b) query the full 7,494 gene- and 19,811 small molecule-perturbations in CMap to identify those most shared with each of the T2D pPS. This is a novel, powerful method to identify relevant disease drugs and regulatory genes. Applicants can connect multiple genes and drugs to T2D pathways, thereby generating a short-list of novel disease-related genes and small molecules for further pharmacologic evaluation.

Starting with the expression signature in SC derived adipocytes for 1) the beta cell pPS and 2) the lipodystrophy pPS, Applicants calculated similarity scores between the expression signatures for each pPS and 27,927 perturbagens in the Connectivity Map (CMap). As a preliminary analysis, Applicants utilized the CMap web-based interface⁶³ to analyze the top 200 most significantly differentially expressed genes in each of the two pPS. Remarkably, the beta-cell pPS expression signature mapped with high connectivity to glipizide, with the third highest score overall (score 0.93 observed in PC3 cells (max score=1.0), Qvalue=0.39). Glipizide is a sulfonylurea (SU) that stimulates insulin secretion by binding to and closing the ATP-sensitive potassium channel on the beta-cell membrane.⁶⁴In the PC3 cells glipizide's connectivity score was in the opposite direction expected, although in pancreatic tissue (YAPC) its score was in the expected direction. SU's are recommended for treating monogenic diabetes caused by HNF1A and HNF4A mutations, where the reduced function of the gene product leads to impaired insulin secretion that can be overcome with sulfonylureas like glipizide.¹⁹In the analysis, the HNF4A gene over-expression perturbation in YAPC cells also had strong connectivity (score=−0.9) in the expected direction of effect.

When Applicants evaluated the lipodystrophy pPS expression signature in CMap, the overall mapping had lower signal than observed with beta-cell pPS, with maximum similarity score of 0.57 for any perturbagen. Given that the lipodystrophy pPS is appreciated to capture the same pathway as genes causing monogenic lipodystrophy^{5,7}, Applicants assessed connectivity with 1) PPARG, a gene whose loss of function (LOF) causes autosomal dominant partial lipodystrophy, and 2) any member of the diabetes drug class thiazolidinediones, which act on this pathway. Indeed, PPARG LOF had the eighth highest gene connectivity scores (0.49, Q=0.26), with directionality of the effect consist with biological expectation. Of all compounds, the fifth highest scoring was ciglitazone (−0.51, Q=0.23 in MCF10A cells), a member of the thiazolidinedione drug class and in the expected direction of effect.

The preliminary results show that there is clear signal for expression profiles of T2D pPS to map to highly relevant gene and drug targets in CMap. As the preliminary results involved unenhanced T2D pPS, the smallest of the tissue expression datasets, and an analysis restricted to the top 200 genes, the proposed analyses are expected to improve substantially using enhanced T2D pPS, additional and larger tissue expression datasets, and inclusion of the full expression gene list in analyses.

Connectivity Map (CMap)⁶⁵ is an expansive catalog of cellular responses to perturbations developed with the goal of finding relationships between diseases, genes, and drugs. CMap is housed at the Broad Institute and contains over 470,000 gene expression signatures from ~20,000 small-molecule compounds, and ~7,500 genetic reagents, tested in 77 possible cell lines (Table 24). The genetic perturbations include gain of function or LOF assays, and the small molecule compounds were compiled from multiple screening libraries. The gene expression data were generated using “L1000,” a high-throughput gene expression assay that measures the mRNA transcript abundance of 978 “landmark” genes selected by a data-driven approach to be able to recover 82% of connections made using the full transcriptome. Thus, expression of 11,350 additional genes in the transcriptome were inferred using L1000 for a total of 12,328 unique genes reported in CMap.

TABLE 24
Selected cell types analyzed in CMap with
corresponding similar tissues as above

| | | Similar tissue in Gtex |
|---|---|---|
| Cell line | Description | or other studies |
| HA1E | Kidney epithelial cells | Kidney, cortex Kidney, |
| | | medulla |
| HEPG2 | Hepatocellular carcinoma | Liver, iPS derived hepatocytes |
| SNU398 | Hepatocellular carcinoma | Liver, iPS derived hepatocytes |
| PC3 | Prostate adenocarcinoma | Prostate |
| PANC1005 | Pancreatic adenocarcinoma | Pancreas, pancreatic islets |
| PANC0813 | Pancreatic ductal | Pancreas, pancreatic islets |
| | adenocarcinoma | |
| CAPAN2 | Pancreatic adenocarcinoma | Pancreas, pancreatic islets |
| YAPC | Pancreatic adenocarcinoma | Pancreas, pancreatic islets |
| HME1 | Breast mammary | Breast |
| MCF10A | Epithelial cell | Skin +/− sun exposure |

Applicants can further determine whether expression signatures of known regulatory genes and diabetes medications map to the T2D pPS. Applicants can test whether T2D pPS expression signatures can further characterize T2D pathways by linking them to T2D regulatory genes and medications. To do this, Applicants can leverage CMap's extensive gene and small molecule perturbations to query 25 T2D drugs (Table 25) and 41 genes predicted to be causal for T2D⁶⁶ (which include eight monogenic diabetes genes). Expression profiles have been generated for these T2D drugs and genes in various cell lines (Table 24) in CMap. Applicants can test for similarity between CMap's perturbagen expression signatures and the T2D pPS expression signatures using “connectivity” scores.

TABLE 25
Known diabetes drugs (not all in active use)
available in CMap by mechanism of action

| Drug class | Number of compounds available in CMap |
|---|---|
| Glucosidase | acarbose |
| inhibitor | |
| DPP4 inhibitor | aloglitpin, anagliptin, linagliptin, saxagliptin, |
| | sitagliptin, teneligliptin, trelagliptin |
| Thiazolidinedione | ciglitazone, pioglitazone, rosiglitazone, troglitazone |
| Biguanides | metformin |
| SGLT2 inhibitor | canagliflozin, dapagliflozin, empagliflozin, |
| | ertugliflozin, tofogliflozin |
| GLP1RA agonist | exenatide, liraglutide |
| Sulfonylurea | gliclazide, glimepiride, glipizide, gliquidone, glyburide |

Connectivity scores can be calculated using the same approach as the CMap standard analysis.⁶⁵ Briefly, the connectivity score captures the degree to which the pPS expression signature's up-regulated genes appear toward the top of the rank-ordered genes in the perturbagen's signature, and the down-regulated query genes appear toward the bottom of the signature (or vice versa for negative connectivity). The similarity score is calculated using the weighted Kolmogorov-Smirnov enrichment statistic⁵⁷ and then normalized to allow for comparison of scores across cell types and perturbation types. Applicants can calculate similarity scores between pPS and perturbagens using the full inferred gene expression profile via an application programming interface (API). Beyond individual connections, Applicants can consider sets of CMap perturbagens connecting to the same pPS collectively by checking their enrichment as a group in existing pathway (e.g., The Molecular Signatures Database (MSigDB)⁵⁷) and biological network (e.g., STRING⁶⁷) databases. This approach, including mapping drugs to pathways through their annotated targets, can improve interpretation of CMap query results in a way analogous to the generation of pPS scores to interpret T2D GWAS.

For each T2D pPS, Applicants can select one or more tissue expression profiles from the multiple generated, prioritizing based on magnitude of mean expression signal, high similarity to other tissues of the same organ type, and significant enrichment for functional programs that are seen across multiple gene enrichment sets. When a matching tissue- or organ-type is present in CMap (Table 24), Applicants can restrict analyses to that organ-type to reduce the analysis space and improve specificity. At the same time, the preliminary results suggested that high connectivity can exist between different tissues (e.g., beta cell pPS expression signature in SC adipose with high similarity to glipizide's signature in PC3 prostate adenocarcinoma cells), so when a matched tissue is not available in CMap, Applicants still proceed with analyses. Applicants can post-process CMap connectivity analyses to candidate enriched pathways with aggregation both within and across CMap cell line results.

Using the selected set of T2D pPS expression signatures, for one pPS at a time, Applicants first assess for any very high confidence connectivity (score >0.9 and FDR<0.1) with any of the 41 T2D genes or 25 drugs. In addition, Applicants can assess for higher connectivity than by chance with the full set of T2D drugs and genes. To do this Applicants can simulate random draws of lists of 25 drugs and 41 genes from CMap to establish a connectivity null distribution empirically. Applicants can use the normalized connectivity scores of each gene or drug as rankings of connectivity with a given pPS expression signature. Applicants can then use a one-side Wilcoxon rank-sum test to assess whether the set of T2D genes or drugs have significantly higher ranks than the comparison sets. Applicants can correct for multiple testing based on the number of pPS examined. For pPS expression profiles with significant enrichment for the set of 25 drugs, Applicants can further assess enrichment by drug class and (by extension) biological pathways (Table 25).

Applicants can further identify novel regulatory genes and drug targets using pPS expression signatures. Applicants can use the T2D pPS expression signatures to discover new drugs and genes acting on the pPS pathways. Applicants can consider all 7,494 gene- and 19,811 small-molecule-perturbations. As described, Applicants can focus on high confidence pPS tissue signature profiles identified so as to reduce the massive analysis space inherent in testing the full CMap database. Each T2D pPS-tissue expression signature can be tested for similarity with perturbagen signatures by computing similarity scores as described herein. For each pPS tissue-expression signature, Applicants can generate a list of any drugs or genes scoring as connected (e.g., >0.95 or <−0.95) and expose the resulting list to pathway or network enrichment analysis.^{57,59,67} Applicants can then validate the pPS-gene or drug relationships by determining if any of the drugs/genes also scored highly (e.g. >0.9) in any of the other tissues for the same pPS. These drugs and genes can be short-listed for further downstream analyses.

Gene and drug perturbations that are short-listed can be evaluated for further evidence of causality. For genes, Applicants can perform rare variant gene burden testing for association with T2D, as described herein, in studies with exome or WGS data (UKB, AMP-T2D-GENES, TOPMed, and All of Us). Short-listed drugs can be queried in compound databases, such as the Broad's Drug Repurposing Hub, an open-access repository of more than 6,000 compounds with documentation on chemical structure, effectiveness, and use in clinical trials.

The methods can allow for connecting multiple drug targets to T2D pPS (pathways). Demonstrating how gain or loss of function of a gene relates to altering a T2D pathway will improve understanding of T2D pathogenesis and potentially identify novel therapeutic targets. Identifying drug perturbations that act on T2D pathways has two impactful possible outcomes: 1) connecting known T2D drugs to specific disease pathways such that targeted treatment strategies could be studied in those at highest pathway-specific risk and 2) generating a short-list of small molecules that are supported by genetics to treat T2D via known pathways, also potentially enabling targeted therapy.

Two possible challenges that are resolvable with computational analysis: (1) the basis of connectivity scores could be a small number of gene-expression features with large effects or a larger number with more modest effects; Applicants therefore can analyze the features (including which were measured versus inferred) to understand how a gene or drug is connected to a pPS, and whether multiple connections to the same pPS are connected due to the same subset of features; (2) relatedly, CMap connections (or sets of them) may represent superpositions of connectivity signals (e.g., from polypharmacology); here, again, analyzing the underlying features (e.g., devising pathway-weighted versions of the connectivity score) may be appropriate. A more substantive issue involves asymmetries in the CMap corpus across time and concentration of drug treatment. In both cases, the value(s) selected for time or concentration by the CMap team might miss key connections in gene-expression space that might have been possible with other choices. This represents essentially a “false-negative” failure mode, Applicants can forestall its consideration unless the proposed approaches yield no significant connections.

Additional follow-up studies include:
1. Longitudinal study of the new T2D subtypes in clinical trials: Applicants can determine how T2D subtypes change clinically over time and also test hypotheses about response to drug treatment in the Diabetes Prevention Program (metformin vs lifestyle vs placebo), DECLARE (dapagliflozin vs placebo), SAVOR (saxagliptin vs placebo), and GRADE (four drugs added on top of metformin) clinical trials.
2. Further characterization of polygenic pathways: Applicants can perform CRISPR validation of the pPS pathways by knocking out genes identified as involved in T2D pathways and confirming that the gene knock out-model and corresponding T2D pPS share similar expression profiles.
3. Rescue of expression profile with targeted drug treatment: Applicants can identify tissues from people in MOBB with high risk T2D pPS and run compound screens using the top corresponding drug candidates to validate that the gene expression profiles, and cellular profiles can be rescued.
4. Investigational drug development: Applicants can validate top drug candidates with in vitro and in vivo model testing.

In summary, the genetics-based approach to characterizing the functional pathways that cause T2D will enable patient stratification, uncovering of complex gene regulatory networks, potential discovery of novel therapeutics that are amenable to targeted therapy, and extension of the approach to other complex diseases.

### Example 2 References

1. IDF Diabetes Atlas, 10th edn. Brussels, Belgium: International Diabetes Federation; 2021.
2. Udler M S, McCarthy M I, Florez J C, Mahajan A. Genetic Risk Scores for Diabetes Diagnosis and Precision Medicine. Endocr Rev 2019; 40(6): 1500-20.
3. Dornbos P, Koesterer R, Ruttenburg A, et al. A combined polygenic score of 21,293 rare and 22 common variants significantly improves diabetes diagnosis based on hemoglobin A1C levels. medRxiv 2021: 2021.11.04.21265868.
4. Goodrich J K, Singer-Berk M, Son R, et al. Determinants of penetrance and variable expressivity in monogenic metabolic conditions across 77,184 exomes. Nat Commun 2021; 12(1): 3505.
5. Udler M S, Kim J, von Grotthuss M, et al. Type 2 diabetes genetic loci informed by multi-trait associations point to disease mechanisms and subtypes: A soft clustering analysis. PLoS medicine 2018; 15(9): e1002654.
6. DiCorpo D, LeClair J, Cole J B, et al. Type 2 Diabetes Partitioned Polygenic Scores Associate With Disease Outcomes in 454,193 individuals Across 13 Cohorts. Diabetes Care 2022: dc211395.
7. Laber S, Strobel S, Mercader J-M, et al. Discovering cellular programs of intrinsic and extrinsic drivers of metabolic traits using LipocyteProfiler. bioRxiv 2021: 2021.07.17.452050.
8. Mahajan A, Wessel J, Willems S M, et al. Refining the accuracy of validated target identification through coding variant fine-mapping in type 2 diabetes. Nat Genet 2018; 50(4): 559-71.
9. Mahajan A, Taliun D, Thurner M, et al. Fine-mapping type 2 diabetes loci to single-variant resolution using high-density imputation and islet-specific epigenome maps. Nat Genet 2018; 50(11): 1505-13.
10. Saeedi P, Petersohn I, Salpea P, et al. Global and regional diabetes prevalence estimates for 2019 and projections for 2030 and 2045: Results from the International Diabetes Federation Diabetes Atlas, 9(th) edition. Diabetes Res Clin Pract 2019; 157: 107843.
11. Apicella M, Campopiano M C, Mantuano M, Mazoni L, Coppelli A, Del Prato S. COVID-19 in people with diabetes: understanding the reasons for worse outcomes. Lancet Diabetes Endocrinol 2020; 8(9): 782-92.
12. Captieux M, Fleetwood K, Kennon B, et al. Epidemiology of type 2 diabetes remission in Scotland in 2019: A cross-sectional population-based study. PLoS Med 2021; 18(11): e1003828.
13. Karter A J, Nundy S, Parker M M, Moffet H H, Huang E S. Incidence of remission in adults with type 2 diabetes: the diabetes & aging study. Diabetes Care 2014; 37(12): 3188-95.
14. McCarthy M I. Painting a new picture of personalised medicine for diabetes. Diabetologia 2017; 60(5): 793-9.
15. American Diabetes A. 2. Classification and Diagnosis of Diabetes: Standards of Medical Care in Diabetes-2020. Diabetes Care 2020; 43(Suppl 1): S14-S31.
16. Draznin B, Aroda V R, Bakris G, et al. 9. Pharmacologic Approaches to Glycemic Treatment: Standards of Medical Care in Diabetes-2022. Diabetes Care 2022; 45(Supplement 1): S125-s43.
17. American Diabetes A. (2) Classification and diagnosis of diabetes. Diabetes Care 2015; 38 Suppl: S8-S16.
18. Hattersley A, Bruining J, Shield J, Njolstad P, Donaghue K C. The diagnosis and management of monogenic diabetes in children and adolescents. Pediatr Diabetes 2009; 10 Suppl 12: 33-42.
19. Pearson E R, Liddell W G, Shepherd M, Corrall R J, Hattersley A T. Sensitivity to sulphonylureas in patients with hepatocyte nuclear factor-lalpha gene mutations: evidence for pharmacogenetics in diabetes. Diabet Med 2000; 17(7): 543-5.
20. Pearson E R, Starkey B J, Powell R J, Gribble F M, Clark P M, Hattersley A T. Genetic cause of hyperglycaemia and response to treatment in diabetes. Lancet 2003; 362(9392): 1275-81.
21. Fuchsberger C, Flannick J, Teslovich T M, et al. The genetic architecture of type 2 diabetes. Nature 2016; 536(7614): 41-7.
22. Bonnefond A, Boissel M, Bolze A, et al. Pathogenic variants in actionable MODY genes are associated with type 2 diabetes. Nat Metab 2020; 2(10): 1126-34.
23. Vujkovic M, Keaton J M, Lynch J A, et al. Discovery of 318 new risk loci for type 2 diabetes and related vascular outcomes among 1.4 million participants in a multi-ancestry meta-analysis. Nat Genet 2020; 52(7): 680-91.
24. Liu X, Li Y I, Pritchard J K. Trans Effects on Gene Expression Can Drive Omnigenic Inheritance. Cell 2019; 177(4): 1022-34.e6.
25. Khera A V, Chaffin M, Aragam K G, et al. Genome-wide polygenic scores for common diseases identify individuals with risk equivalent to monogenic mutations. Nat Genet 2018; 50(9): 1219-24.
26. Devarajan K. Nonnegative matrix factorization: an analytical and interpretive tool in computational biology. PLoS Comput Biol 2008; 4(7): e1000029.
27. Wouters O J, McKee M, Luyten J. Estimated Research and Development Investment Needed to Bring a New Medicine to Market, 2009-2018. JAMA 2020; 323(9): 844-53.
28. Ringel M S, Scannell J W, Baedeker M, Schulze U. Breaking Eroom's Law. Nat Rev Drug Discov 2020; 19(12): 833-4.
29. Pipeline for GWAS clustering using Bayesian non-negative matrix factorization (bNMF). github.com/gwas-partitioning/bnmf-clustering.
30. Common Metabolic Diseases Knowledge Portal. hugeamp.org/.
31. Neale Lab, GWAS analysis of the U K Biobank. www.nealelab.is/uk-biobank (accessed Mar. 18 2021).
32. Mahajan A, Spracklen C N, Zhang W, et al. Trans-ancestry genetic study of type 2 diabetes highlights the power of diverse populations for discovery and translation. medRxiv 2020: 2020.09.22.20198937.
33. Wheeler E, Leong A, Liu C T, et al. Impact of common genetic determinants of Hemoglobin A1c on type 2 diabetes risk and diagnosis in ancestrally diverse populations: A transethnic genome-wide meta-analysis. PLoS medicine 2017; 14(9): e1002383.
34. Flannick J, Mercader J M, Fuchsberger C, et al. Exome sequencing of 20,791 cases of type 2 diabetes and 24,440 controls. Nature 2019; 570(7759): 71-6.
35. Bult C J, Blake J A, Smith C L, Kadin J A, Richardson J E. Mouse Genome Database (MGD) 2019. Nucleic Acids Res 2019; 47(D1): D801-d6.
36. Bycroft C, Freeman C, Petkova D, et al. The U K Biobank resource with deep phenotyping and genomic data. Nature 2018; 562(7726): 203-9.
37. Bycroft C, Freeman C, Petkova D, et al. Genome-wide genetic data on ~500,000 UK Biobank participants. bioRxiv 2017.
38. Smoller J W, Karlson E W, Green R C, et al. An eMERGE Clinical Center at Partners Personalized Medicine. J Pers Med 2016; 6(1).
39. Taliun D, Harris D N, Kessler M D, et al. Sequencing of 53,831 diverse genomes from the NHLBI TOPMed Program. Nature 2021; 590(7845): 290-9.
40. All of Us Research Program I, Denny J C, Rutter J L, et al. The “All of Us” Research Program. N Engl J Med 2019; 381(7): 668-76.
41. Cornelis M C, Qi L, Zhang C, et al. Joint effects of common genetic variants on the risk for type 2 diabetes in U.S. men and women of European ancestry. Ann Intern Med 2009; 150(8): 541-50.
42. The GTEx Consortium atlas of genetic regulatory effects across human tissues. Science 2020; 369(6509): 1318-30.
43. Stegle O, Parts L, Durbin R, Winn J. A Bayesian framework to account for complex non-genetic factors in gene expression levels greatly increases power in eQTL studies. PLoS Comput Biol 2010; 6(5): e1000770.
44. Wessels B, Honecker J, Schottl T, et al. Adipose Mitochondrial Respiratory Capacity in Obesity is Impaired Independently of Glycemic Status of Tissue Donors. Obesity (Silver Spring, Md) 2019; 27(5): 756-66.
45. McCarthy S, Das S, Kretzschmar W, et al. A reference panel of 64,976 haplotypes for genotype imputation. Nat Genet 2016; 48(10): 1279-83.
46. Alonso L, Piron A, Moran I, et al. TIGER: The gene expression regulatory variation landscape of human pancreatic islets. Cell Rep 2021; 37(2): 109807.
47. Scott L J, Erdos M R, Huyghe J R, et al. The genetic regulatory signature of type 2 diabetes in human skeletal muscle. Nat Commun 2016; 7: 11764.
48. Pashos E E, Park Y, Wang X, et al. Large, Diverse Population Cohorts of hiPSCs and Derived Hepatocyte-like Cells Reveal Functional Genetic Variation at Blood Lipid-Associated Loci. Cell Stem Cell2017; 20(4): 558-70.e10.
49. Dobin A, Davis C A, Schlesinger F, et al. STAR: ultrafast universal RNA-seq aligner. Bioinformatics 2013; 29(1): 15-21.
50. Frankish A, Diekhans M, Ferreira A M, et al. GENCODE reference annotation for the human and mouse genomes. Nucleic Acids Res 2019; 47(D1): D766-d73.
51. Võsa U, Claringbould A, Westra H J, et al. Large-scale cis- and trans-eQTL analyses identify thousands of genetic loci and polygenic scores that regulate blood gene expression. Nat Genet 2021; 53(9): 1300-10.
52. The Gene Ontology resource: enriching a GOld mine. Nucleic Acids Res 2021; 49(D1): D325-d34.
53. Ashburner M, Ball C A, Blake J A, et al. Gene ontology: tool for the unification of biology. The Gene Ontology Consortium. Nat Genet 2000; 25(1): 25-9.
54. Jassal B, Matthews L, Viteri G, et al. The reactome pathway knowledgebase. Nucleic Acids Res 2020; 48(D1): D498-D503.
55. Huang R, Grishagin I, Wang Y, et al. The NCATS BioPlanet—An Integrated Platform for Exploring the Universe of Cellular Signaling Pathways for Toxicology, Systems Biology, and Chemical Genomics. Frontiers in Pharmacology 2019; 10.
56. Kanehisa M, Sato Y, Kawashima M. KEGG mapping tools for uncovering hidden features in biological data. Protein Science 2022; 31(1): 47-53.
57. Subramanian A, Tamayo P, Mootha V K, et al. Gene set enrichment analysis: a knowledge-based approach for interpreting genome-wide expression profiles. Proc Natl Acad Sci USA 2005; 102(43): 15545-50.
58. Kramer A, Green J, Pollard J, Jr., Tugendreich S. Causal analysis approaches in Ingenuity Pathway Analysis. Bioinformatics 2014; 30(4): 523-30.
59. Zou Y, Henry W S, Ricq E L, et al. Plasticity of ether lipids promotes ferroptosis susceptibility and evasion. Nature 2020; 585(7826): 603-8.
60. Qin Q, Fan J, Zheng R, et al. Lisa: inferring transcriptional regulators through integrative modeling of public chromatin accessibility and ChIP-seq data. Genome Biology 2020; 21(1): 32.
61. McLaren W, Gil L, Hunt S E, et al. The Ensembl Variant Effect Predictor. Genome Biol 2016; 17(1): 122.
62. Ferreira P G, Munoz-Aguirre M, Reverter F, et al. The effects of death and post-mortem cold ischemia on human tissue transcriptomes. Nat Commun 2018; 9(1): 490.
63. ConnectivityMap. clue.io/ (accessed Feb. 1 2022).
64. Skillman T G, Feldman J M. The pharmacology of sulfonylureas. Am J Med 1981; 70(2): 361-72.
65. Subramanian A, Narayan R, Corsello S M, et al. A Next Generation Connectivity Map: L1000 Platform and the First 1,000,000 Profiles. Cell 2017; 171(6): 1437-52.e17.
66. Predicted type 2 diabetes effector genes. www.type2diabetesgenetics.org/gene/effectorGeneTable (accessed 11 Jun. 2019).
67. Szklarczyk D, Gable A L, Nastou K C, et al. The STRING database in 2021: customizable protein-protein networks, and functional characterization of user-uploaded gene/measurement sets. Nucleic Acids Res 2021; 49(D1): D605-d12.

### Example 3—Genetically-Anchored Molecular Signature for Defining Mechanistic Disease Pathways

Type 2 diabetes (T2D) treatment is rarely curative and could be revolutionized by therapeutic modification of disease-causing pathways. Existing T2D drugs target hyperglycemia, the final symptomatic manifestation of T2D, rather than modifying its underlying pathophysiology. Insulin resistance (IR) is a defining feature of T2D that drives the development of T2D-related complications¹. However, mechanisms of IR are poorly understood, and insulin sensitizing drugs are lacking. T2D has a strong genetic basis with recent discoveries providing an unprecedented opportunity to leverage genetic information to better understand mechanisms of IR.

Tremendous genetic discovery for complex diseases, including T2D, holds immense promise to elucidate disease-causing pathways, but this critical link has not yet been realized at large scale. Genome-wide association studies (GWAS) have yielded hundreds of T2D genetic risk variants, although meaningful translation has been limited by poor mechanistic understanding.^{2,3} Most genetic risk variants have unknown function and, alone, confer minimal risk. Predictive ability is improved by incorporating many variants into polygenic scores (PS); however, traditional P S capture genetic risk across a conglomerate of physiologic processes, and do not inform on the heterogeneity of pathways seen in complex diseases like T2D.⁴

This example describes innovative methods to translate human genetic discovery into improved understanding of T2D causal mechanisms with the potential to transform patient care and disease management. The high-throughput approach to disease pathway discovery and annotation is broadly applicable to other diseases.

Clinical management of T2D is suboptimal, particularly with regard to addressing IR, and could be improved by greater understanding of the mechanistic pathways underlying disease. With at least 8% worldwide prevalence, T2D causes significant global health and economic burden.⁹ It is now well-appreciated that IR is a key driver of devastating complications of T2D¹⁰ as well as a risk factor for cardiovascular disease even in the absence of hyperglycemia.¹¹ Despite multiple therapeutic options available for T2D, a gaping hole exists in terms of true insulin sensitizers to peripheral actions of insulin that are free of major limiting side effects.¹ Even when patients reliably take prescribed T2D medications, remission or cure is rarely achieved.^{11,13} Although different disease processes are known to lead to hyperglycemia (e.g. defects in insulin production, secretion, or action), it is generally not clear for a given patient precisely why the disease has developed or how to individualize treatment.¹⁴ Understanding the heterogeneous mechanisms leading to T2D offers a means to i) subtype disease, ii) identify those at highest risk of subtypes, and iii) develop targeted therapeutics tailored to T2D subtypes, all advancing T2D management through a precision medicine framework. Heterogeneity in disease processes manifesting in clinical presentations is not unique to T2D, but rather a universal feature of complex human genetic diseases. Novel approaches are urgently needed to decipher the many pathways contributing to disease.

Genetic data elucidates disease mechanisms, but there has not been an effective approach to do this at scale for complex genetic conditions like T2D. Genetic contribution to T2D is estimated to account for 25-720^{15,16} of heritability and is exceedingly complicated. There are likely thousands of minor genetic changes collectively contributing to disease via dense polygenic networks converging on pathways.¹⁷ More than five hundred distinct loci contributing to T2D have been identified,^{2,3} yet how they contribute to T2D remains poorly understood. Investigations linking variants to functional mechanism have been insightful for a small subset of variants, but have been overall arduous and slow.¹⁸ Since single common T2D genetic variants typically have very small risk estimates, there have been efforts to aggregate risk across thousands of variants.⁴ These “polygenic scores” capture a person's composite risk of T2D and can identify the top 5% of score carriers as having a 4.5-fold increased T2D risk compared to the rest of the population.^{19,20} However, since this risk captures many different disease processes, the driving disease mechanism(s) in people with the highest full T2D polygenic scores is not known. Limited ability to connect genetic findings to disease pathways is a major bottle neck crippling translation of genetic discovery.

Resolving the missing link between large-scale genetic discovery and disease pathways has the potential to transform disease nosology, drug development, and patient care. Monogenic diabetes, which represents ~0.4% of all diabetes cases,²¹ provides a paradigm for how genetic understanding of disease processes provides insight into patients' clinical course and management.²² For example, people with monogenic diabetes due to mutations in HNF1A, a transcription factor involved in pancreatic beta cell function, suffer from dysfunctional insulin secretion, and are highly sensitive to sulfonylurea treatment, which promotes insulin secretion.²³⁻²⁵ In contrast, for complex diseases like T2D, the underlying disease pathways are expected to have polygenic contribution, and characterization of polygenic T2D processes can identify subsets of people with predominant disease mechanisms. Additionally, characterizing the polygenic mechanisms is critical for identifying pathway-specific drug targets and novel therapeutics. Limited knowledge of disease mechanistic processes precludes advances in disease subtyping, drug development, and targeted patient care.

Prior efforts to connect genetic loci at scale to disease pathways had limited efficacy. Assigning T2D genetic risk variants to disease processes is challenging because the majority of signals fall within non-protein coding regions of the genome and have unknown function.²⁶ Most previous efforts to systemically connect T2D loci to disease mechanisms utilized unsupervised hierarchical clustering.²⁷⁻²⁹ This “hard” clustering approach assigns loci to just one cluster each, such that a locus cannot be a member of more than one cluster. Additionally, there is a challenge of determining the optimal number of clusters; methods existing to assist with this may provide inconsistent determinations. Most notably, these prior approaches suffered in terms of interpretably of the resulting clusters. For example, in one of the most comprehensive analyses, most loci were grouped in an ‘unclassified’ cluster, even though several had known biological mechanisms (e.g., HNF1A, KCNJ11).²⁸

Applicants have developed a pioneering method to connect genetic variants to discrete disease processes,⁵ thus far identifying five polygenic mechanisms of T2D related to IR.⁶ More such processes remain to be discovered and all require further characterization. The described method of genetic clustering enables generation of process-specific “partitioned polygenic scores” (pPS) which capture a person's polygenic risk for a given mechanism of disease.⁵ In prior work, it was demonstrated that T2D pPS can stratify people with T2D into distinct clinical subtypes^{5,7} and define a specific cellular profile.⁸ Using new data available, Applicants can identify additional pPS and characterize pPS-associated molecular signatures to establish how complex genetic variation is reflected in cell profile and tissue function, mechanistically linking the pPS with T2D pathophysiology.

This example describes the development of a comprehensive set of T2D pPS related to IR. Applicants can characterize T2D pPS i) clinically using biobank and cohort datasets and ii) molecularly across diverse tissue types, demonstrating feasibility to identify disease process-specific genetic subtypes, regulatory genes, and therapeutic approaches.

Applicants hypothesized that saturation of discovery of genetic mechanisms of T2D related to IR has not been achieved, and leveraging additional datasets can identify novel mechanisms defining T2D subtypes. Applicants can employ the high-throughput physiologically driven clustering method on more than one thousand multi-ancestral T2D genetic loci to generate T2D pPS related to IR and then evaluate these pPS in independent studies for clinical characterization. This data-driven approach inputs GWAS summary statistics for T2D-associated genetic variants and their associations with diabetes-related traits and generates sets of variants with similar patterns of trait associations, representing putative disease processes. The clustered variants yield T2D IR pPS, which can then be tested for associations with clinical phenotypes defining T2D subtypes in three large studies: Mass General Brigham Biobank, All of Us Research Program, and NHLBI's TOPMed study.

Applicants hypothesized that T2D IR polygenic mechanisms modulate large gene expression networks that are involved in specific cellular processes and are controlled by master regulatory genes. Applicants can demonstrate across multiple tissues that T2D IR pPS define gene expression networks, cellular processes, and master regulatory genes. For each T2D pPS, Applicants can perform regression analysis to generate gene-expression signatures in 49 tissues from the Genotype-Tissue Expression (GTEx) project and additional muscle, adipose, liver, and pancreatic islet expression datasets. To gain further insight into disease mechanisms, Applicants can use the gene expression signatures to identify i) enriched canonical gene set pathways and ii) master regulatory genes. Applicants can corroborate findings using image-based profiles of adipocytes.

Applicants hypothesized that cellular perturbations with i) master regulatory genes and ii) drugs can recapitulate the process-specific T2D IR gene expression and image-profiles, supporting in silico findings. Applicants can demonstrate proof-of-concept that human cellular perturbations with gene- and drug-sets generate molecular signatures shared with T2D IR pPS, motivating therapeutic hypotheses. Applicants can map the T2D gene expression signatures to 7,494 gene- and 19,811 compound-perturbations performed in up to 77 cell lines in the Broad Institute's Connectivity Map (CMap). Applicants can assess similarity between the pPS signatures and cells perturbed by i) overexpression or loss of function of regulatory genes and ii) drugs. Applicants can corroborate findings in two other gene- and drug-perturbation datasets with expression- and image-based readouts.

Applicants developed a successful data-driven method to separate genetic variants into distinct and interpretable T2D mechanistic processes with tissue specificity, defining clinical subtypes.^{5,26} Applicants have recently developed a novel method to connect T2D genetic signals to disease processes using a data-driven approach.⁵ Applicants leveraged published GWAS summary statistics and a soft-clustering algorithm called Bayesian Non-negative Matrix Factorization (bNMF)³⁰ (FIG. 10) to group sets of T2D genetic variants together that share a similar pattern of associations across T2D-related traits. The underlying assumption is that genetic variants associated with multiple phenotypes in a consistent manner are likely part of a shared disease process. A great advantage of bNMF over traditional clustering approaches is that it allows for an element to be part of more than one cluster (“soft” clustering); this model fits with the biological understanding that disease-causing genetic variants may impact more than one biological pathway.⁵

In initial work, Applicants clustered 94 T2D genetic variants using 47 traits into five robust groupings, representing readily interpretable disease processes.⁵ The clusters were labeled based on driving trait patterns. Two clusters signified reduced pancreatic beta-cell function, differing from each other by high vs. low proinsulin levels, representing defective insulin processing vs defective insulin synthesis, respectively (labeled “Beta-cell,” “Proinsulin”). The three other clusters represented mechanisms of insulin resistance: obesity-mediated (“Obesity”), “lipodystrophy-like” body fat distribution (“Lipodystrophy”), and disrupted liver lipid metabolism (“Liver/lipid”). A parallel, independent effort converged on similar clusters,²⁶ further validating these five clusters. Additionally, Applicants found that the clusters were enriched for active DNA regulatory elements in tissues consistent with suspected mechanism (e.g., pancreatic beta cells for the “Beta cell” cluster and adipose for the “Lipodystrophy” cluster, and liver for the “Liver/lipid” cluster, Ps<0.001).⁵

Genetic variants assigned to T2D clusters can be used to generate “partitioned polygenic scores” (pPS), which capture a person's genetic risk for a given disease process.⁵ By identifying people with the highest pPS scores, it is possible to generate T2D subtypes. For example, Applicants found within patients with T2D that those with the top 10% “Lipodystrophy” pPS score had significantly higher triglycerides and fasting c-peptide levels (indicative of IR), but lower body mass index (BMI) and HDL cholesterol; this phenotypic pattern is consistent with Autosomal Dominant Partial Lipodystrophy, a monogenic condition of abnormal fat distribution characterized by IR even in the absence of obesity. Applicants have also shown that T2D pPS are differentially associated with metabolic conditions, such as kidney and coronary artery disease.^{5,7}

The most recent data for one T2D IR genetic mechanism defines a cellular phenotype in adipocytes⁸ which is not seen when using all T2D genetic variation aggregated together. To ascertain the effect of polygenic scores on cellular phenotypes, Applicants have previously profiled subcutaneous (SC) and visceral differentiating adipose-derived mesenchymal stem cells (AMSC's) from 26 donors using high-dimensional image-based profiling resulting in over 3,000 image-based cellular features. The individuals were first stratified by their full T2D polygenic score, and minimal differences in image-based morphological and cellular features were seen (FIG. 11A) (Laber, Strobel et al, Cell Genomics, in press).⁸ In contrast, when stratification was performed using the “Lipodystrophy” T2D pPS, Applicants observed image-based features informative for mitochondrial activity and the actin remodeling process in SC adipocytes. These differences were visible in ground truth images, indicating altered mitochondrial activity, decreased cortical actin, and decreased lipid accumulation in the high pPS group (FIG. 11B, C)⁸. Furthermore, Applicants found that the “Lipodystrophy” pPS image-based cellular signature was shared with genes causing monogenic lipodystrophy, for example PPARG, PLIN1, CIDEC, AKT2, and LIPE. These findings support convergence between the monogenic and polygenic lipodystrophy pathways and demonstrate that the pPS provides insight into T2D biology. These initial results are promising but have only scratched the surface of what the approaches have the potential to uncover about T2D. More genetic mechanisms of disease, particularly related to IR, remain to be discovered. All processes need clinical and molecular characterization to improve interpretation. The molecular characterization should ideally occur across multiple tissue-types and inform on regulatory genes and drug mechanisms of action. Leveraging cutting-edge omics resources, including CMap gene- and drug-perturbations, this work demonstrates how human germline discovery, if appropriately partitioned into disease mechanisms, can provide meaningful insight into T2D pathogenesis and therapeutic hypotheses.

This work provides many innovations. Applicants introduce a novel scalable approach to translate GWAS findings to elucidate disease processes and pathogenesis, providing a key missing link in human genetics. The work overcomes a critical hurdle of time- and labor-intensive investigation to connect single GWAS loci to function (“variant to function”) and addresses fundamental questions about how complex genetic regulatory networks impact disease. Improved genetic stratification of T2D by identifying novel mechanisms of IR could transform T2D care. Applicants generate the most comprehensive set of T2D IR pPS to-date, extend prior work to populations of non-European genetic ancestry, and assess clinical translation in diverse populations. Applicants provide a new method for high-throughput identification of drug targets and mechanisms of action relevant to specific disease pathways. Currently the cost and time required to develop novel drugs creates a major bottleneck.³¹ Human genetic data has been credited with improving this bottleneck,³² and Applicants offer a new approach leveraging thousands of gene- and drug-perturbations in CMap as proof of concept. This approach offers potential for disease-modifying and pathway-tailored treatment strategies.

The central hypothesis of the approach taken is that Applicants can utilize human genetics to uncover T2D mechanistic processes related to IR that i) identify T2D genetic subtypes, ii) define robust molecular signatures of genes related to established pathways and regulatory genes, and iii) identify therapeutic targets that correlate with the signatures (FIG. 12).

Applicants hypothesized that Saturation of discovery of genetic mechanisms of T2D related to IR has not been achieved, and leveraging additional datasets can identify novel mechanisms defining T2D subtypes. Applicants can employ the high-throughput physiologically driven clustering method on more than a thousand of multi-ancestral T2D genetic loci to generate T2D pPS related to IR and then evaluate these pPS in independent studies for clinical characterization. The work deriving genetic clusters of T2D-associated loci provided a groundbreaking method for connecting T2D loci to mechanistic processes and defining initial T2D subtypes.^{5,6} There are now additional relevant GWAS datasets available for T2D and related-traits, such that the prior work could be substantially advanced. The number of known T2D loci has nearly quadrupled since the last published analysis. Many of these loci come from multi-ancestry T2D GWAS, which also importantly allows for improved translation of clustering and pPS results across patient populations. Additionally, based on previous results,⁶ incorporating additional and larger GWAS of key diabetes-related traits (e.g., proinsulin levels³³) is expected to provide better delineation of genetic loci into clusters and identify novel clusters. The objective of this aim is to generate novel and enhanced genetic clusters of T2D related to IR mechanisms using GWAS summary statistic data and bNMF clustering, and then use these clusters to define T2D clinical subtypes.

To facilitate discovery of T2D genetic processes, Applicants have developed a semi-automated high-throughput pipeline to perform genetic clustering at scale. The initial analysis using the pipeline expanded the number of T2D variants involved in genetic clustering from 94 in prior work⁵ to 323.⁶ Employing this pipeline (code available on GitHub³⁴), Applicants aggregated summary statistics from 13 T2D GWAS studies in the Accelerating Medicines Partnership Common Metabolic Disease Knowledge Portal (AMP-CMDKP),³⁵ from which Applicants extracted 21,666 variants associated with T2D reaching genome-wide significance (P<5×10⁻⁸). After performing stringent linkage disequilibrium (LD)-pruning (r²<0.1) and optimizing variants for inclusion in the variant-trait association matrix (e.g., removing those with >80% missing trait associations), 323 T2D variants representing independent signals were taken forward for the clustering. For each T2D variant, Applicants used the T2D-risk increasing allele to extract directionally aligned GWAS summary statistics for 75 T2D-related traits, which included glycemic, anthropometric, and laboratory measures. Of the 75 traits, 64 were associated with at least one T2D variant at Bonferroni-significance level (P<0.05/75) and included in the clustering matrix; thus, the approach was data-driven with genetics guiding trait-inclusion. Soft clustering with bNMF (FIG. 10) was performed using the resulting 323 by 64 variant-trait association matrix and converged optimally on ten clusters. These ten T2D clusters captured the previous five clusters (with beta-cell separating into two clusters), demonstrating reliability of the high-throughput approach (Table 26). Additionally, the recaptured clusters had more than twice the number of variants than the original results.⁵ Importantly, this initial expansion effort identified six novel clusters (including the two new “Beta cell” clusters, Table 26).

TABLE 26
Comparison of clustering results showing improvement with inclusion
of additional T2D genetic variants and related traits

N variants

| | Original | Preliminary |
|---|---|---|
| | Analysis (94 | Results (336 T2D |
| | T2D variants, | variants, 64 |
| Cluster Name | 47 traits) | traits) |
| Beta cell | 30 | Beta Cell 1: 66 |
| | | Beta Cell 2: 27 |
| Proinsulin | 7 | 18 |
| Obesity* | 5 | 37 |
| Lipodystrophy* | 20 | 54 |
| Liver/Lipid* | 5 | 12 |
| ALP negative* | | 4 |
| Hyper Insulin | | 32 |
| secretion* | | |
| Lipoprotein A | | 1 |
| SHBG | | 1 |

*Clusters relating to mechanisms of IR

The initial expanded analysis identified five T2D clusters related to IR, two of which were novel, with all defining distinct clinical subtypes.⁶ For the ten clusters resulting from this initial expanded analysis, Applicants identified five as having mechanisms of IR based on cluster variants being collectively associated with increased fasting insulin (“Obesity”, “Lipodystrophy”, “Liver/Lipid,” “Alk Phos Negative,” and “Hyper Insulin Secretion”). Two IR clusters were novel (“Hyper insulin secretion” and “Alk Phos Negative”) and driven by loci and traits new to this analysis (e.g., ABO, PPP1R3B, and alkaline phosphatase). T2D pPS for each cluster were generated using cluster weights. Applicants analyzed these updated pPS in 25,419 individuals of European genetic ancestry in the Mass General Brigham Biobank (MGBB). Within the subset with T2D (N=1,893), there were significant differences in clinical features across all the pPS and also across just the five pPS related to IR. For example, “Obesity” was the only IR pPS associated with increased BMI (beta per standard deviation [SD]pPS 0.02, P=2.94×10⁻⁵), and in contrast, the “Lipodystrophy” was associated with decreased BMI (beta −0.02, P=3.7×10⁻⁵). The five T2D IR pPS also differed in terms of associations with circulating triglyceride levels (significantly increased for “Lipodystrophy” and decreased for “Liver/lipid” and “Alk Phos Negative”), HDL cholesterol levels (significantly decreased for “Lipodystrophy” and “Hyper Insulin Secretion”), and alkaline phosphatase levels (only significantly decreased for “Alk Phos Negative”). These results demonstrate that 1) increasing the number of variants and traits in clustering analysis can identify novel T2D IR clusters and 2) identifying T2D IR pPS can genetically stratify people with T2D, resulting in significant clinical differences between subgroups.

Applicants use GWAS summary statistics to identify clusters. Applicants can utilize publicly available summary statistics from over 125 GWAS from dbGaP, UK Biobank (UKB),³⁶ and AMP-CMDKP³⁵. Compared to prior work, Applicants can increase the number of T2D GWAS datasets from 13 to 38 (resulting in more than four-fold more T2D loci), and trait GWAS from 75 to 91, and importantly include studies of non-European genetic ancestry. Individual level-datasets are shown below (Table 27).

TABLE 27
Study characteristics

| | MGBB | All of Us* | TOPMed |
|---|---|---|---|
| N total | 64,109 | 165,127 | 81,642 |
| % Male | 45% | 38% | 41% |
| % European | 84% | 53% | 49% |
| ancestry | | | |
| N with T2D | 4,793 | 17,294 | 20,737 |

MGB Biobank (MGBB)³⁷ has genotyped 64,109 consented patients seen at clinical sites across the MGB healthcare system. Phenotypic clinical data are available via patient electronic health records (EHR). QC of the genetic data and imputation to the TOPMed Reference Panel³⁸ has been performed.

All of Us Research Program³⁹ is an NIH-funded diverse cohort (anticipated N=1,000,000; current Tier Dataset v6 N=372,380) with accompanying clinical data including laboratory values (v6 Sample with EHR data N=227,740). Genotype array data are currently available for 165,127 participants.

Trans-Omics for Precision Medicine (TOPMed)³⁸ Program is a precision medicine initiative by the National Heart, Lung, and Blood Institute (NHLBI) consisting of ~186K participants from >85 studies, including biobanks and cohorts. Whole genome sequencing (WGS) data has been released in freezes. The TOPMed Diabetes Working Group has harmonized phenotype and genotype data currently available for 81,642 (Freeze 9).

To perform genetic clustering, Applicants can take a similar approach to previous work^{5,6}, but now expand to incorporate additional GWAS datasets, including more than 40 recently published studies of diverse ancestral populations not included in the prior work, as well as one forthcoming T2D GWAS that has identified 145 novel T2D loci (Type 2 Diabetes Global Genomics Initiative).^{2,3} To enrich the ability to identify genetic clusters related to IR, Applicants can additionally include alleles that are associated with both i) measures of IR (homeostatic model of insulin resistance [HOMA-IR], insulin sensitivity index, fold-change insulin following glucose challenge) at genome-wide significance (P<5×10⁻⁸) and ii) with increased T2D risk at a significance threshold determined by Bonferroni correction for the total number of independent alleles. Applicants can perform clustering of the expanded set of T2D variants using summary statistics from GWAS of at least 91 T2D-related traits, including glycemic, anthropometric, and laboratory measures. The high-throughput pipeline^{6,34} can be used to generate the variant-trait association input matrix and perform bNMF clustering, yielding cluster weights for each variant and trait (FIG. 10). The Bayesian implementation of the NMF clustering has the advantage of letting the data determine the optimal number of clusters (detailed methods available herein and in⁵).

To characterize clinical features of potential genetic subtypes of T2D, Applicants can calculate the T2D IR pPS in individuals from independent validation cohorts, MGBB,³⁷ TOPMed,” and All of Us³⁹ (Table 27). The pPS can be generated for each participant using a standard weighted method whereby the number of risk alleles (0, 1, or 2) at each SNP are multiplied by their cluster weight, then summed, multiplied by the number of SNPs in the genetic risk score, and divided by the sum of all the weights.⁴⁰ To test the association between each pPS and each clinical phenotype (Table 21, Example 2), Applicants can use regression models (logistic for dichotomous outcomes, linear for continuous traits). Analyses can be adjusted for age, sex, reported race/ethnicity, and principal components (PC's) to account for ancestry differences. A Bonferroni level of significance can be used to reduce reporting false positive associations in the context of multiple testing. As sensitivity analyses, Applicants can add to the primary model adjustment for T2D status and also perform testing in data subsets restricted to only T2D cases.

Consideration of sex: When available, GWAS studies that are stratified by sex can be used. Sex-stratified sensitivity analyses can be performed to determine if pPS associations with clinical traits differ between men and women.

Consideration of race/ethnicity/ancestry: Primary analyses can be performed in all individuals adjusted for PC's, as noted above. As a sensitivity analysis, Applicants can stratify by self-reported race/ethnicity categories to determine if pPS associations with clinical traits differ by race/ethnicity.

Power Calculation: Even in the smallest dataset (MGBB restricted to people with T2D, N=4,793), Applicants have at least 80% power to detect a 0.065 correlation between each T2D pPS and continuous traits (e.g., laboratory values) at a level of significance of 0.00025 (accounting for 20 tests for 10 T2D IR pPS, or 0.05/200).

The outcome can be robust clinical characterization of novel and enhanced T2D IR pPS derived from expanded T2D genetic clusters. The variant cluster weights necessary for generating the pPS can be made freely accessible to the greater research community.

Subsequent embodiments are not dependent on expanded pPS since Applicants can also use previously identified pPS. Applicants can also consider employing a more relaxed significance threshold for T2D variant selection. Relaxed significance thresholds are now commonly used in full unpartitioned polygenic scores for many diseases.²⁰ While it is possible that choosing a more relaxed threshold could introduce variants with false-positive T2D associations, the clustering approach takes into account each variant's association with multiple traits, so Applicants expect such variants to not cluster well and thus be unlikely to contribute strongly to any T2D pPS. Applicants note that a limitation of the current approach is that it does not fully consider environmental factors such as diet and lifestyle. Applicants have chosen to focus on genetic risk factors (averaged across such factors) given that downstream analyses relate specifically to translation of genetic findings, for example identification of potential pathway-specific drug perturbagens; using the genetic findings from this work, Applicants can study interactions with environmental factors in future research.

Applicants hypothesized that T2D IR polygenic mechanisms modulate large gene expression networks that are involved in distinct cellular processes and are controlled by master regulatory genes. Applicants can demonstrate across multiple tissues that T2D IR pPS define gene expression networks, cellular processes, and master regulatory genes.

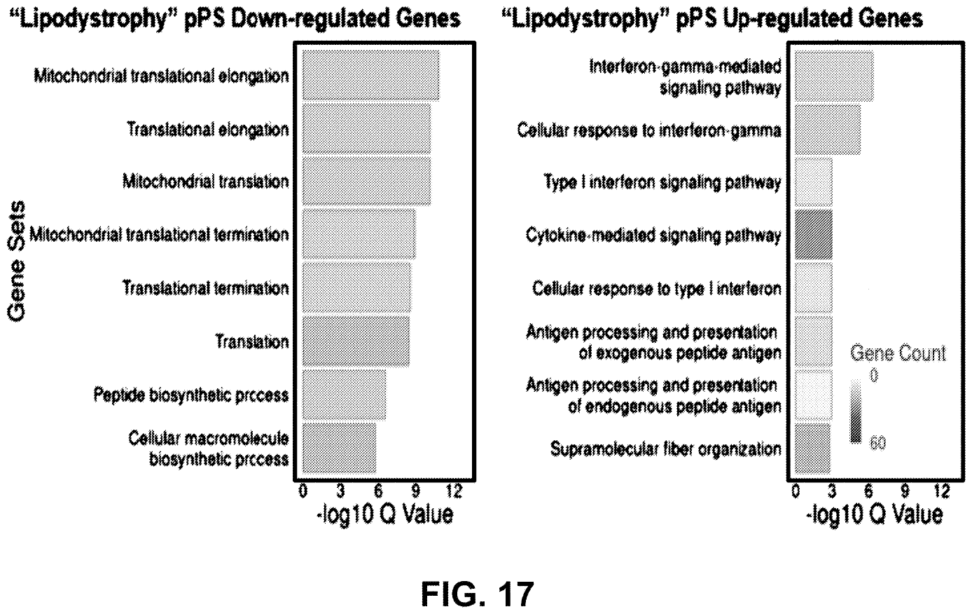

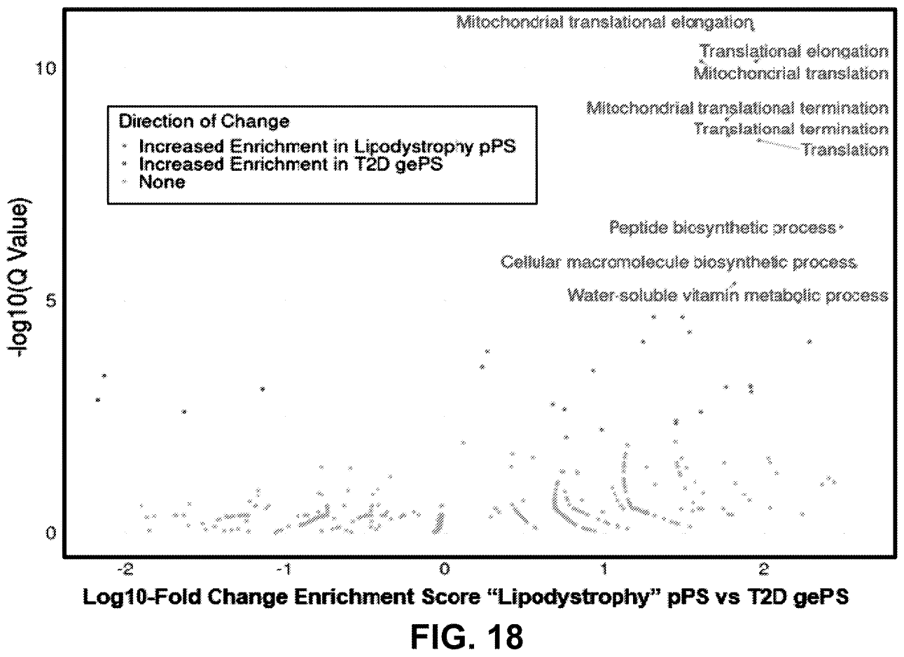

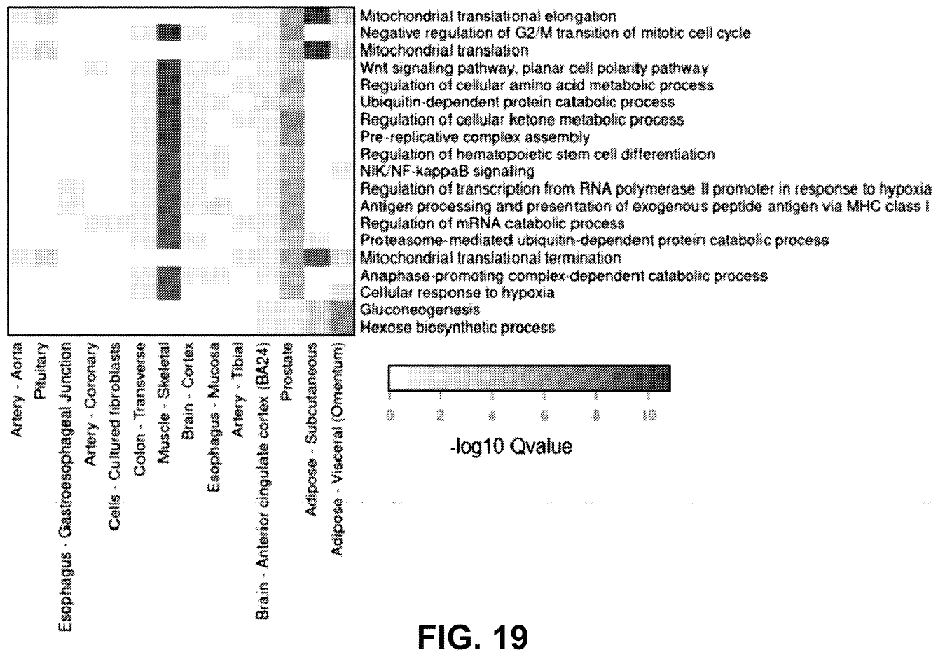

Current understanding of the complex polygenetic networks contributing to T2D pathophysiology is very limited and has prevented translation of genetic discovery. Applicants can identify and characterize T2D IR process-specific gene expression signatures which can capture pathway-specific polygenetic regulatory networks. Applicants can develop these signatures in multiple tissue expression datasets to study how disease processes impact tissues across the body and to identify putative master regulatory genes. Expression signatures derived using the “Lipodystrophy” T2D IR pPS are enriched for relevant canonical pathway gene sets and are consistent with cellular image-based findings. Applicants used the published “Lipodystrophy” T2D pPS⁵ to generate gene expression signatures in data from SC adipose tissue derived from 838 GTEx donors to see if the transcriptional profile would relate to the image-based lipocyte profiling, in which a salient feature in SC adipocytes from individuals with high “Lipodystrophy” pPS was abnormal mitochondrial morphology (FIG. 11B, C).⁸ Applicants calculated the “Lipodystrophy” pPS in the 838 GTEx donors and ran a linear regression model with tissue transcript expression as the outcome and the “Lipodystrophy” pPS as the exposure, adjusting for age, sex, batch, and two genotyping PC's. Applicants selected the top associated transcripts with P<0.01 and separated them by direction of association with the pPS (up- or down-regulated) and then assessed for gene set enrichment using the Enrichr gene set search engine.⁴¹⁻⁴³ The down-regulated transcripts were most significantly enriched for pathways related to mitochondrial translation (Q<10⁻⁹) in both GO Biological Process 2021^{44,45} (FIG. 17) and Reactome 2022⁴⁶, consistent with the adipocyte cellular profile features seen (FIG. 11B, C).⁸ The up-regulated transcripts were most significantly enriched for pathways related to interferon gamma signaling (Q<10⁻⁵, FIG. 17). Notably, the marked enrichment for specific gene sets observed for the “Lipodystrophy” pPS was not observed in analysis of the full T2D global extended polygenic score (gePS) (FIG. 18), despite the “Lipodystrophy” score being derived from just 20 SNPs in comparison to thousands in the T2D gePS. These results, concordant with the adipocyte image-based results (FIG. 11A, B),⁸ highlight the immense value of studying pPS rather than un-partitioned, heterogenous disease polygenic scores. Applicants then generated a “Lipodystrophy” pPS expression signature in all available GTEx tissues and noted multiple other tissues with significantly enriched pathways and pathways shared across tissues (FIG. 19).

The “Lipodystrophy” pPS expression signature can be used to identify putative regulatory genes. Given results showing convergence of the image-based cellular features with genes causing monogenic lipodystrophy, including PPARG, Applicants hypothesized that the “Lipodystrophy” pPS expression signature could be used to identify putative regulatory genes via two different approaches. First, Applicants used the epigenetic Landscape In Silico deletion Analysis (LISA)⁴⁷ to assess for transcriptional regulators predicted to act on the genes in the “Lipodystrophy” pPS expression profile. The input for LISA was the set of down-regulated genes in the expression profile used for the gene-set enrichment analysis (those with P<0.01). LISA's top two predicted regulators were MEDI and PPARG, with P-values<10⁻³⁵ in human adipose experiments;⁴⁸ MED1 was recently identified as a lipogenesis coactivator required for postnatal adipose expansion development.⁴⁹ Second, Applicants assessed across GTEx donors for transcripts significantly (false discovery rate (FDR)<0.01) associated with PPARG in SC adipose to generate a PPARG “natural variation” expression signature. This signature was significantly negatively correlated with the “Lipodystrophy” pPS expression signature (Pearson coefficient −0.3, FDR=1×10⁻²⁰), indicating that the gene expression network related to reduced PPARG expression converges with the pPS-defined gene expression network, suggesting a PPARG LOF mechanism. Note, Pearson correlation was used as an initial approach, but Applicants can use a more sophisticated measure of similarity (“connectivity score”), described below.

These results indicate that 1) Applicants are able to generate an expression signature for a T2D IR pPS that is significantly enriched for genes in canonical pathways consistent with an orthogonal analysis of cellular image-based phenotypes, 2) the enrichment for pathways is specific to the pPS vs the full T2D gePS, 3) expression signatures are significantly enriched for pathway gene sets across multiple tissues, offering exciting potential to study each disease-process across the whole body and also allowing for more than one tissue expression signature to be considered for downstream analyses, and 4) the expression signature can point to key regulatory genes that converge on the polygenic disease process.

Applicants can utilize multiple datasets with tissue expression and linked germline genetic data to generate pPS gene expression signatures (genetically-anchored gene signatures) (Table 28):

TABLE 28
Datasets for generation of pPS gene expression signatures

| | N | | Age | | |
|---|---|---|---|---|---|
| Study | individuals | Tissue(s) | (mean) | % Male | % White |
| GTEx | 838 | 49 including: | 53 | 67% | 85% |
| | | Subcutaneous adipose, | | | |
| | | visceral adipose, | | | |
| | | pancreas, liver, | | | |
| | | skeletal muscle | | | |
| TIGER | 404 | Pancreatic islets | 56 | 53% | 100% |
| MOBB | 26 | Subcutaneous adipose | 44 | 25% | 100% |
| | 31 | Visceral adipose | | | |
| PhLiPS | 89 | iPS derived hepatocytes | 32 | 44% | 55% |
| FUSION | 271 | Skeletal muscle | 80 | 55% | 100% |
| | 251 | Subcutaneous adipose | | | |
| Mexican T2D | 294 | Subcutaneous adipose, visceral | 39 | 24% | 0% |
| Biobank | | adipose, liver, skeletal muscle | | | |

The Genotype-Tissue Expression (GTEx)⁵⁰ is a publicly available resource of 49 non-disease tissues with paired genetic data collected from up to 838 human deceased donors. Expression data were generated with Illumina TruSeq RNA sequencing and WGS with Illumina HiSeq. Analysis covariates include genotyping PCs, sex, sequencing platform, sequencing protocol (PCR-based or PCR-free), and Probabilistic Estimation of Expression Residuals (PEER)⁵¹ factors. Data are available via dbGaP (phs000424.v9).

The Munich Obesity BioBank (MOBB)⁵² contains paired SC and visceral AMSC's derived from 221 donors taken during abdominal laparoscopic surgery. Expression data were generated with Illumina TruSeq RNA sequencing and genotype data with the Illumina Infinium Global Screening Array BeadChip. For 26 and 31 participants Applicants have profiled SC and visceral AMSC's at four stages of differentiation, day 0 (progenitor state), day 3, day 8, and day 14 (fully differentiated). Additionally, Applicants have used image-based profiling to extract 3,005 morphological and cellular features (“LipocyteProfiler”).⁸ Covariates for analysis include genotyping PCs, age, sex, BMI, and batch.

The Translational Human Pancreatic Islet Genotype Tissue-Expression Resource (TIGER)⁵³ has generated a large eQTL resource for pancreatic islets obtained from >500 organ donators. Genotype and expression data platforms varied by contributing study.

The Phenotyping Lipid traits in iPS derived hepatocytes Study (PhLiPS)⁵⁴ focuses on individuals free of cardiovascular or lipoprotein metabolism disorders in the community served by the Hospital of the University of Pennsylvania. Data from 89 individuals has been made available on dbGaP (Accession: phs001341.v1.p1), including RNA-seq data from induced pluripotent stem (iPS) cell-derived hepatocytes using HiSeq 2000. The gene expression data is paired to participant genotype data using Infinium Human CoreExome-24 BeadChip.

The Finland-United States Investigation of NIDDM Genetics (FUSION) Tissue Biopsy Study (FUSION)⁵⁵ involved 271 individuals with variable levels of glucose tolerance: 97 with normal glucose tolerance (NGT), 71 with impaired glucose tolerance (IGT), 36 with impaired fasting glucose (IFG), and 67 with newly-diagnosed T2D. Skeletal muscle biopsies from the 271 individuals and abdominal SC adipose tissue from a subset of 251 are available in dbGaP(phs001048). Expression data was generated with Illumina TruSeq RNA sequencing and genotyping on the Illumina Omni2.5M array.⁵⁶

The Mexican T2D Biobank⁵⁷ includes 294 individuals from Mexico with either prediabetes or T2D. Liver (in 288, 42 with T2D), muscle (in 91, 15 with T2D), SC adipose (in 287, 42 with T2D), and visceral adipose (in 280, 42 with T2D) samples were collected from patients who underwent bariatric surgery. RNA-sequencing using Illumina Sequencing and genotyping using the Multi-Ethnic Genotyping array are expected by Spring 2024.

In each transcriptomic dataset, Applicants can calculate T2D pPS, both published^{5,6} and novel or enhanced as described herein. Additionally, for comparison, Applicants can calculate full T2D and HOMA-IR gePS. Since the expression studies used different tissue isolation protocols, genotyping arrays, and RNA sequencing technology, Applicants can perform QC separately for each (as done in TIGER⁵¹). Genotype imputation using the TOPMed³⁸ reference panel can be performed to obtain the most comprehensive set of genetic variation. Weighted normalization methods can be used to address expression variances across different cell types. RNA-seq data can be aligned with STAR⁵⁸ to the newest GENCODE⁵⁹ reference database. For analysis of each tissue dataset, to remove potential artifact, Applicants can filter the transcripts to those with at least 20% of samples having greater than 7 transcripts per million (TPM).

To generate expression signatures for each pPS in every tissue, Applicants can run linear regression models. Each model can have the pPS as the exposure, transcript expression as an outcome, and adjust for age, sex, genotyping PC's, and study-specific covariates including PEER factors⁵¹ when appropriate. The beta estimate from the regression can be represented as the change in transcript level per SD change in the pPS. The results from this analysis produce a vector of 1,000's of genes with expression changes in relation to the pPS representing the “pPS expression signature” for each tissue.

To determine enriched gene sets/pathways for each pPS tissue expression signature, Applicants can use established tools including GSEA,⁶⁰ Enrichr,⁴¹⁻⁴³ and Gene-List Network Enrichment Analysis (GeLiNEA)⁶¹ and libraries including Gene Ontologies (GO),^{44,45} Reactome,⁴⁶ BioPlanet,⁶² and KEGG.⁶³ For all enrichment analyses, Applicants can focus on results reaching significance after accounting for multiple testing using a FDR<0.05 threshold and expect all real biology to be captured by more than one tool and library. For tissues available from more than one dataset (e.g., liver, adipose, muscle), Applicants can cross-validate results by 1) calculating correlation between expression signatures and presence of overlap in top associated transcripts, and 2) assessing for replication of significant pathway enrichment results. When results for the same tissue in more than one dataset show such concordance (i.e., significant correlation or shared significant pathway enrichment), Applicants can meta-analyze the expression signatures using inverse-variance weighted meta-analysis to produce a more robust expression signature. As a means of orthogonal corroboration of enriched gene sets/pathways for each pPS expression signature, the results can be compared to high-dimensional image-based profiles generated for the same pPS using the MOBB LipocyteProfiler signatures.

To identify putative master regulatory genes for each pPS expression network, Applicants can take two complementary approaches. First, Applicants can use the epigenetic Landscape In Silico deletion Analysis (LISA)⁴⁷ tool to predict transcription factors and chromatin regulators controlling top genes in the pPS expression signature (those with P<0.01). LISA leverages hundreds of publicly available ChIP-seq, DNase-seq, and H3K27ac ChIP-seq experimental studies. Second, Applicants can generate a proxy gene perturbation signature utilizing natural variation in genes across all individuals in GTEx. For a given “gene x”, Applicants can generate a “gene x” “natural variation” expression signature by performing linear regression to obtain change in other genes' expression per SD change in “gene x” expression. Applicants can then use a similarity metric (see “connectivity score” further herein) to determine which genes have high similarity in their “natural variation” expression signature to a given pPS expression signature.

Applicants can further validate the findings by testing for differential expression between T2D and either prediabetes (which includes IGT and IFG) or normal glucose tolerance in the RNA-seq datasets available from FUSION and the Mexican T2D Biobank using DESeq2.⁶⁴ Differentially expressed gene sets can be tested for pathway enrichment/master regulatory genes, as described above, and considered for perturbation mapping as further described herein.

All test statistics can be corrected for multiple testing using Bonferroni correction.

Consideration of sex: When sample size allows, Applicants can perform sex-stratified analyses to understand if gene expression profiles and downstream analysis results differ in men and women.

Power Calculation: Applicants estimate in GTEx (N=838) having at least 80% power to detect a 0.12 SD change in single transcript expression per SD change in pPS at P<0.01. Of note, the downstream analyses consider a multi-gene expression signature rather than a single gene effect. The results indicate that Applicants are well-powered in GTEx to generate pPS expression signatures with significant pathway enrichment.

Expected Outcomes: Applicants can identify pathway- and tissue-specific i) expression signatures, each representing a network of thousands of genes, and ii) putative master regulatory genes. Applicants can test whether in vitro cellular perturbations further validate the findings. The pPS gene expression signatures and the putative regulatory gene sets can be made freely available to the broader research community.

Alternative Strategies: In datasets involving whole tissues, there can be heterogeneity of cell types and cellular states, as well as potential artifacts of tissue collection (e.g., effects of death in GTEx).⁶⁵ Applicants can follow best practice analysis procedures developed by GTEx.⁵⁰ If Applicants find that 1) expression profiles or enrichment results differ greatly between matched tissue types in different datasets or that 2) Applicants fail to see any significant downstream findings from a given expression signature, Applicants can perform deconvolution methods to stratify the whole tissue samples into more homogenesis subsets. For example, Applicants have performed 10× Multiome joint single nuclei transcriptional and chromatin accessibility profiling in differentiating adipocytes and have aggregated single cell data sets from adipose tissue. Applicants have benchmarked the BayesPrism deconvolution algorithm⁶⁶ to calculate the composition of cell sub-populations from RNA-seq data sets using single cell data as an anchor to jointly predict cellular composition and gene expression in each cell type from bulk RNA-seq data. Relative abundance of the cell populations can then be used to regress against polygenic scores.

Applicants hypothesized that cellular perturbations with i) master regulatory genes and ii) drugs can recapitulate the process-specific T2D IR gene expression and image-profiles, supporting in silico findings. Applicants can demonstrate proof-of-concept that human cellular perturbations with gene- and drug-sets generate molecular signatures shared with T2D IR pPS, motivating therapeutic hypotheses.

There is a critical need to identify new T2D therapeutics directed at causal disease mechanisms, particularly IR, and to connect existing T2D drugs to patients most likely to benefit from them. As an initial step, Applicants can study whether molecular signatures of T2D IR pPS map to those resulting from cellular drug- and gene-perturbations. Such direct perturbations provide further support for regulatory gene findings and an important layer of annotation of the pPS related to drug mechanism of action (MOA), both of which can motivate additional research. Applicants can demonstrate feasibility of applying pPS molecular signatures to perturbation datasets to prioritize disease process-specific regulatory genes and drugs.

Cellular perturbations with i) FDA approved thiazolidinediones, ii) insulin sensitizers and iii) PPARG LOF generate gene expression signatures with significant similarity to that of the “lipodystrophy” T2D pPS in SC adipose tissue. Given that the “Lipodystrophy” pPS shares morphological cellular features with genes causing monogenic lipodystrophy^{5,8}, including PPARG, Applicants assessed whether the “Lipodystrophy” pPS expression signature would map to i) the FDA-approved diabetes drug class thiazolidinediones (TZDs), which are PPARG agonists, ii) a broader set of compounds in CMap labeled with mechanisms of action “PPAR receptor agonist/insulin sensitizer” (containing TZDs but also non-FDA approved compounds), and iii) CRISPR loss of function (LOF) perturbation of PPARG. For this preliminary analysis, Applicants utilized the CMap web-based interface⁶⁷ to analyze the top 300 most differentially expressed genes in each pPS rather than the full signature. i) In the HT29 (colorectal cancer) cell line, the four TZD's in CMap (Table 25, Example 2) had stronger median connectivity scores than all other compounds (two-sided Wilcoxon Rank Sum FDR=0.03). ii) The broader set of 35 PPAR receptor agonists/insulin sensitizers were even more significantly enriched in both HT29 and PC3 (prostate cancer) cell lines (FDR=6×10⁻⁶ and 5×10⁻⁵, respectively). iii) Within the HT29 cell line, perturbation with PPARG LOF had significant similarity (FDR=9×10⁻⁴), with the twelfth highest score of all ~5,000 LOF gene perturbations. Of note, the results indicated in GTEx that the “Lipodystrophy” pPS expression signature had significant pathway enrichment in transverse colon and prostate tissues (FDR<0.01), supporting the meaningful perturbation results coming from HT29 and PC3 cell lines. These results clearly support that pPS expression profiles can map to highly relevant perturbations in CMap. The proposed analyses can not only extend these findings to a larger landscape of additional T2D IR pPS, tissue expression profiles, and drug classes, but can also benefit from analysis of full expression signatures.

Connectivity Map (CMap)⁶⁸ is an expansive catalog of cellular responses to perturbations housed at the Broad Institute. It contains over 470,000 gene expression signatures from 19,811 small-molecule compounds, and 7,494 genetic reagents, tested in 77 possible cell lines. The genetic perturbations include gain of function or loss of function assays, and the small molecule compounds were compiled from multiple screening libraries. The gene expression data were generated using “L 1000,” a high-throughput gene expression assay that measures the mRNA transcript abundance of 978 “landmark” genes selected by a data-driven approach to be able to recover 82% of connections made using the full transcriptome. Thus, expression of 11,350 additional genes in the transcriptome were inferred for a total of 12,328 genes reported.

Joint Undertaking in Morphological Profiling (JUMP)-Cell Painting Database⁶⁹ is a public dataset of cell painting in response to genetic and drug perturbations at the Broad Institute. Cell painting is a high-throughput microscopy assay that generates images that reflect cell response to treatments such as drug or genetic perturbations. JUMP contains cell painting data for the U2OS (osteosarcoma) cell line and human white adipocytes. The U2OS cells were perturbed with ~13,000 overexpressed genes, ~8,000 CRISPR knockdowns, and ~120,000 small molecule perturbations. Human white adipocytes have perturbations with ~300 compounds, each in six different disease-related stimulatory conditions.

Munich Obesity BioBank (MOBB),⁵² described previously herein, contains 221 donors from which Applicants have profiled 26 SC and 31 visceral AMSCs across four stages of differentiation using SNP array, RNA-seq, and LipocyteProfiler image-based profiling.⁸ The 26 SC AMSC's have undergone CRISPR-Cas9 knock-out screens for 48 genes related to adipocyte differentiation, lipid metabolism, and insulin responsiveness. CRISPRi for ~2,000 genes in SC and visceral AMSCs can be available by spring 2024.

As proof-of-concept and validation of findings, Applicants can test whether human cellular perturbations with gene- and drug-sets generate molecular signatures shared with T2D IR pPS signatures derived according to the methods described herein. The primary analysis can leverage CMap's extensive set of 7,494 gene- and 19,811 small-molecule-perturbations. Corroboration of top findings can be assessed in JUMP and MOBB.

To test for similarity in expression signatures between CMap's perturbagens and the T2D IR pPS, Applicants can generate “connectivity” scores. Connectivity scores can be calculated using the same approach as the CMap standard analysis.⁶⁸ Briefly, the connectivity score captures the degree to which the pPS expression signature's up-regulated genes appear toward the top of the rank-ordered genes in the perturbagen's signature, and the down-regulated query genes appear toward the bottom of the signature (or vice versa for negative connectivity). The similarity score is calculated using the weighted Kolmogorov-Smirnov enrichment statistic⁶⁰ and then normalized to allow for comparison of scores across cell types and perturbation types. Applicants can calculate similarity scores using full inferred gene expression signatures via an application programming interface (API).

For all analyses, connectivity scores can compare a particular pPS tissue expression profile with the expression profile derived for selected perturbations in CMap. Applicants can only analyze expression signatures with more than 100 contributing transcripts each associated with the pPS at P<0.01 to reduce noise. When possible, Applicants can prioritize tissue expression signatures i) having significant gene-set enrichment results (FDR<0.05) that replicate in more than one dataset allowing generation of a meta-analyzed signature or ii) supported by image-based cellular profiles. Applicants can analyze the pPS expression signature in all CMap cell lines given that the preliminary analyses demonstrated that “positive control” genes and drugs relevant to a pPS can show significant similarity in CMap cell lines that are derived from tissues different from the pPS expression profile tissue-type (e.g., the “Lipodystrophy” pPS expression signature from SC adipose tissue mapped to significantly for insulin sensitizers and PPARG LOF in CMap HT29 cells).

Applicants can perform two structured analyses in CMap, one related to gene sets and the other to drug sets.

Gene-set analysis: For genes implicated in regulation of a given T2D pPS expression signature, Applicants can test whether gene perturbation recreates or recovers the pPS expression signature. For each pPS, Applicants can take the union of the predicted regulatory genes and separate them into predicted overexpression or LOF subsets based on having positive or negative connectivity between the gene “natural variation” and lipodystrophy pPS. Applicants can then identify the perturbations in CMap with these genes and assess perturbations separately for LOF and overexpression. Applicants can test whether the median similarity score (between each gene perturbation and the pPS expression signature) in each gene set is higher or lower than expected by chance using the Wilcoxon Rank Sum test. Significance thresholds can be adjusted for multiple testing. To corroborate results, Applicants can assess genes also perturbed in JUMP or MOBB. In JUMP, the available readout can be cell-painting, and Applicants can qualitatively assess for cellular features consistent with gene set/pathway enrichments. In MOBB for CRISPR gene perturbation, Applicants can assess both expression and image-based readouts. The expression readouts can be assessed quantitatively for similarity to the PS expression signatures using the methods described above.

Drug-set analysis: Using CMap's annotations for MOA, Applicants have sub-classified the 19,811 compounds into 830 MOA subsets. Applicants can test whether the similarity scores between the compounds in each MOA set and the pPS expression signature are enriched for higher or lower connectivity scores than chance using the two-sided Wilcoxon Rank Sum Test. Significance thresholds can be adjusted for multiple testing. If a significant MOA class is identified related to the 25 FDA-approved T2D drugs in CMap (Table 25, Example 2), Applicants can then query the T2D drugs in that class and likewise perform enrichment analysis of the similarity scores, but with a relaxed level of significance (P<0.05). Additional support for the identified drug sets can be provided by JUMP image-based reads, as described above.

Given the vast landscape of >1M perturbations in CMap and resulting multiple testing penalty, queries of single gene or compound perturbations are unlikely to have high confidence similarity with a given pPS expression profile. Nevertheless, Applicants can flag very high confidence connectivity scores (absolute value of score >0.9 and FDR<0.05) with any of the T2D IR pPS. For any such findings, replication can be sought using JUMP and MOBB datasets.

Consideration of sex: Sensitivity analyses can assess sex-specific expression profiles, as available.

Power Calculation: Approximating power for the two-sided Wilcoxon Rank Sum test using a t-test framework and considering standard-normalized connectivity scores in CMap, Applicants have at least 80% power to detect a 0.97 SD difference in mean connectivity scores between two groups (e.g., drugs with a given MOA vs all other drugs) if the test group has >35 perturbagens (e.g., drugs) at P<1×10⁻⁶ (Bonferroni adjusted for tests of 5-tissue expression signatures in 10 cell lines tested for 830 MOAs).

Expected Outcomes: Applicants can identify prioritized genes and drug MOA's specific to each polygenic mechanism of T2D, offering further characterization of disease pathways and proof-of-concept for translational potential.

Alternative Strategies: To reduce dependency on identifying regulatory genes, if for a given pPS there are no predicted regulatory genes, Applicants can test a set of 41 genes predicted to be causal for T2D.⁷⁰ The current approach does not clearly determine the specific contributions of gene-expression elements to a connectivity score. The hypothesis is that a large number of gene-expression features with modest effects form the basis of a high connectivity scores, but to further support this, Applicants can delve into the features to understand how a gene or drug is connected to a pPS. For example, Applicants could subset the pPS expression score to the sets of genes included in enriched gene sets to see how connectivity with genes/drugs is impacted. Finally, limitations of the perturbation datasets include that they involve cell lines which may not be optimal for studying a given pPS and that perturbations have specific exposure times and concentrations that might miss key connections in gene-expression space. Applicants have no way to resolve these issues experimentally, but as they represent essentially a “false-negative” failure mode, Applicants can forestall further consideration unless the proposed approaches yield no significant connections, which seems unlikely given the preliminary results.

These studies will motivate important follow-up studies, including:
1. Longitudinal study of the new T2D subtypes in clinical trials: Through established collaborations, Applicants plan to determine how T2D subtypes change clinically over time and test response to drug treatment in the Diabetes Prevention Program (metformin vs lifestyle vs placebo), DECLARE (dapagliflozin vs placebo), SAVOR (saxagliptin vs placebo), and GRADE (four drugs added on top of metformin) clinical trials.
2. Development of new gene- and compound-perturbation datasets: To validate selected novel findings and improve mapping of expression signatures, Applicants will generate gene- and compound-perturbation datasets in cell lines closer to the most informative expression profile tissues.
3. Rescue of expression profile with targeted drug treatment: Applicants can identify tissues from people in MOBB with high risk-T2D pPS and run compound screens using the top corresponding drug MOA candidates to see if Applicants can rescue the gene expression profiles and cellular profiles.

In summary, the genetics-based approach to characterizing the processes causing IR and T2D can identify patient subtypes, uncover complex gene expression networks underlying disease, and prioritize process-specific sets of regulatory genes and drug MOAs. This work has the potential to offer a new paradigm that is broadly applicable for studying any complex genetic disease and caring for patients afflicted by them.

### Example 3 References

1. James D E, Stockli J, Birnbaum M J. The aetiology and molecular landscape of insulin resistance. Nat Rev Mol Cell Biol 2021; 22(11): 751-71.
2. Vujkovic M, Keaton J M, Lynch J A, et al. Discovery of 318 new risk loci for type 2 diabetes and related vascular outcomes among 1.4 million participants in a multi-ancestry meta-analysis. Nat Genet 2020; 52(7): 680-91.
3. Mahajan A, Spracklen C N, Zhang W, et al. Trans-ancestry genetic study of type 2 diabetes highlights the power of diverse populations for discovery and translation. medRxiv 2020: 2020.09.22.20198937.
4. Udler M S, McCarthy M I, Florez J C, Mahajan A. Genetic Risk Scores for Diabetes Diagnosis and Precision Medicine. Endocr Rev 2019; 40(6): 1500-20.
5. Udler M S, Kim J, von Grotthuss M, et al. Type 2 diabetes genetic loci informed by multi-trait associations point to disease mechanisms and subtypes: A soft clustering analysis. PLoS medicine 2018; 15(9): e1002654.
6. Kim H, Westerman K E, Smith K, et al. High-throughput genetic clustering of type 2 diabetes loci reveals heterogeneous mechanistic pathways of metabolic disease. Diabetologia 2023; 66(3): 495-507.
7. DiCorpo D, LeClair J, Cole J B, et al. Type 2 Diabetes Partitioned Polygenic Scores Associate With Disease Outcomes in 454,193 individuals Across 13 Cohorts. Diabetes Care 2022: dc211395.
8. Laber S, Strobel S, Mercader J-M, et al. Discovering cellular programs of intrinsic and extrinsic drivers of metabolic traits using LipocyteProfiler. bioRxiv 2021: 2021.07.17.452050.
9. Saeedi P, Petersohn I, Salpea P, et al. Global and regional diabetes prevalence estimates for 2019 and projections for 2030 and 2045: Results from the International Diabetes Federation Diabetes Atlas, 9(th) edition. Diabetes Res Clin Pract 2019; 157: 107843.
10. Ahlqvist E, Storm P, Karajamaki A, et al. Novel subgroups of adult-onset diabetes and their association with outcomes: a data-driven cluster analysis of six variables. Lancet Diabetes Endocrinol 2018.
11. Reaven G. Insulin resistance and coronary heart disease in nondiabetic individuals. Arterioscler Thromb Vasc Biol 2012; 32(8): 1754-9.
12. Captieux M, Fleetwood K, Kennon B, et al. Epidemiology of type 2 diabetes remission in Scotland in 2019: A cross-sectional population-based study. PLoS Med 2021; 18(11): e1003828.
13. Karter A J, Nundy S, Parker M M, Moffet H H, Huang E S. Incidence of remission in adults with type 2 diabetes: the diabetes & aging study. Diabetes Care 2014; 37(12): 3188-95.
14. Draznin B, Aroda V R, Bakris G, et al. 9. Pharmacologic Approaches to Glycemic Treatment: Standards of Medical Care in Diabetes-2022. Diabetes Care 2022; 45(Supplement_1): S125-s43.
15. Almgren P, Lehtovirta M, Isomaa B, et al. Heritability and familiality of type 2 diabetes and related quantitative traits in the Botnia Study. Diabetologia 2011; 54(11): 2811-9.
16. Willemsen G, Ward K J, Bell C G, et al. The concordance and heritability of type 2 diabetes in 34,166 twin pairs from international twin registers: The Discordant Twin (DISCOTWIN) Consortium. Twin research and human genetics: the official journal of the International Society for Twin Studies 2015; 18(6): 762-71.
17. Liu X, Li Y I, Pritchard J K. Trans Effects on Gene Expression Can Drive Omnigenic Inheritance. Cell 2019; 177(4): 1022-34.e6.
18. Claussnitzer M, Cho J H, Collins R, et al. A brief history of human disease genetics. Nature 2020; 577(7789): 179-89.
19. Mahajan A, Taliun D, Thurner M, et al. Fine-mapping type 2 diabetes loci to single-variant resolution using high-density imputation and islet-specific epigenome maps. Nat Genet 2018; 50(11): 1505-13.
20. Khera A V, Chaffin M, Aragam K G, et al. Genome-wide polygenic scores for common diseases identify individuals with risk equivalent to monogenic mutations. Nat Genet 2018; 50(9): 1219-24.
21. American Diabetes A. (2) Classification and diagnosis of diabetes. Diabetes Care 2015; 38 Suppl: S8-S16.
22. Murphy R, Ellard S, Hattersley A T. Clinical implications of a molecular genetic classification of monogenic beta-cell diabetes. Nat Clin Pract Endocrinol Metab 2008; 4(4): 200-13.
23. Hattersley A, Bruining J, Shield J, Njolstad P, Donaghue K C. The diagnosis and management of monogenic diabetes in children and adolescents. Pediatr Diabetes 2009; 10 Suppl 12: 33-42.
24. Pearson E R, Liddell W G, Shepherd M, Corrall R J, Hattersley A T. Sensitivity to sulphonylureas in patients with hepatocyte nuclear factor-lalpha gene mutations: evidence for pharmacogenetics in diabetes. Diabet Med 2000; 17(7): 543-5.
25. Pearson E R, Starkey B J, Powell R J, Gribble F M, Clark P M, Hattersley A T. Genetic cause of hyperglycaemia and response to treatment in diabetes. Lancet 2003; 362(9392): 1275-81.
26. Mahajan A, Wessel J, Willems S M, et al. Refining the accuracy of validated target identification through coding variant fine-mapping in type 2 diabetes. Nat Genet 2018; 50(4): 559-71.
27. Dupuis J, Langenberg C, Prokopenko I, et al. New genetic loci implicated in fasting glucose homeostasis and their impact on type 2 diabetes risk. Nat Genet 2010; 42(2): 105-16.
28. Dimas A S, Lagou V, Barker A, et al. Impact of type 2 diabetes susceptibility variants on quantitative glycemic traits reveals mechanistic heterogeneity. Diabetes 2014; 63(6): 2158-71.
29. Scott R A, Scott L J, Magi R, et al. An Expanded Genome-Wide Association Study of Type 2 Diabetes in Europeans. Diabetes 2017.
30. Devarajan K. Nonnegative matrix factorization: an analytical and interpretive tool in computational biology. PLoS Comput Biol 2008; 4(7): e1000029.
31. Wouters O J, McKee M, Luyten J. Estimated Research and Development Investment Needed to Bring a New Medicine to Market, 2009-2018. JAMA 2020; 323(9): 844-53.
32. Ringel M S, Scannell J W, Baedeker M, Schulze U. Breaking Eroom's Law. Nat Rev Drug Discov 2020; 19(12): 833-4.
33. Broadaway K A, Yin X, Williamson A, et al. Loci for insulin processing and secretion provide insight into type 2 diabetes risk. Am J Hum Genet 2023; 110(2): 284-99.
34. Pipeline for GWAS clustering using Bayesian non-negative matrix factorization (bNMF). github.com/gwas-partitioning/bnmf-clustering.
35. Common Metabolic Diseases Knowledge Portal. hugeamp.org/.
36. Neale Lab, GWAS analysis of the U K Biobank. www.nealelab.is/uk-biobank (accessed 06/25/2022).
37. Smoller J W, Karlson E W, Green R C, et al. An eMERGE Clinical Center at Partners Personalized Medicine. J Pers Med 2016; 6(1).
38. Taliun D, Harris D N, Kessler M D, et al. Sequencing of 53,831 diverse genomes from the NHILBI TOPMed Program. Nature 2021; 590(7845): 290-9.
39. All of Us Research Program I, Denny J C, Rutter J L, et al. The “All of Us” Research Program. N Engl J Med 2019; 381(7): 668-76.
40. Cornelis M C, Qi L, Zhang C, et al. Joint effects of common genetic variants on the risk for type 2 diabetes in U.S. men and women of European ancestry. Ann Intern Med 2009; 150(8): 541-50.
41. Chen E Y, Tan C M, Kou Y, et al. Enrichr: interactive and collaborative HTML5 gene list enrichment analysis tool. BMC Bioinformatics 2013; 14: 128.
42. Kuleshov M V, Jones M R, Rouillard A D, et al. Enrichr: a comprehensive gene set enrichment analysis web server 2016 update. Nucleic Acids Res 2016; 44(W1): W90-7.
43. Xie Z, Bailey A, Kuleshov M V, et al. Gene Set Knowledge Discovery with Enrichr. Curr Protoc 2021; 1(3): e90.
44. The Gene Ontology resource: enriching a GOld mine. Nucleic Acids Res 2021; 49(D1): D325-d34.
45. Ashburner M, Ball C A, Blake J A, et al. Gene ontology: tool for the unification of biology. The Gene Ontology Consortium. Nat Genet 2000; 25(1): 25-9.
46. Jassal B, Matthews L, Viteri G, et al. The reactome pathway knowledgebase. Nucleic Acids Res 2020; 48(D1): D498-D503.
47. Qin Q, Fan J, Zheng R, et al. Lisa: inferring transcriptional regulators through integrative modeling of public chromatin accessibility and ChIP-seq data. Genome Biol 2020; 21(1): 32.
48. Loft A, Forss I, Siersbaek M S, et al. Browning of human adipocytes requires KLF11 and reprogramming of PPARgamma superenhancers. Genes Dev 2015; 29(1): 7-22.
49. Jang Y, Park Y K, Lee J E, et al. MED1 is a lipogenesis coactivator required for postnatal adipose expansion. Genes Dev 2021; 35(9-10): 713-28.
50. The GTEx Consortium atlas of genetic regulatory effects across human tissues. Science 2020; 369(6509): 1318-30.
51. Stegle O, Parts L, Durbin R, Winn J. A Bayesian framework to account for complex non-genetic factors in gene expression levels greatly increases power in eQTL studies. PLoS Comput Biol 2010; 6(5): e1000770.
52. Wessels B, Honecker J, Schottl T, et al. Adipose Mitochondrial Respiratory Capacity in Obesity is Impaired Independently of Glycemic Status of Tissue Donors. Obesity (Silver Spring, Md) 2019; 27(5): 756-66.
53. Alonso L, Piron A, Morán I, et al. TIGER: The gene expression regulatory variation landscape of human pancreatic islets. Cell Rep 2021; 37(2): 109807.
54. Pashos E E, Park Y, Wang X, et al. Large, Diverse Population Cohorts of hiPSCs and Derived Hepatocyte-like Cells Reveal Functional Genetic Variation at Blood Lipid-Associated Loci. Cell Stem Cell 2017; 20(4): 558-70.e10.
55. Scott L J, Erdos M R, Huyghe J R, et al. The genetic regulatory signature of type 2 diabetes in human skeletal muscle. Nat Commun 2016; 7: 11764.
56. McCarthy S, Das S, Kretzschmar W, et al. A reference panel of 64,976 haplotypes for genotype imputation. Nat Genet 2016; 48(10): 1279-83.
57. Barajas-Olmos F, Centeno-Cruz F, Zerrweck C, et al. Altered DNA methylation in liver and adipose tissues derived from individuals with obesity and type 2 diabetes. BMC Med Genet 2018; 19(1): 28.
58. Dobin A, Davis C A, Schlesinger F, et al. STAR: ultrafast universal RNA-seq aligner. Bioinformatics 2013; 29(1): 15-21.
59. Frankish A, Diekhans M, Ferreira A M, et al. GENCODE reference annotation for the human and mouse genomes. Nucleic Acids Res 2019; 47(D1): D766-d73.
60. Subramanian A, Tamayo P, Mootha V K, et al. Gene set enrichment analysis: a knowledge-based approach for interpreting genome-wide expression profiles. Proc Nat Acad Sci USA 2005; 102(43): 15545-50.
61. Zou Y, Henry W S, Ricq E L, et al. Plasticity of ether lipids promotes ferroptosis susceptibility and evasion. Nature 2020; 585(7826): 603-8.
62. Huang R, Grishagin I, Wang Y, et al. The NCATS BioPlanet—An Integrated Platform for Exploring the Universe of Cellular Signaling Pathways for Toxicology, Systems Biology, and Chemical Genomics. Frontiers in Pharmacology 2019; 10.
63. Kanehisa M, Sato Y, Kawashima M. KEGG mapping tools for uncovering hidden features in biological data. Protein Science 2022; 31(1): 47-53.
64. Love M I, Huber W, Anders S. Moderated estimation of fold change and dispersion for RNA-seq data with DESeq2. Genome Biol 2014; 15(12): 550.
65. Ferreira P G, Munoz-Aguirre M, Reverter F, et al. The effects of death and post-mortem cold ischemia on human tissue transcriptomes. Nat Commun 2018; 9(1): 490.
66. Chu T, Wang Z, Pe'er D, Danko C G. Cell type and gene expression deconvolution with BayesPrism enables Bayesian integrative analysis across bulk and single-cell RNA sequencing in oncology. Nat Cancer 2022; 3(4): 505-17.
67. ConnectivityMap. clue.io/ (accessed 02/20/2023).
68. Subramanian A, Narayan R, Corsello S M, et al. A Next Generation Connectivity Map: L1000 Platform and the First 1,000,000 Profiles. Cell 2017; 171(6): 1437-52.e17.
69. Haghighi M, Caicedo J C, Cimini B A, Carpenter A E, Singh S. High-dimensional gene expression and morphology profiles of cells across 28,000 genetic and chemical perturbations. Nat Methods 2022; 19(12): 1550-7.
70. Predicted type 2 diabetes effector genes. t2d.hugeamp.org/effectorgenes.html (accessed 02/08/2023).

### Example 4—Genetically-Anchored Molecular Signatures for Lipodystrophy Polygenic Risk Scores (PRS) can Identify Chemical Modulators of Lipodystrophy

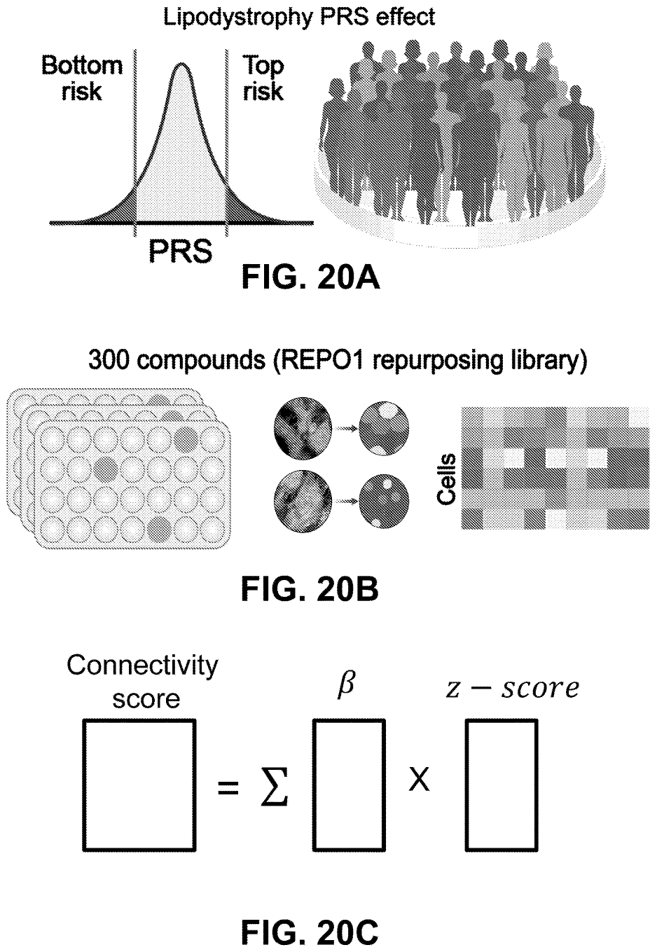

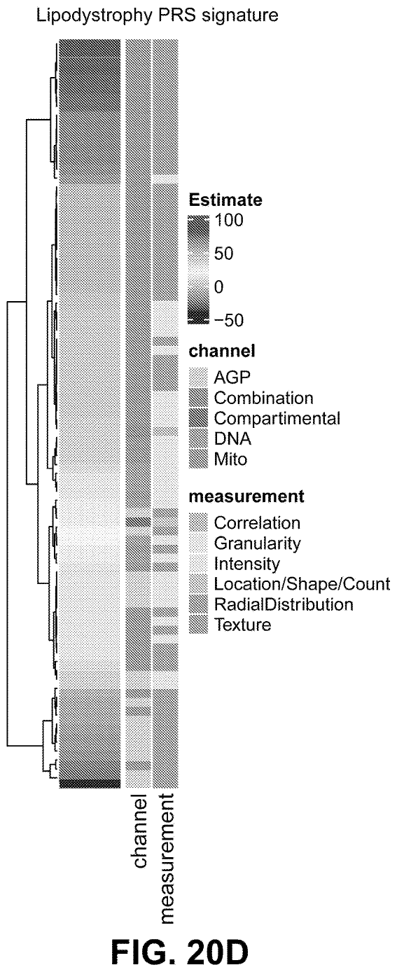

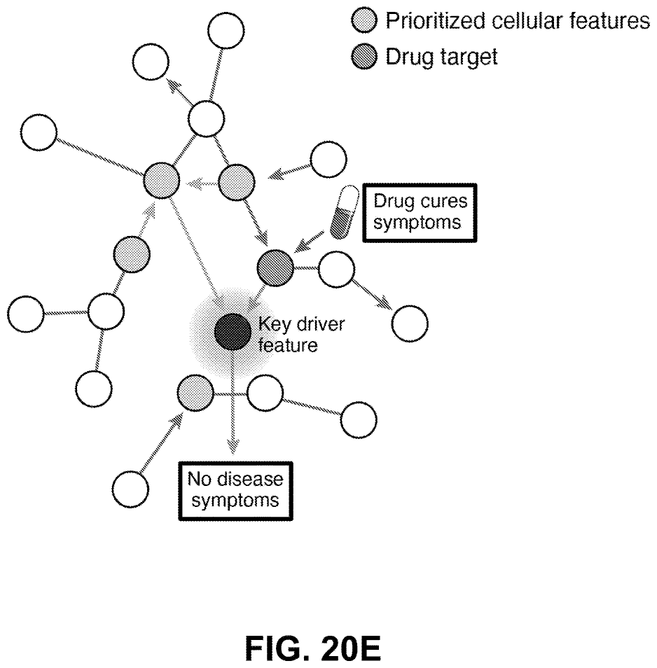

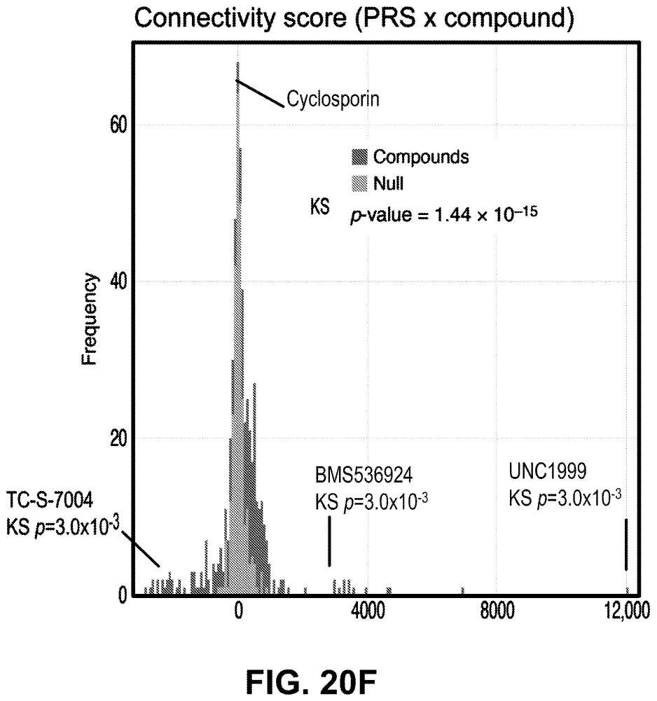

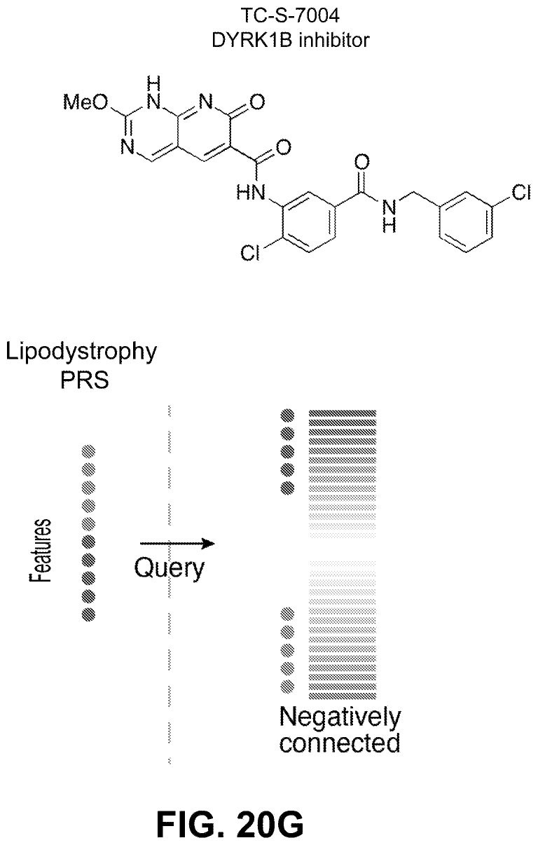

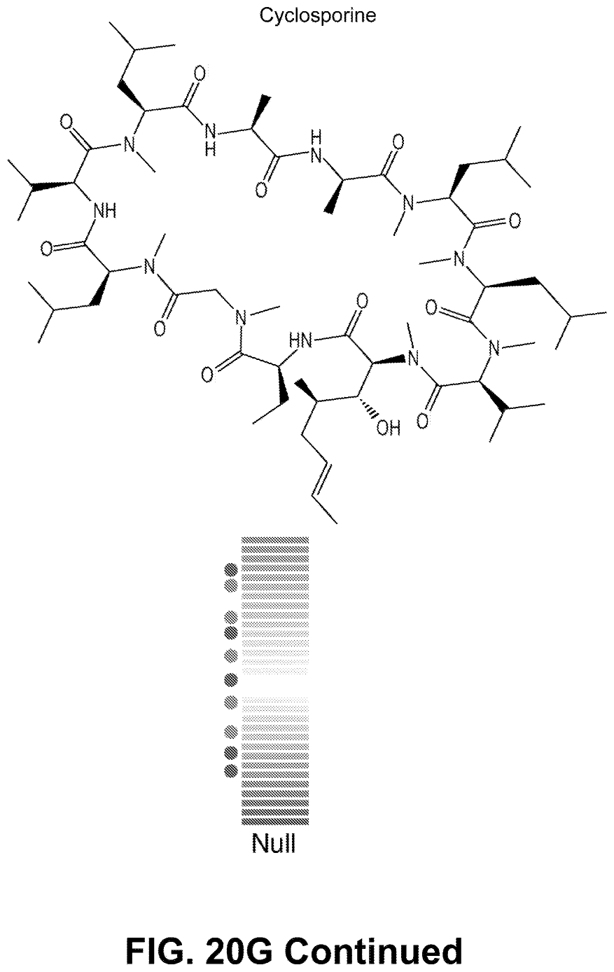

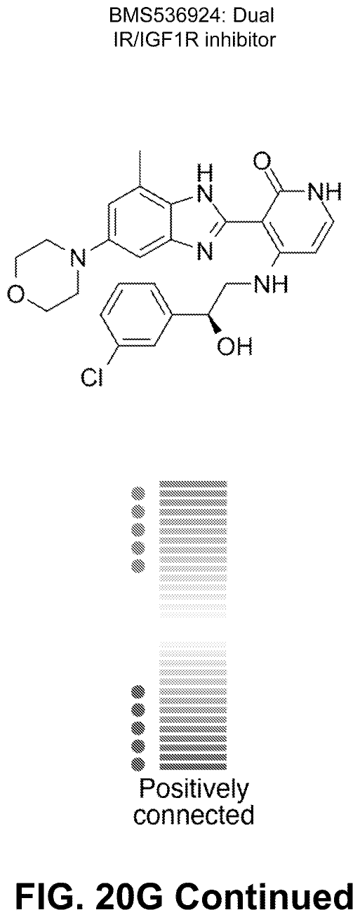

Applicants show further proof-of-principal that not only can pPSs be used to identify drugs, but a PRS for a disease (FIG. 20). Applicants first calculated polygenic risk scores for subjects having lipodystrophy and separated the subjects by the top risk and bottom risk. (FIG. 20A). Lipodystrophy PRS signatures could be obtained for the top risk and bottom risk subjects (FIG. 20D). The signatures include both gene expression signatures and imaging-based signatures that include morphological features obtained using LipocyteProfiler. Signatures are also obtained for cells treated with one of 300 compounds from a compound library (FIG. 20B). The signatures from the subjects and compound treated cells are compared and a connectivity score is calculated (FIG. 20C). The signature features can be placed on a network to understand connections between the PRS and drug signatures (FIG. 20E). Applicants identified connectivity scores for the drugs calculated for the top and bottom PRS scores and the compounds (FIG. 20F). Most of the drugs were null and did not show similar signatures in the cells. Applicants were able to identify negatively connected drugs that correlated negatively towards features observed in the top and bottom lipodystrophy PRS signatures (e.g., TC-S-7004 DYRKIB inhibitor) and positively connected drugs that correlated positively towards features observed in the top and bottom lipodystrophy PRS signatures (e.g., BMS536924 Dual IR/IGF1R inhibitor) (FIG. 20G) (see, e.g., Keramati A R, Fathzadeh M, Go G W, et al. A form of the metabolic syndrome associated with mutations in DYRKIB. N Engl J Med. 2014; 370(20):1909-1919; and Dool C J, Mashhedi H, Zakikhani M, et al. IGF 1/insulin receptor kinase inhibition by BMS-536924 is better tolerated than alloxan-induced hypoinsulinemia and more effective than metformin in the treatment of experimental insulin-responsive breast cancer. Endocr Relat Cancer. 2011; 18(6):699-709).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method for obtaining a genetically-anchored molecular signature for a disease comprising identifying, for a set of endotypes for the disease, a genetically anchored molecular signature correlated with each endotype by a regression/correlation analysis of a molecular data set, the molecular data set comprising at least endotype data type and one or more molecular profiles.

2. The method of claim 1, further comprising identifying one or more key regulatory features of the genetically-anchored molecular signature by matching the genetically-anchored molecular signature with one or more perturbation molecular signatures from a perturbation data set using similarity scoring.

3. The method of claim 1, further comprising conducting a therapeutic agent library screen to identify one or more therapeutic agents that reverse the genetically-anchored molecular signature.

4. The method of claim 1, wherein the endotype is associated with a distinct mechanistic pathway of the disease, or wherein the endotype is defined by a polygenic risk score, thereby linking the polygenic risk score for that endotype to molecular data.

5. (canceled)

6. The method of claim 4, wherein the polygenic risk score is enriched for variants linked to DNA regulatory elements active in a tissue associated with the disease, or wherein the polygenic risk score is a partitioned polygenic risk score (pPS), or wherein the pPS risk score is obtained by clustering variants based on patterns of genome-wide associations across disease related traits for the disease, or wherein the clustering is a soft clustering analysis, optionally wherein the soft clustering analysis is a Bayesian non-negative factorization (bNMF) clustering analysis.

7-10. (canceled)

11. The method of claim 1, wherein the one or more molecular profiles used to derive the genetically-anchored molecular signature comprise a transcriptomic profile, a proteomic profile, a metabolomic profile, a cell-imaging based profile, a spatial transcriptomic profile, a spatial proteomics profile, a spatial metabolomics profile, an epigenomic profile, a clinical imaging profile, or a combination thereof, optionally wherein the epigenomic profile comprises a chromatin accessibility profile, a chromatin modification profile, a chromatin binding profile, a DNA methylation profile, a DNase hypersensitivity profile, or a DNA-DNA contact profile.

12. (canceled)

13. A method for identifying therapeutic agents for treating disease comprising:
conducting a therapeutic agent library screen and generating a molecular signature for each therapeutic agent;
selecting one or more therapeutic agents with a molecular signature that positively or negatively correlates with a genetically-anchored molecular signature for a disease.

14. The method of claim 13, wherein the selected one or more therapeutic agents have a molecular signature that negatively correlates with the genetically anchored molecular signature for the disease, optionally wherein the genetically-anchored molecular signature is derived from a regression/correlation analysis of a molecular data set, the molecular data set comprising at least endotype data and one or more molecular profiles, optionally wherein the genetically-anchored molecular signature is derived from a regression/correlation analysis of a molecular data set, the molecular data set comprising at least endotype data and one or more molecular profiles, optionally wherein the endotype data is associated with distinct mechanistic pathways of a disease or wherein the endotype is defined by a polygenic risk score, optionally wherein the polygenic risk score is enriched for variants linked to DNA regulatory elements active in a tissue associated with the disease, optionally wherein the polygenic risk score is a pPS risk score, optionally wherein the pPS risk score is obtained by clustering variants based on patterns of genome-wide associations across disease related traits for the disease, optionally wherein the clustering is a soft clustering analysis, optionally wherein the soft clustering analysis is a Bayesian non-negative factorization (bNMF) clustering analysis, optionally wherein the one or more molecular profiles comprise a transcriptomic profile, a proteomic profile, a metabolomic profile, a cell-imaging based profile, a spatial transcriptomic profile, a spatial proteomics profile, a spatial metabolomics profile, an epigenomic profile, a clinical imaging profile, or a combination thereof.

15-23. (canceled)

24. The method of claim 23, wherein the epigenomic profile comprises a chromatin accessibility profile, a chromatin modification profile, a chromatin binding profile, a DNase hypersensitivity profile, or a DNA-DNA contact profile.

25. A method of treating a disease comprising:
determining a genetically-anchored molecular signature for a subject in need of treatment; and
administering one or more therapeutic agents capable of reversing the genetically-anchored molecular signature, wherein the one or more therapeutic agents are identified using the method of claim 13.

26. The method of claim 25, wherein the disease is type 2 diabetes (T2D).

27. The method of claim 26, wherein the genetically-anchored molecular signature is correlated to a genetic risk variant cluster of T2D and one or more gene regulatory pathways associated with the genetic risk variant cluster.

28. The method of claim 27, wherein the genetic risk variant clusters comprise a Beta-cell 1, a Beta-cell 2, a proinsulin, a lipoprotein A, an obesity, a lipodystrophy, a liver/lipid, a hyper insulin secretion, an ALP negative, and a SHBG cluster.

29. A method of monitoring and/or modifying a treatment comprising:
(a) determining a genetically-anchored molecular signature for a subject in need of treatment wherein the genetically-anchored molecular signature is correlated to a disease type or disease sub-type;
(b) administering an initial therapy capable of reversing the genetically-anchored molecular signature;
(c) determining if there has been a change in the genetically-anchored molecular signature and
continuing to administer the initial therapy if the genetically-anchored molecular signature has reversed.

30. The method of claim 29, wherein the disease is type 2 diabetes (T2D).

31. The method of claim 30, wherein genetically-anchored molecular signature identifies a genetic risk variant cluster of T2D and one or more gene regulatory pathways associated with the genetic risk variant cluster.

32. The method of claim 31, wherein the genetic risk variant clusters comprise a Beta-cell 1, a Beta-cell 2, a proinsulin, a lipoprotein A, an obesity, a lipodystrophy, a liver/lipid, a hyper insulin secretion, an ALP negative, and a SHBG cluster.


SEQUENCE LISTING (19 sequences)

SEQ ID NO: 1 - protein, 7 aa, Betapolyomavirus macacae
PKKKRKV

SEQ ID NO: 2 - protein, 10 aa, Betapolyomavirus macacae
PKKKRKVEAS

SEQ ID NO: 3 - protein, 16 aa, synthetic construct
KRPAATKKAGQAKKKK

SEQ ID NO: 4 - protein, 9 aa, Homo sapiens
PAAKRVKLD

SEQ ID NO: 5 - protein, 11 aa, Homo sapiens
RQRRNELKRSP

SEQ ID NO: 6 - protein, 38 aa, Homo sapiens
NQSSNFGPMKGGNFGGRSSGPYGGGGQYFAKPRNQGGY

SEQ ID NO: 7 - protein, 42 aa, Homo sapiens
RMRIZFKNKGKDTAELRRRRVEVSVELRKAKKDEQILKRRNV

SEQ ID NO: 8 - protein, 8 aa, Homo sapiens
VSRKRPRP

SEQ ID NO: 9 - protein, 8 aa, Homo sapiens
PPKKARED

SEQ ID NO: 10 - protein, 8 aa, Homo sapiens
PQPKKKPL

SEQ ID NO: 11 - protein, 12 aa, Mus sp.
SALIKKKKKMAP

SEQ ID NO: 12 - protein, 5 aa, Alphainfluenzavirus influenzae
DRLRR

SEQ ID NO: 13 - protein, 7 aa, Alphainfluenzavirus influenzae
PKQKKRK

SEQ ID NO: 14 - protein, 10 aa, Deltavirus Hepatitis delta virus
RKLKKKIKKL

SEQ ID NO: 15 - protein, 10 aa, Mus sp.
REKKKFLKRR

SEQ ID NO: 16 - protein, 20 aa, Homo sapiens
KRKGDEVDGVDEVAKKKSKK

SEQ ID NO: 17 - protein, 17 aa, Homo sapiens
RKCLQAGMNLEARKTKK

SEQ ID NO: 18 - protein, 288 aa, synthetic construct
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 19 - protein, 183 aa, synthetic construct
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS